(19) United States
(12) Patent Application Publication
(10) Pub. No.: US 2005/0192232 A1
(43) Pub. Date: Sep. 1, 2005

(54) Novel acyl-dipeptide-like compounds bearing an accessory functional side chain spacer, a method for preparing the same and pharmaceutical compositions containing such products

(76) Inventors: Jacques Bauer, Saint-Prex (CH); Richard Oliver Martin, Orleans (FR); Sylvain Rodriguez, Gland (CH)

Correspondence Address: HEDMAN & COSTIGAN P.C., 1185 AVENUE OF THE AMERICAS, NEW YORK, NY, 10036, US

(21) Appl. No.: 11/113,443

(22) Filed: Apr. 22, 2005

Related U.S. Application Data
(63) Continuation-in-part of application No. 10/169,053, filed on Aug. 28, 2002, now abandoned.

(30) Foreign Application Priority Data
Dec. 22, 1999 (WO) PCT/IB99/02038

(51) Int. Cl. A61K 38/04; C07K 5/04

(52) U.S. Cl. 514/19; 554/51

(57) ABSTRACT

The present invention is directed in particular to dipeptide-like compounds derived from functionally substituted amino acids, having fatty acid chains bound thereto through amidification of the amine functional groups of said dipeptide-like compounds, one end portion of which bears an accessory functional side chain spacer, with the other end portion being an acid group either in neutral or charged state. Compounds of the present invention have immunomodulating properties like adjuvants, In addition, compounds of the invention can be grafted on a given antigen in order to modulate or tune the immune response or can be equally grafted on a pharmaceutical carrier to enhance the therapeutic effect or targetting thereof. Accordingly, compounds of the invention find use in human and veterinary medicine both as immunogens and diagnostic tools.

### PRIOR APPLICATIONS

This application is a Continuation-in-Part of U.S. patent application Ser. No. 10/169,053 filed Aug. 28, 2002 which is a 371 of PCT/FR00/03650 filed Dec. 21, 2000.

### BACKGROUND ART

The present invention relates to the field of chemistry and more specifically to the field of peptide chemistry. More precisely, it is directed to acyl-dipeptide-like compounds bearing an accessory functionally substituted side chain spacer which can optionally be grafted in the form of conjugates, the accessory side chain spacer of which further imparts original properties to the molecule in terms of biological activity and physical chemical characteristics. Depending on the chemical species involved, the accessory side chain spacer gives the acyl-dipeptide-like compound added functional ability by finely tuning its original properties and by conferring novel ones thereto as well. These molecules bearing an accessory functional side chain spacer can be conjugated to a pharmaceutical carrier, an antigen or a vehicle. Bioconjugation involves coupling two or more chemical species to form a novel molecular complex having properties differing from those of the individual components. Natural or synthetic products, having inherent pharmacological properties, can be mutually combined to make new species having original or improved pharmacological and chemical physical properties as compared to starting compounds. Bioconjugates have a wide range of applications in all fields of human medicine and animal care as well as diagnostics.

A great number of homo- or heterobifunctional coupling agents have already been described and may be used in coupling molecules ranging from amino acids, to peptides, protein, sugars, oligosaccharides, polysaccharides, nucleic acids, oligonucleotides, polynucleotides, lipids, and nearly every single molecule bearing a functional group capable of bonding. Considerable effort has been made in recent years regarding the synthesis of antigenic constructs made from two molecules bearing different messages. Good et al; [(1987), Science 235 : 1059-1062], have for instance reported the synthesis of a peptide containing both T helper and B lymphocyte recognition epitopes. Bessler and Jung [(1992) Res. Immunol., 5 : 548-553] have disclosed conjugates composed of a peptide and an immunostimulant. Hoffmann et al. [(1997) FEMS Immunol. Med. Microbiol., 17 : 225-234] have disclosed conjugates of a lipopeptide and a mellitin derived-synthetic peptide. Ulrich and Meyers [(1995) Vaccine Design, Plenum Press, New York, 495-524], have observed that immune response was inefficient unless the hapten and MPL adjuvant (Monophosphoryl Lipid A) were found in the same liposome. They suggested the possible existence of a covalent bond between MPL and the hapten. In fact, a hapten-adjuvant conjugate may prove to be highly efficient when used as a vaccine adjuvant. Ikeda et al. [(1999) Chem. Pharm. Bull., 47 (4), 563-568] have reported synthesis of a structural analog of Lipid A coupled to a peptide tumour-derived antigen and demonstrated it has in vitro mitogenic activity.

The conjugation concept may equally apply to protein or even protein-polysaccharide conjugates. Indeed, it is well known that use of polysaccharides alone as a vaccine does only give rise to a weak immune response in children aged below 5, as no T cell-mediated response is involved. [Gotschlich et al. (1977); Antibodies in Human Diagnosis and Therapy, Peltola et al., (1977), Pediatrics : 60 : 730-737]. By contrast, linking polysaccharides to protein carriers does result in a much stronger immune response. This phenomenon was discovered in 1931 by Avery and Goebel [(1931), J. Exp. Med., 54 : 437-447]. A variety of recently developped vaccines reflect progress accomplished so far in this field. Mention should be made of vaccines to Haemophilus influenzae and differents serotypes of Streptococcus pneumoniae [Powell and Newman, (1995), Vaccine Design, Plenum Press, New York]. In the latter case, a multivalent vacine has been developped [Sood et Fatton, (1998), Exp. Opin. Invest. Drugs, 7 : (3), 333-347].

A complex composed of an adjuvant bound to a polysaccharide-protein unit raises new opportunities. Chemical synthesis technology as applied to bioconjugates is currently well developped and one can carry out a number of projects which were inconceivable just a few years ago, relying on the wide range of homo- or heterobifunctional reagents now available and using conjugation procedures extensively reported in the litterature [Hermanson, (1996), Bioconjugate Techniques, Academic Press, New York].

Thus, mention can be made, for instance, of the reductive amination method [Roy et al., (1984), Canad. J. Biochem. Cell Biol., 62 : 270-279, Hermansson, p. 472] allowing conjugation of a carrier molecule to an aldehyde functional group, having a primary amine on a peptide or protein molecule, with a protein-polysaccharide conjugate or with a pharmaceutical carrier. This reaction results in the formation of a very stable dialkylamine compound.

### DISCLOSURE OF THE INVENTION

The present invention is more specifically directed to dipeptide-like compounds derived from functionnally substituted amino acids, the free amino functional groups of which are amidified by fatty acids and one end portion of which bears an accessory functional side chain spacer.

More precisely, the invention relates to N-acyl- dipeptide-like compounds bearing an acid group in neutral or charged state at one end portion thereof and an accessory functional side chain spacer at the other end portion thereof, having the general formula I:

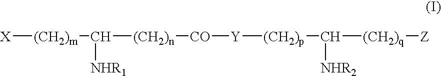

wherein R₁ and R₂ each designate an acyl group derived from a saturated or unsaturated, straight or branched chain-carboxylic acid having from 2 to 24 carbon atoms, which is unsubstituted or bears one or more substituents selected among hydroxyl, alkyl, alkoxy, acyloxy, amino, acylamino, acylthio and (C₁₋₂₄)alkylthio groups,

subscripts m, n are integers ranging from 0 to 10,

subscripts p, q are integers ranging from 1 to 10,

Y designates O or NH,

X and Z designate an accessory functional side chain spacer or an acid group either in neutral or charged state selected. among the following groups:
-carboxyl
-carboxy [(C₁₋₅)alkoxy]
-carboxy [(C₁₋₅)alkylthio]
-phosphono [(C₁₋₅)alkoxy]
-phosphono [(C₁₋₅)alkylthio]
-dihydroxyphosphoryloxy[C₁₋₅)alkoxy]
-dihydroxyphosphoryloxy[C₁₋₅)alkylthio]
-dihydroxyphosphoryloxy
-hydroxysulfonyloxy
-hydroxysulfonyl[(C₁₋₅)alkoxy]
-hydroxysulfonyl [(C₁₋₅)alkylthio]
-hydroxysulfonyloxy [(C₁₋₅)alkoxy]
-hydroxysulfonyloxy [(C₁₋₅)alkylthio
-[carboxy(C₁₋₅)alkyl]aminocarbonyl
-[dicarboxy(C₁₋₅)alkyl]aminocarbonyl
-[ammonio(C₁₋₅)alkyl]aminocarbonyl
-{carboxy[amino(C1-C5)alkyl}aminocarbonyl

provided that at least one of substituents X or Z designates an accessory functional side chain spacer.

The accessory group denoted by X or Z has the general formula (II):
A-(CO)ᵣ—(CH₂)ₛ—W (II)

where A designates either O, S or NH

subscript r is an integer ranging from 0 to 1

subscript s is an integer ranging from 1 to 10

W is selected among the following groups
-formyl
-acetyl
-cyano
-halo
-amino
-bromo- or iodo-acetamido
-acylamido
-diacylimido
-sulfhydril
-alkylthio
-hydroxyl
-1,2-dihydroxyethyl
-alkoxy
-acyloxy
-vinyl
-ethynyl
-free carboxyl
-esterified carboxyl or in the form of a mixed anhydride, amide or hydrazide,
-azido
-thiocyano

The invention is especially directed to novel N-acyl dipeptide-like compounds bearing an acid group either in neutral or charged satete, at one end portion thereof and bearing an accessory functional side chain spacer at the other, having the general formula I:

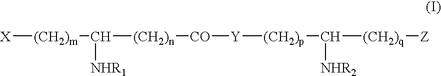

wherein R₁ and R₂ each designate an acyl group derived from a saturated or unsaturated straight or branched chain-carboxylic acid having from 2 to 24 carbon atoms, which is unsubstituted or bears one or more substituents selected among hydroxyl, alkyl, alkoxy, acyloxy, amino, acylamino, acylthio and (C₁₋₂₄)alkylthio groups,

subscripts m, n are integers ranging from 0 to 10,

subscripts p, q are integers ranging from 1 to 10,

X and Z each designate an acid group either in neutral or charged state or an accessory functional side chain spacer,

provided that at least one of substituents X or Z designates an accessory functional side chain spacer,

Y designates O or NH,

The acid group X or Y is preferably selected among the following groups:
-carboxyl
-carboxy [(C₁₋₅)alkoxy]
-carboxy [(C₁₋₅)alkylthio]
-phosphono [(C₁₋₅)alkoxy]
-phosphono [(C₁₋₅)alkylthio]
-dihydroxyphosphoryloxy[C₁₋₅)alkoxy]
-dihydroxyphosphoryloxy
-hydroxysulfonyloxy
-hydroxysulfonyl[(C₁₋₅)alkoxy]
-hydroxysulfonyl [(C₁₋₅)alkylthio]
-hydroxysulfonyloxy [(C₁₋₅)alkoxy]
-hydroxysulfonyloxy [(C₁₋₅)alkylthio]

and the accessory group denoted by X or Z has the general formule II, excluding a dihydroxyethyl radical.

Where substituents X or Z designate an acid group in neutral state, reference is made to the free carboxylic, sulphonic, phosphonic or phosphoric compound. Where the acid group is in a charged state, reference is made to the carboxylic, sulphonic, phosphonic or phosphoric salt form, namely by addition of an organic or mineral base, preferably one intended for therapeutic use. As for bases not intended for therapeutic use, such bases provide a means for easy identification, purification and separation.

Salt forming bases intended for therapeutic use mainly include alkaline bases such as sodium, potassium or lithium hydroxides, ammonium salts, alkali earth metal bases such as calcium or strontium hydroxide, magnesium salts, ferrous metal salts and the like, organic bases such as those derived from primary, secondary, tertiary amines of basic amino acids such as lysine and ornithine or amino sugars.

Examples of bases not intended for therapeutic use are brucine, strychnine, N-methylglucosamine or N-methylmorpholin. As previously stated, salts derived therefrom will serve as separation, identification or purification means.

When m is equal to 1 and n is equal to 0, the molecule of interest derives from serine or aspartic acid. Where m is equal to 2 and n is equal to 0, the molecule being considered mainly derives from homoserine or glutamic acid.

Where Y═NH, p is equal to 3 and q is equal to 1, the product of interest may be a citrulline, ornithine or arginine compound.

Where p is equal to 4 and q is equal to 1, reference is made to a homoarginine or lysine compound.

Where substituents X or Z designate the accessory group of general formula (III)
O—CO—(CH₂)ₛ—W (III)

subscript s is an integer ranging from 1 to 10, being, in particular, equal to 4, 5 or 6

W is preferably selected among the following groups
-formyl
-amino
-hydroxyl
-1,2-dihydroxyethyl
-carboxyl.

Among dipeptide-like compounds which are herein included, special attention is devoted to compounds of general formula IV which are currently preferred

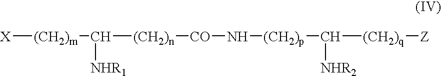

wherein R₁ and R₂ each designate an acyl group derived from a saturated or unsaturated, straight or branched chain-carboxylic acid having from 2 to 24 carbon atoms, which is unsubstituted or bears one or more substituents selected from the group comprised of hydroxyl, alkyl, alkoxy, acyloxy, amino, acylamino, acylthio and (C₁₋₂₄)alkylthio groups,

subscripts m and n are integers ranging from 0 to 10,

subscripts p and q are integers ranging from 1 to 10,

and wherein one of substituents X or Z is a carboxyl or a dihydroxyphosphoryloxy radical or a carboxy[(C₁₋₅)alkoxy] radical or a carboxy[(C₁₋₅)alkylthio] radical or a carboxy[(C₁₋₅)alkyl]aminocarbonyl or a [dicarboxy(C₁₋₅)alkyl]aminocarbonyl or a {carboxy[amino(C₁₋₅)alkyl}-aminocarbonyl radical and wherein the other substituent is an acyloxy radical chosen among one of 6-aminohexanoyloxy, 6-oxohexanoyloxy, 6-hydroxyhexanoyloxy, 6,7-dihydroxyheptanoyloxy or 3-carboxypropanoyloxy groups.

R₁ and R₂ are meant to include saturated or unsaturated, branched or straight chain-acyl derivatives having a variable size chain from 2 to 24 carbon atoms, of distinct or identical nature, which can bear one or more substituents selected from the group comprised of alkyl, amino, acylamino, hydroxyl, alkoxy, acyloxy acylthio and alkylthio groups. Among acyl groups herein of interest, mention should be made of those derived from lauric acid, 2-hydroxyoctanoic acid, 2-decanoyloxyoctanoic acid, 3-lauryloxymyristic acid, 3-hydroxymyristic acid, 3-myristoylmyristic acid and 3-palmitoylmyristic acid which are currently preferred.

The invention relates in particular to the following dipeptide-like compounds listed in the table below as being preferred compounds

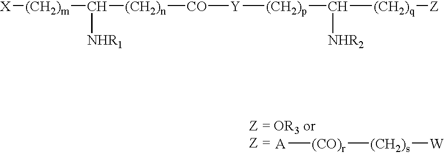

| Ex. # | X | *, ** | Y | m, n, p, q, | R₁ | R₂ | R₃ | A | r | s | W |
|---|---|---|---|---|---|---|---|---|---|---|---|
| 1.2 | HOOC | R, R | NH | 1, 0, 3, 1 | 3(C₁₂O)C₁₄ | 3(HO)C₁₄ | H | — | — | — | — |
| 1.3 | HOOC | R, R | NH | 1, 0, 3 ,1 | 3(C₁₂O)C₁₄ | 3(HO)C₁₄ | P(O)(OH)₂ | — | — | — | — |
| 2.1 | (HO)₂P(O)—O | R, R | NH | 2, 0, 3, 1 | 3(C₁₂O)C₁₄ | 3(HO)C₁₄ | — | O | 1 | 4 | CHOHCH₂OH |
| 2.2 | (HO)₂P(O)—O | R, R | NH | 2, 0, 3, 1 | 3(C₁₂O)C₁₄ | 3(HO)C₁₄ | — | O | 1 | 4 | CHO |
| 2.3 | HOOC | R, R | NH | 1, 0, 3,1 | 3(C₁₂O)C₁₄ | 3(HO)C₁₄ | — | O | 1 | 4 | CHOHCH₂OH |
| 2.4 | HOOC | R, R | NH | 1, 0, 3, 1 | 3(C₁₂O)C₁₄ | 3(HO)C₁₄ | — | O | 1 | 4 | CHO |
| 2.5 | HOOC | R, R | NH | 1, 0, 3,1 | 3(C₁₂O)C₁₄ | 3(HO)C₁₄ | — | O | 1 | 5 | OH |
| 2.6 | (HO)₂P(O)—O | RS, R | NH | 2, 0, 3, 1 | 3(C₁₂O)C₁₄ | 3(HO)C₁₄ | — | O | 1 | 5 | NH₂ |
| 2.7 | (HO)₂P(O)—O | R, R | NH | 2, 0, 3, 1 | 3(C₁₂O)C₁₄ | 3(HO)C₁₄ | — | O | 1 | 5 | NH₂ |
| 2.8 | (HO)₂P(O)—O | S, R | NH | 2, 0, 3, 1 | 3(C₁₂O)C₁₄ | 3(HO)C₁₄ | — | O | 1 | 5 | NH₂ |
| 2.9 | (HO)₂P(O)—O | R, R | NH | 2, 0, 3, 1 | 3(C₁₂O)C₁₄ | 3(HO)C₁₄ | — | O | 1 | 5 | OH |
| 2.10 | (HO)₂P(O)—O | RS, R | O | 2, 0, 1, 3 | 3(C₁₂O)C₁₄ | 3(HO)C₁₄ | — | NH | 0 | 5 | CHO |
| 2.11 | HOOCCH₂O | R, R | NH | 1, 0, 3, 1 | 3(C₁₂O)C₁₄ | 3(HO)C₁₄ | — | O | 1 | 4 | CHO |
| 2.12 | HOOCCH₂S | S, R | NH | 1, 0, 3, 1 | 3(C₁₂O)C₁₄ | 3(HO)C₁₄ | — | O | 1 | 6 | NH₂ |
| 2.13 | (HOOC)₂CHCH₂O | R, R | NH | 1, 0, 3,1 | 3(C₁₂O)C₁₄ | 3(HO)C₁₄ | — | O | 1 | 4 | CHO |
| 2.14 | (HO)₂P(O)—P | RS, R | NH | 2, 0, 3, 1 | 3(C₁₂O)C₁₄ | 3(HO)C₁₄ | — | O | 1 | 5 | NHC(O)CH₂Br |
| 2.15 | (HO)₂P(O)—O | RS, R | NH | 2, 0, 3, 1 | 3(HO)C₁₄ | 3(C₁₂O)C₁₄ | — | O | 1 | 4 | CHO |
| 2.16 | HOOC | R, R | NH | 1, 0, 3, 1 | 3(C₁₂O)C₁₄ | 3(HO)C₁₄ | — | O | 1 | 5 | NH₂ |
| 2.17 | HOOC | R, R | NH | 1, 0, 3, 1 | 3(C₁₂O)C₁₄ | 3(HO)C₁₄ | — | O | 1 | 5 | NHCO(CH₂)₂COOH |
| 2.18 | HOOC | R, R | NH | 1, 0, 3, 1 | 3(C₁₂O)C₁₄ | 3(HO)C₁₄ | — | O | 1 | 5 | NH₂ |
| 2.19 | HOOC | R, R | NH | 1, 0, 3, 1 | 3(C₁₂O)C₁₄ | 3(HO)C₁₄ | — | O | 1 | 2 | COOH |
| 2.20 | H₂N(CH₂)₃NHCO | R, R | NH | 1, 0, 3, 1 | 3(C₁₂O)C₁₄ | 3(HO)C₁₄ | H | — | — | — | — |
| 2.21 | H₂N(CH₂)₄CH(COOH)— | R, R | NH | 1, 0, 3, 1 | 3(C₁₂O)C₁₄ | 3(HO)C₁₄ | H | — | — | — | — |
| | NHCO | | | | | | | | | | |
| 2.22 | H₂N(CH₂)₄CH(COOH)— | R, R | NH | 1, 0, 3, 1 | 3(C₁₂O)C₁₄ | 3(HO)C₁₄ | — | O | 1 | 5 | NH₂ |
| | NHCO | | | | | | | | | | |
| 2.23 | HOOCCH₂CH(COOH)— | R, R | NH | 1, 0, 3, 1 | 3(C₁₂O)C₁₄ | 3(HO)C₁₄ | H | — | — | — | — |
| | NHCO | | | | | | | | | | |
| 2.24 | HOOCCH₂CH(COOH)— | R, R | NH | 1, 0, 3, 1 | 3(C₁₂O)C₁₄ | 3(HO)C₁₄ | — | O | 1 | 5 | NH₂ |
| | NHCO | | | | | | | | | | |
| 2.25 | HOOC | R, R | NH | 1, 0, 3, 1 | C₂ | 3(HO)C₁₄ | — | O | 1 | 5 | NH₂ |
| 2.26 | HOOC | R, R | NH | 1, 0, 3, 1 | 2(C₁₀O)C₈ | 3(HO)C₁₄ | — | O | 1 | 5 | NH₂ |

The invention relates in particular to the following as being currently preferred compounds:
—N—[(R)-3-dodecanoyloxytetradecanoylamino]-D-aspartic acid, α-N-{(4R)-5-hydroxy-4-[(R)-3-hydroxytetradecanoylamino]pentyl}amide and addition salts with a mineral or an organic base thereof (Ex 1.2)
-3-[(R)-3-dodecanoyloxytetradecanoylamino]-4-oxo-5-aza-9-[(R)-3-hydroxytetradecanoylamino]-decan-1,10-diol 1-dihydrogenphosphate 10-(6,7-dihydroxyheptanoate) and addition salts with a mineral or an organic base thereof (Ex 2.1)
-3-[(R)-3-dodecanoyloxytetradecanoylamino]-4-oxo-aza-9-[(R)-3-hydroxytetradecanoylamino]-dedan-1,10-diol 1-dihydrogenphosphate 10-(6-oxohexanoate) and addition salts with a mineral or an organic base thereof (Ex. 2.2)
—N-[(R)-3-dodecanoyloxytetradecanoylamino]-D-aspartic acid, α-N-{(4R)-5-hydroxy-4-[(R)-3-hydroxytetradecanoylamino]pentyl}amide 5-O-(6,7-dihydroxyheptanoate) and addition salts with a mineral or an organic base thereof (Ex. 2.3)
—N-[(R)-3-dodecanoyloxytetradecanoylamino]-D-aspartic acid, α-N-{(4R)-5-hydroxy-4-[(R)-3-hydroxytetradecanoylamino]pentyl}amide 5-O-(6-oxohexanoate) and addition salts with a mineral or an organic base thereof (Ex. 2.4.)
-(3RS, 9R), (3R, 9R) and (3S, 9R)-3-[(R)-3-dodecanoyloxytetradecanoylamino]-4-oxo-5-aza-9-[(R)-3-hydroxy-tetra-decanoylamino]-decan-1,10-diol 1-dihydrogenphosphate 10-(6-aminohexanoate) and addition salts with a mineral or an organic base thereof (Ex. 2.6, 2.7 and 2.8)
-3-[(R)-3-dodecanoyloxytetradecanoylamino]-4-oxo-5-aza-9-[(R)-3-hydroxytetradecanoylamino]-decan-1, 10-diol 1-dihydrogenphosphate 10-(6-hydroxyhexanoate) and addition salts with a mineral or an organic base thereof (Ex 2.9)
-{2-[(R)-3-hydroxytetradecanolamino]-5-(6-oxohexyl)amino}pentyl 2-[(R)-3-dodecanoyloxytetradecanoylamino]-4-(dihydroxyphosphoryloxy)-butanoate and addition salts with a mineral or an organic base thereof (Ex 2.10)
—N-[(R)-3-dodecanoyloxytetradecanoyl]-D-aspartic acid, α-N-{(4R)-5-(6-aminohexanoyloxy)-4-[(R)-3-hydroxytetradecanoylamino]pentyl}amide and addition salts with a mineral or an organic base thereof (Ex. 2.16)
—N-[(R)-3-dodecanoyloxytetradecanoyl]-D-aspartic acid, α-N-{(4 R)-5-succinyloxy-4-[(R)-3-hydroxytetradecanoylamino]pentyl}amide and addition salts with a mineral or an organic base thereof (Ex. 2.19)
—N-[(R)-3-dodecanoyloxytetradecanoyl]-D-aspartic acid, α-N-{(4R)-5-(6-aminohexanoyloxy)-4-[(R)-3-hydroxytetradecanoylamino]pentyl}amide β-N-[(1S)-1-carboxy-5-aminopentyl]amide and addition salts with a mineral or an organic base thereof (Ex. 2.22)
—N-[(R)-3-dodecanoyloxytetradecanoyl]-D-aspartic acid, α-N-{(4R)-5-(6-aminohexanoyloxy)-4-[(R)-3-hydroxytetradecanoylamino]pentyl}amide β-N-[(1S)-1,2-dicarboxyethyl]amide and addition salts with a mineral or an organic base thereof (Ex. 2.24)

These compounds have distinctive interesting pharmacological properties, mainly with regard to immunomodulation. They are particularly relevant in the preparation of vaccine compositions in admixture formulations or as covalent conjugates with polypeptide or polysaccharide antigens or with compounds made of polysaccharide-conjugated polypeptides. They can be used mainly in prevention of protozoal, viral and microbial infections or in treatment of certain autoimmune disease.

Other applications include use as vectors for molecules of therapeutic interest due to their ability to form non covalent complexes based on the variably hydrophilic or hydrophobic character of their accessory side chain spacer. Their chemical properties allow coupling thereof with molecules of therapeutic interest. Likewise, their amphophilic character enhances formulation and transport of molecules associated therewith to the membrane receptors, as well as to the cell membranes and cytoplasm.

They can be used alone or either in covalent linkage or not with a molecule of therapeutic interest by means of administration through oral, parenteral, rectal, topical, subcutaneous or submucosal route.

They can be used solely or either in covalent linkage or not with a molecule of therapeutic interest by carrying out extemporaneous incubation ex vivo with blood cells in order to promote formation of immunocompetent cells before injecting them back in vivo using parenteral administration.

The molecules of interest display similar properties, as adjuvants for the immune system when used for example in vaccination, either in covalent association or not with the appropriate antigens, against disease of viral, parasitic, microbial or fungal origin. These optionally conjugated molecules can further be used in treatment of certain autoimmune disease.

In contrast, some compounds according to the invention show following covalent association or not utterly different properties regarding their capacity to induce cytokine production or maturation of immunocompetent stem cells derived from hematopoietic and lymphoid organs.

Some compounds in accordance with the invention promote maturation and differenciation of monocytes into functional dendritic cells, in presence or absence of the appropriate antigen and act in promoting humoral and cell mediated immunity.

Certain compounds herein contemplated promote differenciation of cells which are part of the hematopoietic system especially within the bone marrow and result in improvement or correction of certain immune system disorders. They display in particular antiviral properties.

The compounds in accordance with the invention are particularly interesting due to their low toxicity. They are used either in covalent association or not with antigens in prevention or treatment of infectious disease in humans and animals at doses ranging from 0.005 mg to 100 mg per unit dosage and from 0.005 to 200 mg daily depending on the particular indication and the individual's body weight.

The present invention is equally directed to a method for obtaining N-acyl dipeptide-like compounds bearing an acid group either in neutral or charged state at one end portion thereof and bearing an accessory functional side chain spacer at the other, having the general formula I, which comprises the steps of blocking amine functional groups in position (q+1) and YH in position ω of an ω-functionally derivatized amino acid by transverse blocking reagents, reacting the still free carboxylic functional group with a reducing agent to yield a corresponding alcohol, freeing the amine functional group in position (q+1) and then acylating the same by means of a carboxylic acid functional derivative of formula R₂OH, wherein R₂ is as defined above, and subsequently freeing the terminal amine functional group, to yield the functionnally derivatized amino alcohol of general formula V

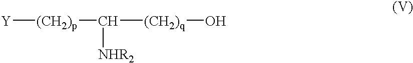

wherein Y designates HO or preferably NH₂,

R₂ designates an acyl group derived from a saturated or unsaturated carboxylic acid having from 2 to 24 carbon atoms, which is unsubstituted or bears one or more substituents as defined above,

p and q each designate an integer ranging from 1 to 10,

which amino alcohol is condensed in presence of a peptide condensing agent in an inert solvant, together with a ω-functionally derivatized amino acid compound of general formula VI

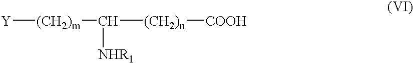

wherein R₁ is an acyl group derived from a saturated or unsaturated carboxylic acid having from 2 to 24 carbon atoms, which is unsubstituted or bears one or more substituents as specified above,

m and n are integers ranging from 0 to 10,

and X is an acid group as defined above which can be in the form of an ester,

to yield the dipeptide-like compound of general formula VII

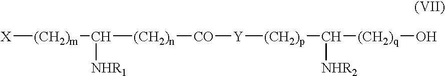

wherein substituents R₁ and R₂ and subscripts m, n, p and q are as specified above,

the free terminal alcohol functional group of which can be, if needed, alkyl-, acyl- or otherwise substituted by an alkyl-, acyl- or an otherwise substitution reagent of general formula VIII,
A-(CO)ᵣ—(CH₂)ₛ—W (VIII)

with A being a leaving group, an OH, SH or NH₂ functional group

subscript r preferably being equal to 1, or optionally equal to 0

subscript s preferably ranging from 2 to 6, optionally being included between 1 and 10

W being preferably chosen among the following groups, -formyl, -acetyl, -cyano, -halo, -amino, -bromo- or iodoacetamido, -acylamido, -diacylimido, -sulfhydril, -alkythio, -hydroxyl, -1,2-dihydroxyethyl, -acyloxy, -vinyl, ethynyl, free or esterified carboxyl or in the form a mixed anhydride, amide or hydrazide, -azido, -thiocyano or precursors thereof.
if needed, in the presence of a coupling agent, and subjecting the product to a catalytic hydrogenation or some other deprotection process in order to obtain the derivative of general formula I:

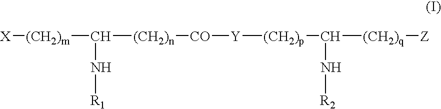

wherein substituents and subscripts X, Y, Z, R₁, R₂, n, m, p and q have the same meanings as those given above.

The invention is also directed to a method for obtaining phosphodipeptide-like compounds of general formula IV

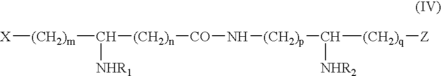

wherein R₁ and R₂ each designate an acyl group derived from a saturated or unsaturated, straight or branched chain-carboxylic acid having from 2 to 24 carbon atoms, which is unsubstituted or bears one or more substituents selected from the group comprised of hydroxyl, alkyl, alkoxy, acyloxy, amino, acylamino, acylthio and (C₁₋₂₄)alkylthio groups,

subscripts m, p and q are integers ranging from 1 to 10,

subscript n is an integer ranging from 0 to 10,

X and Z each designate an acid group in neutral or charged state or an accessory functional side chain spacer,

characterized in that it consists in blocking amine functional groups in positions (q+1) and ω of a diamino acid of formula H₂N(CH₂)ₚCHNH₂(CH₂)_{q-1}COOH by blocking reagents which readily undergo acidolysis and hydrogenolysis, respectively, reacting the still free carboxylic functional group with a reducing agent to yield a corresponding alcohol, freeing the amine functional group in position (q+1) and then acylating the same by means of a carboxylic acid functional derivative of formula R₂OH wherein R₂ is as defined above, then freeing the terminal amine functional group by hydrogenolysis to obtain an amino alcohol of general formula IX

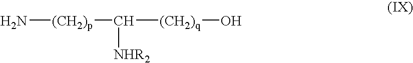

wherein R₂ designates an acyl group derived from a saturated or unsaturated carboxylic acid having from 2 to 24 carbon atoms, which is unsubstituted or bears one or more substituents as specified above,

p and q designate an integer ranging from 1 to 10

which amino alcohol is condensed in presence of a peptide condensing agent in an inert solvant, together with an co-hydroxy amino acid derivative of general formula VI:

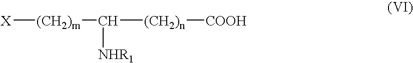

wherein R₁ is an acyl group derived from a saturated or unsaturated carboxylic acid having from 2 to 24 carbon atoms, which is unsubstituted or bears one or more substituents,

m is an integer ranging from 1 to 10,

n is an integer ranging from 0 to 10,

and X is a dialkyloxy- or diaryloxy- phosphoryloxy radical of formula

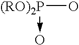

to yield the dipeptide-like compound of general formula X

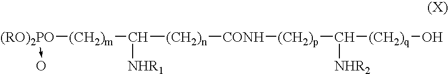

wherein substituents R₁, R₂, and subscripts m, n, p and q are as defined above, and R is a radical which readily undergoes hydrogenolysis, the free terminal alcohol functional group of which can be—if desired -alkyl- or acyl- or otherwise substituted by an alkyl- or acyl- or otherwise substitution reagent of general formula VIII
A-(CO)ᵣ—(CH₂)ₛ—W (VIII)

where A can be a leaving group, an OH, SH or NH₂ functional group

subscript r is an integer preferably equal to 1, optionally equal to 0,

subscript s is an integer ranging from 2 to 6, optionally ranging from 1 to 10,

W is preferably selected among the following groups
-formyl, -acetyl, -cyano, -halo, -amino, -bromo- or iodo-acetamido, -acylamido, -diacylimido, -sulfhydril, -alkylthio, -hydroxyl, 1,2-dihydroxyethyl, -acyloxy, -vinyl, -ethynyl, -free or esterified carboxyl or in the form of a mixed anhydride, amide or hydrazide, azido, -thiocyano or precursors thereof,

if needed, in presence of a coupling agent, and subjecting the same to a catalytic hydrogenation or some other deprotection process so as to obtain the derivative of general formula XI

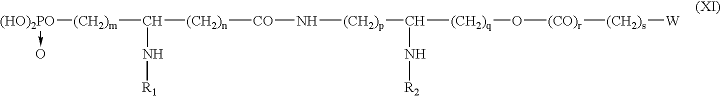

wherein substituents W, R₁, R₂ and subscripts m, n, p, q, r, s have the same meanings as above.

The invention still relates to a method for obtaining phosphodipeptide-like compounds of general formula XII

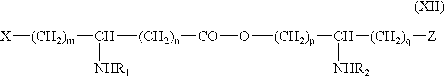

wherein R₁ and R₂ each designate an acyl group derived from a saturated or unsaturated, straight or branched chain-carboxylic acid having from 2 to 24 carbon atoms, which is unsubstituted or bears one or more substituents selected from the group comprised of hydroxyl, alkyl, alkoxy, acyloxy, amino, acylamino, acylthio and (C₁₋₂₄)alkylthio groups,

subscripts m, p and q are integers ranging from 1 to 10,

subscript n is an integer ranging from 0 to 10,

and wherein X and Z each designate an acid group or an accessory functional side chain spacer,

which consists in blocking amine functional groups in positions (q+1) and ω of a diamino acid of formula H₂N(CH₂)ₚCHNH₂(CH₂)_{q-1}COOH by blocking reagents which readily undergo acidolysis and hydrogenolysis, respectively, reacting the still free carboxylic functional group with a reducing agent to yield a corresponding alcohol, freeing the amine functional group in position (q+1) and then acylating the same by means of a carboxylic acid functional derivative of formula R₂OH wherein R₂ is as defined above, then freeing the terminal amine functional group by hydrogenolysis and thereafter alkyl substituting the amine functional group by an ω-functionally derivatized alkyl triflate to obtain an amino alcohol of general formula XIII

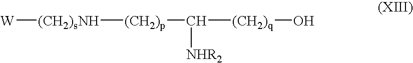

wherein R₂ designates an acyl group derived from a saturated or unsaturated carboxylic acid having from 2 to 24 carbon atoms, which is unsubstituted or bears one or more substituents as specified above,

p and q designate an integer ranging from 1 to 10,

subscript s is preferably included between 2 and 7, but may range from 1 to 10,

which amino alcohol is condensed in presence of a condensing agent in an inert solvant, together with an ω-hydroxy amino acid derivative of general formula VI:

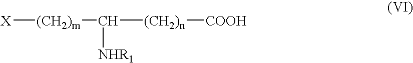

wherein R₁ is an acyl group derived from a saturated or unsaturated carboxylic acid having from 2 to 24 carbon atoms, which is unsubstituted or bears one or more substituents,

m is an integer ranging from 1 to 10,

n est an integer ranging from 0 to 10,

and X is a dialkyloxy- or diaryloxy- phosphoryloxy radical of formula

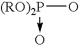

to yield the dipeptide-like compound of general formula XIV

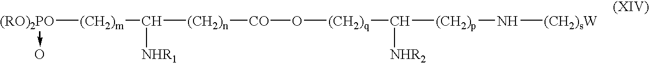

wherein substituents R₁, R₂, and subscripts m, n, p, q and s are as defined above, and R is a radical which readily undergoes hydrogenolysis,

and then subjecting the product to a catalytic hydrogenation or some other deprotection process so as to obtain the derivative of general formula XV:

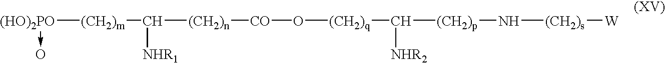

wherein substituents W, R₁, R₂, and subscripts m, n, p, q, r and s are as specified above,

W being chosen among the following groups, -formyl, -acetyl, -cyano, -halo, -bromo- or iodoacetamido, -acylamido, -diacylimido, -acyloxy, -vinyl, ethynyl, free or esterified carboxyl or having the form of a mixed anhydride, amide or hydrazide, -azido, -thiocyano or precursors thereof.

The invention further relates to a method for producing carboxydipeptide-like compounds of general formula IV

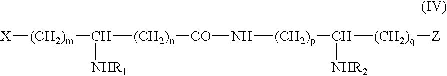

wherein R₁ and R₂ each designate an acyl group derived from a saturated or unsaturated, straight or branched chain-carboxylic acid having from 2 to 24 carbon atoms, which is unsubstituted or bears one or more substituents selected from the group comprised of hydroxyl, alkyl, alkoxy, acyloxy, amino, acylamino, acylthio and (C₁₋₂₄)alkylthio groups,

subscripts m, p and q are integers ranging from 1 to 10,

subscript n is an integer ranging from 0 to 10,

and wherein X and Z each designate an acid group or an accessory functional side chain spacer,

which consists in blocking amine functional groups in positions (q+1) and ω of a diamino acid of formula H₂N(CH₂)ₚCHNH₂(CH₂)_{q-1}COOH by blocking reagents which readily undergo acidolysis and hydrogenolysis, respectively, reacting the still free carboxylic functional group with a reducing agent to yield a corresponding alcohol, freeing the amine functional group in position (q+1) and then acylating the same by means of a carboxylic acid functional derivative of formula R₂OH wherein R₂ is as defined above, then freeing the terminal amine functional group by hydrogenolysis to obtain an amino alcohol of formula IX

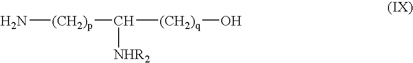

wherein R₂ designates an acyl group derived from a saturated or unsaturated carboxylic acid having from 2 to 24 carbon atoms, which is unsubstituted or bears one or more substituents as specified above,

p and q designate an integer ranging from 1 to 10

which amino alcohol is condensed in presence of a peptide condensing agent in an inert solvant, together with an ω-carboxy amino acid functional derivative of general formula VI:

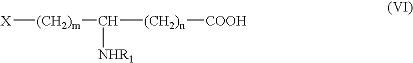

wherein R₁ is an acyl group derived from a saturated or unsaturated carboxylic acid having from 2 to 24 carbon atoms, which is unsubstituted or bears one or more substituents,

m is an integer ranging from 1 to 10,

n is an integer ranging from 0 to 10,

and X is an RO—CO— radical

to form the dipeptide-like compound of general formula XVI

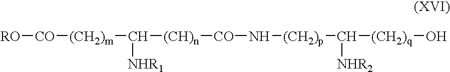

wherein substituents R₁, R₂ and subscripts m, n, p and q have the same meanings as above, and R is a group which readily undergoes hydrogenolysis, such as a benzyl group for instance.

the free terminal alcohol functional group of which may be, if needed, alkyl or acyl or otherwise substituted by an alkyl or acyl or otherwise substitution reagent of general formula VIII,
A-(CO)ᵣ—(CH₂)ₛ—W (VIII)

where A is a leaving group, an OH, SH or NH₂ functional group,

subscript r is an integer preferably equal to 1, optionally equal to 0,

subscript s is an integer ranging from 2 to 6, optionally ranging from 1 to 10,

W is preferably selected among the following groups -formyl, -acetyl, -cyano, -halo, -amino, -bromo- or iodoacetamido, -acylamido, -diacylimido, -sulfhydril, -alkylthio, -hydroxyl, -1,2-dihydroxyethyl, -acyloxy, -vinyl, -ethynyl, -free or esterified carboxyl or in the form of some other derivative

if needed, in presence of a coupling agent, and subjecting the same to a catalytic hydrogenation or some other deprotection process so as to obtain the derivative of general formula XI

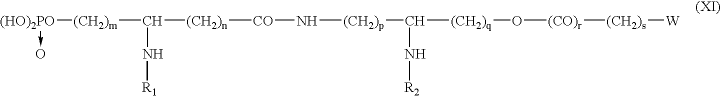

wherein substituents W, R₁, R₂, and subscripts m, n, p, q, r and s are as specified above.

The invention still relates to a method for obtaining phosphodipeptide-like compounds of general formula IV

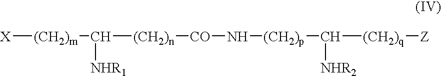

wherein R₁ and R₂ each designate an acyl group derived from a saturated or unsaturated, straight or branched chain-carboxylic acid having from 2 to 24 carbon atoms, which is unsubstituted or bears one or more substituents selected from the group comprised of hydroxyl, alkyl, alkoxy, acyloxy, amino, acylamino, acylthio and (C₁₋₂₄)alkylthio groups,

subscripts m, p and q are integers ranging from 1 to 10,

subscript n is an integer ranging from 0 to 10,

and wherein X and Z each designate an acid group or an accessory functional side chain spacer,

which consists in deprotecting the esterified carboxyl functional group of a dipeptide-like compound of formula XVI

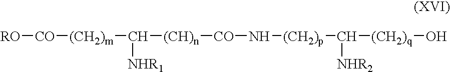

wherein substituents R₁, R₂ and subscripts m, n, p and q have the same meanings as above, and R is a group which readily undergoes hydrogenolysis such as a benzyl group for instance,

then subjecting the resulting acid to a peptide coupling reaction with a partially protected amino acid or diaminoalkane such as, for example, 3-benzyloxycarbonylamino-1-aminopropane, dibenzyl L-aspartate or ε-N-benzyloxycarbonyl-L-lysine, in presence of a peptide coupling agent, and thereafter subjecting, if needed, the coupling reaction product to an acyl-, alkyl- or otherwise substitution reaction with a reagent of general formule VIII
A-(CO)ᵣ—(CH₂)ₛ—W (VIII)

where A can be a leaving group, an OH, SH or NH₂ functional group,

subscript r is an integer equal to 1 or 0,

subscript s is an integer ranging in general from 1 to 10, preferably from 2 to 6,

W is selected among the following groups -formyl, -acetyl, -cyano, -halo, -amino, -bromo- or iodo-acetamido, -acylamido, -diacylimido, -sulfhydril, -alkylthio, -hydroxyl, 1, 2-dihydroxyethyl-, -acyloxy, -vinyl, -ethynyl, -free or esterified or otherwise derivatized carboxyl group.

if needed, in presence of an activating agent, and deprotecting the resulting product, for example by hydrogenolysis, so as to obtain the product of general formula XVIII

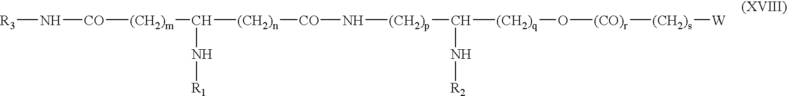

wherein substituents W, R₁, R₂, and subscripts m, n, p, q, r and s are as specified above and groupe R₃ designates an aminoalkyl, carboxyalkyl, dicarboxyalkyl or aminocarboxyalkyl group.

Stereochemistry of chiral centers bearing acylamino groups is determined by initially used amino acid configuration whereas stereochemistry of acylamino groups depends on initially used fatty acid configuration. One can start from a diamino acid having L or D configuration or of a racemic mixture. One can start from a hydroxylated amino acid of L, D configuration or of a racemic mixture. All such stereoisomers or diastereoisomers of compounds of general formulae I or IV or XII or still XVI are included in the scope of the invention.

Generally, the compounds of the invention are prepared by coupling an acid functional group of an N-acyl-, ω-functionally substituted amino acid with the amine functional group of an amino alcohol resulting from the reduction of a carboxyl group of a mono-N-acyl-diamino acid, followed by O-acyl or -alkyl substitution of the still free alcohol functional group in order to introduce an accessory functional side chain spacer, which can be optionally modified after binding to expose a reactive functional group. Deprotection of the final product frees an acid functional group.

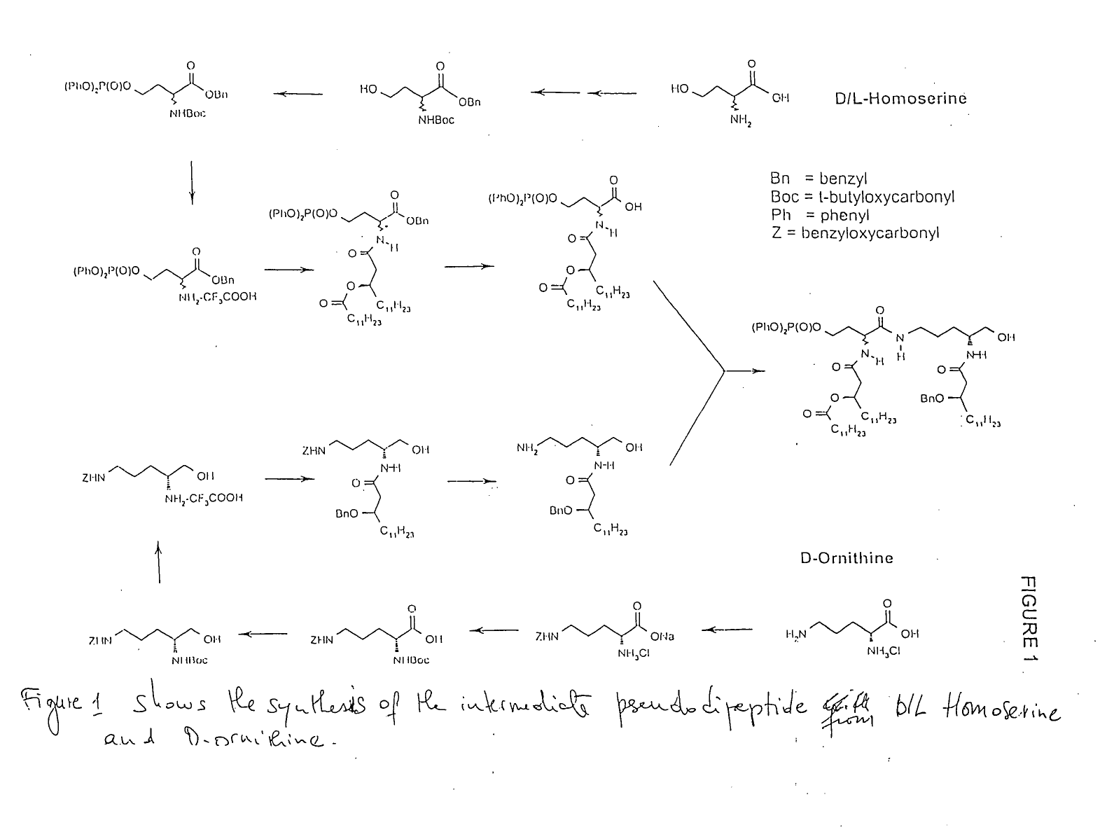

In a currently preferred method for preparing the compounds of the invention (FIG. 1), the ω-functionally derivatized amino acid is an α-amino ω-hydroxyl acid such as serine or homoserine, which is subjected to a series of N-protection reactions (for example, in the form of a t-butoxycarbonyl derivative), benzyl ester formation by O-alkyl substitution of a carboxylate and phosphorylation of the OH functional group in order to introduce a protected phosphate group. Typical protective groups of phosphates may be phenyl, benzyl or o-xylyl groups. A phenyl group is the currently preferred one. Next, the amine functional group is freed by removal of the protective group (for example by treatment of the t-butyoxycarbonyl derivative with trifluoroacetic acid), then acyl-substituted by an activated fatty acid derivative, preferably a derivative of 3-hydroxytetradecanoic acid such as 3-dodecanoyloxytetradecanoic acid. The activated form may be an acyl chloride, an activated ester, a mixed anhydride or any other species allowing formation of an amide bond. Next, benzyl ester is removed by selective hydrogenolysis to yield a carboxylic acid bearing an acylamido group in a position and a (RO)₂P(O) O— group in ω position.

The peptide coupling reaction partner is obtained in preference from an α,ω-diaminoacid such as ornithine or lysine through a series of protective reaction steps which are selective of the amine functional group in ω position, for example in the form a benzyloxycarbonyl derivative, by means of a copper complex according to the method disclosed in [Organic Preparations and Procedures International, 23 (1992) :191-194], removal of the copper complex and protection of the amine functional group in α position, for example in the form of a t-butyoxycarboxyl derivative. Other protective groups could be used. The free carboxylic functional group is converted by reduction into a primary alcool using for example the borane-dimethylsulfide complex or by treatment of a preformed mixed anhydride by sodium borohydride, according to a method disclosed in [Tetrahedron Letters, 32 (1991) 923-926]. The amine functional group in α position is freed an in acidic medium (for example through treatment by trifluoroacetic acid), then N-acyl-substituted by an activated fatty acid derivative, preferably a 3-hydroxytetradecanoic acid derivative such as 3-benzyoxytetradecanoic acid. If needed, the free OH functional group is protected at this stage, for example in the form of a benzyloxymethyl ether. The ω-amino functional group. is freed by treatment with a reagent compatible with other protective groups still present, for example by selective hydrogenolysis in an alcohol type solvent containing triethylamine if the protective group is a benzyloxycarbonyl one.

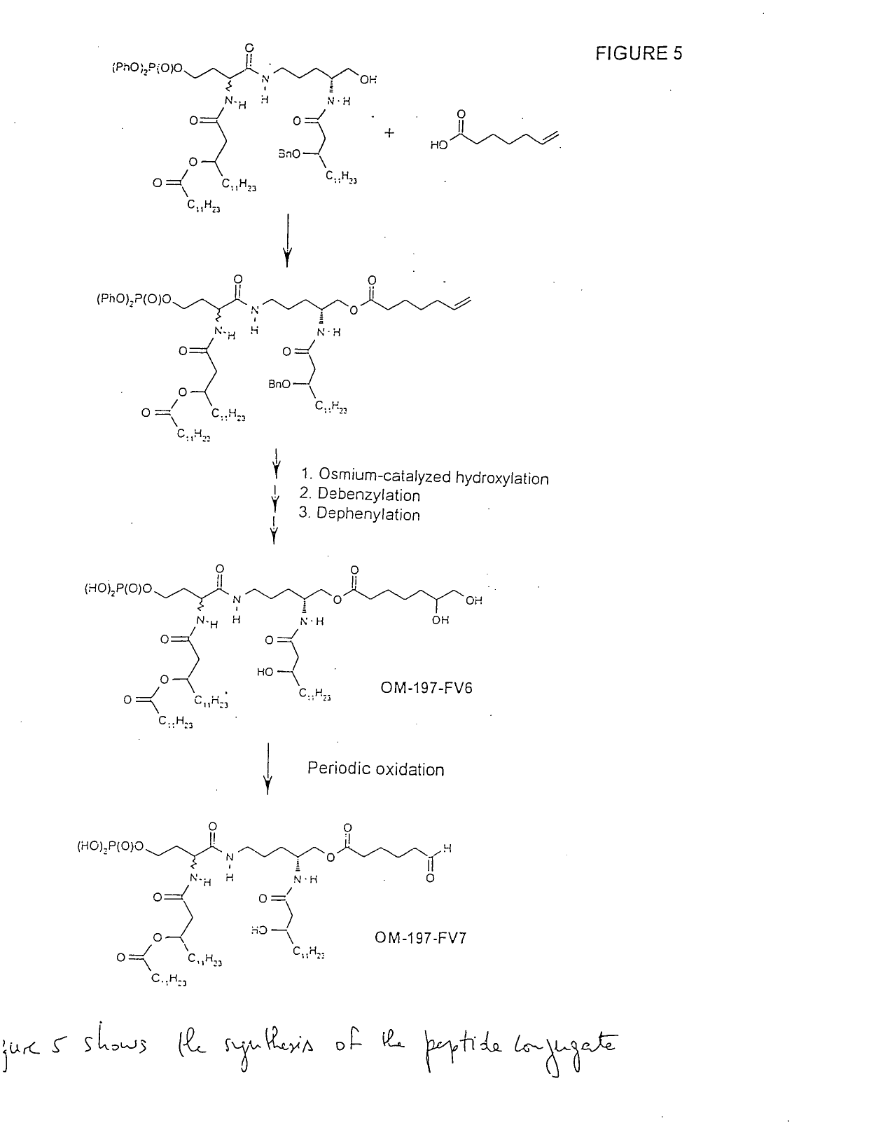

In the currently preferred synthesis method, the amine thus obtained is coupled by means of an α-acylamine-, ω-phosphoryl-carboxylic acid prepared as described above in presence of IIDQ or another peptide coupling reagent, to yield a protected phosphorylated dipeptide-like compound. This product is O-acyl substituted at the still free hydroxyl functional group with an ω-functionally derivatized acid such as 6-heptenoic acid in presence of EDCl or another esterifying reagent (FIG. 5). The alcenyl functional group of this ester is subjected to a dihydroxylation reaction in presence of osmium tetraoxide either in catalytic or stoechiometric amount, then the protective groups of the phosphate and the hydroxyl functional group optionally present in the form of a benzyl ether, are removed by hydrogenolysis in presence of a suitable catalyst (EX 2.1.). In the last step, the vicinal diol group is subjected to an oxidation reaction such as for example using periodic acid to generate a reactive aldehyde functional group (E.x. 2.2) Reduction of the aldehyde functional group into a primary alcohol results in a derivative bearing an ω(-hydroxyacyl group (Ex. 2.9).

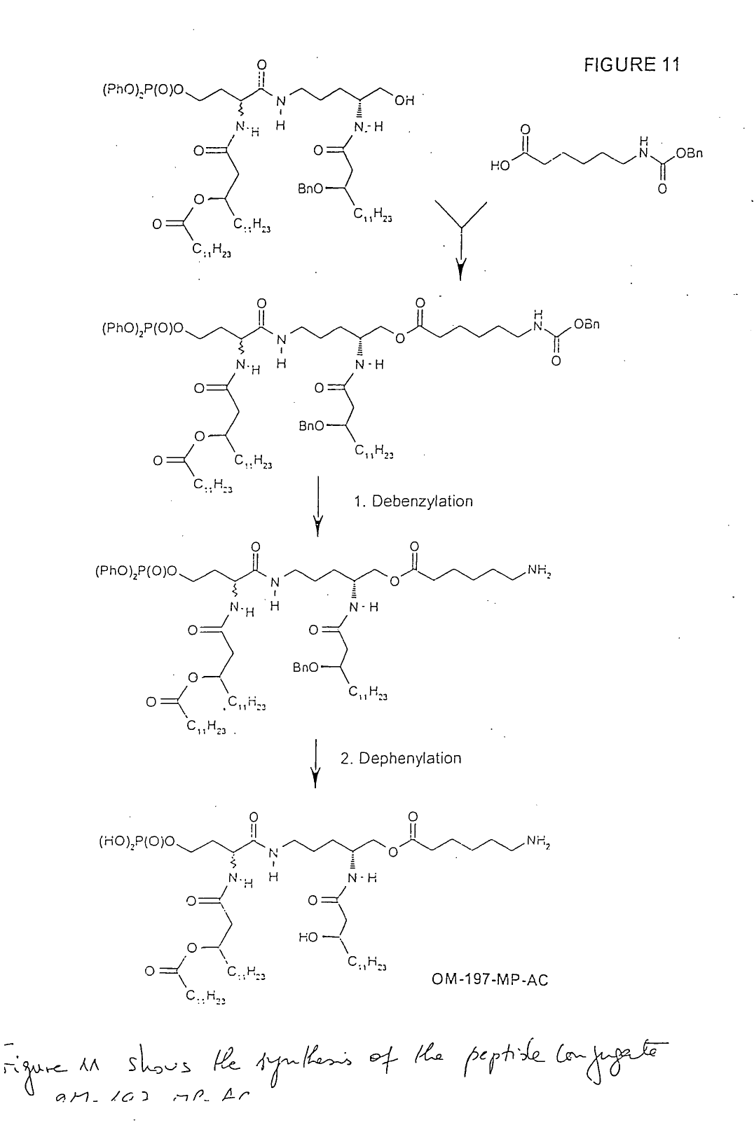

As one alternative to this method, the resulting peptide conjugate product is O-acyl substituted using an ω-aminoalkanoic acid derivative, such as 6-benzyloxycarbanoylaminohexanoic acid (FIG. 11). The product thus obtained is subjected to a full deprotection reaction by hydrogenolysis in presence of a suitable catalyst to provide a dipeptide-like compound bearing an aminoalkanoyl accessory side chain spacer (Ex. 2.6).

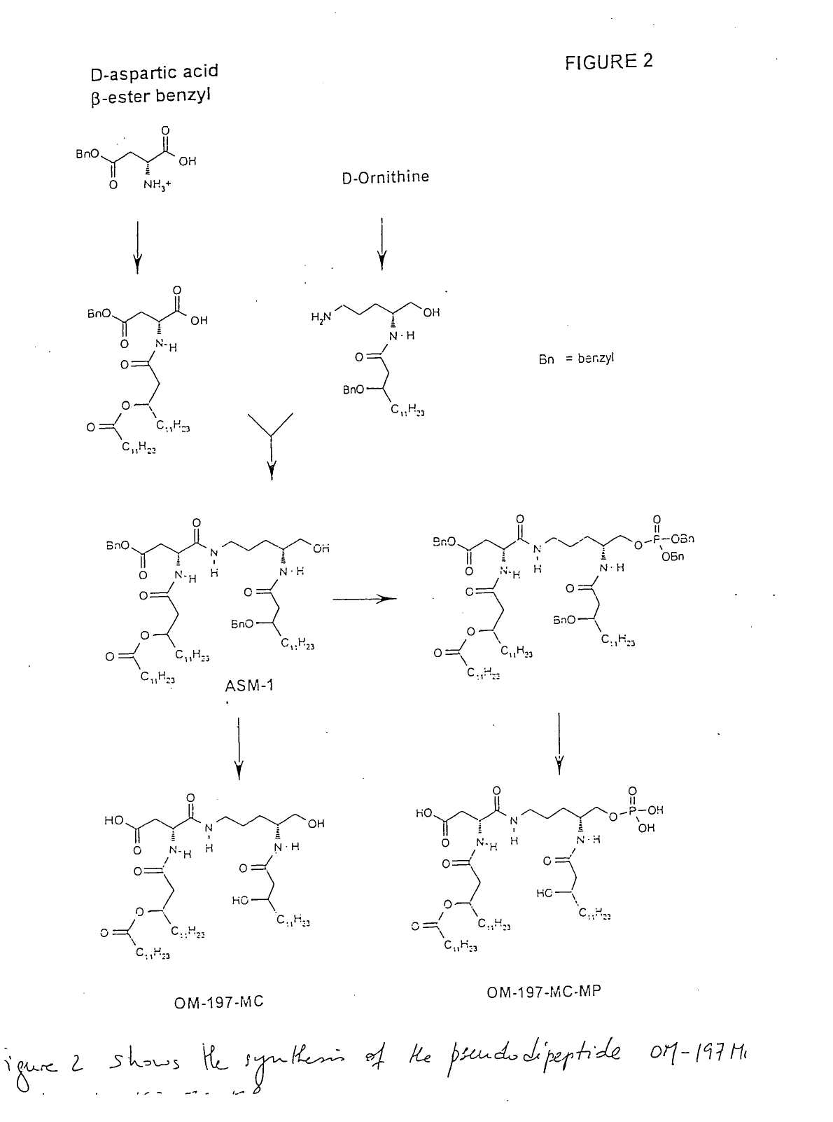

In a second preferred method, the peptide coupling reaction acid partner is a D- or L-aspartic acid or optionally a D- or L-glutamic acid derivative (FIG. 2). The aspartic acid derivative is obtained by. N-acylation of the amine functional group of this amino acid β-benzyl ester by reaction with a fatty acid, preferably a 3-hydroxy fatty acid derivative such as 3-dodecanoyloxytetradecanoic acid, in presence of an acyl substitution agent. This product is coupled to the amino alcohol of the previously mentioned type, and the dipeptide-like compound thus obtained is then subjected either to a hydrogenolysis reaction in presence of a catalyst such as palladium on carbon or a platinium coated substrate to provide the dipeptide-like compound bearing the carboxylic acid functional group (Ex. 1.2) or to a phosphorylation reaction, for example by the phosphoramidite method, followed by a hydrogenolysis reaction to yield the dipeptide-like compound bearing a carboxylic acid and a phosphoric acid functional group (Ex 1.3.).

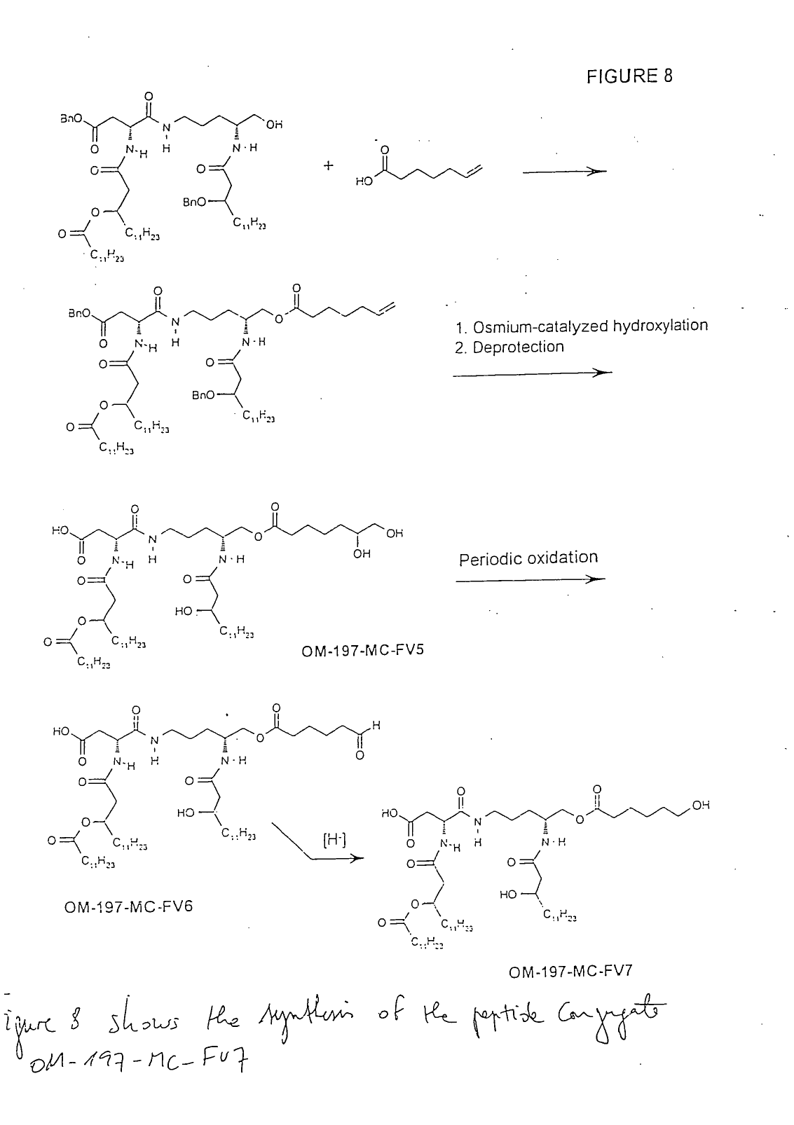

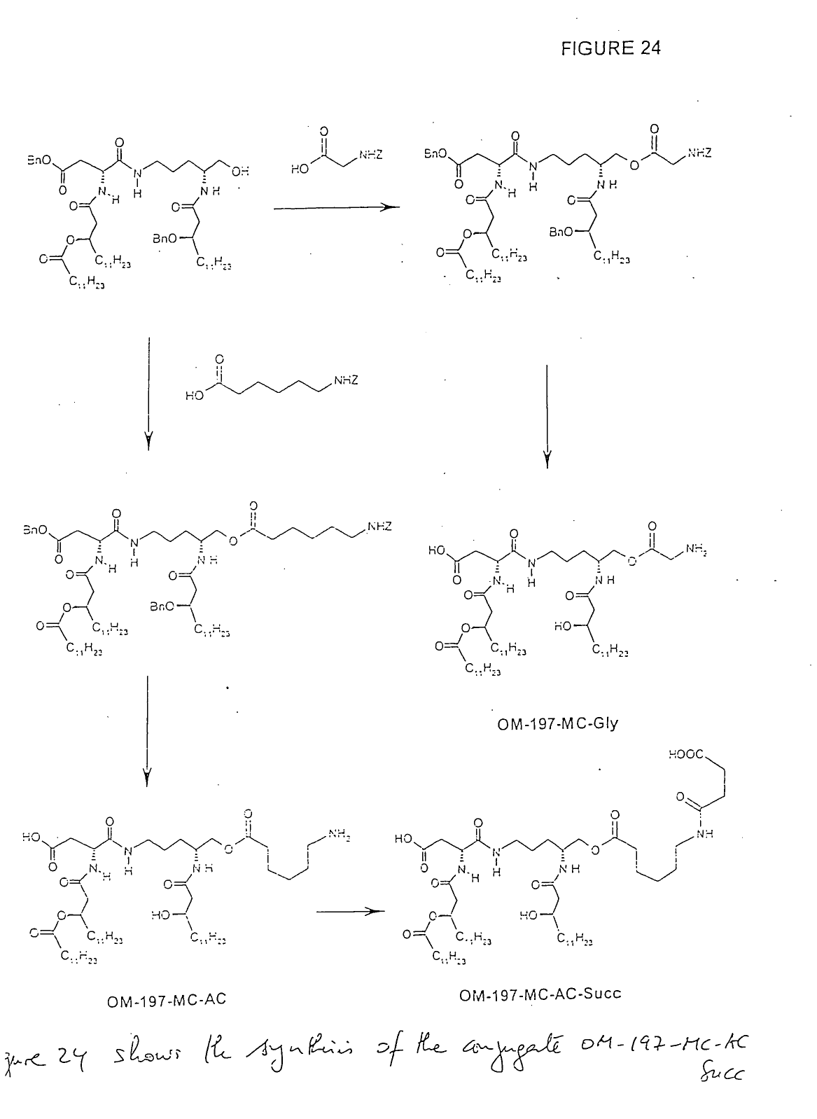

Alternatively, the hydroxyl functional group of the previously obtained dipeptide-like compound (FIG. 2) is acylated by an ω-functionally substituted acid. Preferably, this ω-functionally substituted acid is an ω-alcenoic acid or an ω-aminoalkanoic acid. In case heptenoic acid is used (FIG. 8), the dipeptide-like compound finally results in an O-heptenoyl derivative which is sequentially subjected to dihydroxylation reactions in presence of osmium tetraoxide, and thereafter to deprotection (Ex 2.3) and periodic oxidation reactions (Ex. 2.4), whereby reduction of the thus formed aldehyde functional group with a reducing agent such as for example sodium borohydride yields the dipeptide-like compound bearing an ω-hydroxyalkanoyl functional group (Ex. 2.5). In case ω-aminoalkanoic acid such as N-benzyloxycarbonyl 6-aminohexanoic acid or glycine derivative is used (FIG. 24), acylation of the dipeptide-like compound results in protected O-aminoacyl derivatives which are thereafter subjected to a deprotection reaction through hydrogenolysis (Ex. 2.13 and 2.18).

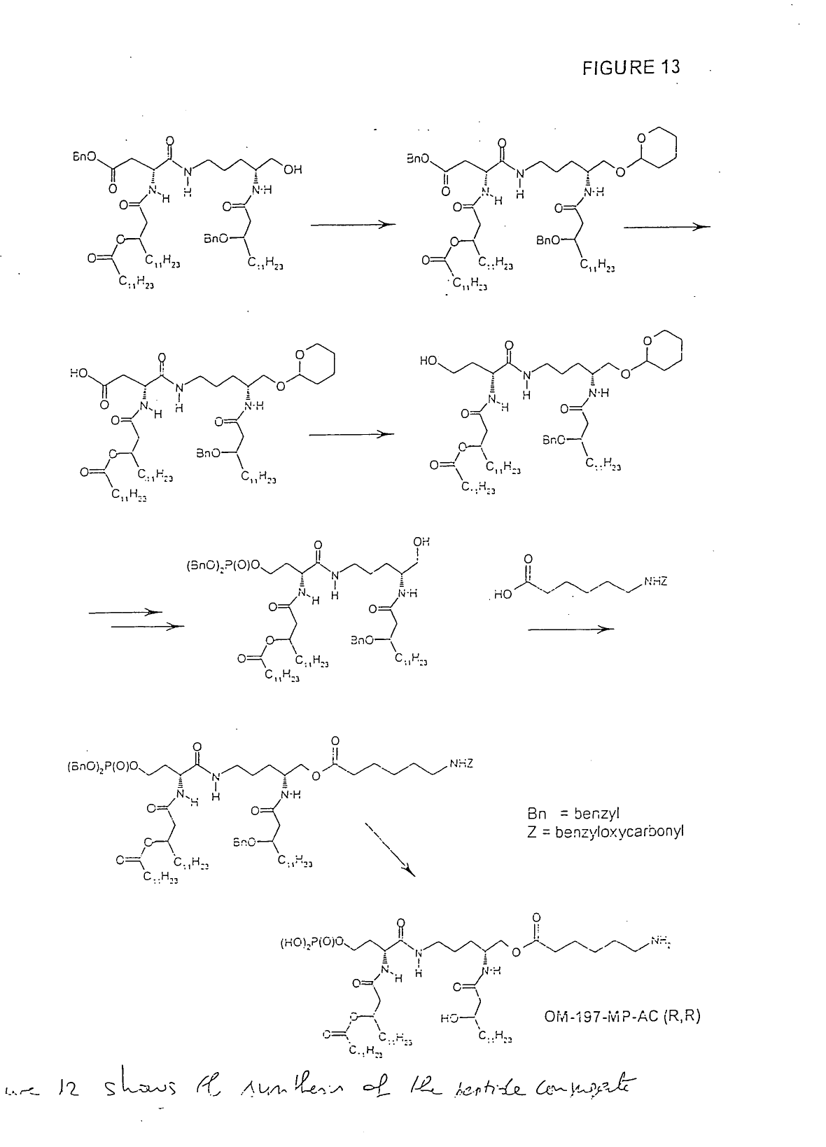

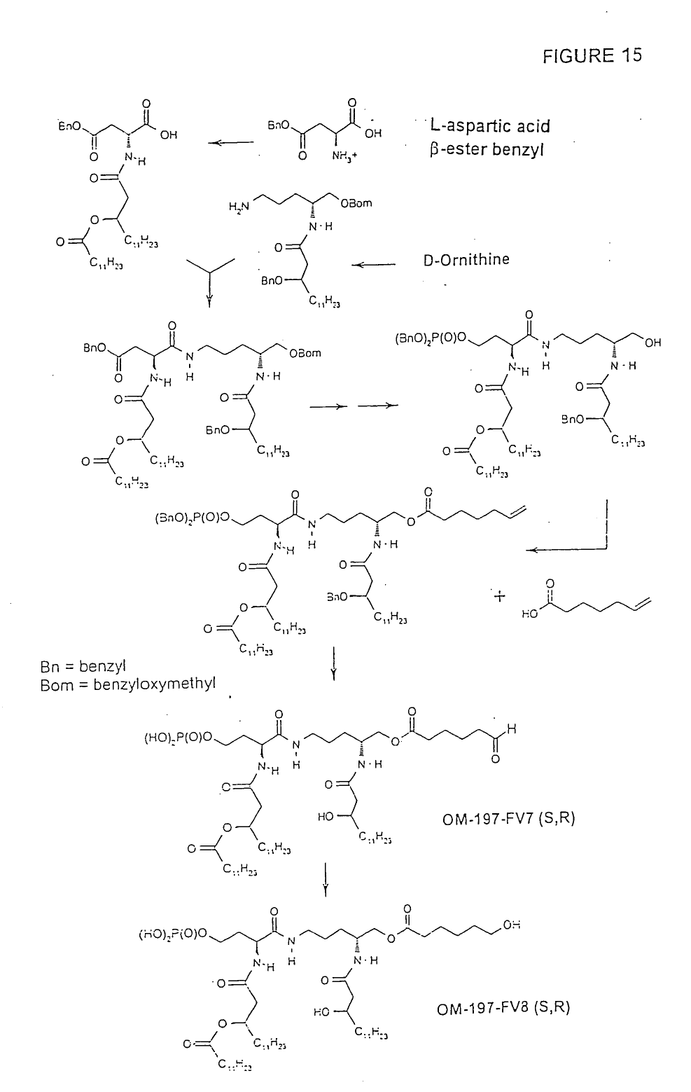

In a second variant to this procedure (FIG. 13), the intermediate dipeptide-like compound described in FIG. 2 is subjected to a protection reaction at the free hydroxyl functional group, preferably in the form of a tetrahydropyranyl ether, then the esterified carboxyl functional group is successively deprotected by hydrogenolysis or some other deprotection method, reduced, preferably after activation into a mixed anhydride, with a reducing agent such as sodium borohydride; the resulting hydroxyl fonctional group is subjected to a phosphorylation reaction, preferably by the phosphoramidite method, and thereafter the tetrahydropyranyl ether is hydrolyzed in acidic conditions and the hydroxyl functional group thereby regenerated is subjected to an acylation reaction with an ω-functionally substituted carboxylic acid. In preference, the ω-functionnally substituted acid is an ω-alcenoic acid or an ω-aminoalkanoic acid. In the case of 6-benzyloxycarbonylaminohexanoic acid, the dipeptide-like compound results into a protected O-aminoacyl derivative which is subsequently subjected to a hydrogenolysis reaction (Ex. 2.7, Ex. 2.8). Alternatively, the monophosphorylated intermediate of the previous section can be obtained (FIG. 15) by peptide coupling of an N-acyl aspartic acid β-benzylester derivative (FIG. 2) with a protected species, for example in the form of either benzyloxymethyl or tetrahydrofuranyl ether or still silyl- ornithine-aminoalcohol derivative (FIG. 1), followed by freeing the acid functional group in the form of a benzyl ester, reducing the same into a primary alcohol, phosphorylating said functional group and deprotecting the protected alcohol functional group in the form of either benzyloxymethyl, tetrahydropyranyl or silyl ether.

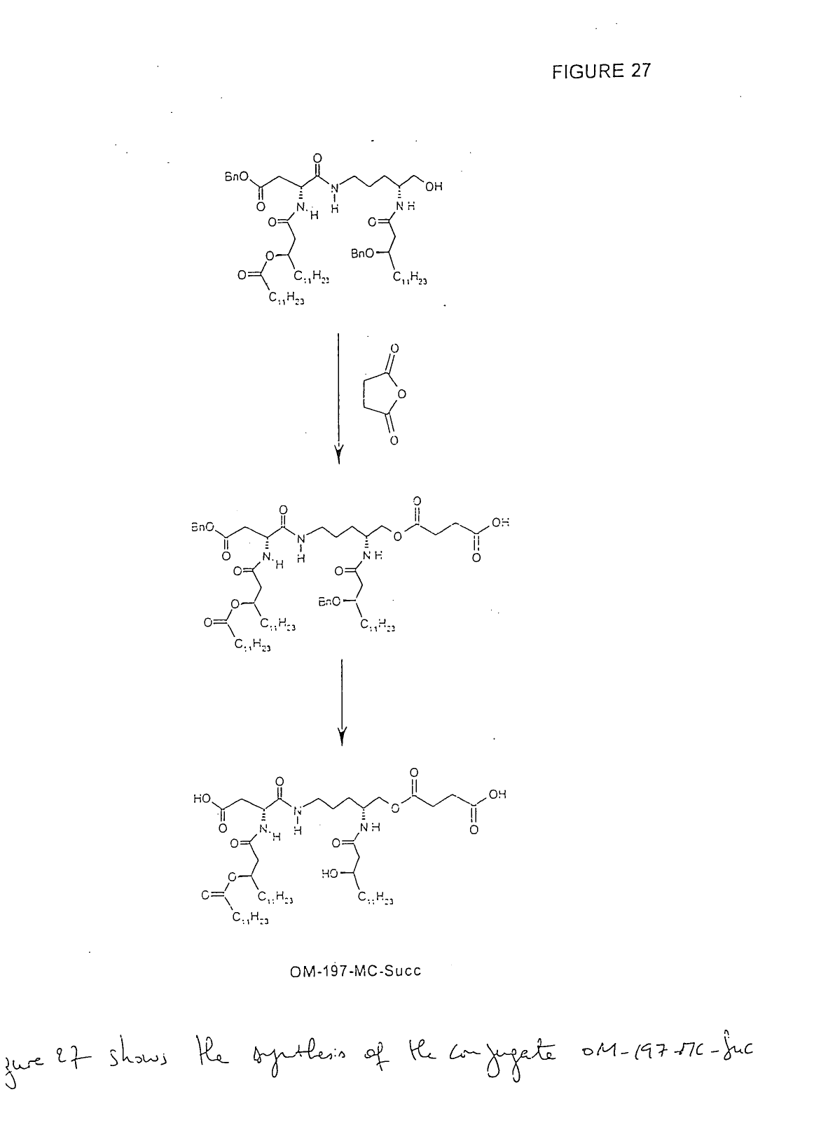

In a third variant to this procedure, the intermediate dipeptide-like compound described in FIG. 2 is subjected to an O-acylation reaction (FIG. 27) using a dicarboxylic acid derivative, preferably succinic anhydride, in presence of a base or a coupling agent, then the newly formed ester is subjected to a deprotection reaction by hydrogenolysis (Ex. 2.19) Treatment of deprotected O-aminoacyl derivatives with succinic anhydride gives dipeptide-like compounds bearing a succinylaminoacyl group (Ex. 2.17).

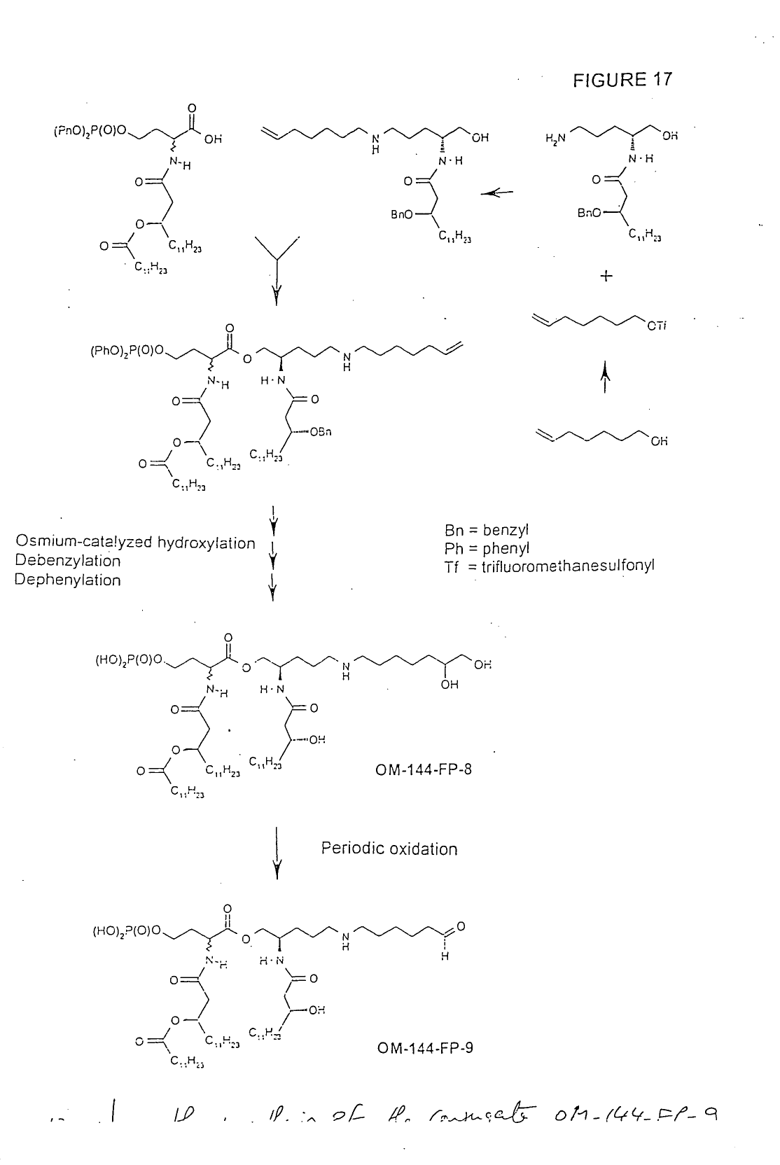

According to a third preferred method, the amino alcohol obtained from ornithine or lysine as described above, is alkyl-substituted by an ω-alcenyl triflate, such as hept-6-enyl triflate (FIG. 17). The coupling of this amino alcohol bearing a secondary amine with α-acylamine ω-phosphoryl acid described above in presence of EDCl or another acylating agent gives an ester. The alcenyl group is then subjected to a dihydroxylation reaction in presence of osmium tetraoxide, and then the protective groups are removed by hydrogenolysis in presence of appropriate catalysts, and the vicinal diol functional group is oxidized by sodium periodate to form a reactive aldehyde functional group (Ex. 2.10).

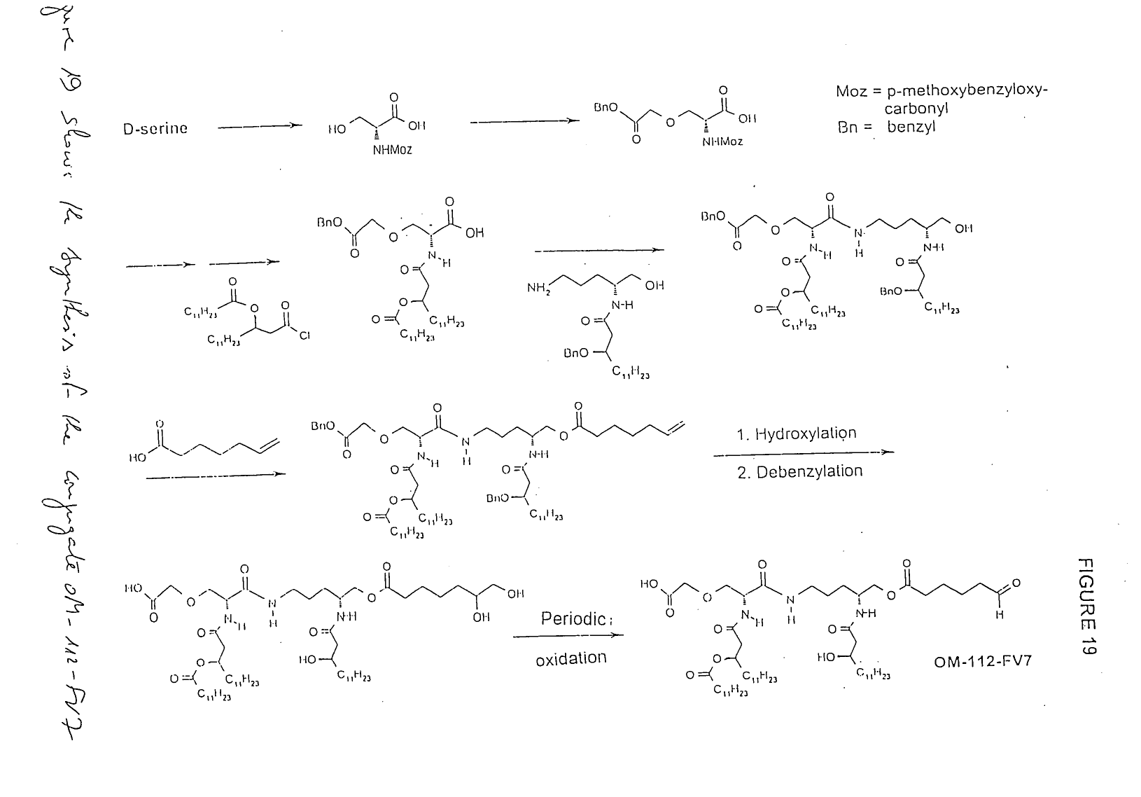

According to a fourth preferred method (FIG. 19), an N-protected serine derivative, for example a p-methoxybenzyloxycarbonyl derivative prepared according to a method disclosed in Synthesis (1989), 36-37 is O-alkyl substituted with benzyl bromoacetate. The protective group of the amine functional group is removed, for example by treatment with trifluoroacetic acid in dichloromethane, and the amine functional group is N-acylated, preferably with a 3-hydroxy fatty acid derivative such as 3-dodecanoyloxytetradecanoic acid, in presence of an acylating agent or using an acid chloride or any other activated form of the fatty acid. The N-acyl O-alkyl serine derivative thus obtained is coupled with the amino alcohol described above (FIG. 1) in presence of a peptide coupling agent such as IIDQ, and the free OH functional group is then acyl substituted with an ω-functionally derivatized alkanoic acid in presence of a reagent such carbodiimide. Preferably, this functionnally derivatized acid is an ω-alcenoic acid or an ω-aminoalkanoic acid derivative. For instance, using hetp-6-enoic acid, the dipeptide-like compound finally results in an O-(6-heptenoyl) derivative which is subjected in succession to dihydroxylation reactions in presence of osmium tetraoxide, followed by hydrogenolysis and periodic oxidation deprotection reactions (Ex 2.11.).

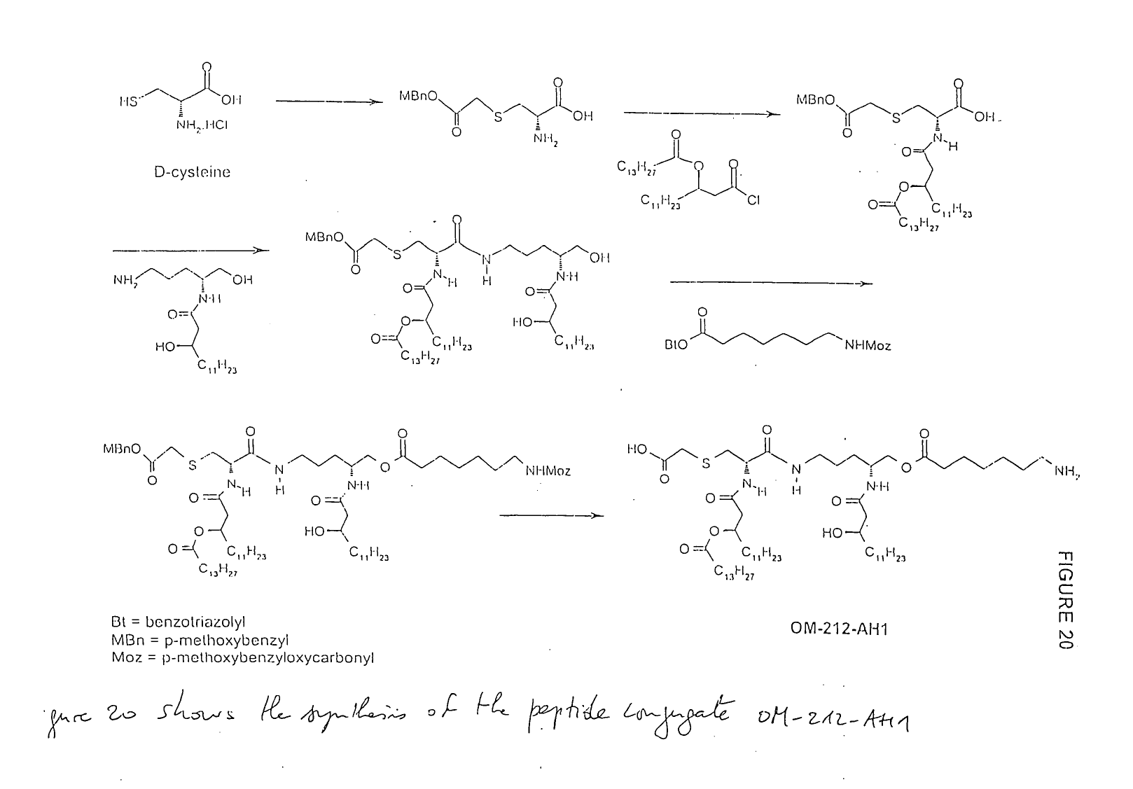

According to a fifth currently preferred method, the starting amino acid is cysteine or homocysteine (FIG. 20). For example, cysteine is S-alkyl substituted with p-methoxybenzyl bromoacetate, then N-acyl substituted, preferably with a 3-hydroxy fatty acid derivative such as 3-tetradecanoyloxytetradecanoic acid, in presence of an acylating agent or using acid halide or any other activated form of such a fatty acid. The S-alkyl N-acyl-cysteine derivative thus obtained is coupled to 5-amino-2-[(R)-3-hydroxytetradecanoylamino]pentan-1-ol in presence of IIDQ or another peptide coupling agent. The free OH functional group is then O-acyl substituted with an co-functionally derivatized alkanoic acid, used in the form of an activated ester such as O-benzotriazolyl ester. Preferably, this acid is an ω-aminoalkanoic acid such as 7-(p-methoxy-benzyloxycarbonylamino)heptanoic acid. The product thus obtained is subjected to a full deprotection reaction in an aqueous acidic environment to thereby provide a dipeptide-like compound bearing a 7-aminoheptanoyl accessory side chain spacer (Ex. 2.12).

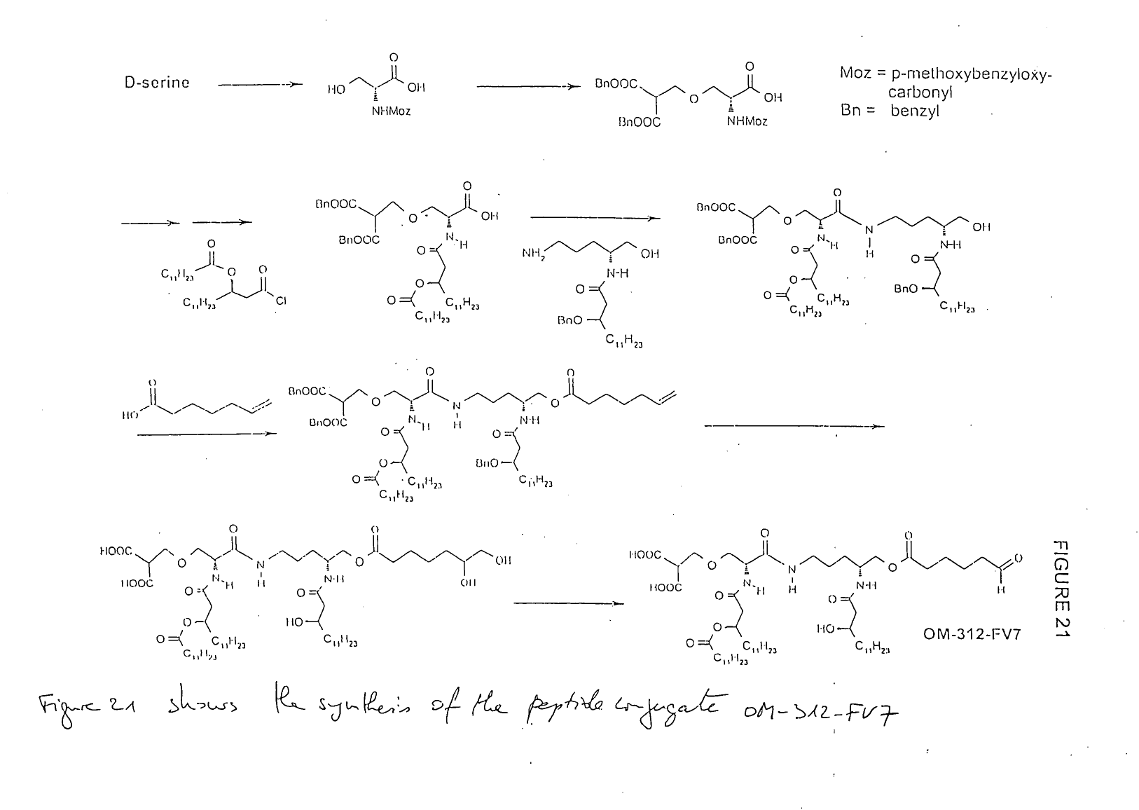

In another preferred method (FIG. 21), the serine N-p-methoxybenzyloxycarbonyl derivative is O-alkyl substituted wth dibenzyl methylenemalonate in alkaline conditions. The amino protective group is removed by acid treatment, then the amine functional group thus freed is N-acyl substituted, preferably with a 3-acylhydroxy fatty acid derivative such as 3-dodecanoyloxytetradecanoic acid, in presence of an acylating agent or a functional fatty acid derivative such as acid chloride or any other activated form of such a fatty acid. The N-acyl O-alkyl serine derivative thus obtained is coupled to the amino alcohol described above (FIG. 1) in presence of IIDQ or any other peptide coupling agent. The dipeptide-like compound is then O-acyl substituted at the free OH functional group with an co-functionally derivatized alkanoic acid. In preference, this acid is an ω-alcenoic acid or an ω-aminoalkanoic acid derivative, for example, using the hept-6-enoic acid. The dipeptide-like compound accordingly results in the O-(6-heptenoyl) derivative which is sequentially subjected to dihydroxylation reactions in presence of osmium tetraoxide, then to deprotection reactions by hydrogenolysis and periodic oxidation reaction, to finally yield a derivative having a 6-oxohexanoyl accessory side chain spacer as well as a malonyl group (Ex. 2.13).

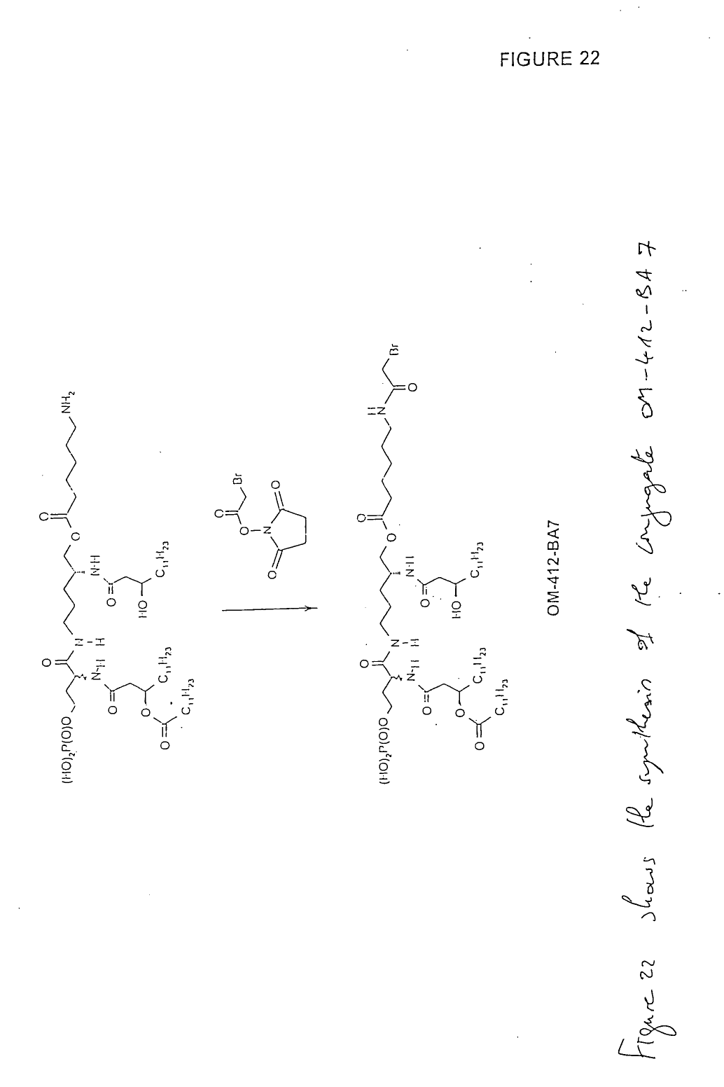

In an equally preferred alternative method (FIG. 22), the 6-aminohexanoyl derivative mentioned above (FIG. 11), is N-acylated with a bromoacetamido group using for example bromoacetic acid O-succinimidyl ester as an acylating agent. This reaction provides a derivative bearing a 6-(bromoacetamido)hexanoyl accessory side chain spacer. (Ex 2.14.).

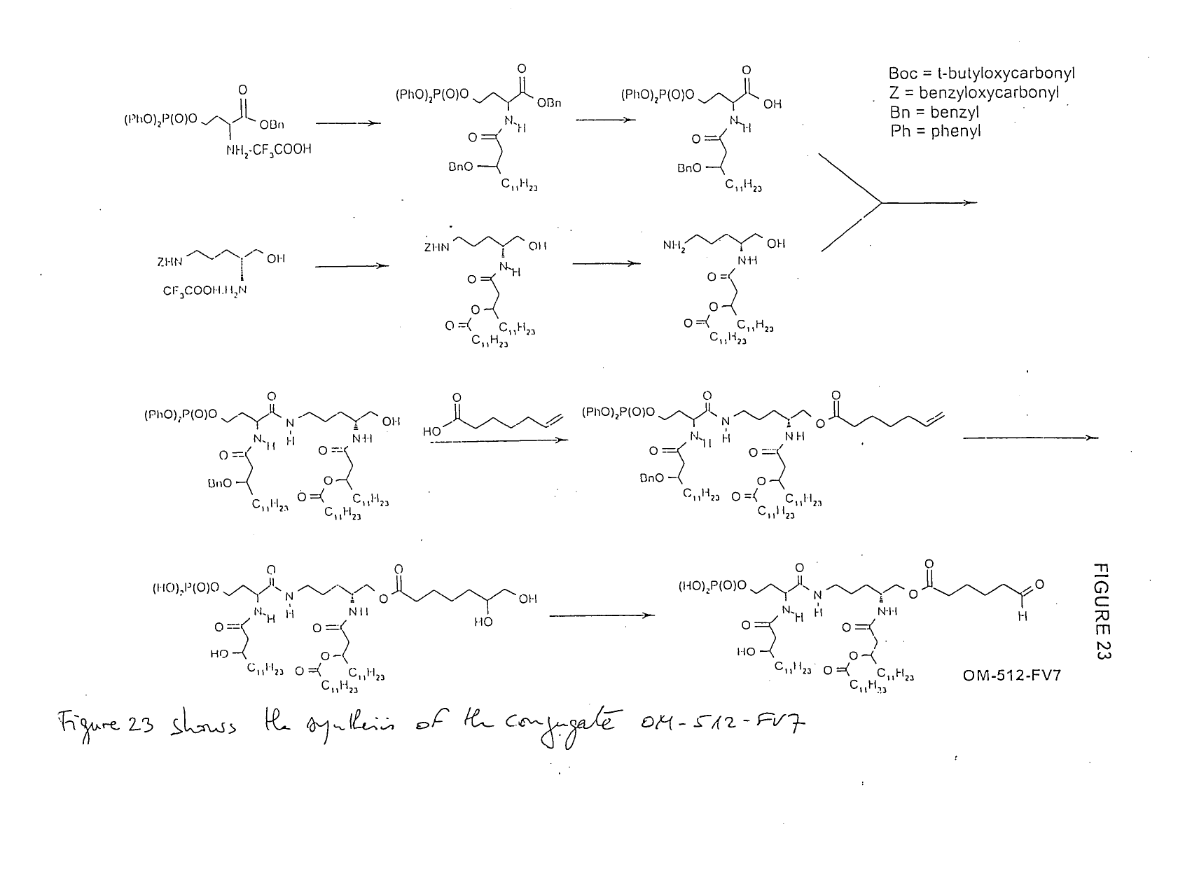

According to an eighth preferred method (FIG. 23), the O-phosphoryl-serine benzyl ester (see FIG. 1) is N-acyl substituted with 3-benzyloxytetradecanoic acid, preferably using the mixed anhydride prepared from 3-benzyloxytetradecanoic acid and isobutyl chloroformate. The benzyl ester is then subjected to selective hydrogenolysis as stated above. The ω-N-benzyloxycarbonyl diamino alcohol derived from ornithine (see FIG. 1) is N_{α}-acyl substituted with 3-dodecanoyloxytetradecanoic acid, in presence of a coupling agent or using an activated ester or some other activated form of the fatty acid, and the ω-amino functional group is freed by selective hydrogenolysis. This amine is coupled to the N-(3-benzyloxytetradecanoyl) O-(diphenyl-oxyphosphoryl) homoserine derivative in presence of IIDQ or another peptide coupling agent. The dipeptide-like compound thus obtained is O-acyl substituted with an ω-functionally derivatized alkanoic acid, for example in presence of carbodiimide. Preferably, this acid is an ω-alcenoic acid or an ω-aminoalkanoic acid derivative. For example, using hept-6-enoic acid, the dipeptide-like compound results in an O-(6-heptenoyl) derivative which is sequentially subjected to dihydroxylation reactions in presence of osmium tetraoxide, then to a deprotection process by hydrogenolysis in presence of suitable catalysts and to a periodic oxidation reaction, to ultimately provide the derivative bearing a 6-oxohexanoyl accessory group.

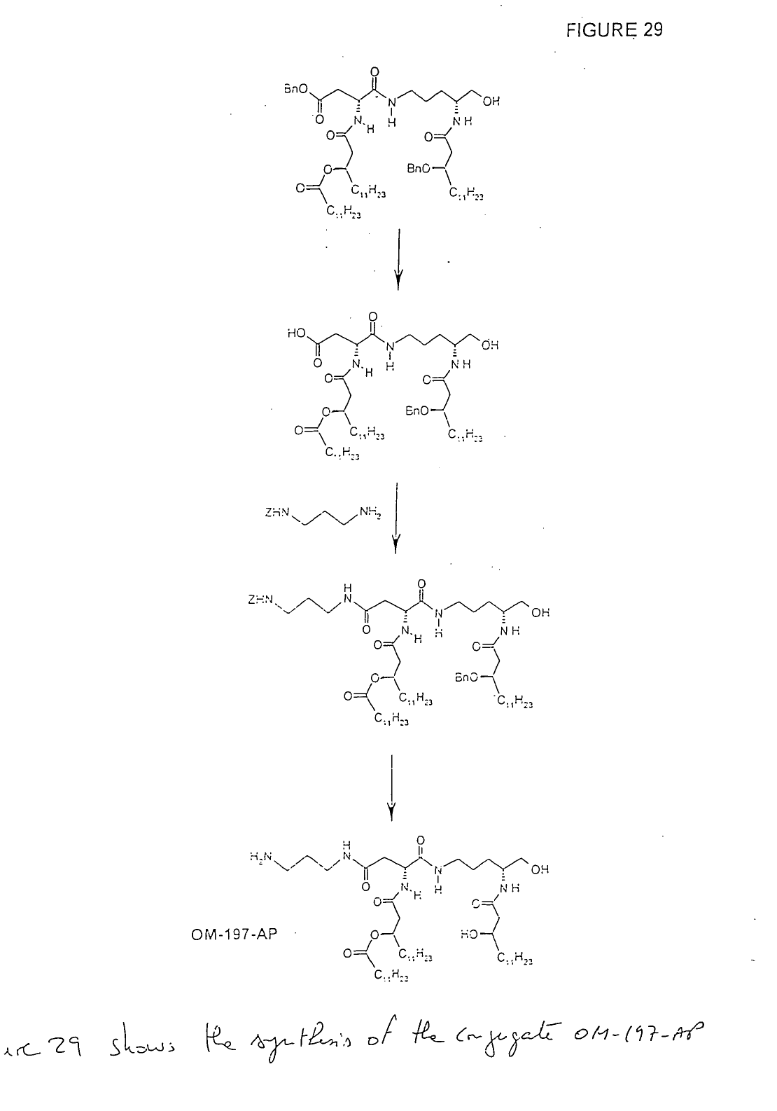

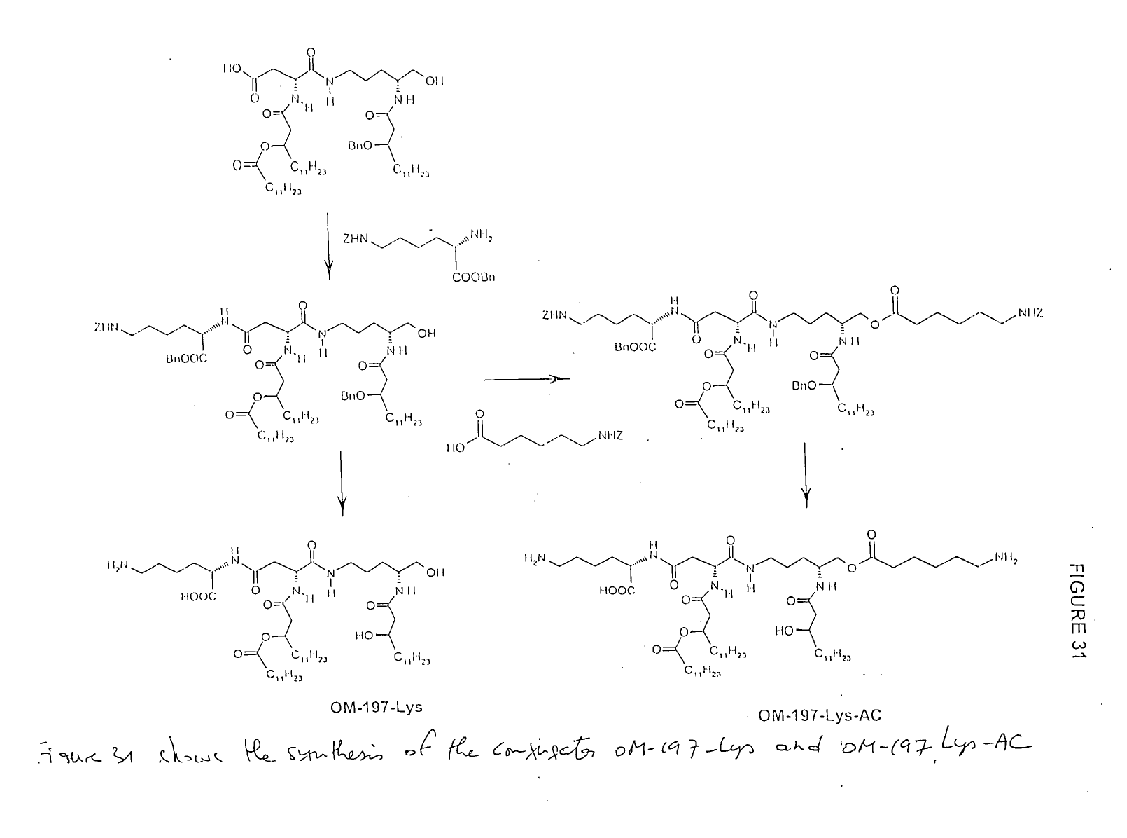

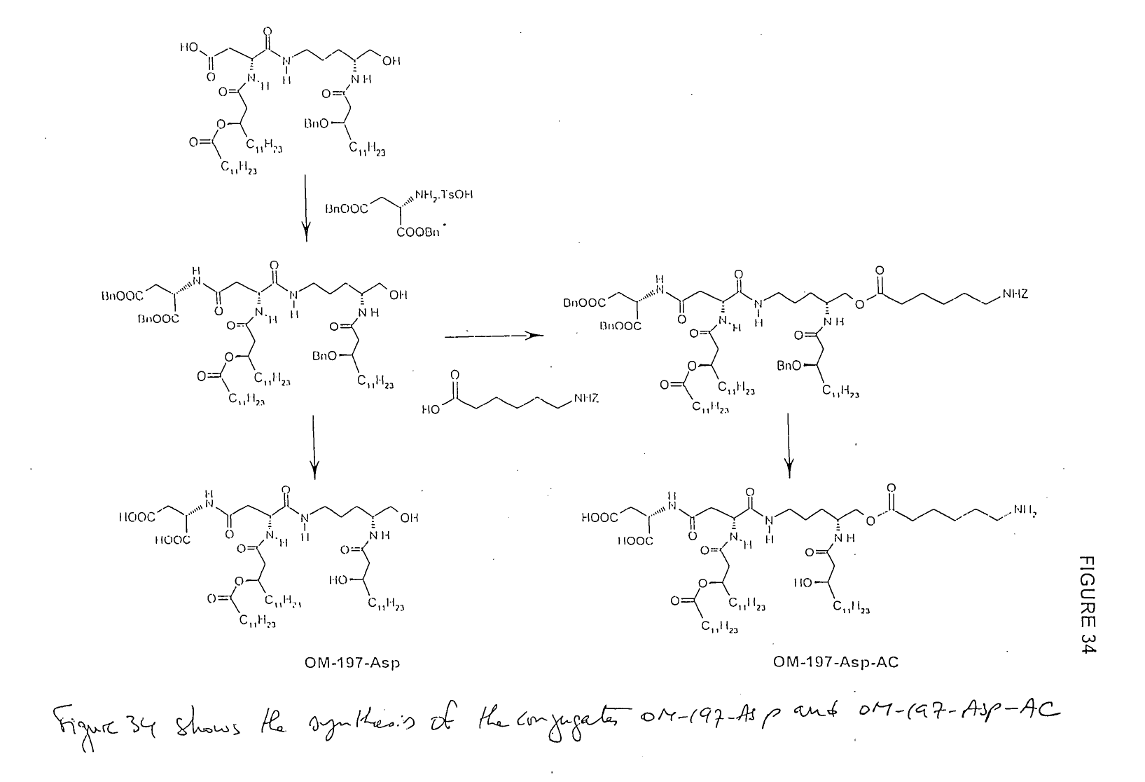

According to a ninth preferred method (FIGS. 29, 31 and 34), the intermediate dipeptide-like compound described in FIG. 2 is subjected to a deprotection reaction at the esterified carboxyl functional group, and then this carboxyl functional group is coupled to a functionally substituted aminoalkane, in presence of a coupling agent. Preferably, the functionnally substituted aminoalkane is an α, ω-diaminoalkane or amino acid derivative. In case of 1-amino-3-benzyloxycarbonylaminopropane (FIG. 29), the coupling reaction product is thereafter subjected to a hydrogenolysis reaction (Ex. 2.20). In case of N^{ε}, Z-lysine benzyl ester, (FIG. 31), and α,β-dibenzyl aspartate (FIG. 34), the coupling reaction product is subjected either to a deprotection reaction, preferably by hydrogenolysis (Ex. 2.21 and 2.23), or an O-acylation reaction by means of an ω-functionnally substituted alkanoic acid, preferably the N-benzyloxycarbonyl 6-aminohexanoic acid derivative. The resulting ester is then deprotected by hydrogenlysis or some other deprotection method (Ex. 2.22 and 2.24).

The invention further relates to intermediates of general formulae V, VI, IX, XIII and XVI either in the form of a pure enantiomer and/or stereoisomer or a mixture of stereoisomers.

The invention still relates to pharmaceutical compositions containing as an active ingredient at least one compound of general formula I, either in neutral or charged state, in combination or in admixture with a non toxic, pharmaceutically acceptable, inert vehicle or excipient.

The invention relates more specifically to pharmaceutical compositions containing as an active ingredient at least one salt of a compound of general formula I, together with an organic or mineral base intended for therapeutic use.

The invention still further relates to pharmaceutical compositions based on a compound of general formula I, either in the form of a pure enantiomer or in the form of a mixture of stereoisomers, in combination or in admixture with a pharmaceutical excipient or vehicle.

Among pharmaceutical formulations herein contemplated, mention should be made of those which are suitable to administration by mucosal, transcutaneous, topical, parenteral, digestive route or inhalation such as for instance coated or uncoated tablets, capsules, injection solutes or suspensions, spray, gels, plasters or rapid absorption solutes.

In preference, the compounds according to the invention can be grafted on an antigen to modulate the immune response or also be grafted 20 on a pharmaceutical carrier to enhance therapeutic effect or targetting thereof and be administered by injection as conjugates in aqueous solutions or suspensions, optionally neutralized by an amine or a hydroxyalkylamine. For instance, mention is made of H1N1 antigen, SYVPSAEQI nonapeptide antigen of Plasmodium and pharmaceutical carriers such as AZT, d4T as well as antibiotics such as macrolids and substances which act on the central nervous system CNS.

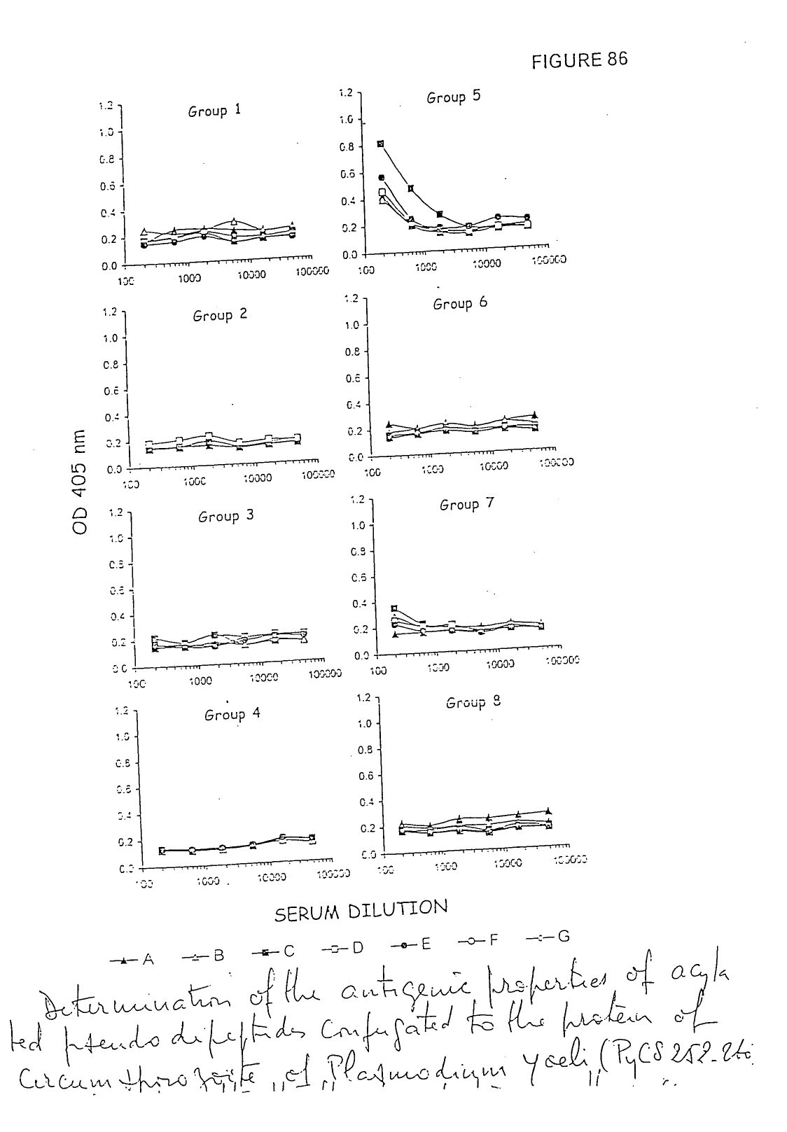

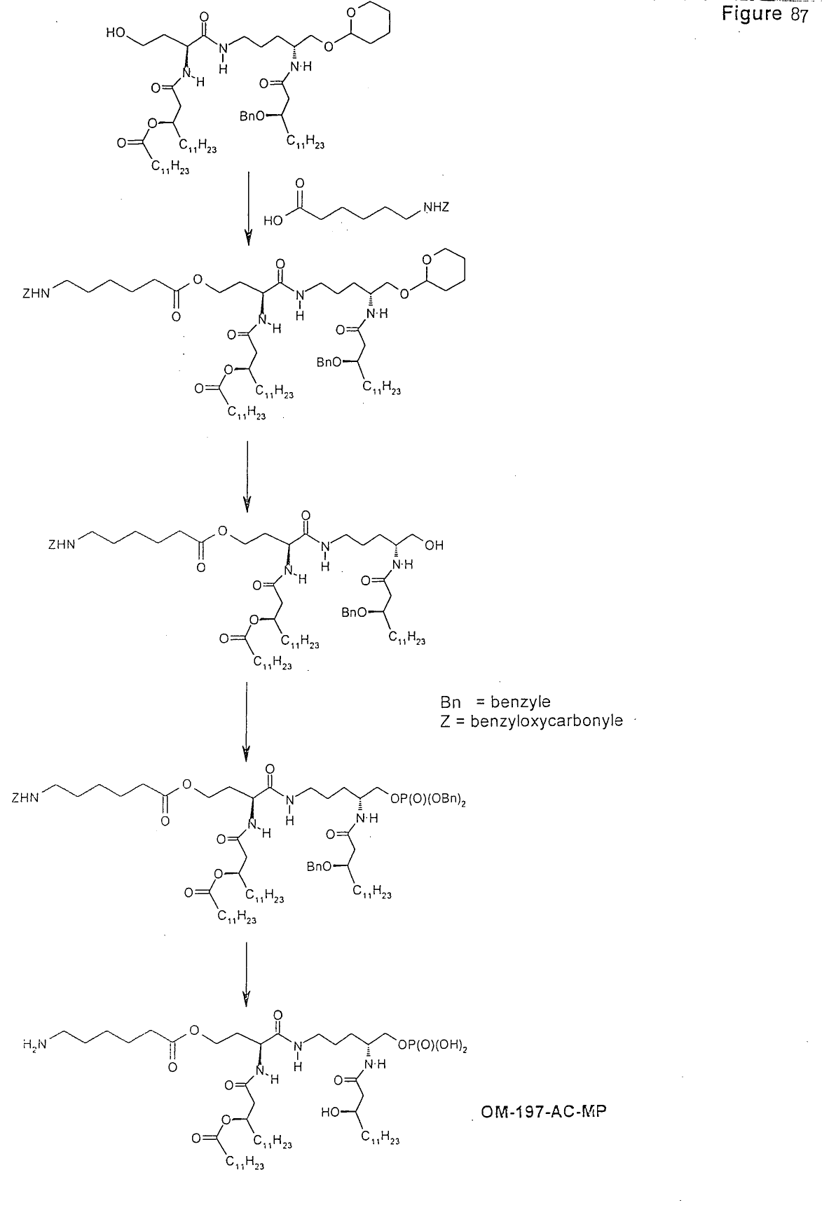

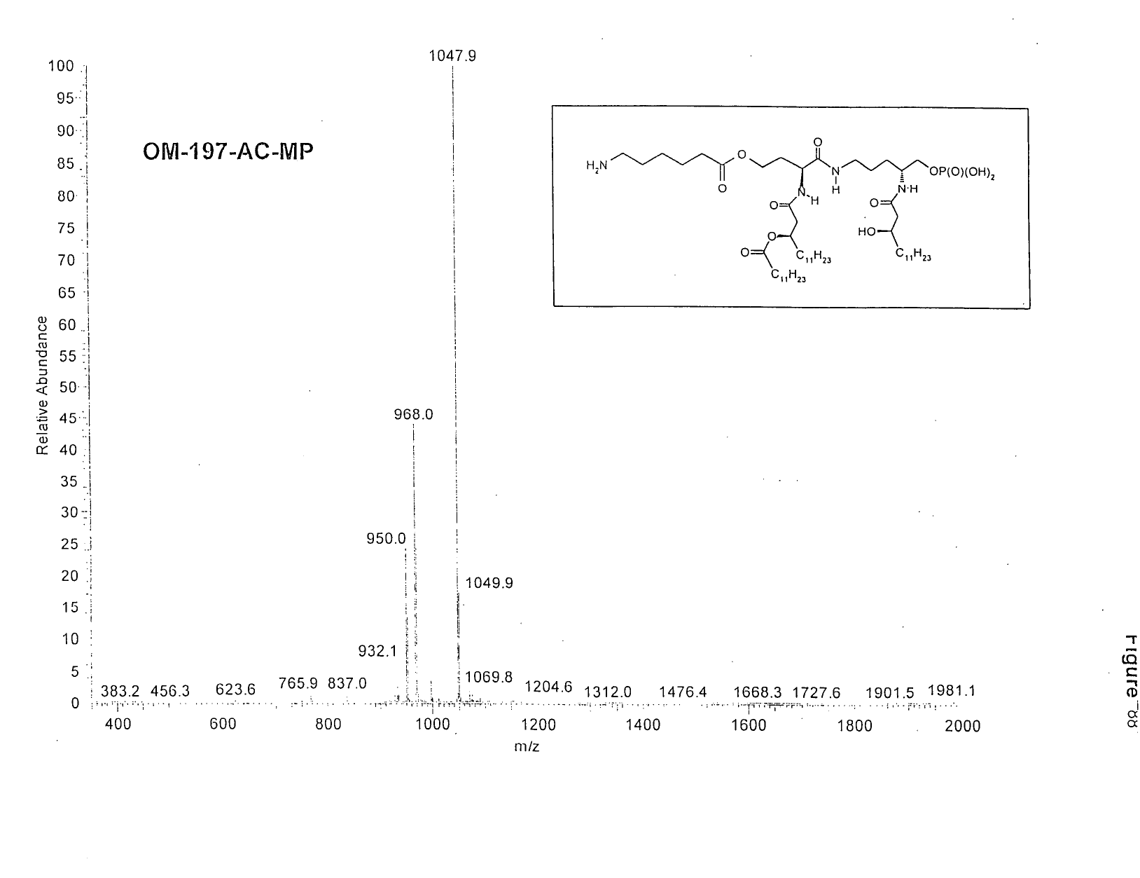

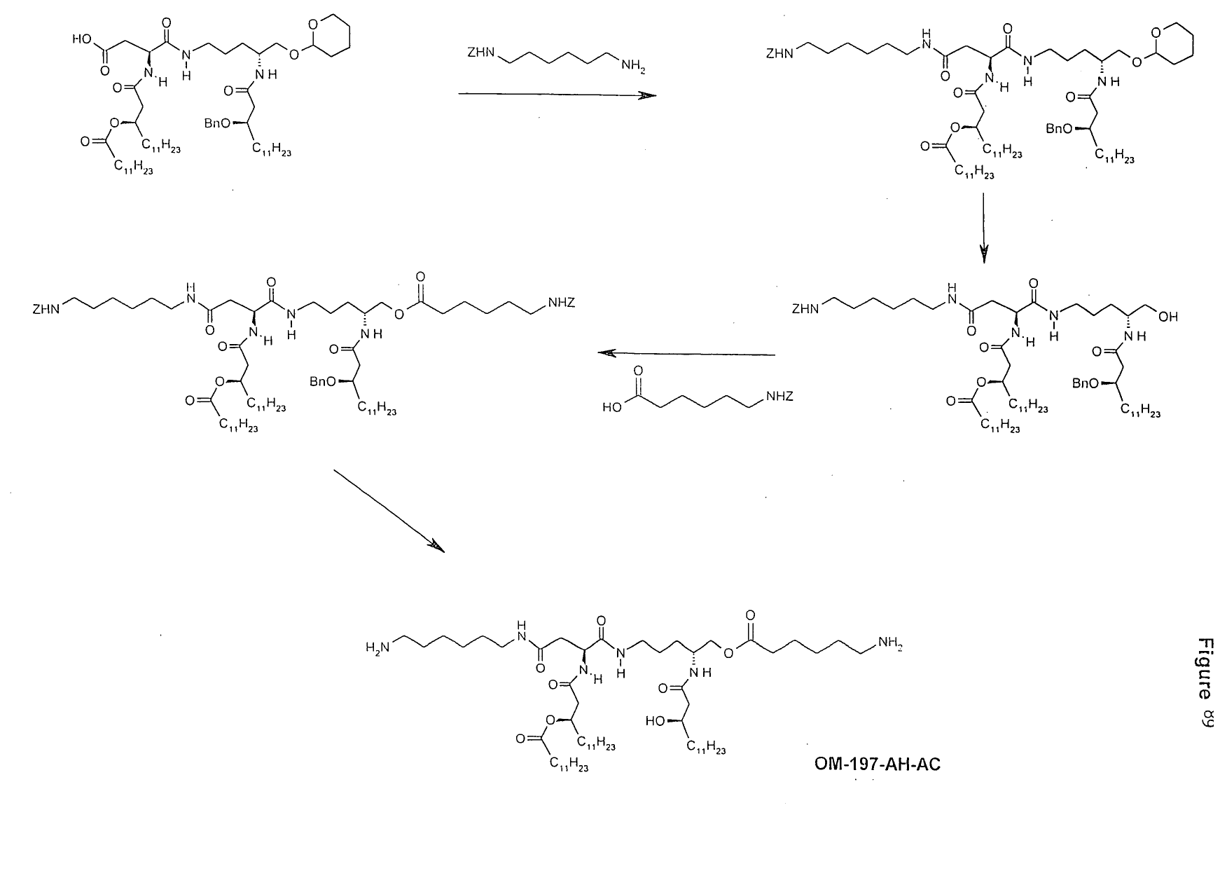

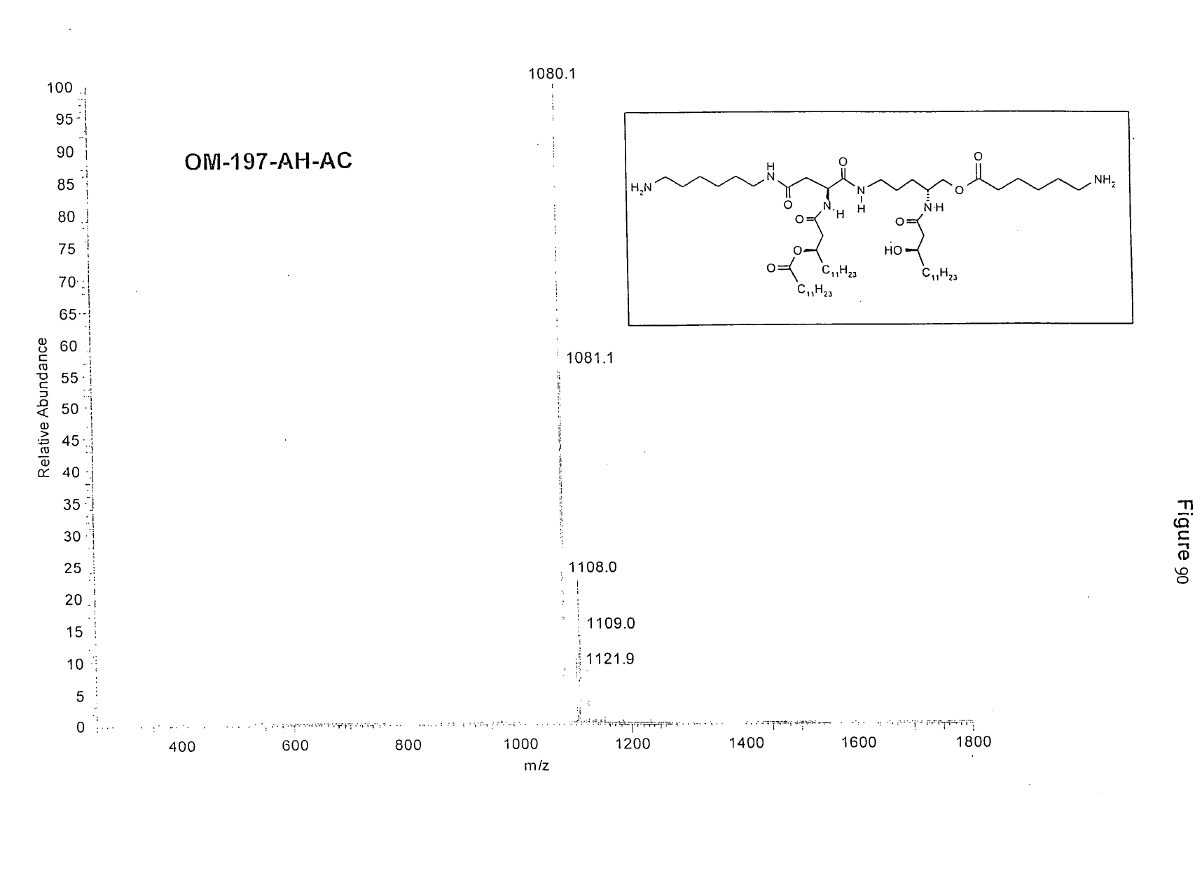

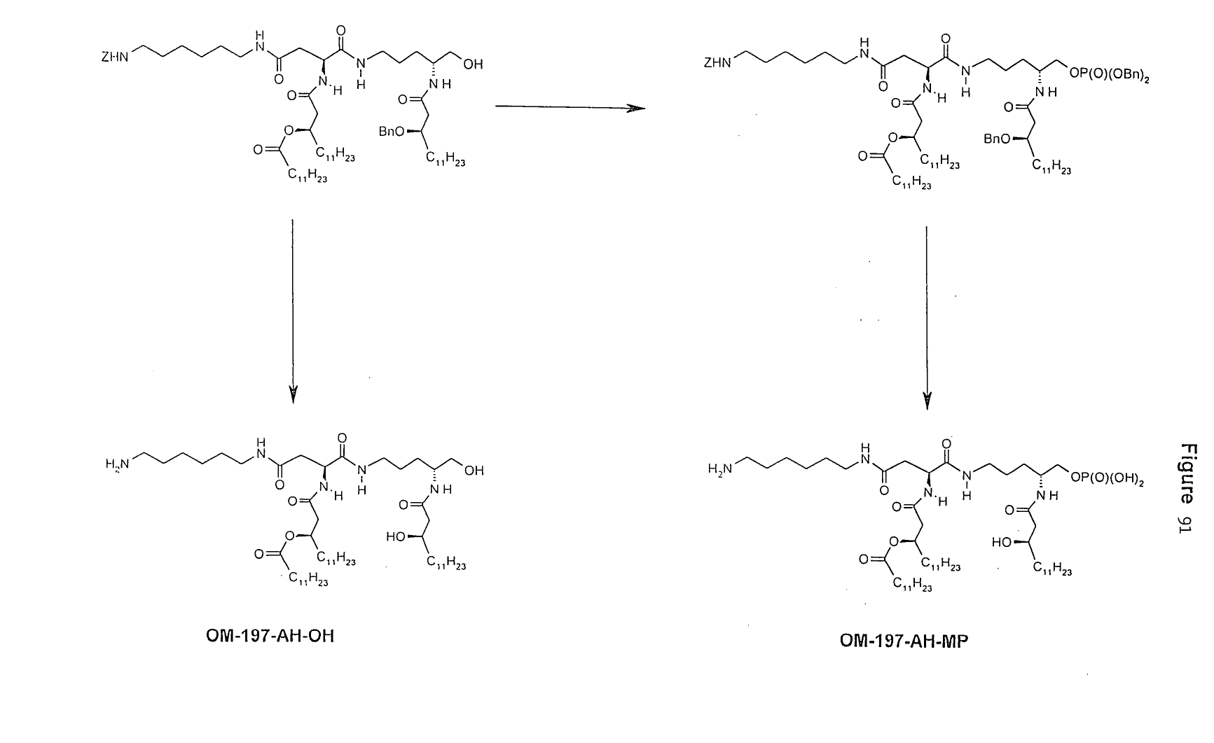

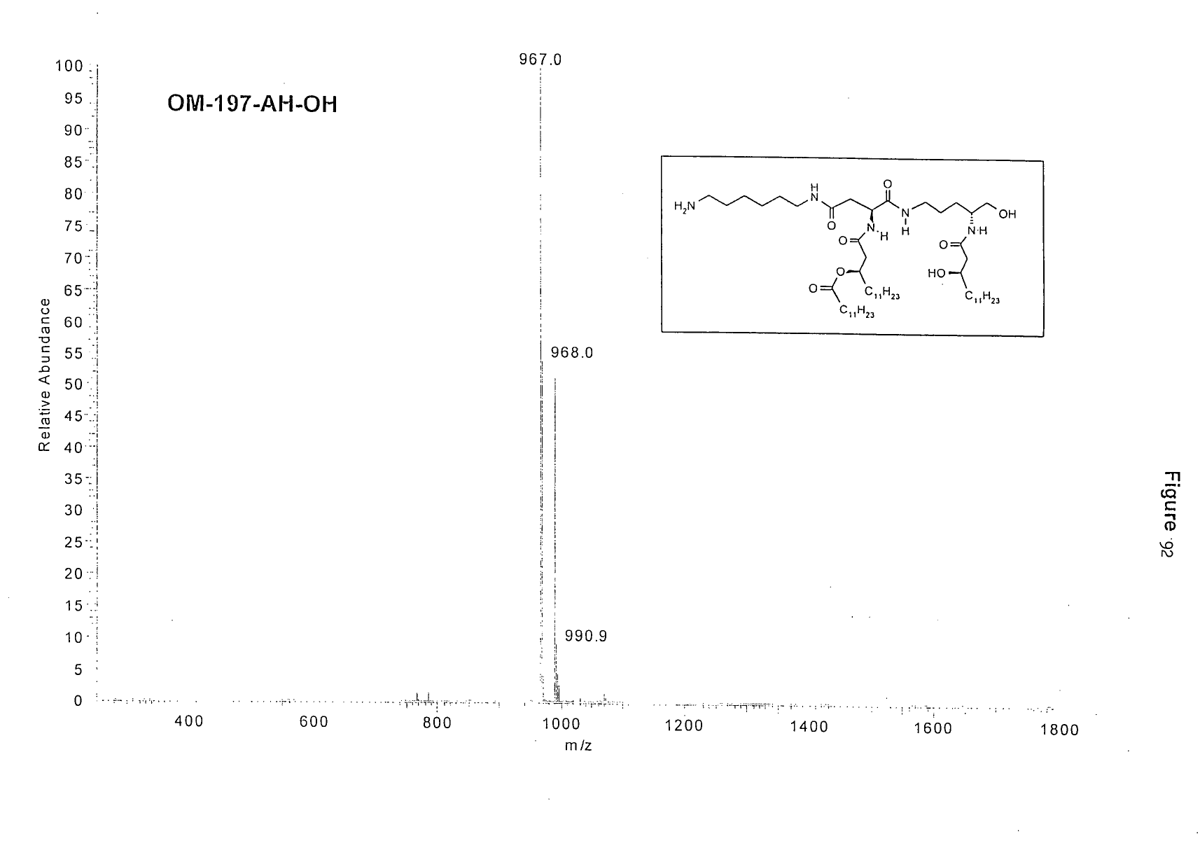

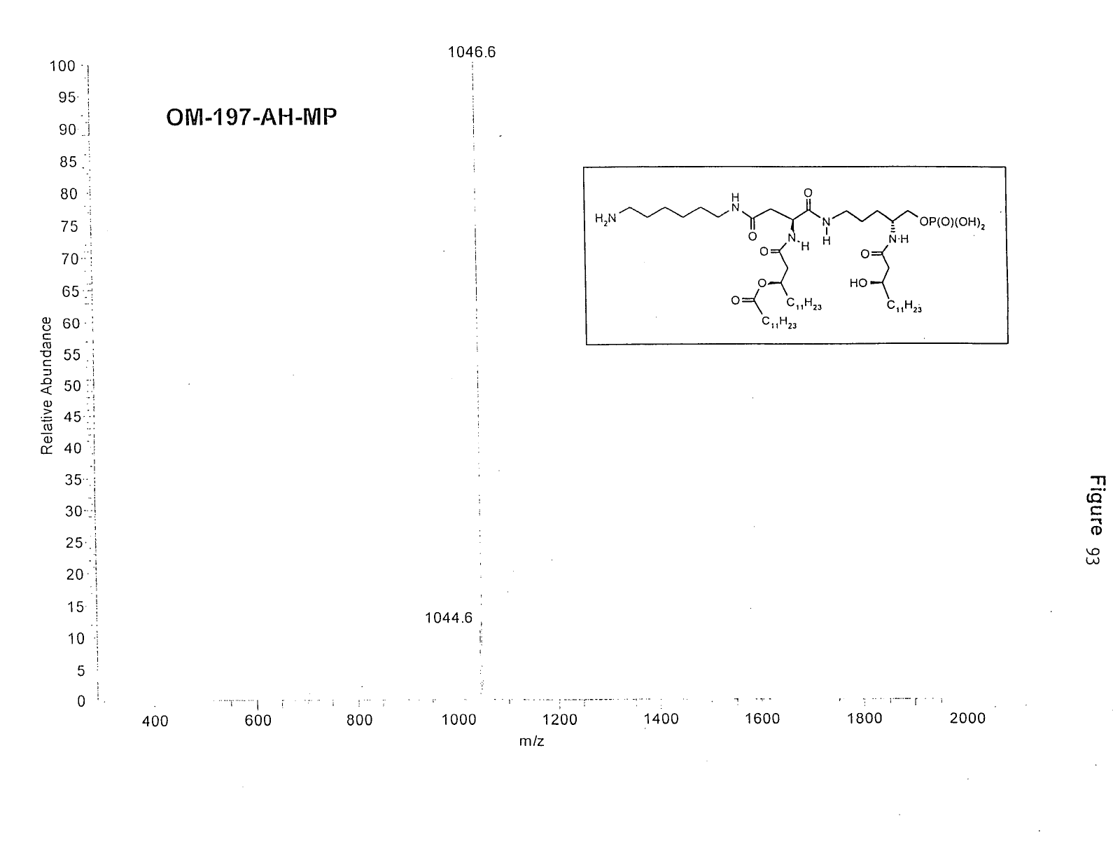

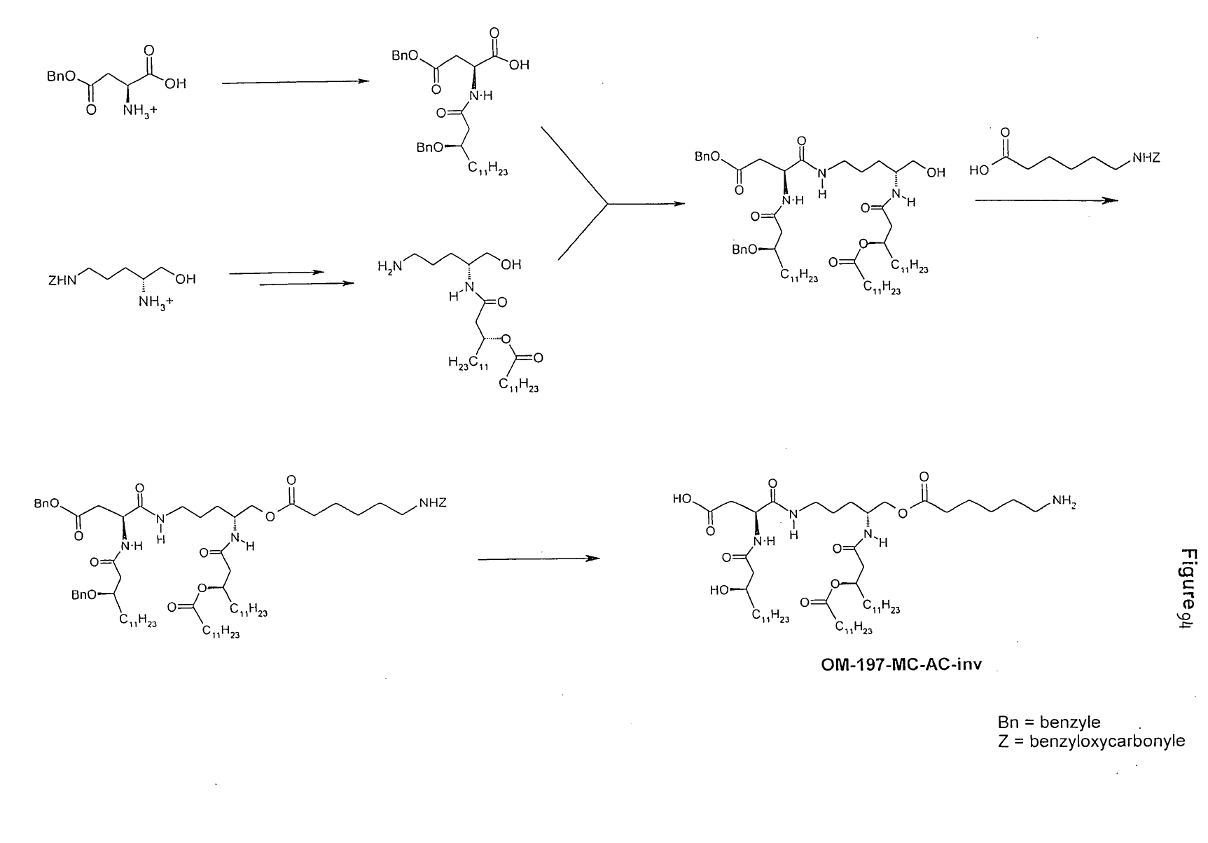

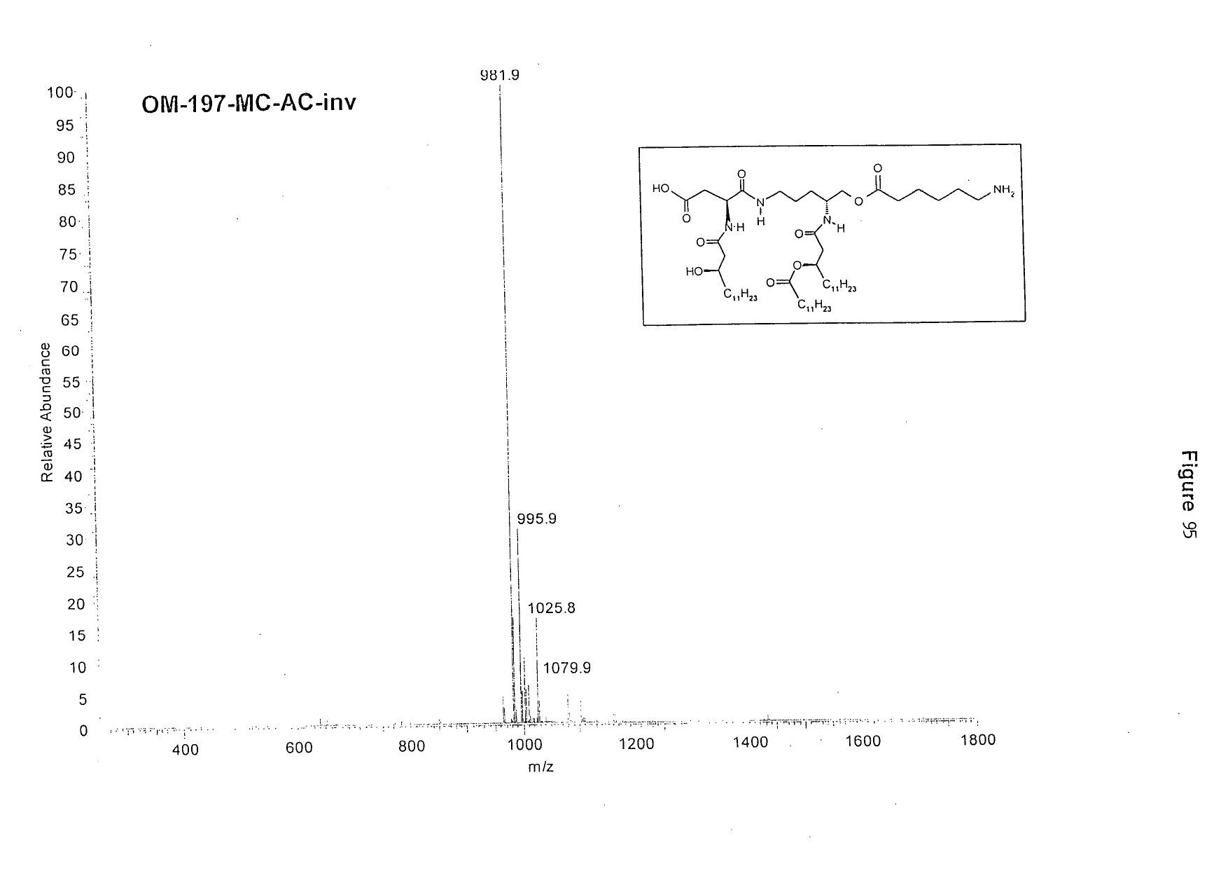

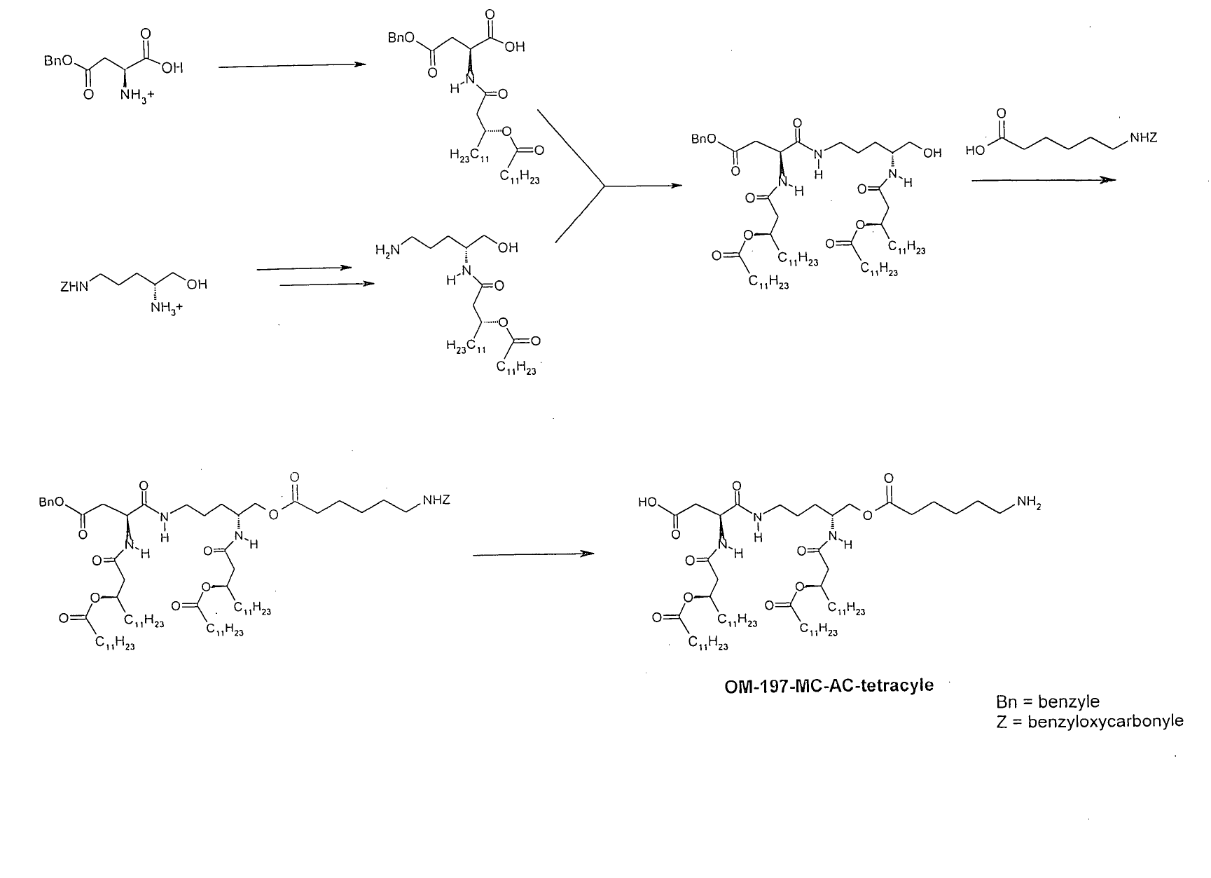

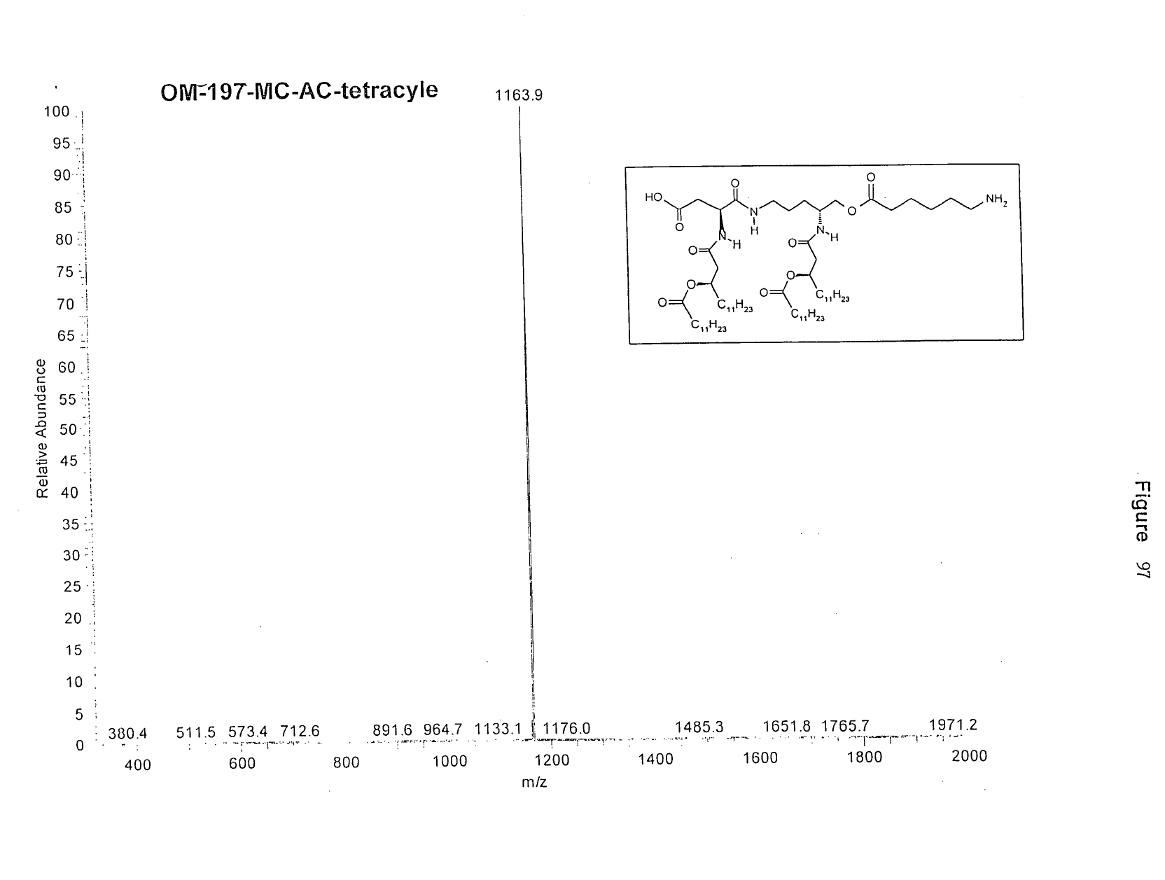

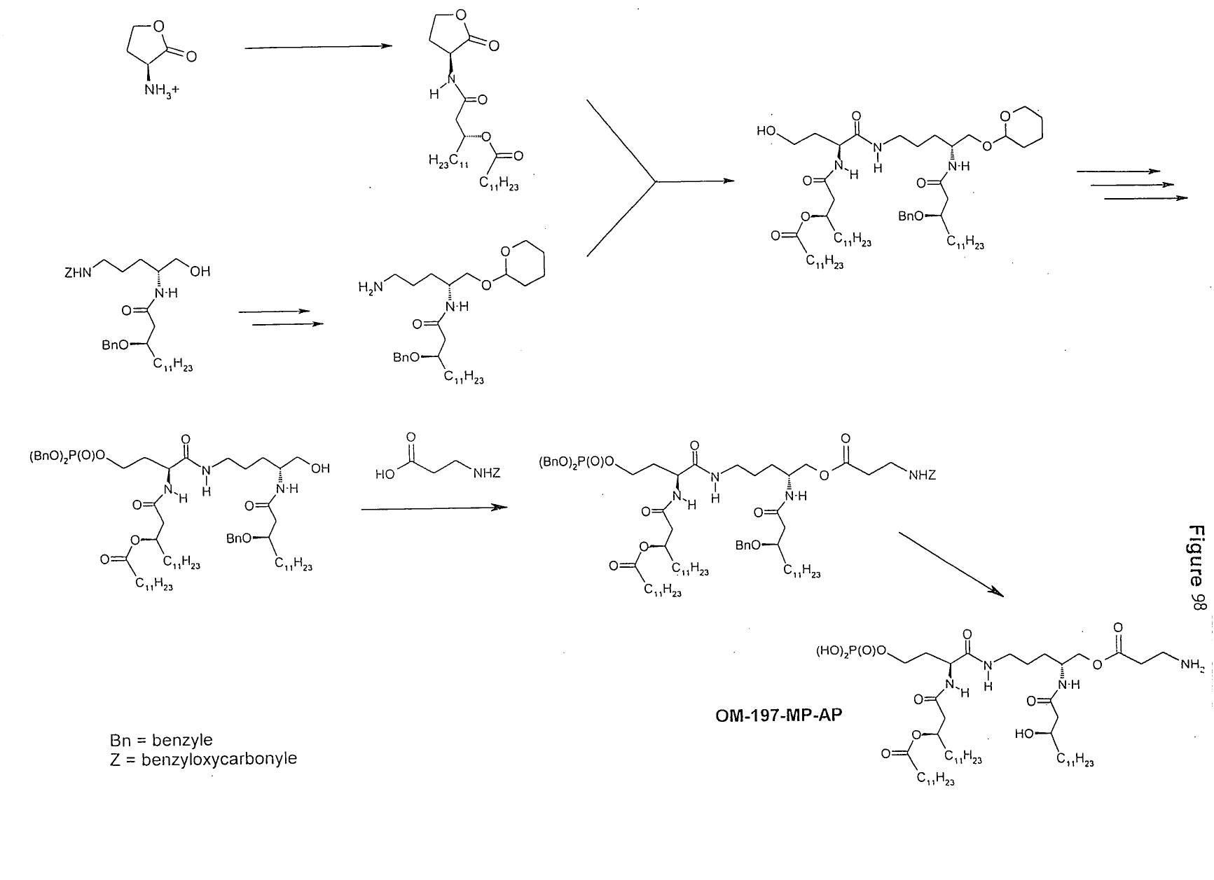

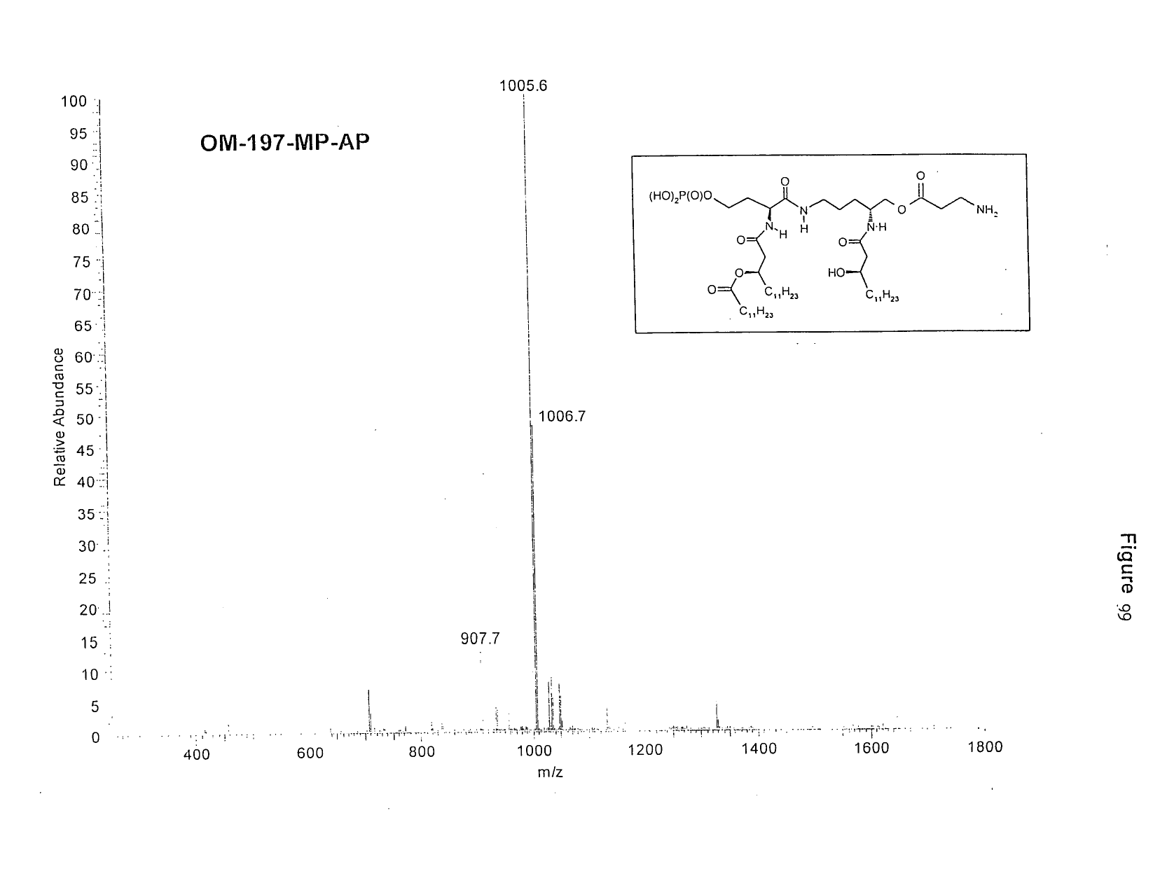

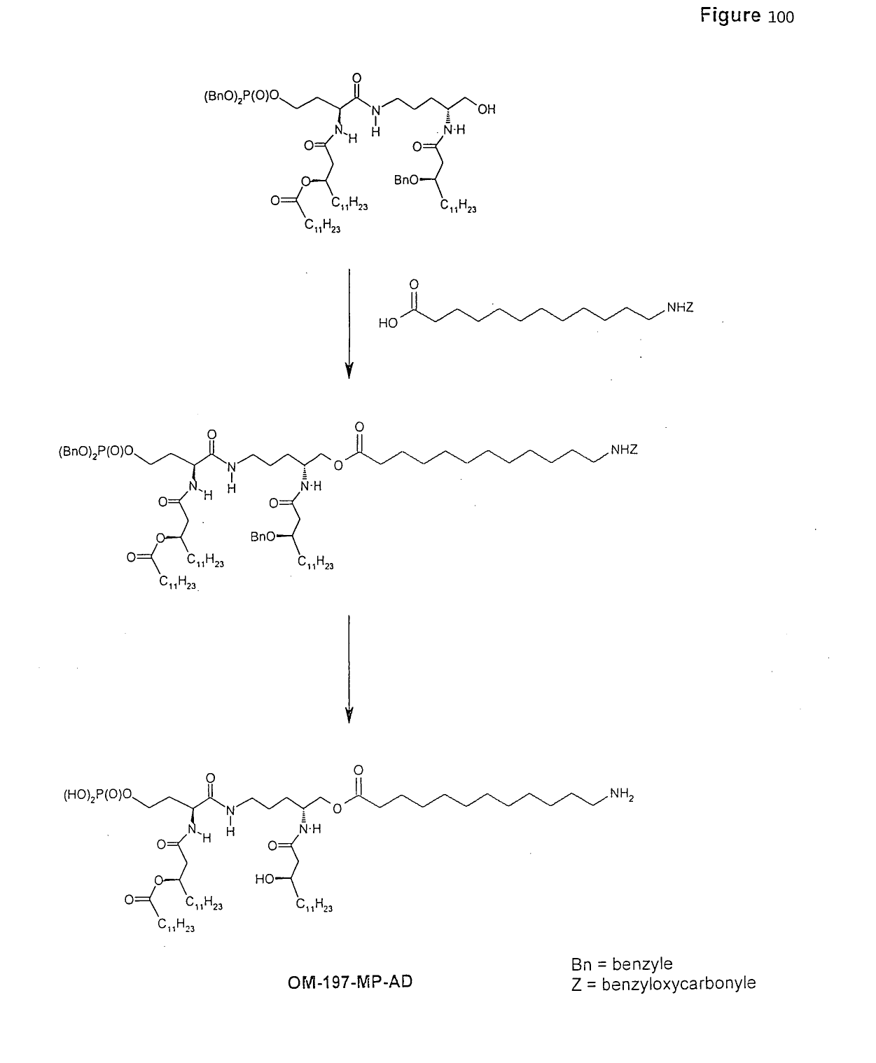

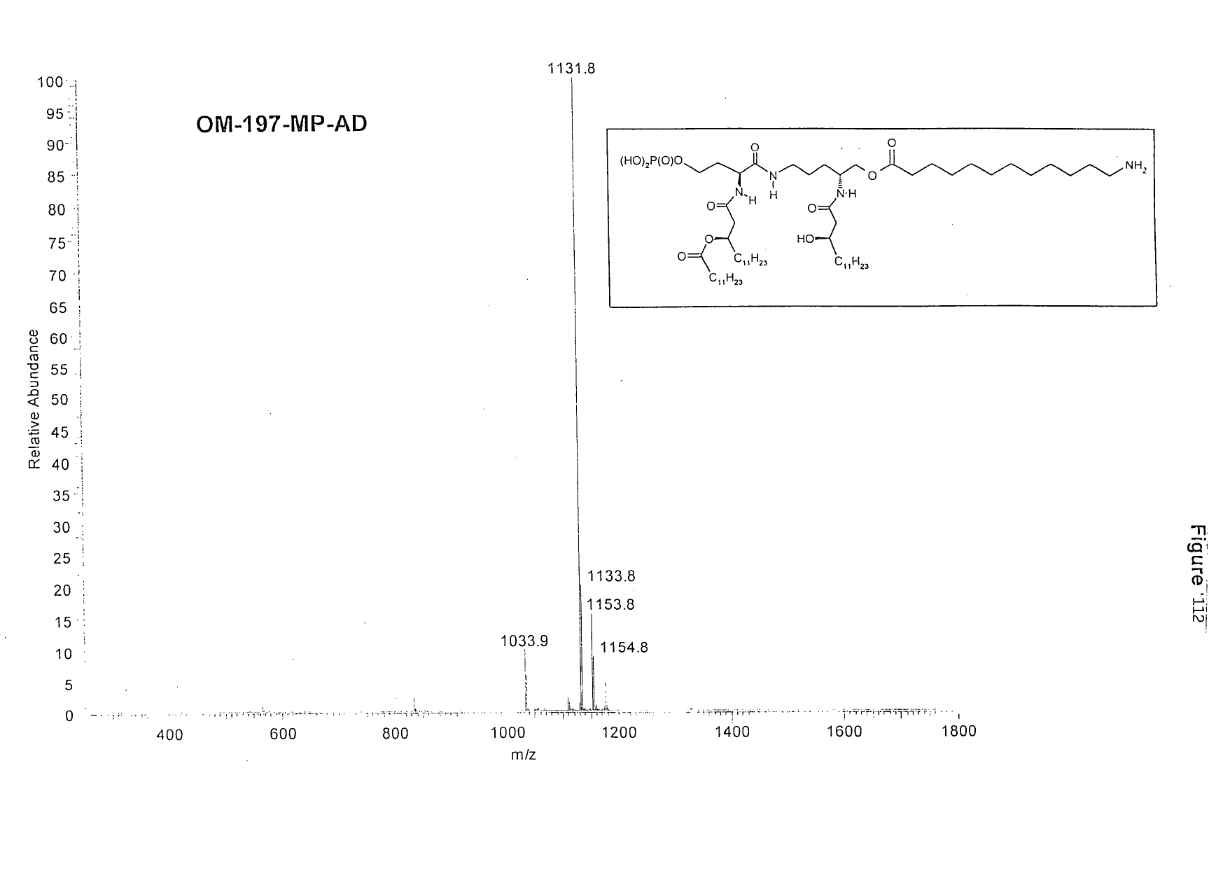

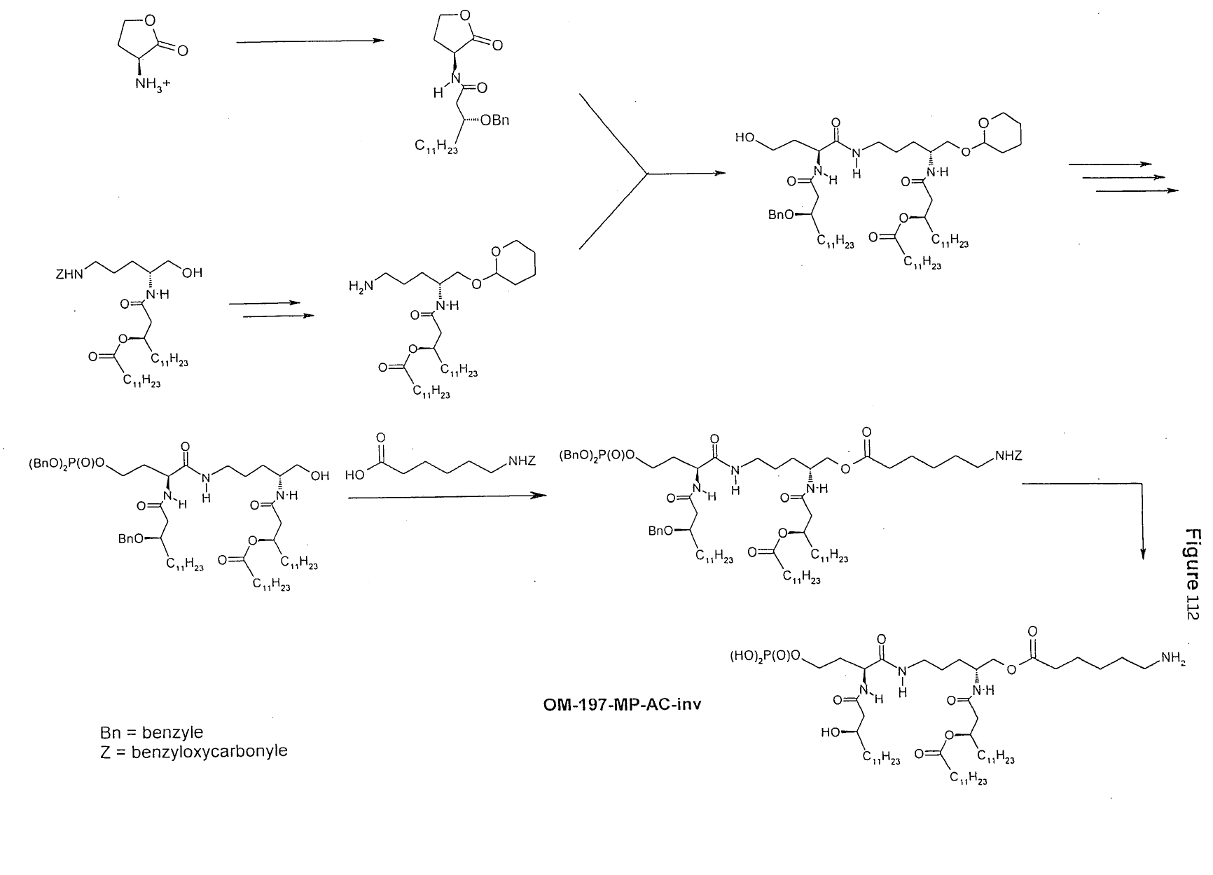

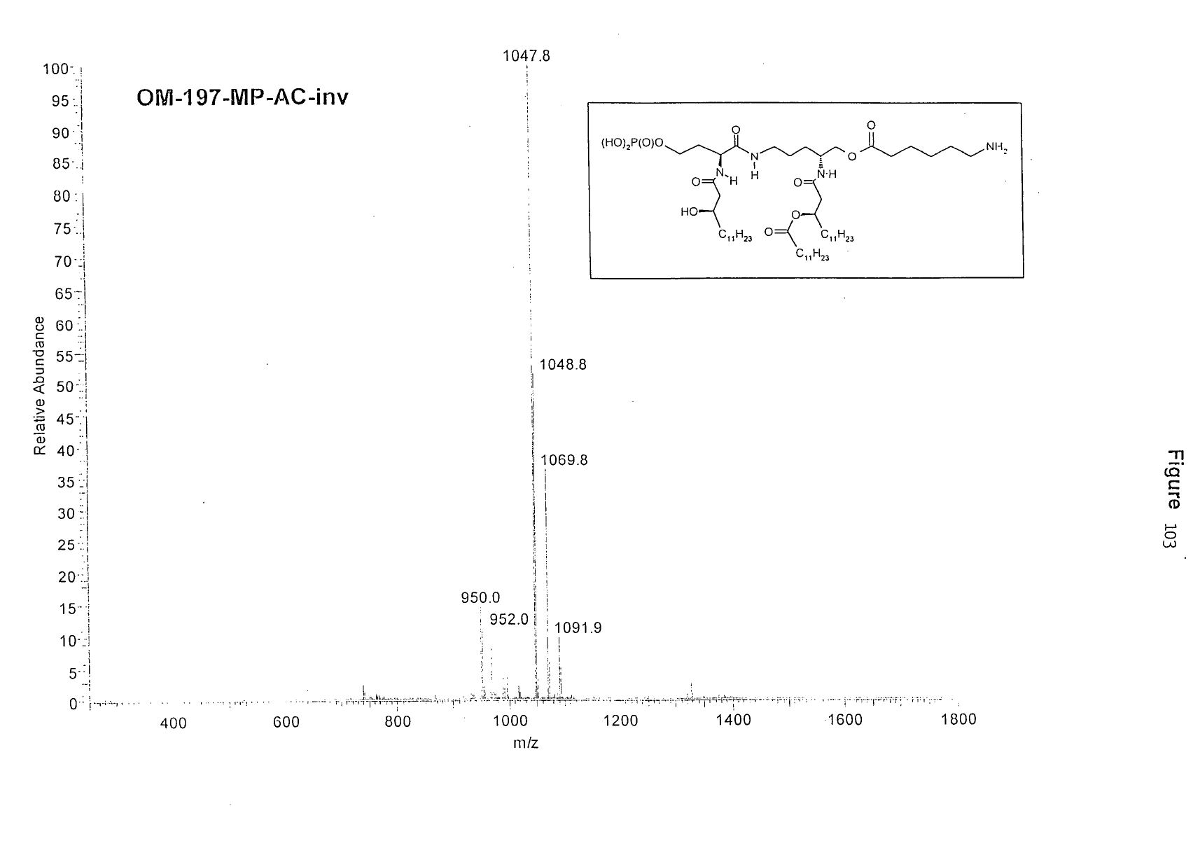

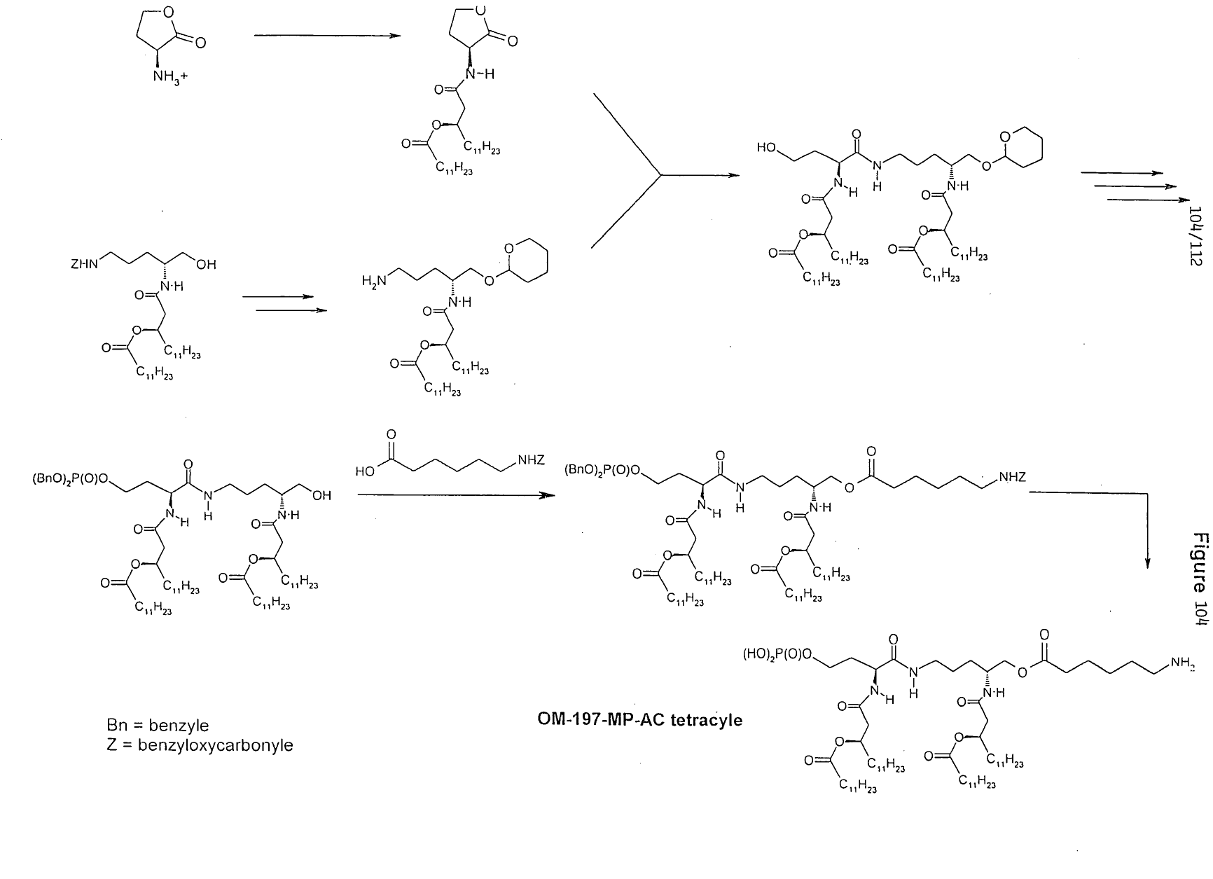

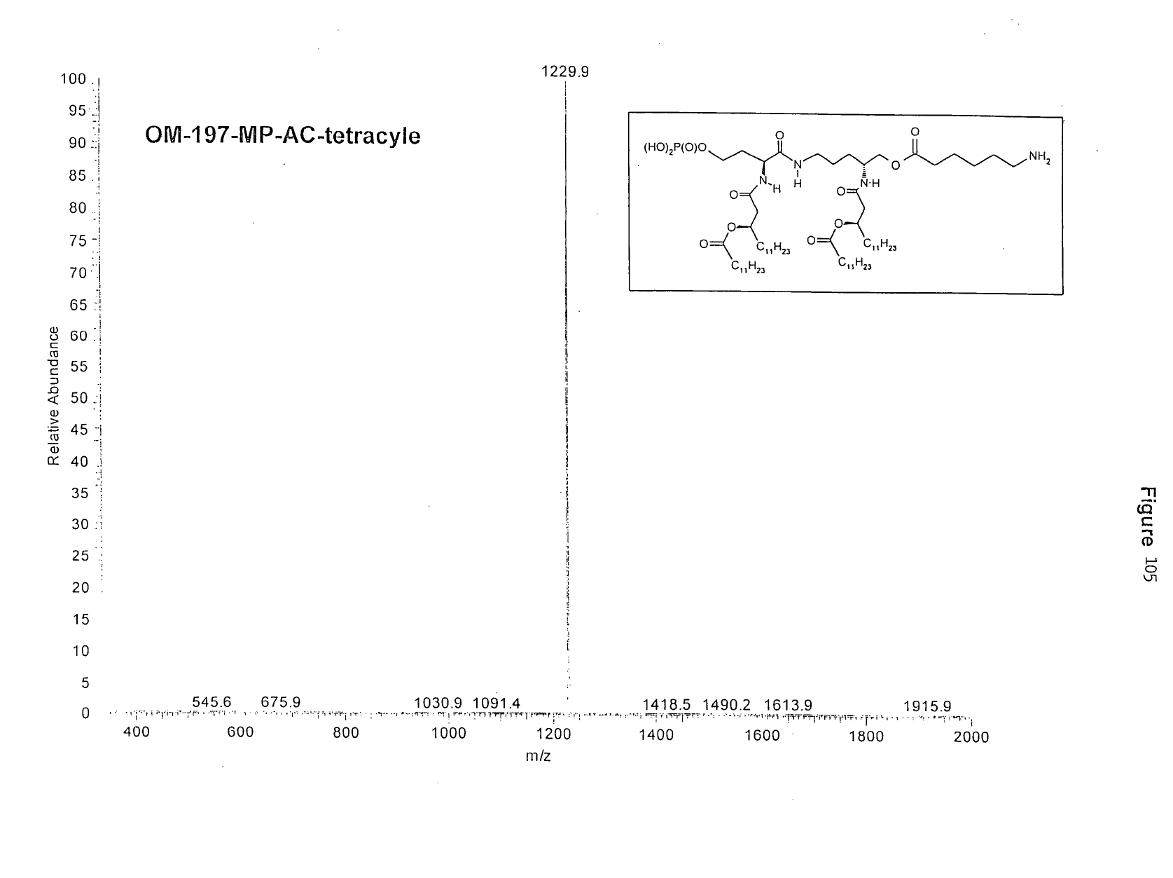

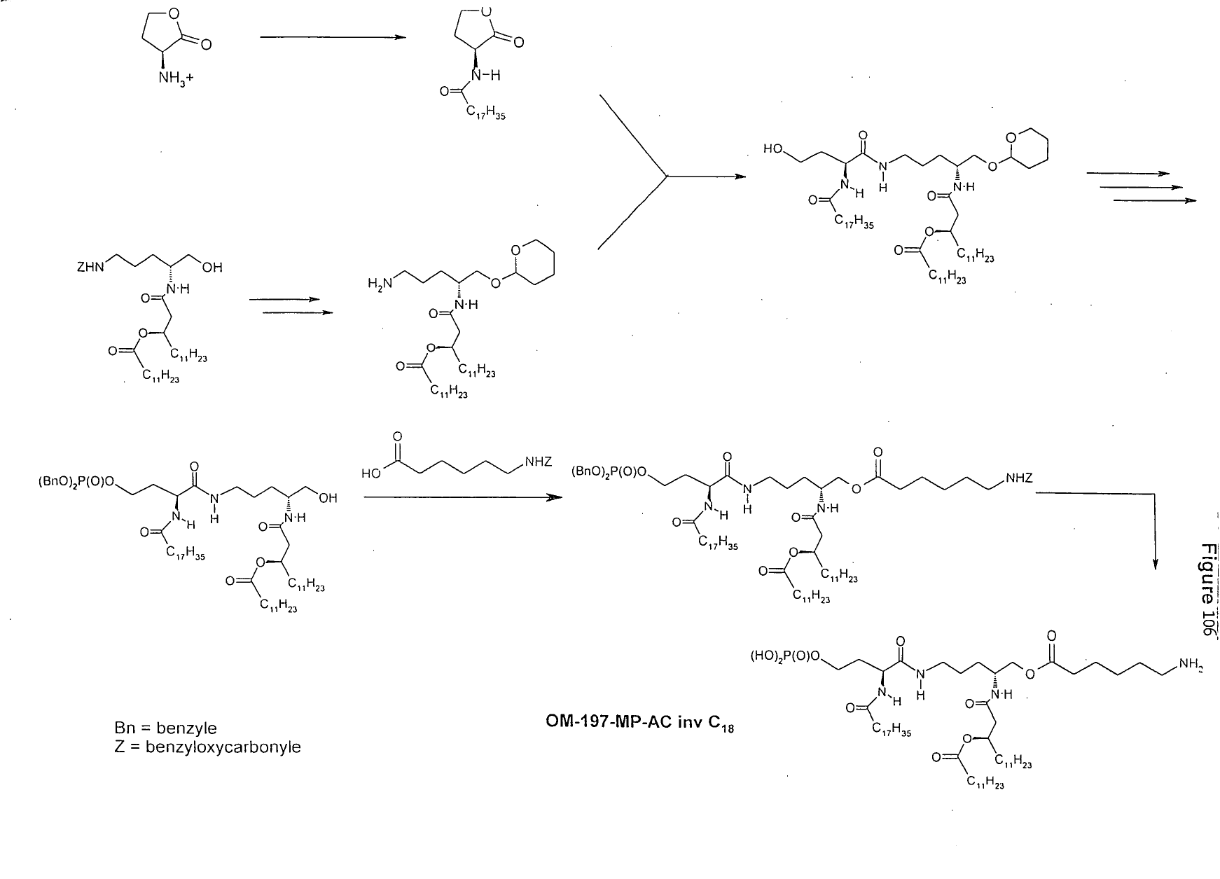

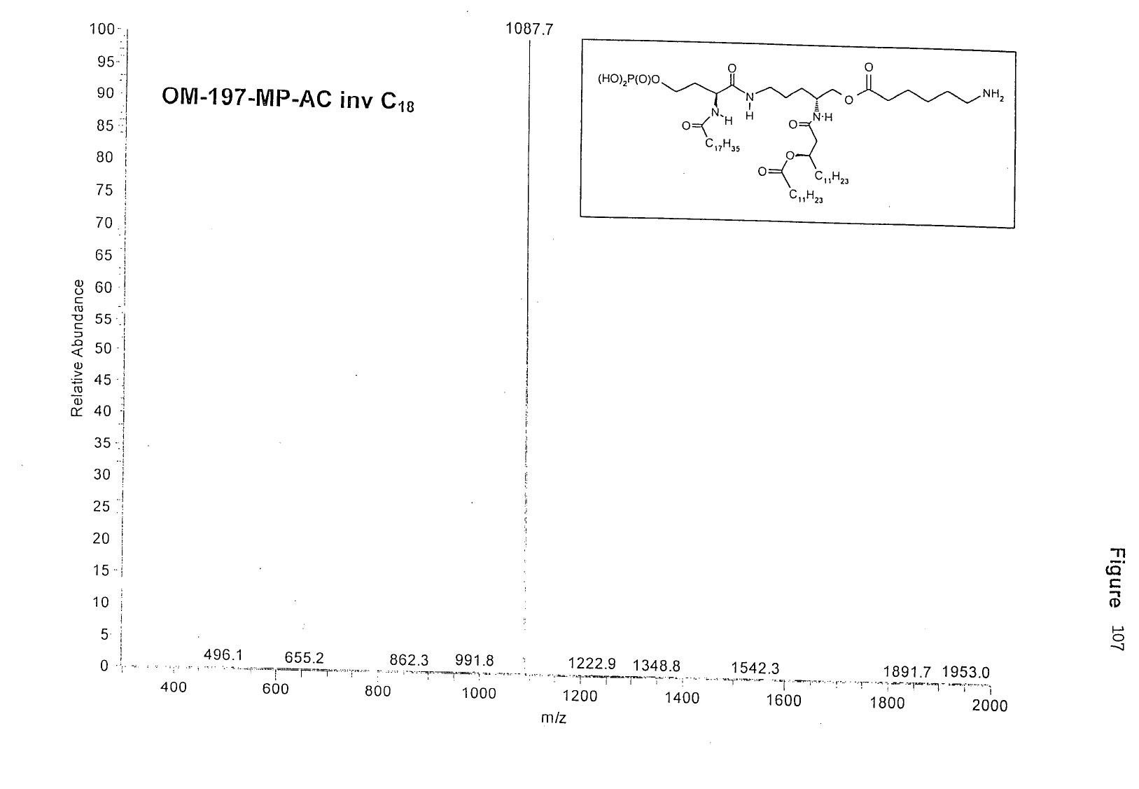

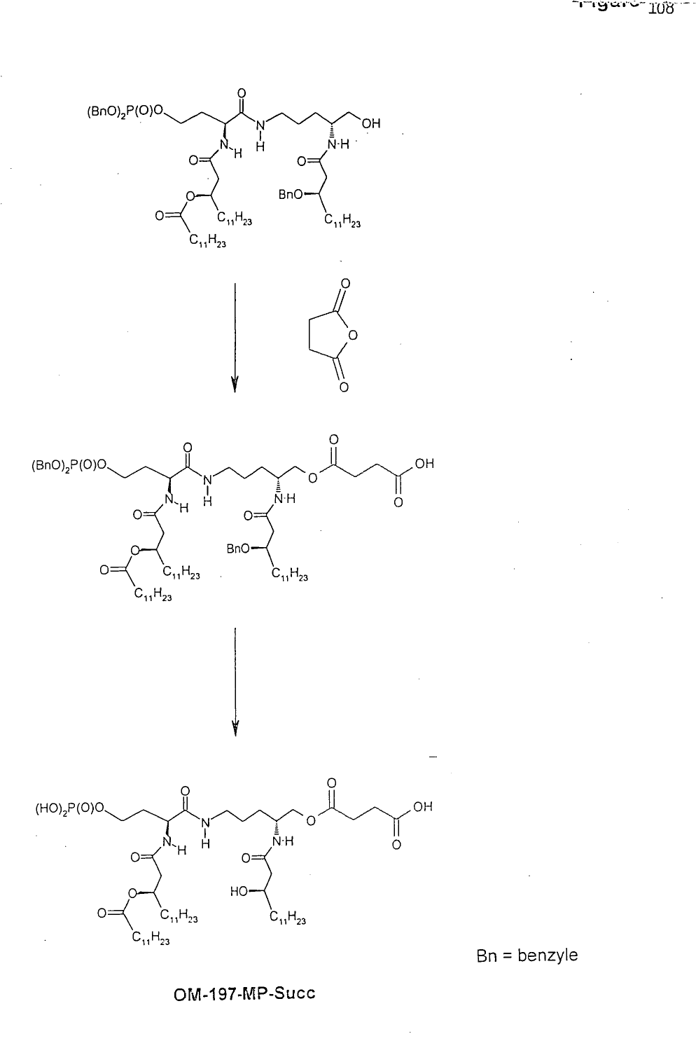

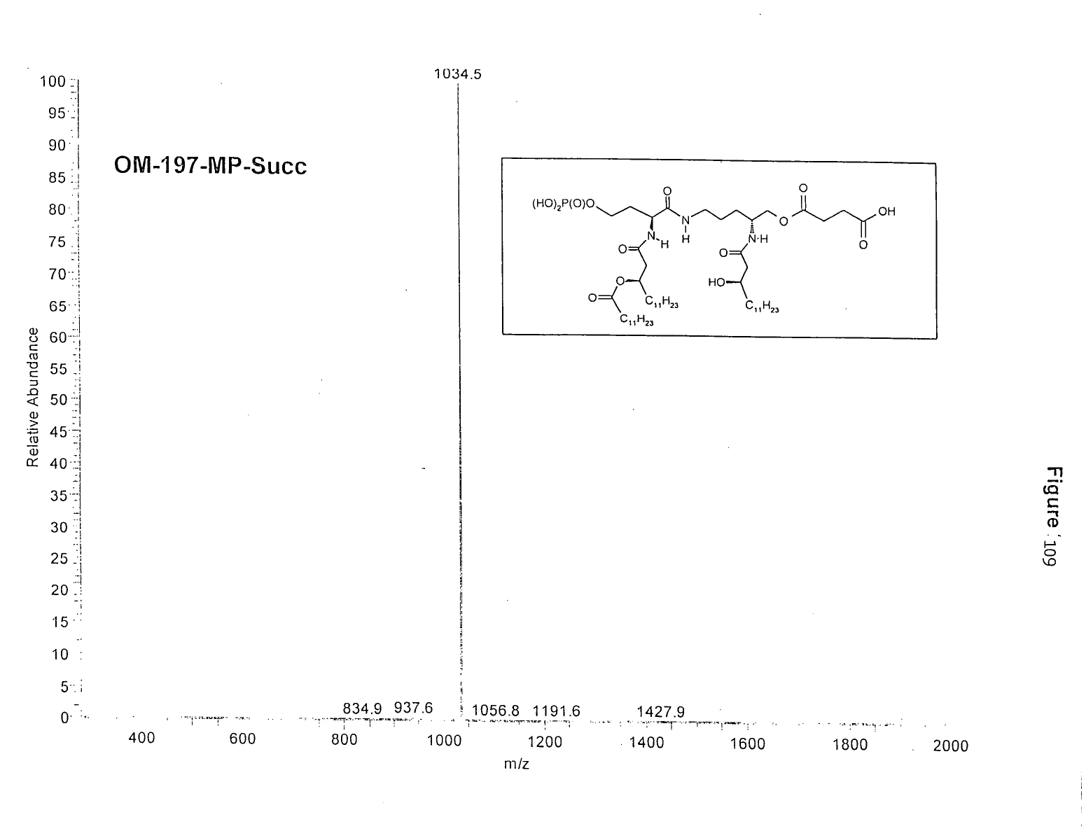

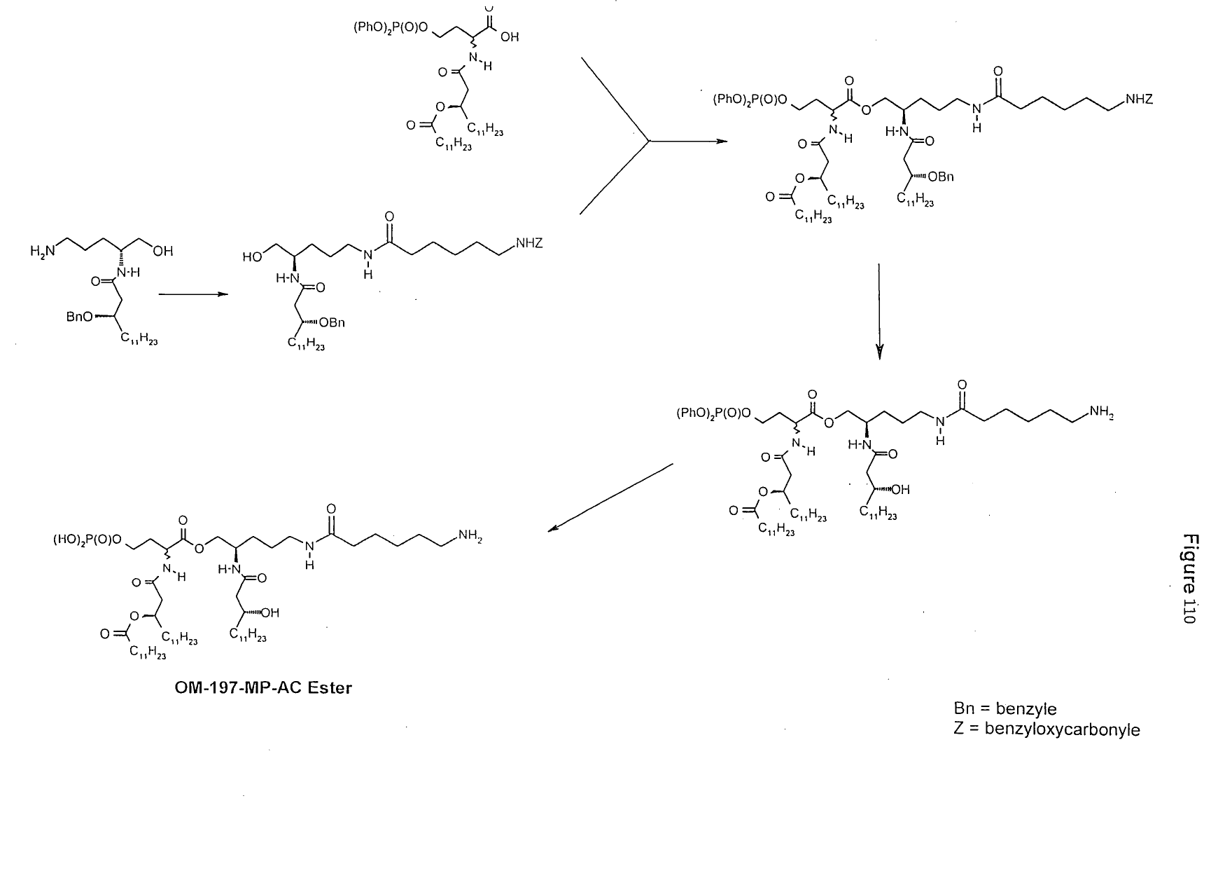

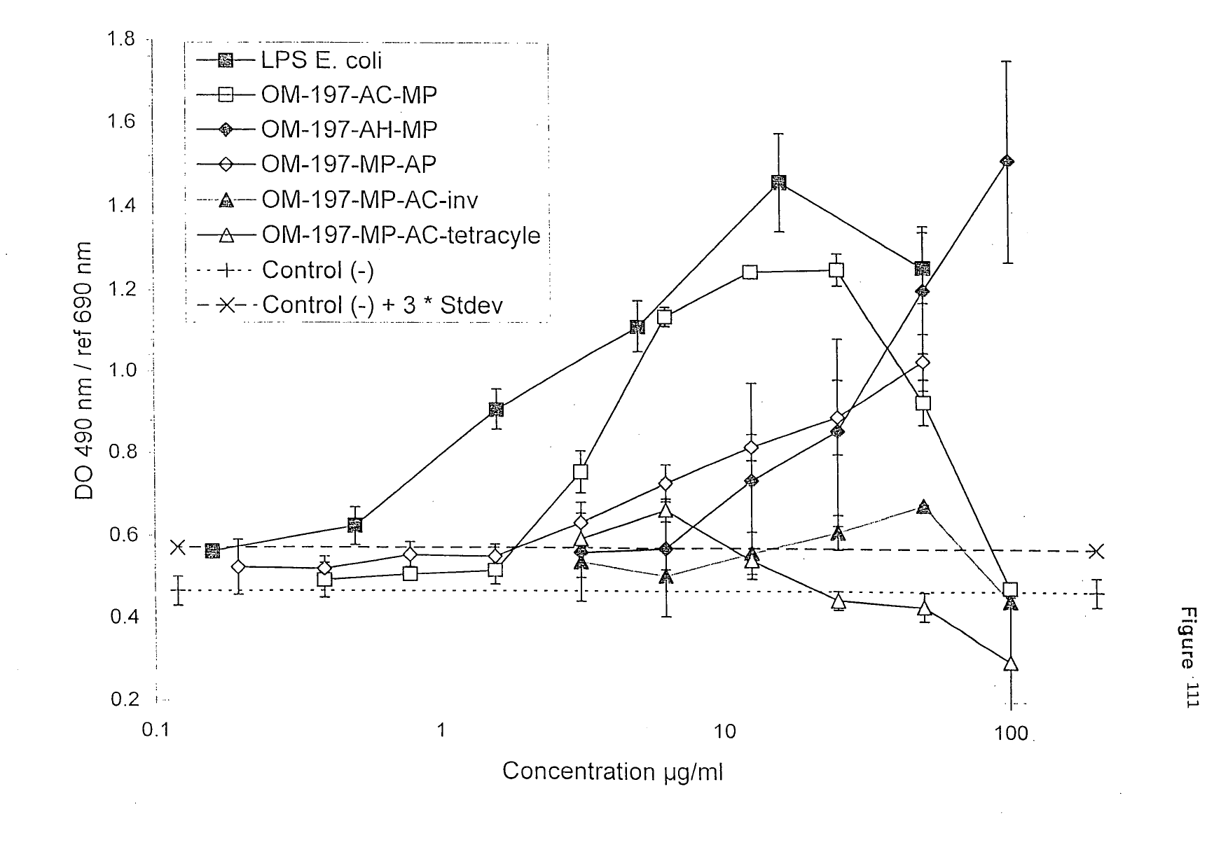

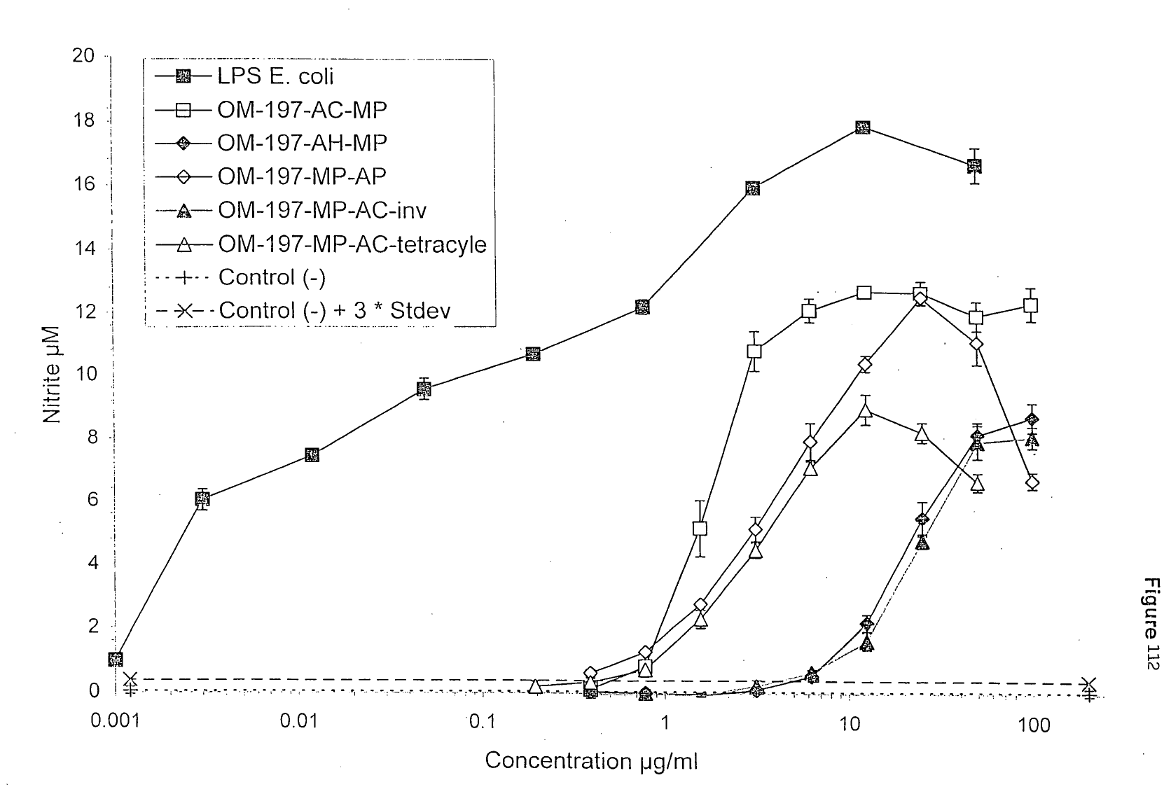

The following non limiting examples are intended to further illustrate the invention. They are outlined in FIGS. 1 to 86.

### EXAMPLES

### 1^{st} Series of Examples

### Preparation of Synthesis Intermediates

### Example 1.1

### 1-(diphenyloxyphosphoryloxy)-3-[(R)-dodecanoyloxytetra-decanoyl-amino]-4-oxo-5-aza-9-[(R)-3-benzyloxytetradecanoylamino]-decan-10-ol (FIG. 1).

1.1.1. 4-(diphenyloxvphosphoryloxy)-2-[(R)-3-dodecanoloxy-tetra-decanoyl-amino]butanoic Acid

a. N-Terbutyloxycarbonyl-DL-homoserine

To a solution of homoserine bromohydrate (2 g; 16.78 mmol.) in H₂O (20 ml), addition was made in succession of a 1 M NaOH solution (16.78 ml) and cesium carbonate (3.01 g ; 9.23 mmol.). After stirring for 5 minutes, the solution was cooled in an ice/water bath. Dioxane (60 ml) and t-butyl pyrocarbonate were then added. The reaction mixture was kept under stirring in an ice-cold water bath for 1 hour and thereafter at room temperature for 5 hours. The solvent was subsequently evaporated under vacuum and the dry residue was directly used in the next step.

b. Benzyl N-t-butyloxycarbonyl-DL-homoserinate

To the residue of the previous step, dimethylformamide (20 ml) was added and the solvent was evaporated to dryness. Then, to the reaction mixture, dimethylformamide (60 ml) and benzyl bromide (4.5 ml; 20.13 mmol) were added. At this point, a white precipitate formed. The mixture was kept under stirring for 16 hours. The solvent was then driven away under vacuum and the residue was extracted with ethyl acetate (2×20 ml). The organic layer was successively washed with H₂O (20 ml) and with brine (20 ml). After drying over MgSO₄, the solvent was evaporated and the residue was used as such in the next step.

c. Benzyl N-t-butyloxycarbonyl-O-(diphenyloxyphosphoryl)-DL-homoserinate

To the dried residue of the previous step dissolved in CH₂Cl₂ (60 ml), 4-N, N-dimethylaminopyridine (DMAP) (4,11 g; 33.56 mmol) was added. The reaction mixture was stirred for 10 minutes, and pyridine (12 ml) and diphenylchlorophosphate (6.95 ml; 33.56 mmol) were then added. The solution was stirred at room temperature for 18 hours and then washed successively with 1N HCl (5×20 ml), H₂O (30 ml) and brine (30 ml). The organic layer was dried over MgSO₄ and the solvent was driven away under vacuum. By performing flash chromatography purification on a silica gel (elution with 4/1 hexane/ethyl acetate mixture), the phosphorylated product (7.49 g; 82.4%) was recovered as a cristalline solid. Melting point: 63.5-64.0° C.

d. Benzyl O-(diphenyloxyphosphoryl)-DL-homoserinate, Trifluoroacetic Salt

The phosphorylated product of the previous step (7.88 g; 15.4 mmol) dissolved in trifluoroacetic acid (15 ml) was kept under stirring at room temperature for 2.5 hours. The solvent was then driven away under high vacuum and a purification step by flash chromatrography on a silica gel (10/1 MeOH/CH₂Cl₂ eluent) was conducted to recover the deprotected amine trifluoroacetic salt (7.17 g; 88.9%) as a cristalline solid. m.p.=73.0−73.5° C.

e. Benzyl 2-[(R)-3-Dodecanoyloxytetradecanoylamino]-4-(diphenyloxyphosphoryloxy)butanoate

(R)-3-dodecanoyloxytetradecanoic acid (4.284 g; 10.07 mmol, 1 eq.) prepared acccording to the method disclosed in [Bull. Chem. Soc. Jpn., 60 (1987), 2205-2214], was dissolved in tetrahydrofurane THF (30 ml) and the solution was cooled down to −15° C. N-methylmorpholin (1,108 ml; 10.07 mmol, 1 eq.) and isobutyl chloroformate (1.31 ml; 10.07 mmol; 1 eq.) were then added. The reaction mixture was kept under stirring for 30 minutes at −15° C. To the reaction mixture, there was next added benzyl O-(diphenyloxyphosphoryl)-DL-homoserinate (5.724 g; 10.07 mmol; 1 eq.) in a THF/Et₃N mixture (30 ml/5 ml). After stirring for 18 hours at room temperature, the solvent was driven away under vacuum. The residue was diluted with H₂O (20 ml) and then extracted with ethyl acetate (2×30 ml). The organic layers were pooled, washed in succession with water (20 ml) and brine (20 ml) and dried over MgSO₄ before evaporating the solvent. By running flash chromatography purification on a silica gel (2/1 hexane/ethyl acetate eluent), the required benzyl ester was recovered (7.455 g ; 87%) as a cristalline solid. m.p.=31.0°−32.1° C., ¹H-NMR (CDCl₃, 250 MHz), δ in ppm:7.4-7.1 (m, 15H), 6.90 (2d, 1H, ³J=7.6 Hz, NH), 5.3-5.1 (m, 3H), 4.7 (m, 1H), 4.35 (m, 2H), 2.45 (m, 2H), 2.4-2.1 (m, 4H), 1.6 (m, 4H), 1.4-1.1 (m, 34H), 0.9 (t, 6H). ¹³C-NMR (CDCl₃, 63 MHz), δ in ppm:173.01, 171.08, 169.66, 150.18, (d, ²J_{P,C}=7.1 Hz), 135.01, 129.60, 128.33, 128.14, 127.96, 125.21, 119.80 (d, ³J_{P,C}=5.0 Hz), 70.69, 67.05, 65.19 (d, ²J_{P, C}=5.6 Hz), 49.13, 40.97, 40.77 (2 diast.), 34.20, 33.98, 33.82, 31.70, 29.42, 29.34, 29.14, 28.94, 25.01, 24.77, 22.47, 13.91.

f. 4-(diphenyloxyphosphoryloxy)-2-[(R)-3-dodecanoyloxytetradecanoylamino]-butanoic Acid

A solution of the benzyl ester obtained in the previous step (2.23 g; 2.6 mmol) in HPLC-grade methanol MeOH (300 ml) in a three neck-round flask was hydrogenated in presence of carbon-10% palladium (1 g) at room temperature and under atmospheric pressure hydrogen for 1 hour. The catalyst was then filtered off and the filtrate was concentrated to obtain a colorless liquor. This product was homogeneous as assessed by thin layer chromatography and NMR, and was used directly with no further purification treatment in the coupling step; Rf=0.75 (CH₂Cl₂—MeOH—Et₃N, 10/1/0.5). ¹H-NMR (CDCl₃, 250 MHz), δ in ppm: 7.4-7.1 (m, 10H), 6.85 (2d, 1H, NH), 5.15 (m, 1H), 4.6 (m, 1H), 4.35 (m, 2H), 2.45 (m, 2H), 2.4-2.15 (m, 4H), 1.6 (m, 4H), 1.4-1.1 (m, 34H), 0.9 (t, 6H). ¹³C-NMR (CDCl₃, 63 MHz), δ in ppm: 173.35, 173.30 (2 diast.), 172.75, 170.37, 150.0 (d, ²J_{P,C}=7.5 Hz), 129.55, 125.28, 119.71 (d, ³J_{P,C}=4.4 Hz), 70.78, 65.65, (d, ²J_{P,C}=5.9 Hz), 49.00, 40.77, 40.63 (2 diast.), 34.13, 33.86, 33.76, 31.59, 29.31, 29.25, 29.03, 28.82, 24.88, 24.68, 22.36, 13.76.

1.1.2 (2R)-5-amino-2-[(R)-3-benzyloxytetradecanoylamino]-pentan-1-ol

1. Copper Salt of D-ornithine

To a solution of D-ornithine (5.25 g ; 30 mmol) in 1M NaOH (30 ml), a solution of cupric sulfate pentahydrate (3.814 g; 15.3 mmol) in water (50 ml) was added. After stirring for 2 hours at room temperature, the solvent was evaporated to dryness. Methanol (60 ml) was added to the residue to form a purple-colored solid which was separated, washed in succession with dioxane and methanol to be directly used in the next step.

b. Copper (2R)-2-Amino-5-(benzyloxycarbonylamino) pentanoate

The purple-colored solid was dissolved in 1N NaOH (40 ml) and dioxane (70 ml), The reaction mixture was cooled in an ice-cold water bath and benzyl chloroformate (5.14 ml; 36 mmol) was then added. After stirring in an ice-cold water bath for 3 hours and thereafter at room temperature for 15 hours, the purple-colored precipitate was recovered by filtration and sequentially washed with 95% EtOH (40 ml), H₂O (50 ml) and EtOH (60 ml). The precipitate was dried in an oven (T<45° C., under vacuum); the yield of the two-step process was 8.27 g, i.e. 93% of the predicted yield. (Reference: Organic Preparations and Procedures international, 23:(1992)191-194).

c. (2R)-5-(benzyloxycarbonylamino)-2-(terbutyloxycarbonylamino)pentanoic Acid

The copper salt obtained in the previous step was dissolved in 2N HCl (400 ml) and ethylenediaminetetraacetic acid (EDTA) was added (8.15 g, 27.8 mmol.) thereto. The mixture was stirred for 2.5 hours, and neutralized to pH 7 by adding NaOH 5N (about 160 ml). A white precipitate was formed at this point. The mixture was then stirred for 2.5 hours in an ice-cold water bath. The precipitate was filtered, washed with cold water until washing effluents were colorless, then dried in an oven below 60° C. This solid was dissolved in 1N NaOH (156 ml) and the solution was cooled down into an ice-cold water bath. To this solution, t-butyl pyrocarbonate (7.7 g ; 35.2 mmol.) in dioxane (160 ml) was added. The mixture was stirred at 0° C. for 45 minutes and thereafter for 16 hours at room temperature. The organic solvent was evaporated and ethyl acetate (70 ml) was added to dissolve the residue. The aqueous layer was then acidified by adding 2N HCl down to pH 3, washed with AcOEt (100 ml). The organic layers were combined and washed with H₂O (30 ml) and with brine (30 ml), then evaporated under vacuum. By conducting flash chromatography purification on a silica gel (20/1 CH₂Cl₂/MeOH eluent), the required product was recovered as a colorless oil (Yield:8,42 g. in two steps i.e. 76,6% of the predicted yield) (Rf=0,19, 20/1 CH₂Cl₂/MeOH)

d. (2R)-5-(Benzyloxycarbonylamino)-2-(terbutyloxycarbonylamino)pentan-1ol

To a cold solution (−15° C.) of the diamino pentanoic acid derivative obtained previously (5.45 g ; 14.8 mmol) in THF (60 ml), N-methylmorpholin (1.654 ml; 14.8 mmol) and isobutyl chloroformate (IBCF) (9.6 ml; 14.8 mmol) were added. The reaction mixture was stirred at −15° C. for 1 minute followed by addition of sodium borohydride (5.1 g ; 44.6 mmol) in 10 ml of water. After further stirring for 10 minutes at −15° C., H₂O (400 ml) was added to the mixture to stop the reaction. The solution was subsequently washed with ethyl acetate AcOEt (100 ml×2). The organic layers were combined and washed with H₂O (50 ml) and with brine (60 ml) then dried over MgSO₄. The solvent was removed and the residue recristallized from an ethyl acetate/hexane mixture to provide the expected cristalline product (4.94 g; 94.9% yield) m.p.=47.5-48° C.

e. (2R)-5-(benzyloxycarbonylamino-2-amino-pentan-1-ol, Trifluoroacetic Salt

A solution of (2R)-5-(benzyloxycarbonylamino)-2-(terbutyloxy-carbonylamino)-pentan-1-ol (6.32 g; 18 mmol) as obtained above in trifluoroacetic acid (25 ml) was prepared then subjected to stirring for 2.5 hours at room temperature. The solvent was thereafter evaporated and the residue purified by flash chromatrography on a silica gel (10/1 MeOH/CH₂Cl₂ eluent) to recover the trifluoroacetic salt as an oil (5,45 g; 82.7%). The hydrochloride compound melts at 133.0°-134.3° C. (recristallization from methanol).

f. (2R)-5-(Benzyloxycarbonylamino)-2-[(R)-3-benzyloxy-tetra-decanoylamino]pentan-1-ol

To a solution of (R)-3-benzyloxytetradecanoic acid (5.27 g; 15.8 mmol) [Bull. Chem. Soc. Jpn., 60:(1987), 2197-2204] in THF (30 ml) which had been cooled down to −15° C., addition was made of N-methylmorpholin (1.89 ml 15.8 mmol) and of isobutyl chloroformate (2.21 ml, 15.8 mmol). After stirring the reaction mixture at −15° C. for 30 minutes, there was added a trifluoroacetate salt solution as obtained above (15.25 g; 14,4 mmol.) in a THF/Et₃N mixture (30 ml, 1.44 ml) . Stirring was continued at room temperature for 16 hours and then the reaction mixture was diluted with H₂O (30 ml) and AcOEt (60 ml). The organic layer was separated and the aqueous layer was washed with ethyl acetate AcOEt (60 ml). The organic layers were pooled and washed with H₂O (30 ml) and with brine (30 ml) then dried over MgSO₄ before carrying out solvent evaporation under vacuum. The residue was recristallized from an ethyl acetate/hexane mixture to finally yield the expected product in a cristalline state (5.8 g; 71.2% yield), m.p.=117.5−118° C. Rf=0.32, 3/1 ethyl acetate—petroleum ether. ¹H-NMR (CDCl₃, 250 MHz), δ in ppm:7.4-7.2 (m, 10H), 6.5 (d, 1H, NH), 5.1 (s, 2H), 4.9 (m, 1H, NH), 4.5 (2d, AB, 2H), 3.8 (m, 2H), 3.5 (m, 2H), 3.1 (m, 2H), 2.4 (m, 2H) 1.6-1.4 (m, 6H), 1.4-1.2 (m, 18H), 0.9 (t, 3H). ¹³C-NMR (CDCl₃, 63 MHz), δ in ppm: 172.24, 156.49, 138.06, 136.53, 128.46, 128.04, 127.87, 76.76, 71.39, 66.60, 65.44, 51.54, 41.43, 40.65, 33.76, 31.87, 29.61, 29.30, 28.01, 26.47, 25.05, 22.65, 14.09.

g. (2R)-5-Amino-2-[(R)-3-benzyloxytetradecanoylamino]pentan-1-ol

In a three-neck flask, the catalyst (150 mg of 20% palladium/carbon) was added to the solution of (2R)-5-(benzyloxycarbonylamino)-2-[(R)-3-benzyloxytetradecanoylamino]pentan-1-ol (3.0 g; 5.27 mmol) in a mixture of triethylamine Et₃N (6 ml)/HPLC-grade ethanol EtOH (300 ml). Air was discharged under vacuum then the flask was loaded with hydrogen. The reaction mixture was hydrogenated at room temperature for 2 hours then the catalyst was filtered off and the filtrate was concentrated to provide the desired product as a homogenous white solid. Rf=0.2, 5/1 0/0.5 CH₂Cl₂—MeOH—Et₃N, m.p.=47-48° C.

¹H-NMR (CDCl₃, 250 MHz), δ in ppm:7.4-7.2 (m, 5H), 6.75 (d, 1H, NH)4.5 (2d, AB, 2H), 3.9 (m, 2H), 3.5 (m, 2H), 2.3-2.6 (m, 7H), 1.7-1.2 (m, 24H), 0.9 (t, 3H). ¹³C-NMR (CDCl₃, 63 MHz), δ in ppm:171.86, 138.13, 128.37, 127.87, 127.75, 76.81, 71.50, 64.57, 51.38, 41.51, 41.17, 33.89, 31.82, 29.26, 28.57, 28.03, 25.07, 22.60, 14.04.

1.1.3. 1-(Diphenyloxyphosphoryloxy)-3-[(R)-3-dodcanoyloxy-tetra-decanoylamino]-4-oxo-5-aza-9-[(R)-3-benzyloxytetradecanoylamino]-decan-10-ol

IIDQ (2-isobutoxy-1-isobutoxycarbonyl-1,2-dihydroquinoline) 364 g; 1,2 mmol., 1.2 eq.) was added to a solution of (2RS)-4-(diphenyloxyphosphoryloxy)-2-[(R)-3-dodecanoyloxy-tetra-decanoylamino]-butanoic acid (850 g; 1.0 mmol; 1 eq.) in anhydrous methylene chloride CH₂Cl₂ (20 ml) at room temperature and under argon flow. After stirring for 15 minutes, addition was made of a solution of (2R)-5-amino-2-[(R)-3-benzyloxytetradecanoylamino]pentan-1-ol (757; 1.0 mmol.; 1 eq.) in anhydrous CH₂Cl₂ (10 ml). After stirring for 4 hours, the solution was evaporated to dryness. By running a flash chromatography purification on a silica gel (5/2 CH₂Cl₂/acetone eluent), there was recovered the phosphorylated dipeptide-like compound (620 mg; 53%) as an amorphous solid. (Rf=0.49, in dichloromethane—methanol—triethylamine, 10/1/0.5). ¹H-NMR (CDCl₃, 250 MHz), δ in ppm:7.40-7.15 (m, 15H), 7.00 (m, 1H), 6.90 and 6.80 (2d, 2 diast., 1H), 6.65 (d, 1H) (3×NH), 5.15 (m, 1H), 4.50 (m, 3H), 4.30 (m, 2H), 3.85 (m, 2H), 3.45 (m, 2H), 3.15 (m, 2H), 2.41-2.14 (m, 8H), 1.6-1.4 (m, 8H), 1.4-1.1 (m, 54H), 0.9 (t, 9H, 3CH₃). ¹³C-NMR (CDCl₃, 63 MHz), δ in ppm:173.11, 171.68, 170.52 (2 diast.), 169.94 (2 diast), 150.0 (d, ²J_{P,C}=7.2 Hz), 138.0 (2 diast.), 129.58, 127.99, 127.49, 127.26, 125.24, 119.73 (t, ³J_{P,C}=5.0 Hz), 76.48, 71.12, 70.71, 65.86 (broad spin), 64.22, 50.96, 49.71 (broad spin), 41.46, 41.05, 39.07, 34.13, 34.00, 32.70, 31.61, 29.34, 29.06, 28.87, 27.98, 25.25, 24.92, 24.72, 22.38, 13.80.

### Example 1.2

### N-[(R)-3-dodecanoyloxytetradecanoyl]-D-aspartic acid,α-N-{(4R)-5-hydroxy-4-[(R)-3-hydroxytetradecanoylamino]-pentyl}amide (=OM-197-MC) (FIG. 2)

1.2.1 N-[(R)-3-dodecanoyloxytetradecanoyl]-D-aspartic Acid, β-benzyl Ester

To a solution of (R)-3-dodecanoyloxytetradecanoic acid (3,35 g; 7.85 mmol.) in anhydrous THF (25 ml) at −15° C. and under argon flow, there were added in succession N-methylmorpholin (0.86 ml; 7.85 mmol; 1 eq.) and isobutyl chloroformate (1.02 ml; 7.85 mmol.; 1 eq.). Rapid formation of a N-methylmorpholin hydrochloride precipitate was observed. After stirring for 30 minutes at −15° C., a commercially available solution of H-D-Asp(OBn)-OH (Senn Chemicals AG, Switzerland-Dielsdorf) (1.75 g; 7.85 mmol. ; 1 eq.) in a 3.5/1 CH₃CN/H₂O mixture (85 ml) containing Et₃N (3.7 ml) was added. The reaction mixture was then stirred overnight at room temperature. The organic solvent was then evaporated and the aqueous layer was cooled down to 0° C., acidified with a 10% aqueous solution of citric acid down to pH=3 and extracted with AcOEt (2×). The organic layer was dried over MgSO₄, filtered and evaporated. By running a flash chromatography purification on a silica gel (2/1 petroleum ether/AcOEt eluent containing 2% of acetic acid) followed by coevaporation of toluene, there was recovered [(R)-3-dodecanoyloxytetradecanoylamino]-D-aspartic acid β-benzyl ester (4.00 g; 81%) as a white cristalline solid (Rf=0.42 in 1/1 petroleum ether/EtOAc containing 2% of acetic acid; phosphomolybdinium compound and U.V. color development agent). m.p.=67−69° C.

1.2.2. N-[(R)-3-dodecanoyloxytetradecanoyl]-D-aspartic Acid, α-N-{(4R)-5-hydroxy-4-[(R)-3-benzyloxytetradecanoylamino pentyl}amide β-benzyl Ester (ASM-1)

To a solution of β-benzyl ester as obtained above (363 mg; 0.57 mmol.) and (2R)-5-amino-2-[(R)-3-benzyloxy-tetradecanoylamino]pentan-1-ol (Section 1.1.2) (250 mg; 0.57 mmol.; 1.0 eq) in anhydrous CH₂Cl₂ (6 ml) at 0° C. (ice/water bath), addition was made in succession under argon flow of commercially available HOAt (1-hydroxy-7-azabenzotriazol) (94 mg, 0.69 mmol., 1.2 eq.) and commercially available N, N′-diisopropylcarbodiimide (109 μl, 0.69 mmol., 1.2 eq.). The reaction mixture was stirred for 1 hour at 0° C. and thereafter overnight at room temperature. The reaction mixture was subsequently washed with water, a 1N HCl solution, and a saturated solution of NaHCO₃ followed by layer separation. The organic layer was dried on MgSO₄, filtered and evaporated. By running a flash chromatography purification on a silica gel (3/1 CH₂Cl₂/acetone eluent), there was recovered the coupling reaction product (436 mg; 72%) as a white cristalline solid (Rf=0.27 in 5/1 CH₂Cl₂-acetone; phosphomolybdinium compound and U.V. color development agent ). m.p.=106-108° C.; ¹³C-NMR (62.89 MHz, CDCl₃), δ in ppm: 173.66; 172.09; 171.73; 170.33; 170.12; 138.23; 135.28; 128.53; 128.37; 128.13; 127.81; 127.71; 125.81; 76.71; 71.40; 71.16; 66.77; 65.01; 51.36; 49.39; 41.66; 39.25; 34.40; 33.98; 31.85; 29.58; 29.47; 29.29; 29.11; 28.00; 25.57; 25.17; 25.08; 24.94; 22.62; 14.05.

1.2.3. N-[(R)-3-dodecanoyloxytetradecanoylamino]-D-aspartic Acid, α-N-{(4R)-5-hydroxy-4-[(R)-3-benzyloxytetradecanoylamino pentyl}amide (=OM-1 97-MC)

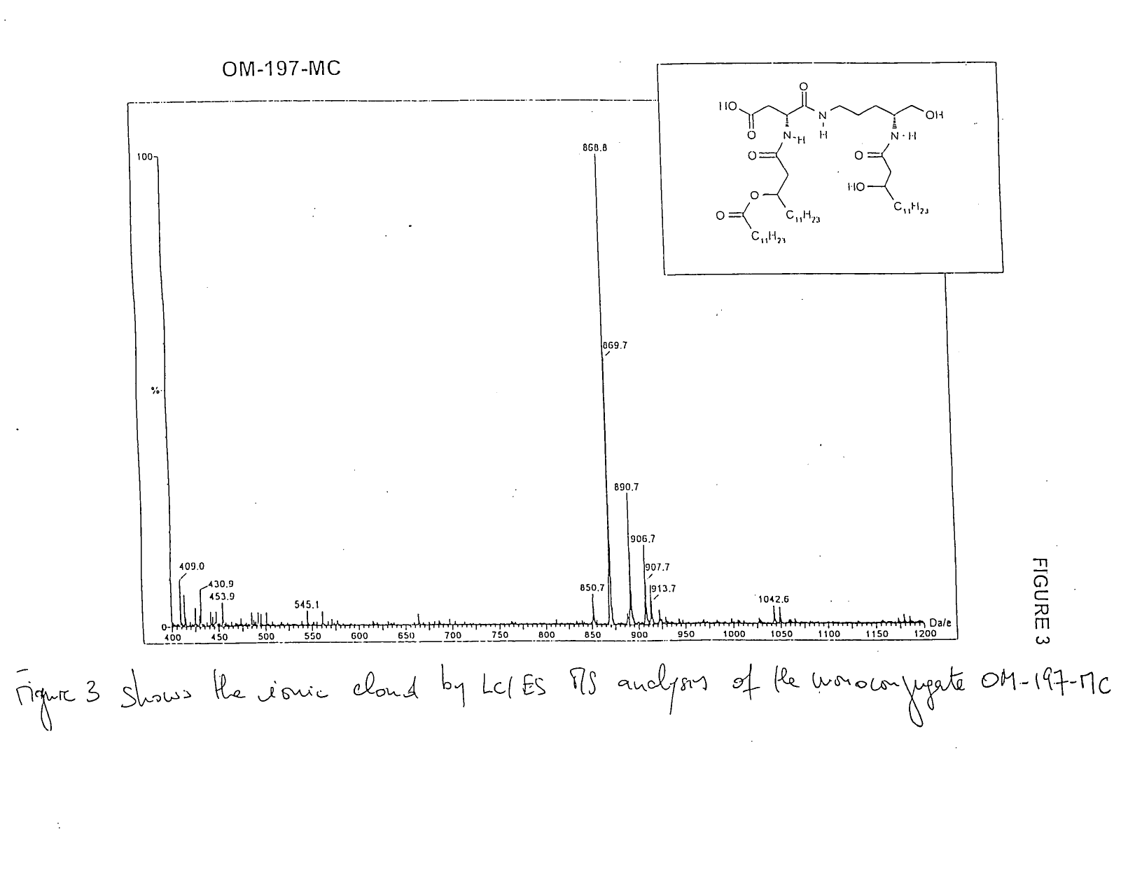

A solution of N-[(R)-3-dodecanoyloxytetradecanoyl]-D-aspartic acid β-benzyl ester, α-N-{(4R)-5-hydroxy-4-[(R)-3-benzyloxytetradecanoylamino pentyl}amide (417 mg; 0.40 mmol.) in a 1/1 MeOH/EtOAc mixture (36 ml) was hydrogenated in presence of 10% Pd/carbon (20 mg) at room temperature and under atmospheric pressure hydrogen for 3 hours. The catalyst was filtered off, washed with a 4/1 CH₂Cl₂/MeOH mixture (50 ml) and the filtrate was evaporated to dryness by suction from a vacuum pump to yield the free acid (345 mg ; 100%) as a white cristalline solid (Rf=0.30 in 9/1 CH₂Cl₂/MeOH containing 0.5% of acetic acid ; phosphomolybdinium compound color developer). m.p.135-137° C. ES/MS: m/z ratio 868.7 [M+H]⁺, 890.7 [M+Na]⁺, 868.7 [M+K]⁺912.7 [M−H+2Na]⁺ (FIG. 3).

1.2.4. N-[(R)-3-dodecanoyloxytetradecanoyl]-L-aspartic Acid, α-N-{(4R)-5-hydroxy-4-[(R)-3-benzyloxytetradecanoylamino Pentyl}amide

The same reaction scheme was followed using commercially available H-L-Asp(Obn)-OH (Fluka, Buchs, Switzerland) to finally obtain an epimer product of L-aspartic series.

### Example 1.3.

### N-[(R)-3-dodecanoyloxytetradecanoyl]-D-aspartic acid, α-N-{(4R)-5-dihydroxyphosphoryloxy-4-[(R)-3-hydroxytetradecanoyl-amino]pentyl}amide (=OM-197-MC-MP) (FIG. 2)

1.3.1. N-[(R)-3-dodecanoyloxytetradecanoyl]-D-aspartic Acid, α-N-{(4R)-5-dibenzyloxyphosphoryloxy-4-[(R)-3-benzyloxytetradecanoylamino Pentyl}amide β-benzyl Ester

To a solution of N-[(R)-3-dodecanoyloxytetradecanoyl]-D-aspartic acid, α-N-{(4R)-5-hydroxy-4-[(R)-3-benzyloxytetradecanoylamino]-pentyl}amide β-benzyl ester as obtained above (300 mg; 0.29 mmol.) and 1H-tetrazol (60 mg; 0.86 mmol.) in anhydrous THF (12 ml) at room temperature and under argon flow, there was added 85% dibenzyl-diethyl phosphoramidite (231 μl; 0.66 mmol.). Rapid formation of white cristals in the reaction medium was observed. After stirring for 30 minutes, the reaction mixture was cooled down to −20° C. then a solution of mCPBA (57-86%; 183 mg; 1.06 mmol.) in CH₂Cl₂ (8 ml) was added. Disappearance of cristals was noted. After stirring for 45 minutes at room temperature, a saturated solution of Na₂S₂O₃ was added then the reaction mixture was stirred again for 10 minutes. The solution was diluted with ether, then the organic layer was separated and washed with a saturated solution of Na₂S₂O₃ (5×), a saturated solution of NaHCO₃ (2×) and a solution of 1M HCl (1×). The organic layer was dried over MgSO₄, filtered and evaporated. By running a flash chromatography purification on a silica gel (5/1 CH₂Cl₂/acetone eluent), there was recovered the phosphortriester (294 mg; 79%) as a white solid (Rf=0.27 in 5/1 CH₂Cl₂-acetone; phosphomolybdinium compound and U.V. color development agent) ¹³C-NMR (62.89 MHz, CDCl₃), δ in ppm:173.66; 171.73; 171.01; 170.60; 170.03; 138.22; 135.50; 135.40; 135.28; 128.40; 128.33; 128.16; 128.08; 127.94; 127.82; 127.76; 127.53; 127.41; 76.43; 71.03; 70.90; 69.28; 66.47; 49.09; 48.37; 41.62; 41.36; 41.24; 39.02; 38.88; 25.05; 24.94; 24.82; 22.48; 13.90.

1.3.2. N-[(R)-3-dodecanoyloxytetradecanoyl]-D-aspartic Acid, α-N-{(4R)-5-dihydroxyphosphoryloxy-4-[(R)-3-hydroxytetradecanoyl-amino]pentyl}amide (=OM-197-MC-MP)

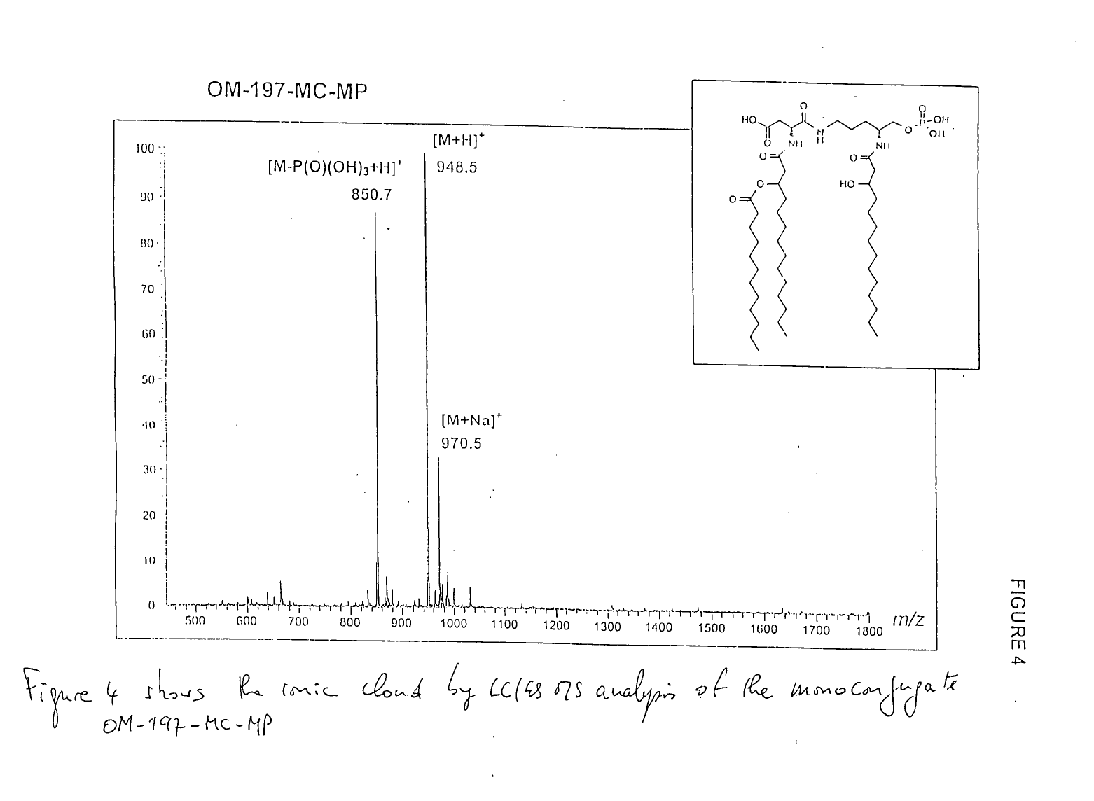

A solution of dibenzylphosphate as prepared above (249 mg; 190 μl) in HPLC-grade EtOH (12 ml) is hydrogenated in presence of 10%Pd/carbon (30 mg) at room temperature and under atmospheric pressure hydrogen for 4 hours. The catalyst is filtered off on a millipore filter and the filtrate is evaporated to dryness then the residue is dried by a vacuum pump to obtain the crude free phosphate (180 mg; 100%) ES/MS:m/z 850.7 [M+H-P(O)(OH)₃⁺], 948.5 [M+H]⁺ 970.5 [M+Na]⁺ (FIG. 4).

### Example 1.1.

### Determining Epimerization Rate of Synthesis Intermediates

Stereochemistry of chiral centers bearing acylamino groups is determined by initially used amino acid configuration. However, peptide coupling performed between the aspartic or glutamic acid derivative and the aminoalcohol obtained from ornithine or lysine can lead, under certain conditions, to epimerization at C_{α} of the acid partner involved in the coupling reaction. In order to assess epimerization rate of such a reaction, the following method was followed for compounds derived from aspartic acid.

The sample (30 μg) was evaporated into a microvial, then redissolved in 40 μl of 6M HCl Hydrolysis was allowed to proceed for 24 hours at 110° C. under argon atmosphere. The sample was then evaporated to dryness, and thereafter redissolved in 100 ml of 0.1 M tetraborate buffer, pH 9.2. A pre-column derivatization was subsequently run with OPA-IBLC reagent (o-phtaldialdehyde—N-isobutyryl-L-cysteine) in the following proportions:

5 μl of a 0.1 M sodium tetraborate buffer solution, pH 9.2

2 μl of a methanol 170 mM OPA, 260 mM IBLC solution

2 μl of the solution to be assayed

By running an HPLC separation on Hypersil ODS column (250×4.6 mm, 5 μm, Supelco), both derivatives arising from L- and D-forms of aspartic acid present in the initial sample were quantified (Bruckner et al., 1995, J. Chromatography. A 711, 201-215). HPLC operating conditions used were as follows:

Column: Hypersil ODS (250×4.6, 5 μm, Supelco)

Mobile phase: A: 23 mM sodium acetate, pH 5.9

B : Methanol -acetonitrile (121)

Injection: 5 μl

Flow rate: 1 ml/min

Elution step: 96:4 to 80:20 A:B gradient within 25 minutes

Detection: UV: 338

Under these chromatographic conditions, retention times of 16.1 and 17.2 minutes were observed for the L- and D-aspartic acid derivatives, respectively.

2^{nd} Series of Examples

Preparation of dipeptide-like Compounds Bearing an Accessory Functional Side Chain Spacer

### Example 2.1.

### 3-[(R)-dodecanoyloxytetradecanoylamino]-4-oxo-5-aza-9-[(R)-3-hydroxytetradecanoylamino]-decan-1, 10-diol 1-dihydrogenphate 10-(6,7-dihydroxyheptanoate) (=OM-197-FV6) (FIG. 5)

2.1.1. 1-(Diphenyloxyphosphoryloxy)-3-[(R)-3-dodecanoyloxytetra-decanoylamino]-4-oxo-5-aza-9-[(R)-3-benzyl-oxytetradecanoylamino]-decan-10-ol (6-heptenoate)

To a solution of 1-(diphenyloxyphosphoryloxy)-3-[(R)-3-dodecanoyloxytetradecanoylamino]-4-oxo-5-aza-9-[(R)-3-benzyloxytetra-decanoylamino]-10-ol (FIG. 1.1) (875 mg; 0.74 mmol.) in anhydrous CH₂Cl₂ (25 ml), there was added 6-heptenoic acid (141 μl; 1.04 mmol.; 1.4 eq.). The solution was cooled down to 0° C. Next, there was added EDCl (64 mg; 0.33 mmol.; 1.4 eq.) and DMAP (41 mg; 0.33 mmol.; 0.14 eq.) The reaction mixture was stirred for 30 minutes at 0° C. and thereafter 3 hours at room temperature. After dilution with CH₂Cl₂, the organic layer was washed in succession with H₂O, a 1N HCl solution and H₂O. The organic layer was then dried over MgSO₄, then evaporated at 40° C. under vacuum. By running a flash chromatography purification on a silica gel (petroleum ether/AcOEt 1/1 eluent), there was recovered 1-(Diphenyloxyphosphoryloxy)-3-[(R)-3-dodecanoyloxytetradecanoylamino]-4-oxo-5-aza-9-[(R)-3-benzyloxytetradecanoylamino]-dcan-10-ol 6-heptenoate (820 mg ; 85%) as a white foam (Rf=0.18 in 1/1 petroleum ether/AcOEt; phosphomolybdinium acid color developer). ¹³C-NMR (62.89 MHz, CDCl₃), δ in ppm:173.30; 171.18; 170.4; 169.91; 169.73; 150.10; 138.21; 138.14; 129.77; 128.25; 127.49; 125.44; 119.88; 114.56; 76.48; 71.11; 70.90; 66.01; 65.52; 49.90; 47.80; 41.34; 39.07; 33.92; 33.76; 33.69; 33.61; 33.17; 31.75; 29.47; 29.19; 29.00; 28.46; 28.11; 25.34; 25.05; 24.85; 22.52; 13.96.

2.1.2. 1-(Diphenyloxyphosphoryloxy)-3-[(R)-3-dodecanoyloxy-tetradecanoylamino]-4-oxo-5-aza-9-[f R)-3-benzyloxytetradecanoylamino]-10-ol (6, 7-dihydroxyheptenoate)

To a solution containing K₃Fe(CN)₆ (373 mg; 1.13 mmol; 3 eq.) K₂CO₃ (157 mg; 1.13 mmol.; 3 eq.) and 1,4-diazabicyclo[2.2.2.]octane (DABCO) (10.7 mg; 0.095 mmol.; 0.25 eq.) in a t-butanol/water mixture (5 ml/5ml), there was added the compound obtained above (486 mg, 0.38 mmol.), then osmium tetraoxide dissolved in 2.5% t-butanol (48 μl; 4.75 μmol.; 0.0125 eq.). The reaction mixture was strongly stirred for 16 hours at room temperature (27° C.). Na₂S₂O₅ (60 mg) was added and stirring was continued for nearly 1 hour until the medium shifted in color from brown to green or blue. The reaction mixture was diluted with ether and the organic layer was separated. The aqueous layer was thoroughly washed with ether and the organic layers were pooled, dried over MgSO₄, then evaporated at 40° C. under vacuum. The expected raw diol was thus obtained as a green oil. By running flash chromatography purification on a silica gel (5/2 CH₂Cl₂/acetone), there was recovered pure 1-(diphenyloxyphosphoryloxy)-3-[(R)-3-dodecanoyloxy-tetradecanoylamino]-4-oxo-5-aza-9-[(R)-3-benzyloxytetradecanoylamino]-10-ol 6,7-dihydroxyheptanoate (198 mg 40%) as an amorphous solid (Rf=0.24 in 5/2 CH₂Cl₂/acetone; phosphomolybdinium acid color developer). ¹³C-NMR (62.89 MHz, CDCl₃), δ in ppm:173.46; 173.33; 171.34; 170.58; 170.02; 150.13; 138.23; 129.80; 128.26; 127.87; 127.54; 125.50; 120.01; 119.80; 71.79; 71.23; 70.97; 66.63; 66.03; 65.54; 49.93; 47.90; 41.46; 39.17; 33.98; 33.79; 32.86; 32.45; 31.77; 29.49; 29.21; 29.03; 28.51; 25.50; 25.07; 24.87; 24.77; 24.66; 22.54; 13.99. HPLC (210 nm): T_{R}=32.535 min. (retention time) ES/MS: m/z ratio 1343.0 (M+Na⁺); 1321.0 (M+H⁺) 1071.0 (M+H⁺ monophenyl phosphate).

2.1.3. 1(-Diphenyloxyphosphoryloxy)-3-[(R)-3-dodecanoyloxy-tetradecanoylamino]-4-oxo-5-aza-9-[(R)-3-hydroxytetradecanoylamino]-10-ol (6, 7-dihydroxyheptanoate).

A solution of the above diol (198 mg, 0.15 mmol.) in HPLC grade EtOH(20 ml)/AcOEt (1.4 ml) mixture is hydrogenated in presence of Pd on carbon containing 10% Pd (70 mg) at room temperature and under atmospheric pressure hydrogen for 2.5 hours. The catalyst is filtered off. The filtrate is evaporated to dryness and the residue is then dried by suction from a vacuum pump to provide the crude debenzylated product (168 mg ; 91%) as an amorphous solid. ¹³C-NMR (62.89 MHz, CDCl₃), δ in ppm: 173.15; 173.51; 172.82; 171.04; 170.49; 170.35; 150.05; 129.79; 129.42; 125.53; 119.91; 119.83; 119.72; 71.80; 70.93; 68.58; 66.47; 65.94; 65.52; 50.02; 48.12; 42.59; 41.26; 39.13; 36.92; 34.29; 33.85; 32.32; 31.74; 29.50; 29.18; 29.00; 28.15; 25.47; 25.07; 24.85; 24.73; 24.56; 22.51; 13.99. HPLC (210 nm):T_{R}=30.7 min. ES/MS: m/z ratio 1253.0 [M+Na]⁺; 1231.0[M+H]⁺.

2.1.4. 3-[(R)-3-dodecanoyloxytetradecanoylamino]-4-oxo-5-aza-9-[(R)-3-hydroxytetradecanoylamino]-decan-1, 10-diol-1-dihydrogen-phosphate 10-(6, 7-dihydroxyheptanoate) (=OM-197-FV6)

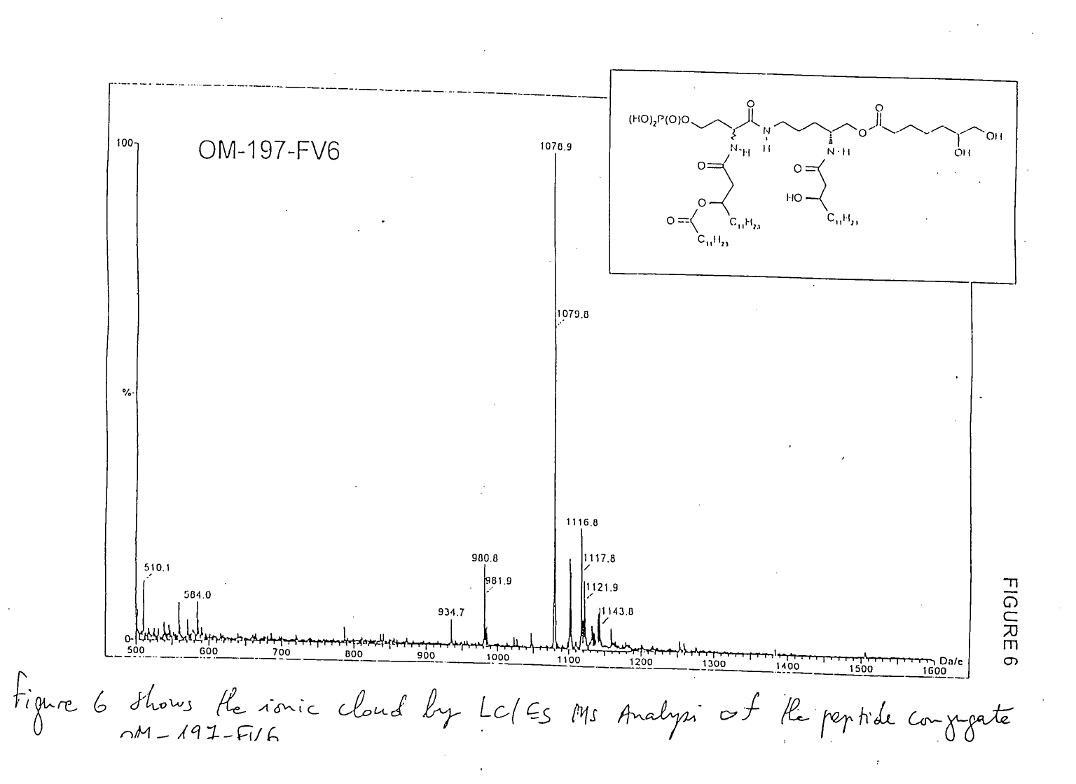

A suspension of platinium oxide PtO₂ (91 mg) (preactivated to yield platinium black under atmospheric pressure hydrogen for 10 min) in HPLC-grade ethanol (1 ml) is added to a solution of the previously obtained trivalent alcohol (168 mg; 0.14 mmol.) in HPLC-grade EtOH (8ml)/AcOEt mixture (8 ml). The solution is hydrogenated at room temperature (27° C.) under atmospheric pressure hydrogen for 24 hours. The catalyst is filtered off. The filtrate is evaporated to dryness then the residue is dried by suction from a vacuum pump to thereby obtain the crude phosphate product (130 mg; 88%). HPLC (210 nm):T_{R}=23.6 min. ES/MS: m/z ratio 1078.9 [M+H]⁺, 1100.8 [M+Na]⁺, 116.8 [M+K]⁺ (FIG. 6).

### Example 2.2.

### 3-[(R)-3-dodecanoyloxytetradecanoylamino]-4-oxo-5-aza-9-[(R)-3-hydroxytetradecanoylamino]-decan-1, 10-diol 1-dihydrogen-phosphate 10-(6-oxohexanoate) (=OM-197-FV7)

2.2.1. 3-[(R)-3-dodecanoyloxytetradecanoylamino]-4-oxo-5-aza-9-[(R)-3-hydroxytetradecanoylamino]-decan-1,10-diol 1-dihydrogen-phosphate 10-(6-oxohexanoate) (=OM-197-FV7) (FIG. 5)

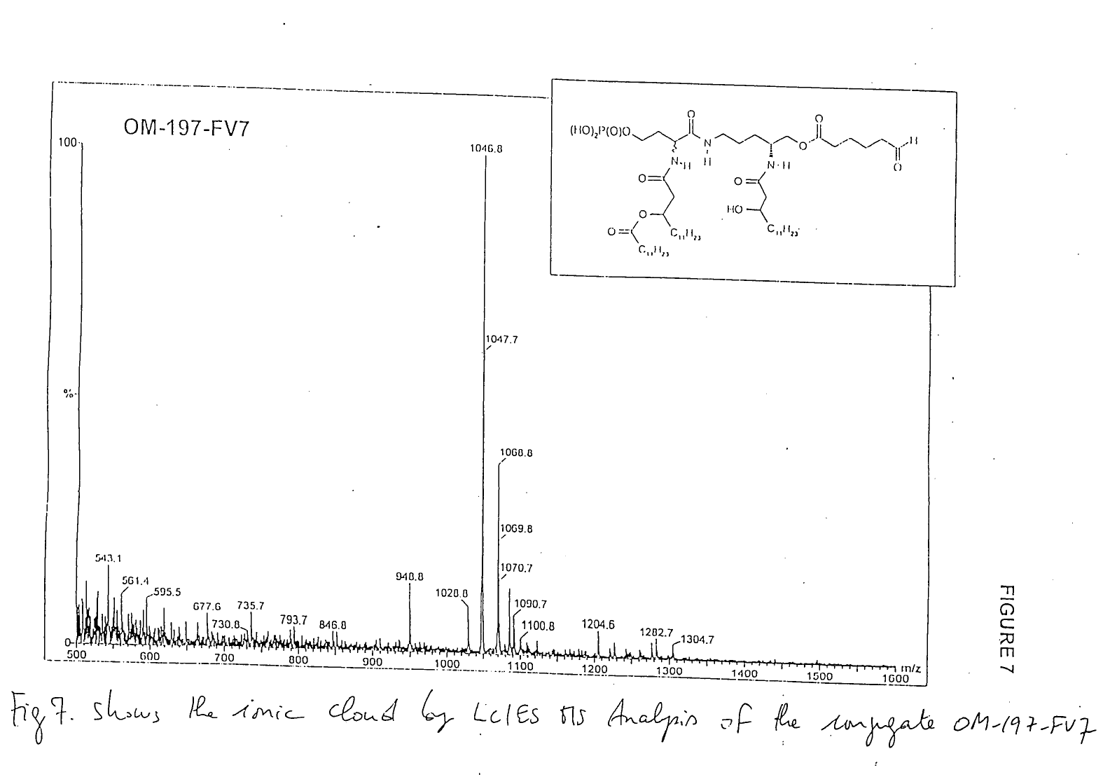

A periodic oxidation reaction was carried out through addition of 1.86 ml of 0.1 M NaIO₄ (39.62 mg, 20 eq.) to 10 mg (9.28 μmol., 1 eq.) of deprotected triol as prepared above in a (1:1) water/isopropanol mixture. The reaction is monitored by LC/UV detection and shows quantitative conversion of the diol functional group into aldehyde two hours later. The expected reaction is shown to occur by observing a 1046.8 m/z ratio molecular ion on the ES/MS spectrum following the periodic oxidation step (FIG. 7). By looking at the spectrum, 1068.8 m/z ratio ([M+Na]⁺) sodium and 1084.8 m/z ratio ([M+K]⁺) potassium adducts are clearly visible, as well as a fragment corresponding to the loss of a 948.8 m/z ratio phosphoryl group. The reaction is terminated by adding 1 to 2 drops of ethylene glycol. Purification of the product can be carried out by SPE on a C18 phase with a yield of 90% (5 mg diol analyte)

### Example 2.3.

### N-[(R)-3-dodecanoyloxytetradecanoylamino]-D-aspartic acid, α-N-{(4R)-5-hydroxy-4-[(R)-3-hydroxytetra-decanoylamino]-pentyl}amide 5-O-(6, 7-dihydroxyheptanoate) (=OM-197-MC-FV5) (FIG. 8)

2.3.1. N-[(R)-3-dodecanoyloxytetradecanoylamino]-D-aspartic acid, α-N-{(4R)-5-hydroxy-4-[(R)-3-benzyloxytetra-decanoylamino]pentyl}-amide-β-benzyl ester, 5-O-(6-heptenoate)

To a solution of N-[(R)-3-dodecanoyloxytetradecanoylamino]-D-aspartic acid, α-N-{(4R)-5-hydroxy-4-[(R)-3-benzyloxy-tetradecanoylamino]pentyl}amide-β-benzyl ester (Section 1.2.2.) (122 mg 0.116 mmol.) in anhydrous dichloromethane (5 ml), there is added 6-heptenoic acid (22 μl ; 0.160 mmol; 1.4 eq.). The solution is cooled down to 0° C. Addition is then made of EDCl (49.3 mg; 0.25 mmol.; 2.1 eq.) and DMAP (5.6 mg; 0.046 mmol.; 0.4 eq.). The reaction mixture is stirred at 0° C. for 30 minutes, and thereafter for 6 hours at room temperature. After CH₂Cl₂ dilution, the organic layer is washed in succession with H₂O, 1N HCl (2×), NaHCO₃ (2×) and H₂O (2×). The expected heptenoic acid ester is thereby obtained in a pure state (119 mg; 89%) as a white solid (R_{f}=0.62 in 5/1 CH₂Cl₂/acetone mixture, color developer:phosphomolybdinium acid) ¹³C-NMR (62.89 MHz, CDCl₃), δ in ppm: 173.63; 173.38, 171.72; 171.11, 170.08; 138.14; 135.28; 128.46, 128.34; 128.24; 128.05; 127.64; 127.53; 114.62; 76.49; 71.14, 66.66; 65.49; 49.17; 47.79; 41.69; 41.35; 39.09; 35.46; 34.33; 33.80; 33.65; 33.21; 31.79, 29.52; 29.23; 29.06; 28.46; 28.16; 22.56; 14.00. HPLC (210 nm):T_{R}=36.9 min. ES/MS: m/z ratio 1159.0 [M+H]⁺; 1176.0 [M+NH₄]⁺. Mp=81.5-84° C. [α]_{D}²⁰=+7.3 (CHCl₃).

2.3.2. N-[(R)-3-dodecanoyloxytetradecanoylamino]-D-aspartic acid, α-N-{(4R )-5-hydroxy-4-[(R)-3-benzyloxytetradecanoylamino]pentyl}amide-β-benzyl ester, 5-O-(6,7-dihydroxyheptanoate)

To a solution of heptenoic acid ester as obtained above (60 mg; 0.052 mmol.) in a (5/3 ml) H₂O/acetone mixture, addition is made of N-methylmorpholin oxide (9 mg; 0.076 mmol.; 1.5 eq.), and thereafter of a solution of OsO₄ in 2.5% t-butanol (123 μl; 0.012 mmol.; 0.23 eq.) in a dropwise fashion. The reaction mixture is stirred for 24 hours at room temperature. Na₂S₂O₅ (20 mg) is added to the reaction mixture which is stirred thereafter for 1 hr to 2 hr at room temperature. The solution is then extracted several times with ether, and the organic layers are subsequently pooled, dried, over MgSO₄ and concentrated. By running a flash chromatography treatment on a silica gel (5/2 CH₂Cl₂/acetone eluent), there is recovered the expected diol in a pure state (35 mg; 57%) as a white solid (Rf=0.15 in a 5/1 CH₂Cl₂/acetone mixture ; color developer:phosphomolybdinium acid). ¹³C-NMR (62.89 MHz, CDCl₃), δ in ppm: 173.67; 173.39; 171.81; 171.34; 170.27; 170.01; 138.18; 135.27; 128.56; 128.42; 128.18; 127.50; 127.68; 76.68; 71.82; 71.37; 71.17; 66.85; 66.74; 65.66; 49.27; 47.99; 41.88; 41.51; 39.31; 35.60; 34.42; 33.95; 33.51; 31.80; 29.60; 29.49; 29.30; 28.55; 25.65; 25.60; 25.19; 25.12; 24.97; 24.77; 24.14; 22.65; 14.08. HPLC (210 nm):T_{R}=34.16 min. ES/MS: m/z ratio 1193.0 [M+H⁺]; 1212.0 [M+NH₄]⁺.

2.3.3. N-[(R)-3-dodecanoyloxytetradecanoylamino]-D-aspartic acid, α-N-{(4R)-5-hydroxy-4-[(R)-3-hydroxytetradecanoylamino]pentyl}amide 5-O-(6, 7-dihydroxyheptanoate) (=OM-197-MC-FV5)

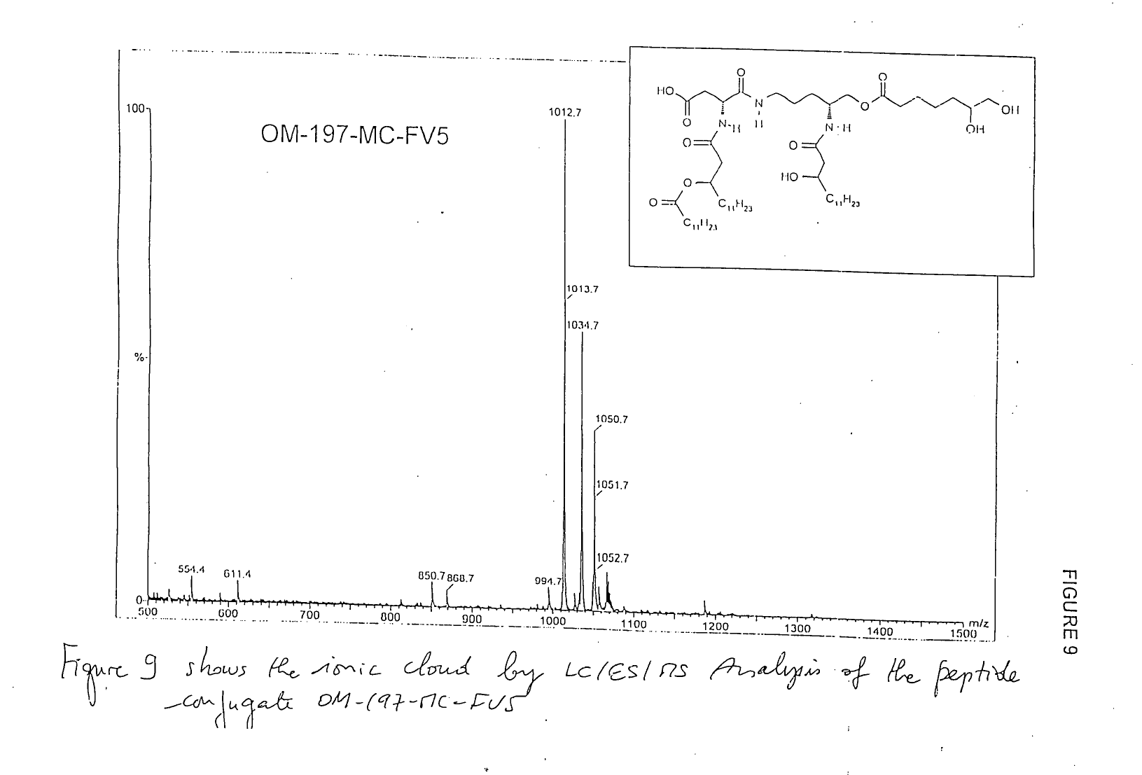

A solution of the above obtained diol (35 mg; 0.029 mmol.) in a HPLC-grade MeOH (2 ml)/AcOEt (2 ml) mixture is hydrogenated in presence of Pd on carbon containing 10% palladium (10 mg) at room temperature and under atmospheric pressure hydrogen for 2.5 hours. The catalyst is filtered off. The filtrate is evaporated to dryness then the residue is dried by suction from a vacuum pump to obtain crude N-[(R)-3-dodecanoyloxytetradecanoylamino]-D-aspartic acid, α-N-{(4R)-5-hydroxy-4-[(R)-3-hydroxy-tetra-decanoylamino]-pentyl}amide 5-O-(6,7-dihydroxyheptanoate) (26 mg; 88%) as a white solid. HPLC (210 nm):T_{R}=26.90 min. m.p.=94-97° C. [α]_{D}²⁰=+11.1° (CHCl₃/MeOH=1:0.1). ES/MS: m/z ratio 1012.7 [M+H]⁺; 1034, 7 [M+Na]⁺, 1050.7 [M+K]⁺ (FIG. 9).

### Example 2.4.

### N-[(R)-3-dodecanoyloxytetradecanoylamino]-D-aspartic acid, α-N-{(4R)-5-hydroxy-4-[(R)-3-hydroxytetradecanoylamino]pentyl}amide 5-O-(6-oxohexanoate) (=OM-197-MC-FV7) (FIG. 8).

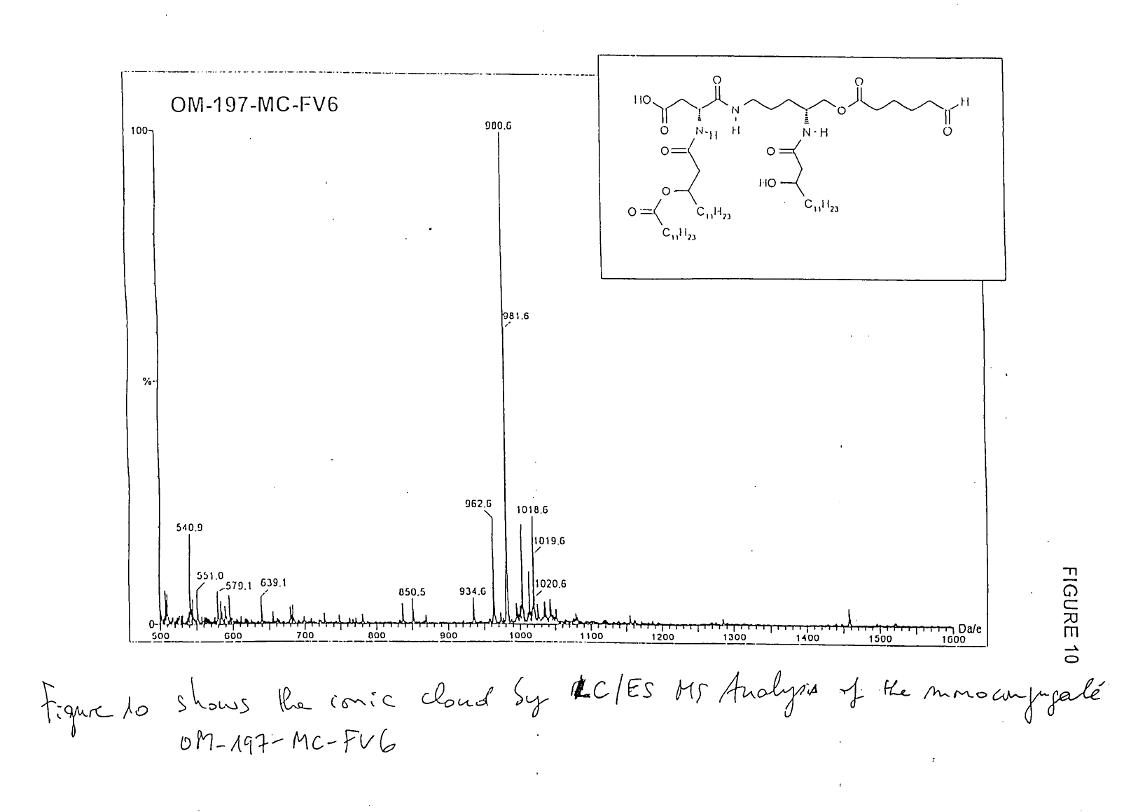

1.98 ml of 0.1 M NaIO₄ (42.25 mg, 20 eq.) are added to 10 mg (9.88 μM, 1 eq.) of deprotected triol prepared above (Section 2.3.3.) in a (1:1) isopropanol-water mixture. The solution is stirred for 2 hours at room temperature. The reaction is stopped by adding 1 to 2 drops of ethylene glycol. Upon this periodic oxidation step, an m/z ratio 980.6 [M+H]⁺ molecular ion is observed on the ES/MS spectrum (FIG. 10), demonstrating the presence of an aldehyde functional group. Peaks corresponding to 1002.8 m/z ratio ([M+Na]⁺) sodium and 1018.6 m/z ratio ([M+K]⁺) potassium adducts are also visible. SPE purification on a C18 phase affords recovery of the OM-197-MC-FV6 product with 90% yield.

### Example 2.5.

### N-[(R)-3-dodecanoyloxytetradecanoylamino]-D-aspartic acid, α-N-{(4R)-5-hydroxy-4-[(R)-3-hydroxytetradecanoylamino]pentyl}amide 5-O-(6-hydroxyhexanoate) (=OM-197-MC-FV7) (FIG. 8).

2.5.1. N-[(R)-3-dodecanoyloxytetradecanoylamino]-D-aspartic acid, α-N-{(4R)-5-hydroxy-4-[(R)-3-hydroxytetradecanoylamino]pentyl}amide 5-O-(6-hydroxyhexanoate) (=OM-197-MC-FV7)

To a solution of 6-oxohexanoic acid derivative as obtained above (4.5 mg, 0.0043 mmol.) in 90% isopropanol (20 ml), there is added a NaBH₄ solution in methanol (1 mg/ml) (0.2 ml). The mixture is stirred for 3 min. at 25° C., then excess acetic acid (0.2 ml) is added. Purification by preparative HPLC on a C18 column affords recovery of the product OM-197-MC-FV7 (2.3 mg, 51%) dissolved in 90% isopropanol.

### Example 2.6

### (3RS, 9R)-3-[(R)-3-dodecanoyloxytetradecanoylamino]-4-oxo-5-aza-9-[(R)-3-hydroxytetra-decanoylamino]-decan-10-ol 1-dihydrogen-phosphate (6-aminohexanoate) (=OM-287-AC-RS, R) (FIG. 11)

2.6.1. 1-(Diphenyloxyphosphoryloxy)-3-[(R)-dodecanoyloxy-tetradecanoylamino]-4-oxo-5-aza-9-[(R)-3-benzyloxy-tetra-decanoylamino]-decan-10-ol (6-benzVloxycarbonyl aminohexanoate)

To a solution of 1-(diphenyloxyphosphoryloxy)-3-[(R)-3-dodecanoyloxytetradecanoylamino]-4-oxo-5-aza-9-[(R)-3-benzyloxy-tetra-decanoylamino]-decan-10-ol (FIG. 1) (230 mg; 0.20 mmol.) in anhydrous CH₂Cl₂ (8 ml), addition is made of 6-benzyloxycarbanoylaminohexanoic acid (88 mg; 0.33 mmol.; 1.65 eq.). The solution is cooled down to 0° C.

This is followed by addition of EDCl (200 mg, 1.04 mmol, 5,2 eq.) and DMAP (13 mg; 0.10 mmol.; 0.5 eq.). The reaction mixture is stirred for 30 minutes at 0° C., and thereafter for 4 hours at room temperature. After dilution with CH₂Cl₂, the organic layer is separated and washed in succession with H₂O, 1N HCl, H₂O. The organic layer is then dried over MgSO₄, and evaporated at 40° C. under vacuum. By running a flash chromatography purification on a silica gel (7/1.2 CH₂Cl2/Acetone eluent), there is recovered the pure ester (115 mg; 41%) as an amorphous solid (Rf=0.77 in 5/2 CH₂Cl2/Acetone, phosphomolybdinium acid color developer) ¹³C-NMR (62.89 MHz, CDCl₃), δ in ppm: 173.27; 171.20; 170.34; 169.88; 156.36; 150.16; 150.13; 138.28; 136.56 129.80; 128.35; 128.26; 127.90; 127.51; 125.45; 119.97; 119.83; 71.05; 66.37; 65.97; 65.61; 49.94; 47.81; 41.39; 40.66; 39.09; 34.32; 34.25; 33.95; 33.65; 31.78; 29.50; 29.38; 29.22; 29.03; 28.55; 28.44; 25.95; 25.06; 24.87; 24.26; 22.55; 14.00. HPLC (210 nm):T_{R}=33.497 min. ES/MS: m/z ratio 1424.0 [M+H]⁺; 1174.0 [M+H−(PhO)₂OPOH]⁺.

2.6.2.1-(Diphenyloxyphosphoryloxy)-3-[(R)-3-dodecanoyloxy-tetradecanoylamino]-4-oxo-5-aza-9-[(R)-3-hydroxy-tetradecanoylamino]-decan-10-ol 10-(6-aminohexanoate)

A solution of the above obtained product (115 mg; 0.81 μmol.) in an HPLC-grade EtOH (15 ml)/glacial acetic acid (0.4 ml) mixture is hydrogenated in presence of palladium (10% Pd—carbon) (80 mg) at room temperature and under atmospheric pressure hydrogen for 4 hours. The catalyst is filtered off. The filtrate is evaporated to dryness and the residue is then dried by suction from a vacuum pump to obtain the debenzylated product (90 mg, 97%) as an amorphous solid. HPLC (210 nm):T_{R}=29.185 min. ES/MS:m/z ratio 1200.0 [M+H]⁺; 952.0 [M+H−(PhO)₂OPOH]⁺.

2.6.3. 3-[(R)-3-dodecanoyloxytetra-decanoylamino]-4-oxo-5-aza-9-[(R)-3-hydroxytetradecanoylamino]-1,10-diol-dihydrogenphosphate 10-(6-aminohexanoate) (=OM-197-MP-AC)

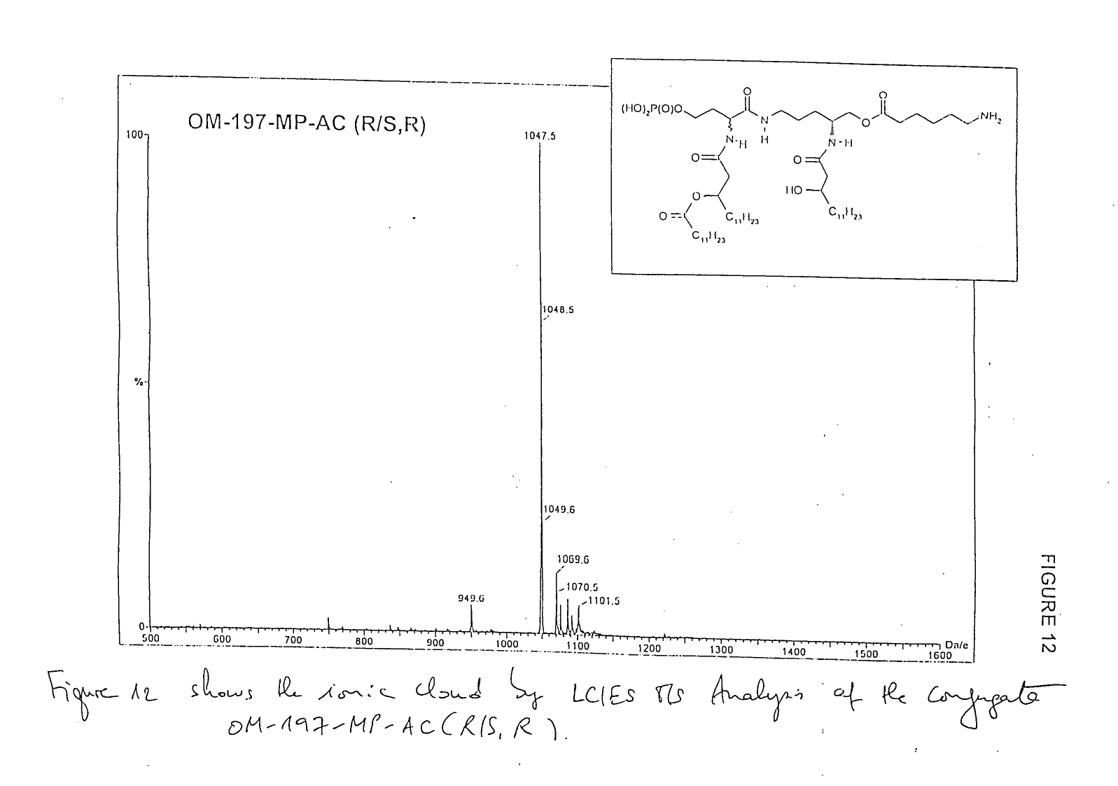

To a solution of platinium oxide PtO₂ (30 mg) in (HPLC-grade) ethanol (1 ml) (preactivated to yield platinium black under atmospheric pressure hydrogen for 10 minutes), addition is made of a solution of amino alcohol as obtained above (90 mg, 75 μmol.) in an HPLC-grade EtOH (5 ml)/1N HCl (0.1 ml) mixture. The solution is hydrogenated at room temperature, under atmospheric pressure hydrogen for 24 hours. The catalyst is filtered off. The filtrate is evaporated to dryness and the residue is then dried by suction from a vacuum pump to obtain the desired aminophosphate (60 mg, 79%). HPLC (210 nm):T_{R}=28.51 min. ES/MS: m/z ratio 1047.5 [M+H]⁺; 1069.6 (M+Na)⁺, 949.6 [M+H-(HO)₂OPOH]⁺ (FIG. 12).

### Example 2.7

### (3R, 9R)-3-[(R)-3-dodecanoyloxytetradecanoylamino]-4-oxo-5-aza-9-[(R)-3-hydroxytetradecanoylamino]-1,10-diol 1-dihydrogenphosphate 10-(6-aminohexanoate) (OM-197-MP-A C) (FIG. 13)

2.7.1. N-[(R)-3-dodecanoyloxytetradecanoyl]-D-aspartic acid, α-N-{(4R)-5-(2-tetrahydroypyranyl)oxy-4-[(R)-3-benzyloxytetradecanoyl-amino]pentyl}amide β-benzyl ester (ASM-O-THP)

To a solution of N-[(R)-3-dodecanoyloxytetradecanoyl]-D-aspartic acid, α-N-{(4R)-5-hydroxy-4-[(R)-3-benzyloxytetradecanoylamino]-pentyl}amide β-benzyl ester (1.5 g; 1.43 mmol.) in anhydrous CH₂Cl₂ (30 ml), there are added in succession at room temperature and under argon flow 3,4-Dihydro-2H-pyran (DHP) (327 μl, 3.58 mmol.) and pyridinium p-toluenesulfonate (PPTS) (108 mg, 429 μmol.). After stirring for 18 hours at room temperature, addition is made again of 3,4-Dihydro-2 H-pyran (DHP) (130 μl, 1.43 mmol.). The solution is then stirred for further 9 hours at room temperature then diluted with CH₂Cl₂ and successively washed with a 5% NaHCO₃ solution and H₂O. The organic layer is dried over MgSO₄, filtered and evaporated. By running flash chromatography purification on a silica gel (successively 6/1 and 7/1 CH₂Cl₂/acetone eluent), there is recovered the N-[(R)-3-dodecanoyloxytetradecanoyl]-D-aspartic acid, α-N-{(4R)-5-(2-tetrahydroxypyranyl)oxy-4-[(R)-3-benzyloxytetradecanoylamino]pentyl}-amide β-benzyl ester (1.45 g; 90%) as a white cristalline solid (Rf=0.57 in 5/1 CH₂Cl₂/acetone; phosphomolybdinium compound and U.V. color development agent

2.7.2 N-[(R)-3-dodecanoyloxytetradecanoyl]-D-aspartic acid, α-N-{(4R)-5-(2-tetrahydroypyranyl)oxy-4-[(R)-3-benzyloxytetradecanoyl-amino]pentyl}amide

A solution of the compound obtained above (250 mg; 0.22 mmol.) in a 1/1 EtOH/EtOAc mixture (16 ml) containing Et₃N (0.4 ml) is hydrogenated in presence of Pd on carbon containing 10% Pd (10 mg) at room temperature and under atmospheric pressure hydrogen for 2 hours. The catalyst is filtered off and the filtrate is evaporated to dryness and then dried by suction from a vacuum pump to recover the acid as a triethylammonium salt (250 mg).

2.7.3. 3-[(R)-3-dodecanoyloxytetradecanoylamino]-4-oxo-5-aza-9-[(R)-3-benzyloxytetradecanoylamino]-10-(2-tetrahydropyranyl)oxy-decan-1-ol

To a solution of the acid obtained above (≈250 mg) in a 2/1 CH₂Cl₂/anhydrous THF mixture (3 ml) at 0° C. and under argon flow, there are added N-methylmorpholin (72 μl; 0.66 mmol.; 3 eq.) then isobutyl chloroformate (86 μl; 0.66 mmol.; 3 eq.). Rapid formation of an N-methylmorpholin hydrochloride precipitate is observed. The reaction progress is monitored by TLC. (Rf=0.90 in 2/1 CH₂Cl₂/acetone). After stirring for 30 minutes at room temperature, the reaction mixture is cooled down to 0° C. and a solution of NaBH₄ (33 mg; 0.88 mmol.; 4 eq.) in H₂O (1 ml) is then quickly added. As soon as gaz emission has ceased (5 min.), the solution is diluted with H₂O (1 ml) and THF (1 ml) and then stirred for 5 minutes at room temperature. The solution is concentrated, diluted with CH₂Cl₂ and H₂O, followed by layer separation. The organic layer is dried over MgSO₄, filtered and evaporated. By running a flash chromatography purification on a silica gel (2/1 CH₂Cl₂/acetone eluent), the alcohol is recovered (138 mg; 61% in two steps) as a white cristalline solid. (Rf=0.33 in 3/1 CH₂Cl₂/acetone; phosphomolybdinium compound and U.V. color development agent).

2.7.4. 3-[(R)-3-dodecanoyloxytetradecanoylamino]-4-oxo-5-aza-9-[(R)-3-benzyloxytetradecanoylamino]-10-(2-tetrahydropyranyl)oxy-decan-1-ol dibenzyl phosphate

To a solution of this alcohol as prepared above (210 mg; 0.12 mmol.) and 1H-tetrazole (25 mg; 0.35 mmol.; 3 eq.) in anhydrous THF (5 ml) at room temperature and under argon flow, there is added 85% dibenzyl-diethyl phosphoramidite (95 μl; 0.27 mmol. 2.3 eq.). Rapid formation of white cristals in the reaction medium can be seen. After stirring for 30 minutes, the reaction mixture is cooled down to −20° C. and an mCPBA solution (57-86%; 75 mg; 0.43 mmol.; 3.7 eq.) in CH₂Cl₂ (3 ml) is then added. Disappearance of cristals is noted. After stirring for 45 minutes at room temperature; a saturated Na₂S₂O₃ solution (3 ml) is added and the reaction mixture is then stirred for 10 minutes. The solution is next diluted with ether, and the organic layer is then separated and washed with a saturated solution of Na₂S₂O₃ (5×), and a saturated solution of NaHCO₃ (2×). The organic layer is dried over MgSO₄, filtered and evaporated. By running a flash chromatography purification on a silica gel (4/1 then 2/1 CH₂Cl₂/acetone eluent), there is recovered the dibenzylphosphate (126 mg; 84%) in the form of an amorphous solid (Rf=0.53 in 3/1 CH₂Cl₂/acetone; phosphomolybdinium compound and U.V. color development agent).

2.7.5. 3-[(R)-3-dodecanoyloxytetradecanoylamino]-4-oxo-5-aza-9-[(R)-3-benzyloxytetradecanoylamino]-1,10-diol 1-(dibenzyl phosphate)

A solution of 1% HCl in methanol (25 ml) at 0° C. is added to a solution of the compound prepared above (700 mg, 0.54 mmol.) in CH₂Cl₂ (2.5 ml). After stirring for 45 minutes at 0° C., the reaction medium is neutralized with a 5% NaHCO₃ solution, diluted with CH₂Cl₂ and the organic layer is separated. The resulting aqueous phase is then extracted with CH₂Cl₂ (3×) and the organic layers are pooled. The organic layer is dried over MgSO₄, filtered and evaporated to yield the crude alcohol (640 mg; 98%) (Rf=0.50 in 3/1 CH₂Cl₂/acetone; phosphomolybdinium compound and U.V. color development agent).

2.7.6. 3-[(R)-3-dodecanoyloxytetradecanoylamino]-4-oxo-5-aza-9-[(R)-3-benzyloxytetradecanoylamino]-decan-1,10-diol 1-dibenzyl phosphate 10-(6-benzyloxycarbonylaminohexanoate)

To a solution of the compound prepared above (640 mg, 0.53 mmol.) and 6-(benzyloxycarbonylamino)hexanoic acid (423 mg, 1.60 mmol.) in dry CH₂Cl₂(25 ml) at 0° C. and under argon flow, there are added in succession commercially available 1-(3-dimethylaminopropyl)-3-ethylcarbodiimide hydrochloride (306 mg, 1.60 mmol.) and 4-dimethylaminopyridine (20 mg, 160 μmol.). The reaction mixture is then stirred for 30 minutes at 0° C. and thereafter overnight at room temperature. The reaction medium is then washed with water and a solution of 1N HCl followed by layer separation. The organic layer is dried over MgSO₄, filtered and evaporated. By running a flash chromatography purification on a silica gel (4/1 then 2/1 CH₂Cl₂/acetone eluent), there is recovered the coupling reaction product (537 mg; 71%). ¹³C-NMR (62,89 MHz, CDCl₃), δ in ppm: 173.18, 171.16, 170.38, 169.60, 156.30, 138.23, 136.50, 135.38, 135.28, 128.42, 128.26, 128.17, 127.79, 127.74, 127.44, 76.48, 71.15, 70.84, 69.47, 69.39, 69.31, 66.25, 65.62, 64.37, 49.78, 47.76, 41.41, 41.34, 40.57, 38.97, 34.22, 34.16, 33.96, 33.57, 32.95, 31.70 , 29.15, 28.95, 28.32, 25.87, 25.46,25.02, 28.80, 24.18, 22.49, 13.94.

2.7.7. 3-[(R)-3-dodecanoyloxytetradecanoylamino]-4-oxo-5-aza-9-[(R)-3-hydroxytetradecanoylamino]-decan-1,10-diol 1-dihydrogenphosphate 10-(6-aminohexanoate) (=OM-197-MP-AC-R, R) (FIG. 13)

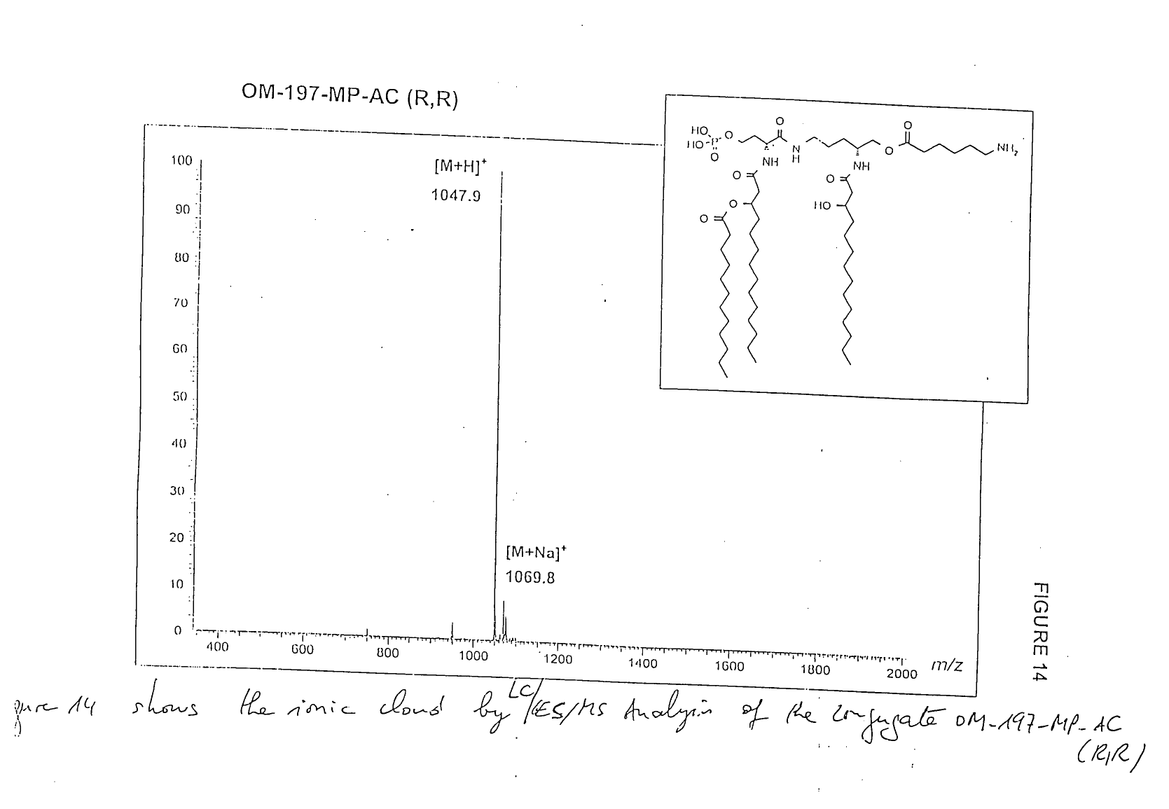

A solution of the compound as prepared above (500 mg, 0.35 mmol.) in a 5/2 CH₂Cl₂/ethanol mixture (70 ml) containing acetic acid (10 ml) is hydrogenated in presence of Pd on carbon containing 10% Pd at room temperature and under atmospheric pressure hydrogen for 12 to 24 hours. The catalyst is filtered off. The filtrate is evaporated to dryness and the residue is then dried by suction from a vacuum pump to obtain 3-[(R)-3-dodecanoyloxytetradecanoylamino]-4-oxo-5-aza-9-[(R)-3-hydroxytetra-decanoylamino]-decan-1,10-diol 1-dihydrogenphosphate 10-(6-aminohexanoate) (368 mg, quantitative yield). ES/MS : m/z ratio 1047.9 [M+H]⁺; 1069.8 (FIG. 14).

### Example 2.8

### (3S, 9R)-3-[(R)-3-dodecanoyloxytetradecanoylamino]-4-oxo-5-aza-9-[(R)-3-hydroxytetradecanoylamino]-1,10-diol 1-dihydrogenphosphate 10-(6-hydroxyhexanoate)(-OM-197-MP-A C-S, R)

Following the same reaction scheme for N-[(R)-3-dodecanoyloxytetradecanoyl]-L-aspartic acid, α-N-{(4R)-5-hydroxyoxy-4-[(R)-3-hydroxytetradecanoylamino]pentyl}amide β-benzyl ester (Section 1.2.4.), synthesis of the product of example 2.8. is finally achieved.

### Example 2.9.

### 3-[(R)-3-dodecanoyloxytetradecanoylamino]-4-oxo-5-aza-9-[(R)-3-hydroxytetradecanoylamino]-decan-1, 10-diol 1-dihydrogenphosphate 10-(6-hydroxyhexanoate) (=OM-197-FV8) (FIG. 15)

2.9.1. (2R)-5-(amino)-2-[(R)-3-benzyloxytetradecanoylamino]-pentan-1-ol Benzyloxymethyl Ether

To a solution of (2R)-5-(benzyloxycarbonylamino)-2-[(R)-3-benzyloxytetradecanoylamino]pentan-1-ol (Section 1.1.2.) (2.05 g; 3.60 mmol.) in anhydrous CH₂Cl₂ (40 ml) at room temperature and under argon flow, there are added in succession BOMCl (benzyl chloromethyl ether) (reagent grade 60%, 1.25 ml; 5.41 mmol; 1.5 eq.) and diisopropylethylamine (942 μl; 5.41 mmol.; 1.5 eq.). The reaction mixture is then stirred overnight at room temperature and evaporated thereafter to dryness. By running a flash chromatography purification on a silica gel (2/1 petroleum ether/EtOAc eluent), there is recovered the O-benzyloxymethyl derivative (2.28 g; 92%) as. a white cristalline solid. (Rf=0.70 in 1/3 petroleum ether/EtOAc mixture, phosphomolybdinium acid and UV color developer) m.p.=97-100° C. A solution of this product (2.00 g; 2.90 mmol.) in HPLC-grade EtOH (220 ml) containing Et₃N (4 ml) is hydrogenated in presence of 20% Pd(OH)₂ on carbon (200 mg) at room temperature and under atmospheric pressure hydrogen for a period of 3 hours. The catalyst is filtered off. The filtrate is evaporated to dryness and the residue is then dried by suction from a vacuum pump to obtain the free amine (1.58 g; 98%) as an amorphous solid. [α]_{D}=1° (c=1.20; CHCl₃); ¹H-NMR (250 MHz, CDCl₃), δ in ppm: 7.45-7.21 (m, 10H, Ar), 6.52 (d,1H, NH), 4.80-4.45 (m, 6H, 2× CH₂-ph, O—CH₂—O), 4.10 (m, 1H, H-3′), 3.83 (m, 1H, H-2), 3.62 (dd, 1H, H-1), 3.47 (dd, 1H, H-1), 2.65 (t, 2×H-5), 2.40 (m, 2H, 2×H-2′), 1.80-1.40 (m, 8H, 2×H-4, 2×H-3, 2×H-4′, NH₂), 1.40-1.20 (m, 18H, 9×CH₂), 0.88 (t, 3H, CH₃); ¹³C-NMR (62.89 MHz, CDCl₃), δ in ppm: 170.78; 138.24; 137.63; 128.38; 128.32; 127.66; 127.62; 94.82; 76.70; 71.26; 69.63; 69.44; 48.48; 41.82; 41.47; 33.83; 31.84; 29.88; 29.58; 29.56; 29.51; 29.27; 29.04; 25.11; 22.62; 14.06.

2.9.2. N-[(R)-3-dodecanoyloxytetradecanoylamino]-L-aspartic acid, α-N-{(4R)-5-(benzyloxymethoxy)-4-[(R)-3-benzyloxytetradecanoyl-amino]-pentyl}amide β-benzyl ester

The amine prepared as set forth above is coupled with the N-[(R)-3-dodecanoyloxytetradecanoylamino]-L-aspartic acid β-benzyl ester (prepared from the L-aspartic acid β-benzyl ester, see section 1.2.1) in presence of IIDQ following the same conditions as specified in section 1.2.2. Purification of the product on a silica gel (2:1 then 1:1 petroleum ether/EtOAc) afforded the corresponding amide with a 65% yield. ES/MS:m/z ratio 1169.7 ([M+H]⁺).

2.9.3. (3S, 9R)-3-[(R)-3-dodecanoyloxytetradecanoylamino]-4-oxo-5-aza-9-[(R)-3-benzyloxytetradecanoylamino]-decan-1,10-diol 1-dibenzyl-phosphate

A solution of the coupling reaction product obtained above (1.05 g; 0.90 mmol.) in a 1/1 EtOH/EtOAc mixture (65 ml) containing Et₃N (1.5 ml) is hydrogenated in presence of Pd on carbon containing 10% Pd (50 mg) at room temperature and under atmospheric pressure hydrogen for 1 hour. The catalyst is filtered off and the filtrate is evaporated to dryness and then dried by suction from a vacuum pump. The residue is then dissolved in a 1/1 i-PrOH/CH₂Cl₂ mixture (50 ml) and stirred for 10 minutes at room temperature with an Amberlite IR-120 (H⁺) resin (3 ml). The resin is filtered off and the filtrate is evaporated to dryness to provide the free acid (956 mg ; 99%) as a white cristalline solid.

To a solution of the acid obtained above (855 mg; 0.79 mmol.) in anhydrous THF (5 ml) at 0° C. and under argon flow, there are added N-methylmorpholin (87 μl; 0.79 mmol.; 1 eq.) then isobutyl chloroformate (103 μl; 0.79 mmol.; 1 eq.). Rapid formation of an N-methylmorpholin hydrochloride precipitate is observed. After stirring for 30 minutes at room temperature, the reaction mixture is cooled down to 0° C. and a solution of NaBH₄ (60 mg; 1.58 mmol.; 2 eq.) in H₂O (2 ml) is then quickly added. As soon as gaz emission has ceased (5 min.), the solution is diluted with H₂O (2 ml) and THF (2 ml) and then stirred for 5 minutes at room temperature. The solution is concentrated, diluted with CH₂Cl₂ and H₂O, neutralized with a solution of 1M HCl followed by layer separation. The organic layer is dried over MgSO₄, filtered and evaporated. By running a flash chromatography purification on a silica gel (4/1 CH₂Cl2/acetone eluent), the reduced-state product is recovered (387 mg; 46%) as a white cristalline solid.

To a solution of this alcohol (313 mg; 0.29 mmol.) and 1H-tetrazole (62 mg; 0.88 mmol.; 3 eq.) in anhydrous THF (12 ml) at room temperature and under argon flow, there is added 85% dibenzyl-diethyl phosphoramidite (267 μl; 0.67 mmol.; 2.3 eq.). Rapid formation of white cristals in the reaction medium can be seen. After stirring for 30 minutes, the reaction mixture is cooled down to −20° C. and an mCPBA solution (57-85%; 187 mg; 1.08 mmol.; 3.7 eq.) in CH₂Cl₂ (8 ml) is then added. Disappearance of cristals is noted. After stirring for 45 minutes at room temperature; a saturated Na₂S₂O₃ solution (5 ml) is added and the reaction mixture is then stirred for 10 minutes. The solution is next diluted with ether, and the organic layer is then separated and washed with a saturated solution of Na₂S₂O₃ (5×), a saturated solution of NaHCO₃ (2×) and a 1M HCl solution (1×). The organic layer is dried over MgSO₄, filtered and evaporated. By running a flash chromatography purification on a silica gel (8/1 then 5/1 CH₂Cl₂/acetone eluent), there is recovered the phosphotriester (361 mg 93%) as an amorphous solid.

This product is subjected to a hydrolysis reaction to split the benzyloxymethyl acetal in THF-HCl aqueous medium to thereby obtain (3S, 9R)-3-[(R)-3-dodecanoyloxytetradecanoylamino]-4-oxo-5-aza-9-[(R)-3-benzyloxytetradecanoylamino]-decan-1,10-diol 1-dibenzylphoshate.

2.9.4. (3S, 9R)-[(R)-3-dodecanoyloxytetradecanoylamino]-4-oxo-5-aza-9-[(R)-3-hydroxytetradecanoylamino]-decan-1,10-diol 1-dihydrogenphosphate 10-(6,7-dihydroxyheptanoate)

The product obtained above is O-acylated on carbon number 10 with hept-6-enoic acid in presence of both EDCl in dichloromethane at 0° C. and DMAP (see section 2.3.1.). This heptenoic acid ester is then subjected to a hydroxylation reaction in presence of (catalytic) osmium tetraoxide and N-methylmorpholin oxide (see section 2.2.2.) to thereby yield the corresponding diol (6, 7-dihydroxyheptanoic acid ester). This product is deprotected by hydrogenolysis in ethanol under atmospheric pressure hydrogen in presence of a palladium on carbon catalyst (see section 2.2.3.).

2.9.5. (3S, 9R)-3-[(R)-3-dodecanoyloxytetradecanoylamino]-4-oxo-5-aza-9-[(R)-3-hydroxytetradecanoylamino]-decan-1,10-diol 1-dihydrogenphosphate 10-(6-oxohexanoate)

The above deprotected diol is subjected to a periodic oxidation reaction in an isopropanol-water mixture (experimental procedure, see section 2.2.1.)

2.9.6. (3S, 9R)-3-[(R)-3-dodecanoyloxytetradecanoylamino]-4-oxo-5-aza-9-[(R)-3-hydroxytetradecanoylamino]-decan-1,10-diol 1-dihydrogenphosphate 10-(6-hydroxyhexanoate) (=OM-197-FV8)

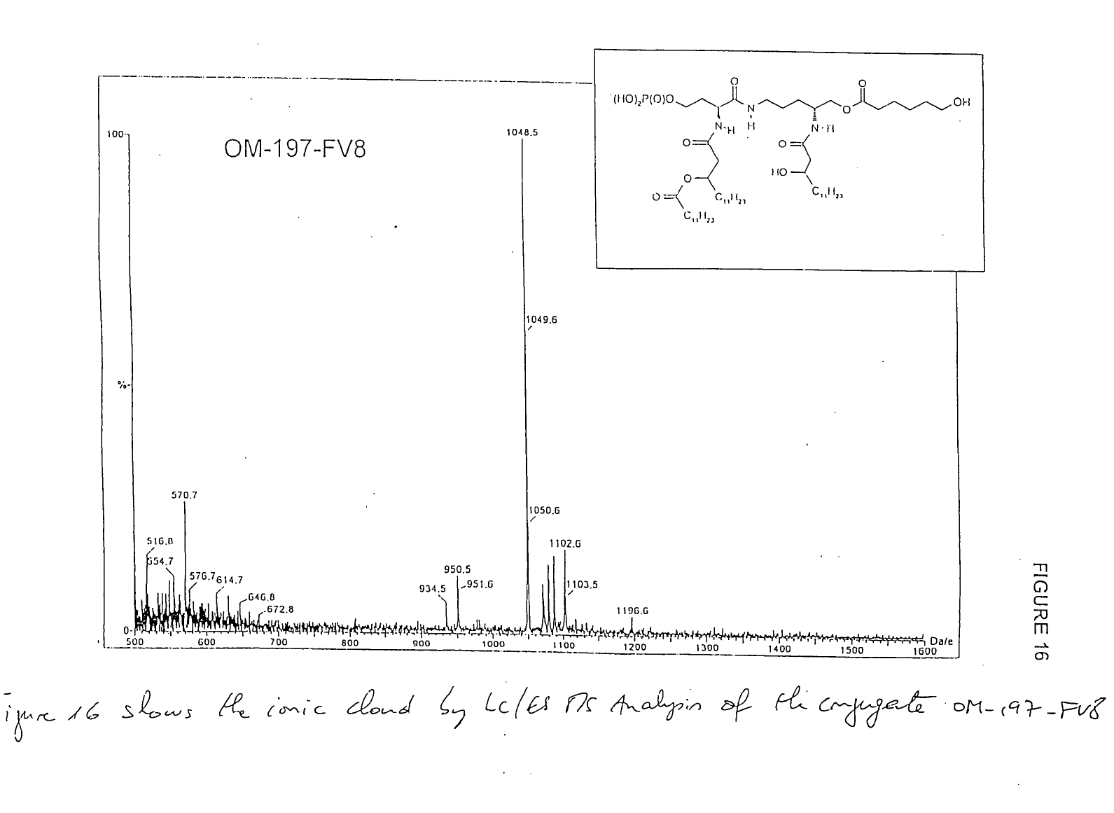

To a solution of 6-oxohexanoic acid derivative as obtained above (4.5 mg, 0.0043 mmol.) in 90% isopropanol (20 ml), there is added a solution of NaBH₄ (0.2 ml) in methanol (1 mg/ml). The mixture is stirred for 3 min. at 25° C., then excess acetic acid (0.2 ml) is added. By running a preparative HPLC purification on a C18 column, there is recovered the product OM-197-FV8 (2 mg, 44%), in 90% isopropanol. ES/MS: m/z 1048.5 [M+H}⁺; 950.5 [M+H−(HO)₂OPOH]+(FIG. 16).

### Example 2.10.

### 2-[(R)-3-hydroxytetradecanoylamino]-5-(6-oxohexyl)amino{-pentyl 2-((R)-3-dodecanoyloxytetradecanoylamino]-4-(dihydroxy-phosphoryloxy)butanoate (=OM-144-FP9) (FIG. 17)

2.10.1. Hept-6-enyl Trifluoromethanesulfonate

Triflic anhydride Tf₂O ((CF₃.SO₂)₂O, 11 ml; 6.67 mmol.) is added dropwise at −15° C. to a solution of hept-6-en-1-ol (515 mg ; 4.51 mmol.) and Et₃N (627 μl; 4.51 mmol.) in CH₂Cl₂ (10 ml) and the mixture is stirred for 30-45 minutes at −15° C. until no alcohol is left. After warming up to room temperature, the medium is diluted with CH₂Cl₂ and washed in succession with H₂O, a saturated aqueous solution of NaHCO₃, a saturated aqueous solution of NaCl. The organic layer thus obtained is dried over MgSO₄ and concentrated under vacuum to finally yield a residue which is taken up in a (1/2) ethyl acetate/petroleum ether mixture and filtered on a silica gel (removal of triethylamine salts formed during the reaction). After evaporation of the filtrate, the desired triflate compound is obtained with a 87% yield. (956 mg) and is used in the next step with no further purification. Rf=0.8 in 1/2 ethyl acetate/petroleum ether. ¹H-NMR (CDCl₃, 250 MHz), δ in ppm: 5.7, 5.0, 4.45, 2.0, 1.8, 1.4, ¹³C-NMR (CDCl₃, 62.89 MHz), δ in ppm: 138.21, 114.95, 77.68, 33.40, 29.13, 28.10, 24.53

2.10.2. (2R)-2-[(R)-3-benzyloxytetradecanoylamino]-5-(hept-6-enyl)amino-pentan-1-ol

A solution of freshly prepared triflate hereinabove (956 mg ; 3.88 mmol.) in CH₂Cl₂ (10 ml) is added dropwise to a solution of (2R)-5-amino-2-[(R)-3-benzyloxytetradecanoylamino]pentan-1-ol (Section 1.1.2.) (1.69 g; 3.88 mmol) in CH₂Cl₂ (10 ml) and the mixture is stirred for 4 hours at room temperature under argon flow. After dilution with CH₂Cl₂, the reaction medium is successively washed with an aqueous saturated solution of NaHCO₃ and with H₂O. The organic layer thus obtained is dried over MgSO₄ and concentrated under vacuum. By running a flash chromatography purification on a silica gel (15/1 CH₂Cl₂/MeOH eluent), there is recovered the desired secondary amine (862 mg; 43%) Rf=0.3 (8/1 CH₂Cl₂/MeOH). ES/MS: m/z ratio 532.0 [M+H]⁺. ¹H-NMR (CDCl₃, 250 MHz), δ in ppm: 7.2-7.4; 7.1; 5.8; 5.0; 4.6; 3.95; 3.5; 2.9; 2.5; 2.1; 1.9-1.5; 1.5-1.2; 0.9. ¹³C-NMR (CDCl₃, 62.89 MHz), δ in ppm: 172.17; 138.28; 138.21; 128.39; 127.96; 127.71; 114.82; 76.80; 71.40; 63.81; 50.87; 48.30; 47.75; 41.57; 34.10; 33.39; 31.89; 29.66; 29.62; 29.33; 28.19; 28.03; 25.21 26.11; 25.78; 22.82; 22.66; 14.10. ¹H-NMR (CDCl₃, 250 MHz), δ in ppm: 7.2-7.4; 6.75; 5.8; 5.0; 4.5; 3.9; 3.5; 2.5; 2.0; 2.1; 1.7-1.2; 0.9. ¹³C-NMR (CDCl_{3, 62.89} MHz), δ in ppm: 172.77; 138.68; 138.14; 128.23; 127.72; 127.56; 114.22; 76.64; 71.37; 64.58; 51.45; 49.79; 49.37; 41.53; 33.90; 33.53; 31.77; 29.65; 29.20; 28.64; 28.57; 25.00; 26.68; 25.93; 22.53; 13.98 (ammonium salt and free base spectra, respectively).

2.10.3. {2-[(R)-3-benzyloxytetradecanoylamino]-5-(hept-6-enyl)amino}pentyl 2-((R)-3-dodecanoyloxytetradecanoylamino]-4-(diphenyloxyphosphoryloxy)butanoate

To a solution of the secondary amine obtained above (163 mg; 0.307 mmol.; 1 eq.) and 4-(diphenyloxyphosphoryloxy)-2-[(R)-3-dodecanoyloxytetradecanoylamino]butanoic acid (Section 1.1.1.) (278 mg; 0.368 mmol.; 1.2 eq.) in CH₂Cl₂ (25 ml), there is added N, N-diisopropylethylamine (DIEA) (54 μl; 1 eq.). The medium is cooled down to 0° C. and EDCl (71 mg; 1.2 eq.) and 1-hydroxy-7-azabenzotriazole (HOAt) (41 mg 1 eq.) are then added. The reaction mixture is stirred for 2 hours at 0° C. and thereafter for 90 hours at room temperature. The reaction medium is then washed with H₂O and the organic layer is dried over MgSO₄ and subsequently evaporated under vacuum at 40° C. By running a silica gel flash chromatography purification (15/1 CH₂Cl₂/MeOH eluent), recovery of the O-acylated product (126 mg; 32%) is achieved.

2.10.4. {2-[(R)-3-benzyloxytetradecanoylamino]-5-(6,7-dihydroxy-heptyl)amino}pentyl 2-[(R)-3-dodecanoyloxy-tetradecanoyl-amino]-4-(diphenyloxyphosphoryloxy)-butanoate

A freshly prepared 2.5% OsO₄ solution in pyridine (1.1 ml; 1.9 eq.) is added dropwise at 25° C. to a solution of the O-acylation reaction product prepared above (70 mg, 0.055 mmol.) in anhydrous pyridine (5 ml). The mixture is stirred for 24 to 48 hours at room temperature and then treated by addition of Na₂S₂O₅ and finally diluted with CH₂Cl₂, washed in succession with H₂O, an aqueous solution of 1N HCl and H₂O again. The resulting organic layer is dried over MgSO₄, filtered, evaporated and the residue is subjected to purification by flash chromatography treatment on a silica gel (15/1-10/1 CH₂Cl₂/MeOH eluent gradient) to thereby recover the desired diol (27 mg ; 38%). HPLC (210 nm): T_{R}=37.25 min0 ES/MS: m/z ratio 1307.0 [M+H]⁺.

2.10.5. {2-[(R)-3-hydroxytetradecanoylamino]-5-(6,7-dihydroxy-heptyl)amino}pentyl 2-[(R)-3-dodecanoyloxytetradecanoylamino]-4-(dihydroxyphosphoryloxy)butanoate

a. Debenzylation

To a solution of the above obtained diol (50 mg; 0.038 mmol.) in a HPLC-grade MeOH/AcOH mixture (5/0.2 ml), addition of a catalyst (10% Pd/C) (10 mg) is made. The reaction mixture is stirred for 4 hours under H₂ (atmospheric pressure) at room temperature. The catalyst is filtered off on a millipore filter and the filtrate is evaporated to finally recover, with no further purification, the debenzylated product (46 mg; 99%). HPLC (210 nm): T_{R}=35.13 and 35.51 min. (two diastereoisomers were detected). ES/MS: m/z ratio 1217.0 [M+H]⁺.

b. Dephenylation:

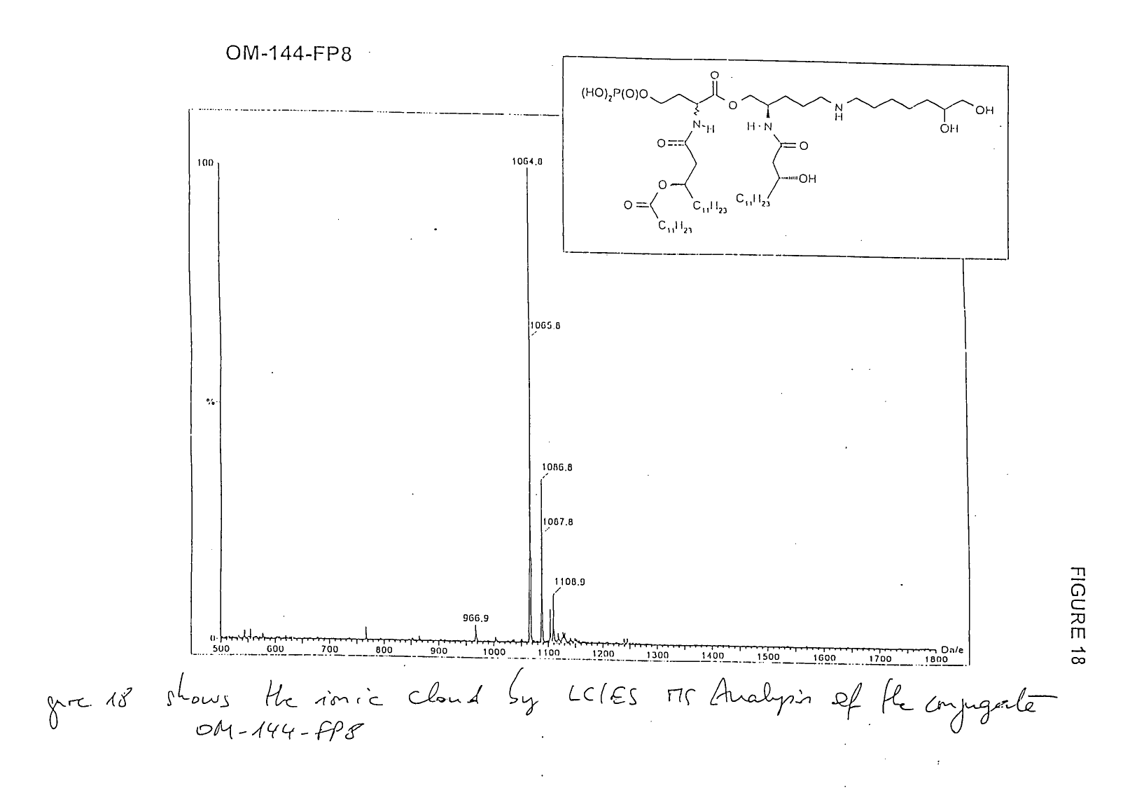

Preactivation of a catalyst suspension (PtO₂) (25 mg; 3.8 eq.) in HPLC-grade EtOH (0.5 ml) is conducted under H₂ for 10 minutes. A solution of the debenzylated product obtained above (35 mg, 0.029 mmol.) in HPLC-grade EtOH (2 ml) from which air was flushed under argon, is added to the catalyst suspension. The reaction mixture is then stirred for 2 hours under H₂ at room temperature. The catalyst is filtered off on a millipore filter and the filtrate is evaporated to finally recover, with no further purification, the desired phosphate ester. (26 mg; 87%). HPLC (210 nm): T_{R}=29.3 and 30.9 min. (two diastereoisomers were detected): ES/MS: m/z ratio 1064.8 [M+H]⁺, 1086.8 [M+Na]⁺, 1108.8 [M−H+2Na]⁺(FIG. 18).

2.10.6. {2-[(R)-3-hydroxytetradecanoylamino]-5-(6,7-oxohexyl)amino}pentyl 2-[(R)-3-dodecanoyloxytetradecanoylamino]-4-(dihydroxyphosphoryloxy)butanoate (=OM-144-FP9)

1.87 ml of 0.1 M NaIO₄ (40.17 mg, 20 eq.) are added to 10 mg (9.39 μmol. , 1 eq.) of the deprotected product prepared above in an isopropanol-water mixture (1:1). The solution is stirred for 2 hours at room temperature. The reaction is stopped by addition of 1 to 2 drops of ethylene glycol. T_{R}=30.0 and 31.5 min. (two diastereoisomers were detected). ES/MS: m/z ratio 1032.8 [M+H]⁺, 1054.8 [M+Na]⁺.

### Example 2.11.

### (2R, 8R)-2-[(R)-3-dodecanoyloxytetradecanoylamino]-3-oxo-4-aza-8-[(R)-3-hydroxytetradecanoylamino]-nonan-1,9-diol 1-O-carboxymethyl ether 9-O-(6-oxohexanoate) (=OM-112-FV7) (FIG. 19)

D-serine is protected in the form of an N-(p-methoxybenzyloxycarbonyl) derivative [Reference. Chen. and Wang, Synthesis (1989): 36-37], then the OH functional group is alkyl-substituted with benzyl bromoacetate in presence of NaH (2 eq.). The amine functional group is freed by trifluoroacetic acid treatment in dichloromethane, then acylated with (R)-3-dodecanoyloxytetradecanoic acid chloride treatment in presence of triethylamine. The serine derivative thus obtained is coupled with (2R)-5-amino-2-[(R)-3-benzyloxytetradecanoylamino]pentan-1-ol amine (see section 4.1.1.) in presence of IIDQ to yield the (2R, 8R)-2-[(R)-3-dodecanoyloxytetradecanoylamino]-3-oxo-4-aza-8-[(R)-3-benzyloxytetra-decanoylamino]nonane-1,9-diol 1-O-benzyloxycarbonylmethylether. This product is then O-acylated with hept-6-enoic acid in presence of EDCl. The double bond of the accessory ester is hydroxylated with osmium tetraoxide (catalytic amount, in presence of N-methylmorpholin N-oxide), and the diol is then deprotected through hydrogenolysis in presence of palladium on carbon in an ethanol solution. The OM-112-FV-7 product is obtained by treatment with sodium periodate in an isopropanol-water mixture. C₆₅H₁₀₃N₃O₁₂. MM: 1010.45.

### Example 2.12.

### (2S, 8R)-1-(carboxymethyl)-thio-2-[(R)-3-tetradecanoyloxy-tetradecanoylamino]-3-oxo-4-aza-8-[(R)-3-hydroxy-tetradecanoyl-amino)-nonan-9-ol 9-O-(7-aminoheptanoate) (=OM-212-AH1) (FIG. 20)

D-cysteine is S-alkyl substituted with p-methoxybenzyl bromoacetate in presence of sodium carbonate in a THF-water medium. The S-benzyloxycarbonylmethylcysteine thus obtained is N-acylated with (R)-3-tetradecanoyloxytetradecanoic acid chloride, and the S-alkyl-N-acyl-D-cysteine derivative is then coupled with (2R)-5-amino-2-[(R)-3-hydroxytetradecanoylamino]pentan-1-ol amine {obtained by hydrogenolysis of the (2R)-5-amino-2-[(R)-3-benzyloxy-tetradecanoyl-amino]pentan-1-ol, see 4.1.1.} in presence of IIDQ. The thus obtained product is selectively O-acylated in its primary position with the ester derived from reacting HOBt with 7-(p-methoxybenzyloxy-carbonylamino)heptanoic acid. The two p-methoxybenzyl groups are then removed by treating the ester with aqueous trifluoroacetic acid. C₅₉H₁₁₂N₄O₁₀S. MM 1069.64.

2.16.

### Example 2.13.

### (2R,8R)-2-[(R)-3-dodecanoyloxytetradecanoylamino]-3-oxo-4-aza-8-[(R)-3-hydroxytetradecanoylamino]-nonane-1,9-diol 1-O-(2,2-di-carboxyethyl) ether 9-O-(6-oxohexanoate) (=OM-312-FV7) (FIG. 21).

The N-(p-methoxybenzyloxycarbonyl) D-serine derivative is O-alkyl substituted with dibenzyl methylenemalonate in presence of NaH. The amine functional group is freed by trifluoroacetic acid treatment in dichloromethane, and then acylated by (R)-3-dodecanyloxytetradecanoic acid chloride treatment in presence of triethylamine. The serine derivative thus obtained is coupled with (2R)-5-amino-2-[(R)-3-benzyloxy-tetosm

radecanoylamino]pentan-1-ol amine (see section 4.1.1.) in presence of IIDQ to yield the (2R, 8R)-2-[(R)-3-dodecanoyloxy-tetradecanoylamino]-3-oxo-4-aza-8-[(R)-3-benzyloxytetradecanoyl-amino]nonan-1,9-diol 1-O-[2,2-bis-(benzyloxycarbonyl)ethyl]ether. This product is O-acylated with hept-6-enoic acid in presence of EDCl, then the heptenoyl ester is subjected to a hydroxylation reaction with the aid of osmium tetraoxide. The benzyl groups are cleaved by hydrogenolysis in presence of palladium on carbon in an ethanol solution. The OM-312-FV-7 product is obtained by treating the debenzylated product with sodium periodate in an isopropanol-water mixture. C₅₈H₁₀₅N₃O₁₄. MM: 1068.49.

### Example 2.14.

### 3-[(R)-3-dodecanoyloxytetradecanoylamino]-4-oxo-5-aza-9-[(R)-3-hydroxytetradecanoylamino]-1,10-diol-1-dihydrogenphate 10-(6-bromoacetamidohexanoate) (=OM-412-BA7) (FIG. 22).

The 3-[(R)-dodecanoyloxytetradecanoylamino]-4-oxo-5-aza-9-[(R)-3-hydroxytetradecanoylamino]-1,10-diol-1-dihydrogenphate 10-(6-aminohexanoate) (see section 4.2.3.3.) is subjected to a bromoacetylation reaction by means of bromoacetic acid succinimidyloxy ester compound in a water-DHF medium in presence of triethylamine. The final product is purified by HPLC. C₅₇H₁₀₈BrN₄O₁₃P. MM: 1168.4.

(3R, 9R)-3-[(R)-hydroxytetradecanoylamino]-4-oxo-5-aza-9-[(R)-dodecanoyloxytetradecanoylamino]-decan-1,10-diol 1-dihydrogenphate 10-O-(6-oxohexanoate) (OM-512-FV7) (FIG. 23)

The 2-[(R)-3-benzyloxytetradecanoylamino]-4-diphenyloxy-phosphoryloxy-butanoic acid [obtained by N-acylation of benzyl O-(diphenyloxyphosphoryl)-DL-homoserinate trifluoroacetic salt with (R)-3-benzyloxytetradecanoic acid chloride, then cleavage of the benzyl ester by hydrogenolysis in ethanol in presence of triethylamine and a palladium on carbon catalyst] is coupled with (2R)-5-amino-2-[(R)-3-dodecanoyloxytetradecanoylamino]pentan-1-ol [obtained by N-acylation of (2R)-5-(benzyloxycarbonylamino)-2-amino-pentan-ol trifluoroacetic salt with (R)-3-dodecanoyloxytetradecanoic acid chloride, then deprotection of the amino group in C₅ position by hydrogenolysis in ethanol in presence of triethylamine and a palladium on carbon catalyst] in presence of IIDQ. The thus formed amide is O-acylated at the free OH functional group with 6-heptenoic acid in presence of EDCl to yield the corresponding ester. The double bond of the additional ester is subjected to a hydroxylation reaction with osmium tetraoxide; and the benzyl group is then removed through hydrogenolysis by using a palladium on carbon catalyst and the phosphate compound is freed through hydrogenolysis over platinium black. The diol functional group is subjected to a periodic oxidation reaction to form the 6-oxohexanoyl derivative OM-512-FV7. C₅₅H₁₀₄N₃O₁₃P. MM: 1046.42.

### Example 2.16.

### N-[(R)-3-dodecanoyloxytetradecanoyl]-D-aspartic acid, α-N-{(4R)-5-(6-benzyloxycarbonylaminohexanoyloxy)-4-[(R)-3-hydroxytetradecanoyl-amino]pentyl}amide (=OM-197-MC-AC) (FIG. 24)

2.16.1. N-[(R)-3-dodecanoyloxytetradecanoyl]-D-aspartic acid, α-N-{(4R)-5-(aminohexanoyloxy)-4-[(R)-3-benzyloxytetradecanoyl-amino]pentyl}amide β-benzyl Ester

To a solution of N-[(R)-3-dodecanoyloxytetradecanoyl]-D-aspartic acid, α-N-{(4R)-5-hydroxy-4-[(R)-3-benzyloxytetradecanoyl-amino]pentyl}amide β-benzyl ester (400 mg, 0.38 mmol.) and 6(benzyloxycarbonylamino)hexanoic acid (220 mg, 0.83 mmol.) in dry CH₂Cl₂ (15 ml) at 0° C. and under argon flow, there are added in succession commercially available 1-(3-dimethylaminopropyl)-3-ethylcarbodiimide hydrochloride (162 mg, 0.85 mmol.) and 4-dimethylaminopyridine (13 mg, 98 μmol.). The reaction mixture is then stirred for 30 minutes at 0° C. and thereafter overnight at room temperature. The reaction medium is then washed with water and a solution of 1N HCl followed by layer separation. The organic layer is dried over MgSO₄, filtered and evaporated. By running a flash chromatography purification on a silica gel (9/1 then 4/1 CH₂Cl₂/acetone eluent), there is recovered the coupling reaction product (395 mg; 81%) as a white solid. ¹³C-NMR (62,89 MHz, CDCl₃), δ in ppm: 173.46, 173.29, 171.83, 171.14, 170.12, 156.38, 138.19, 136.60, 133.30, 128.51, 128.42, 128.31, 127.70, 127.58, 119.97, 76.49, 71.23, 71.06, 66.73, 66.47, 49.21, 47.83, 41.42, 40.73, 39.14, 35.46, 34.86, 33.70, 31.84, 29.57, 29.46, 29.28, 29.10, 26.01, 25.54, 25.17, 25.08, 24.94, 24.31, 22.61, 14.05

2.16.2. N-[(R)-3-dodecanoyloxytetradecanoyl]-D-aspartic acid, α-N-{(4R )-5-(aminohexanoyloxy)-4-[(R)-3-hydroxytetradecanoylamino-pentyl}amide

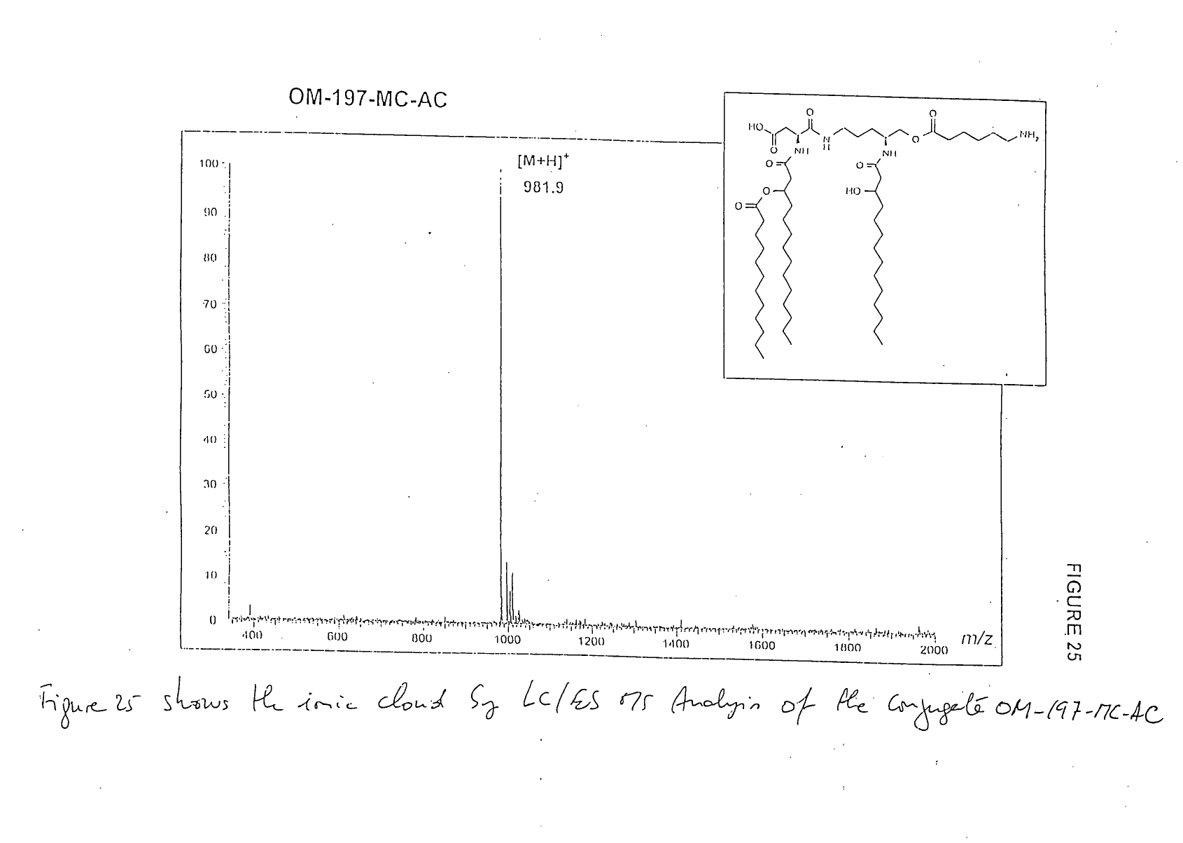

A solution of the compound prepared above (340 mg, 0.26 mmol.) in a 5/1 CH₂Cl₂/ethanol mixture (24 ml) containing acetic acid (2 ml) is hydrogenated in presence of Pd on carbon containing 10% Pd (40 mg) at room temperature and under atmospheric pressure hydrogen for 12 to 24 hours. The catalyst is filtered off. The filtrate is evaporated to dryness and the residue is then dried by suction from a vacuum pump to obtain N-3-[(R)-3-dodecanoyloxytetradecanoyl]-D-aspartic acid, α-N-{(4R)-5-(6-aminohexanoyloxy)-4-[(R)-3-hydroxytetradecanoylamino]-pentyl}amide (238 mg, quantitative yield). C₅₅H₁₀₄N₄O₁₀: ES/MS: m/z ratio 981.9 ([M+H]⁺); (FIG. 25).

### Example 2.17.

### N-[(R)-3-dodecanoyloxytetradecanoyl]-D-aspartic acid, α-N-{(4R)-5-(6-succinylamidohexanoxyloxy)-4-[(R)-3-hydroxy-tetradecanoylamino]pentyl}amide (=OM-197-MC-AC-Succ) (FIG. 24)

To a solution of product of example 2.16. (Section 2.16.2) (50 mg; 0.051 mmol.) in pyridine (3 ml), there are added in succession succinic anhydride (11 mg, 0.11 mmol.) and 4-N, N′-dimethylaminopyridine (7 mg; 0.57 mmol.). After stirring for 6 hours at 50° C. under Ar, methanol (2 ml) is added and the reaction medium is stirred for further 15 min. at room temperature. The solvent is evaporated and the product is purified by a flash chromatography treatment on a silica gel (7:1 then 5:1 CH₂Cl₂/MeOH eluent); thereby obtaining the product as a white solid (42 mg; 74%). C₅₉H₁₀₈N₄O₁₃ ES/MS; m/z ratio 1082 ([M+H])⁺, Rf=0.1 (4:1 CH₂Cl₂/MeOH)

### Example 2.18.

### N-[(R)-3-dodecanoyloxytetradecanoyl]-D-aspartic acid, α-N-{(4R)-5-glycinyloxy)-4-[(R)-3-hydroxy-tetradecanoylamino]pentyl}-amide (=OM-197-MC-AC-Gly) (FIG. 24)

2.18.1. N-[(R)-3-dodecanoyloxytetradecanoyl]-D-aspartic acid, α-N-{(4R)-5-(benzyloxvcarbonylaminoacetoxy)-4-[(R)-3-benzyloxytetradecanoyl-amino]pentyl}amide β-benzyl Ester

To a solution of N-[(R)-3-dodecanoyloxytetradecanoyl]-D-aspartic acid, α-N-{(4R)-5-hydroxy-4-[(R)-3-benzyloxytetradecanoylamino]pentyl}-amide β-benzyl ester (300 mg, 0.29 mmol.) and commercially available N-benzyloxycarbonylglycine (101 mg, 0.49 mmol.) in dry CH₂Cl₂ (10 ml) at 0° C. and under argon flow, there are added in succession commercially available 1-(3-dimethylaminopropyl)-3-ethylcarbodiimide hydrochloride (93 mg, 0.48 mmol.) and 4-dimethylaminopyridine (6 mg, 49 μmol.). The reaction mixture is then stirred for 30 minutes at 0° C. and thereafter overnight at room temperature. The reaction medium is then washed with water and a solution of 1N HCl followed by layer separation. The organic layer is dried over MgSO₄, filtered and evaporated. By running a flash chromatography purification on a silica gel (8/1 then 6/1 CH₂Cl₂/acetone eluent), there is recovered the coupling reaction product (313 mg; 88%) as a white solid. ¹³C-NMR (62,89 MHz, CDCl₃), δ in ppm: 173.71, 173.49, 171.80, 171.68,171.21, 170.21, 170.06, 169.94, 169.84, 156.41, 138.18, 136.16, 135.26, 128.46, 128.33, 128.26, 128.04, 127.93, 127.65, 127.55, 76.49, 71.14, 71.07, 70.93, 66.90, 66.67, 66.38, 66.26, 49.21, 49.03, 47.72, 47.66, 42.58, 41.83, 41.68, 41.32, 39.02, 35.58, 35.39, 34.34, 33.84, 31.81, 29.52, 29.24, 29.06, 28.31, 28.13, 25.34, 25.12, 25.03, 24.89, 22.57, 14.01.

2.18.2 N-3-[(R)-3-dodecanoyloxytetradecanoyl]-D-aspartic acid, α-N-{(4R)-5-(glycinyloxy)-4-[(R)-3-hydroxytetradecanoylamino]-pentyl}amide

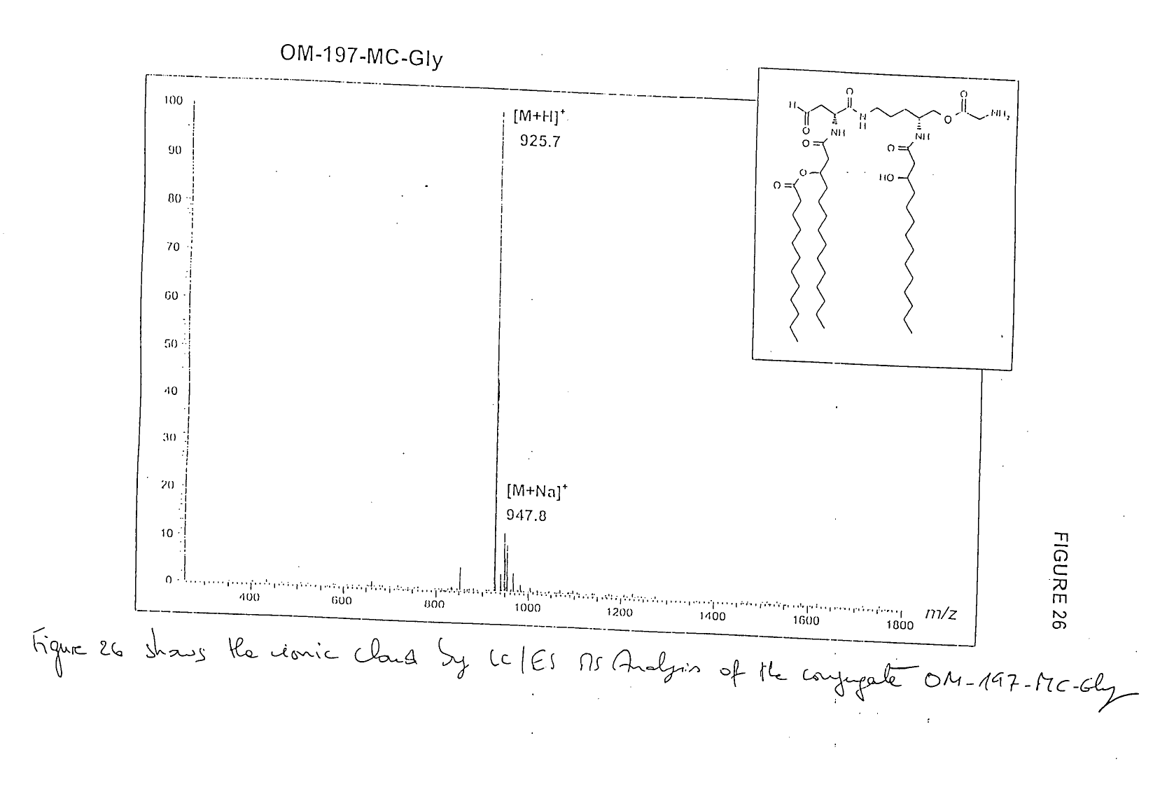

A solution of the compound prepared above (268 mg, 0.22 mmol.) in a 5/1 CH₂Cl₂/ethanol mixture (12 ml) containing acetic acid (2 ml) is hydrogenated in presence of Pd on carbon containing 10% Pd (30 mg) at room temperature and under atmospheric pressure hydrogen for 12 to 24 hours. The catalyst is filtered off. The filtrate is evaporated to dryness and the residue is then dried by suction from a vacuum pump to obtain N-3-[(R)-3-dodecanoyloxytetradecanoyl]-D-aspartic acid, α-N-{(4R)-5-(glycinyloxy)-4-[(R)-3-hydroxytetradecanoylamino]-pentyl}amide (200 mg, quantitative yield). C₅₁H₁₀₄N₄O₁₀: ES/MS: m/z ratio 925.7 ([M+H]⁺), 947.8 ([M+Na]⁺); (FIG. 26).

### Example 2.19.

### N-[(R)-3-dodecanoyloxytetradecanoyl]-D-aspartic acid, α-N-{(4R)-5-succinyloxy)-4-[(R)-3-hydroxy-tetradecanoylamino]pentyl)-amide (=OM-197-MC-Succ) (FIG. 27)

2.19.1. N-[(R)-3-dodecanoyloxytetradecanoyl]-D-aspartic acid, α-N-{(4R)-5-succinyloxy-4-[(R)-3-benzyloxytetradecanoylamino]pentyl}amide β-benzyl ester

To a solution of succinic anhydride (25 mg, 0.25 mmol) in dry CH₂Cl₂ (2 ml) in presence of Et₃N (40 μl, 0.29 mmol.) at 0° C., addition is made of a solution of N-[(R)-3-dodecanoyloxytetradecanoyl]-D-aspartic acid, α-N-{(4R)-5-hydroxy-4-[(R)-3-benzyloxytetradecanoylamino]pentyl}-amide β-benzyl ester (Section 1.2.2.) (150 mg, 0.14 mmol) in CH₂Cl₂ (5 ml). The reaction mixture is stirred for 10 minutes at 0° C. and thereafter at room temperature. The reaction medium is then diluted with CH₂Cl₂, washed with a 1N HCl solution followed by layer separation. The organic layer is dried over MgSO₄, filtered and evaporated. By running a flash chromatography purification on a silica gel (9/1 CH₂Cl₂/acetone eluent containing 2% acetic acid), there is recovered the acid product (148 mg; 90%) as a white solid. ¹³C-NMR (62,89 MHz, CDCl₃), δ in ppm: 175.03, 173.55, 173.35, 171.68, 171.92, 171.72, 171.36, 170.97, 170.60, 170.46, 170.41, 138.05, 135.24, 128.85, 128.76, 128.41, 128.27, 128.20, 128.03, 127.59; 76.48, 71.31, 70.77, 66.67, 65.08, 49.20, 48.11, 41.48, 41.31, 39.02, 35.80, 34.26, 34.13, 33.84, 31.77, 29.50, 29.21, 29.03, 25.05, 24.99, 24.83, 22.54, 13.98.

2.19.2 N-[(R)-3-dodecanoyloxytetradecanoyl]-D-aspartic acid, α-N-{(4R)-5-succinyloxy-4-[(R)-3-hydroxytetradecanoylamino]pentyl}amide (=OM-197-MC-Succ) (FIG. 27)

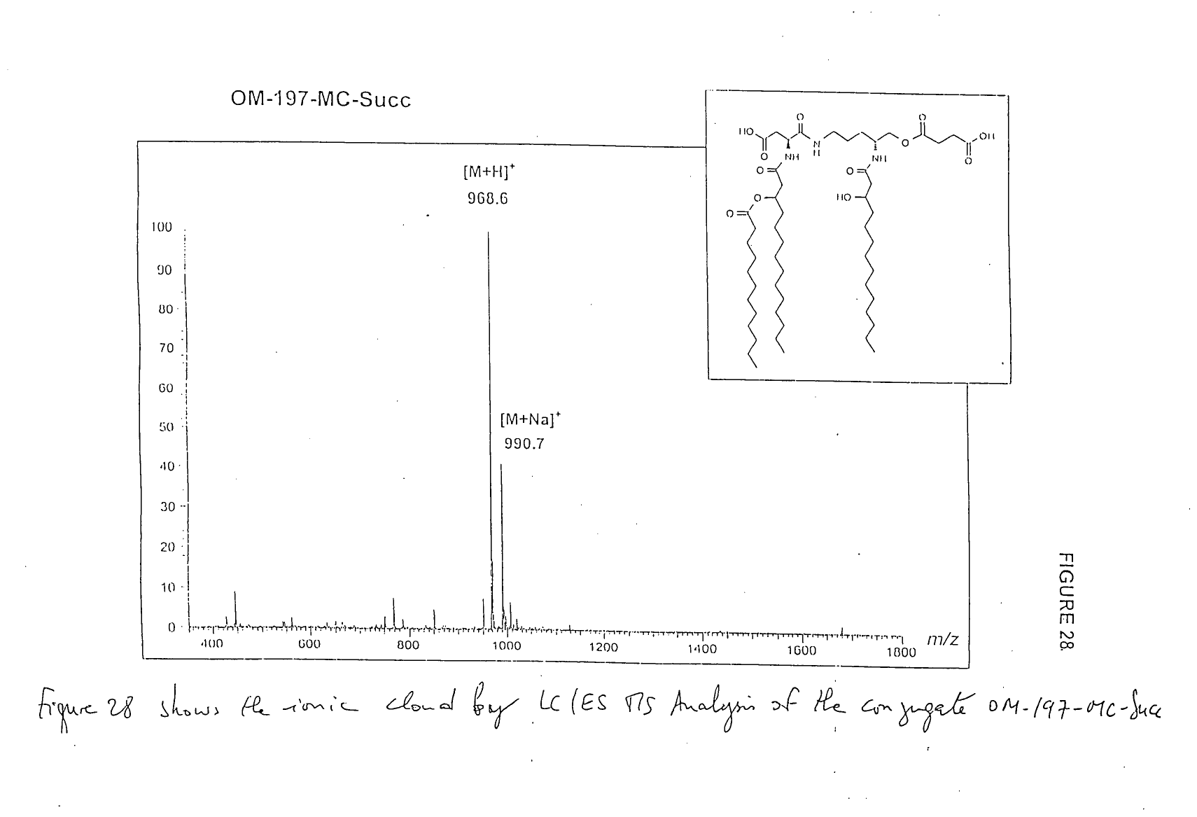

A solution of the compound prepared above (124 mg, 0.11 mmol.) is dissolved in hot .(HPLC-grade) EtOH (12 ml) containing acetic acid (1 ml) and then hydrogenated in presence of Pd on carbon containing 10% Pd (15 mg) at room temperature and under atmospheric pressure hydrogen for 10 hours. The catalyst is filtered off. The filtrate is evaporated to dryness and the residue is then dried by suction from a vacuum pump to recover the diacid product (102 mg, 97%). C₅₃H₉₇N₃O₁₂: ES/MS: m/z ratio 968.6 ([M+H]⁺), 990.7 ([M+Na]⁺); (FIG. 28).

### Example 2.20.

### N-[(R)-3-dodecanoyloxytetradecanoyl]-D-aspartic acid, α-N-{(4R)-5-hydroxy-4-[(R)-3-hydroxy-tetradecanoylamino]pentyl}-amide β-N-(3-aminopropyl)amide (=OM-197-AP) (FIG. 29)

2.20.1. N-[(R)-3-dodecanoyloxytetradecanoyl]-D-aspartic acid, α-N-{(4R)-5-hydroxy-4-[(R)-3-benzyloxytetradecanoylamino]pentyl}-amide

A solution of N-[(R)-3-dodecanoyloxytetradecanoylamino]-D-aspartic acid, α-N-{(4R)-5-hydroxy-4-[(R)-3-benzyloxytetradecanoylamino]-pentyl}amide β-benzyl ester (Section 1.2.2.) (2.53 g; 2.4 mmol.) in a 1/1 EtOH/EtOAc mixture (150 ml) containing Et₃N (4 ml) is hydrogenated in presence of Pd on carbon containing 10% Pd (120 mg) at room temperature and under atmospheric pressure hydrogen for 2 hours. The catalyst is filtered off and the filtrate is evaporated to dryness and then dried by suction from a vacuum pump. The residue is then dissolved in a 1/1 i-PrOH/CH₂Cl₂ mixture (100 ml) and stirred for 10 minutes at room temperature with an Amberlite IR-120 (H⁺) resin (5 ml). The resin is filtered off and the filtrate is evaporated to dryness to provide the free acid (2.25 g 97%) as a white cristalline solid. (Rf=0.45 in 9/1 CH₂Cl₂/MeOH containing 1% of acetic acid, phosphomolybdinium compound and U.V. color development agent). m.p.=115-117° C.

2.20.2 N-[(R)-3-dodecanoyloxytetradecanoyl]-D-aspartic acid, α-N-{(4R)-5-hydroxy-4-[(R)-3-benzyloxytetradecanoylaminolpentyl}amide β-N-(3-benzyloxycarbonylaminopropyl))amide

A solution of IIDQ (2-isobutoxy-1-isobutoxycarbonyl-1,2-dihydroquinoline) (98 g; 0.32 mmol.) in anhydrous CH₂Cl₂ (3 ml) is added to a solution of the compound prepared above (250 mg, 0.26 mmol.) in anhydrous CH₂Cl₂ (15 ml) at 0° C. and under argon flow. The reaction mixture is stirred for 15 minutes at 0° C. and a solution of commercially available 3-benzyloxycarbonylamino-propylamine hydrochloride (70 mg, 0.29 mmol.) in anhydrous CH₂Cl₂ (7 ml) containing triethylamine (40 μl, 0.29 mmol.) is added. After stirring for 18 hours, the solution is evaporated to dryness. By running a flash chromatography purification on a silica gel (20/1 CH₂Cl₂/MeOH eluent), there is recovered the coupling reaction product (217 mg; 78%) as a white solid.

2.20.3. N-[(R)-3-dodecanoyloxytetradecanoyl]-D-aspartic acid, α-N-{(4R)-5-hydroxy-4-[(R)-3-hydroxy-tetradecanoylamino]-pentyl}amide β-N-(3-aminopropyl)amide (=OM- 197-AP)

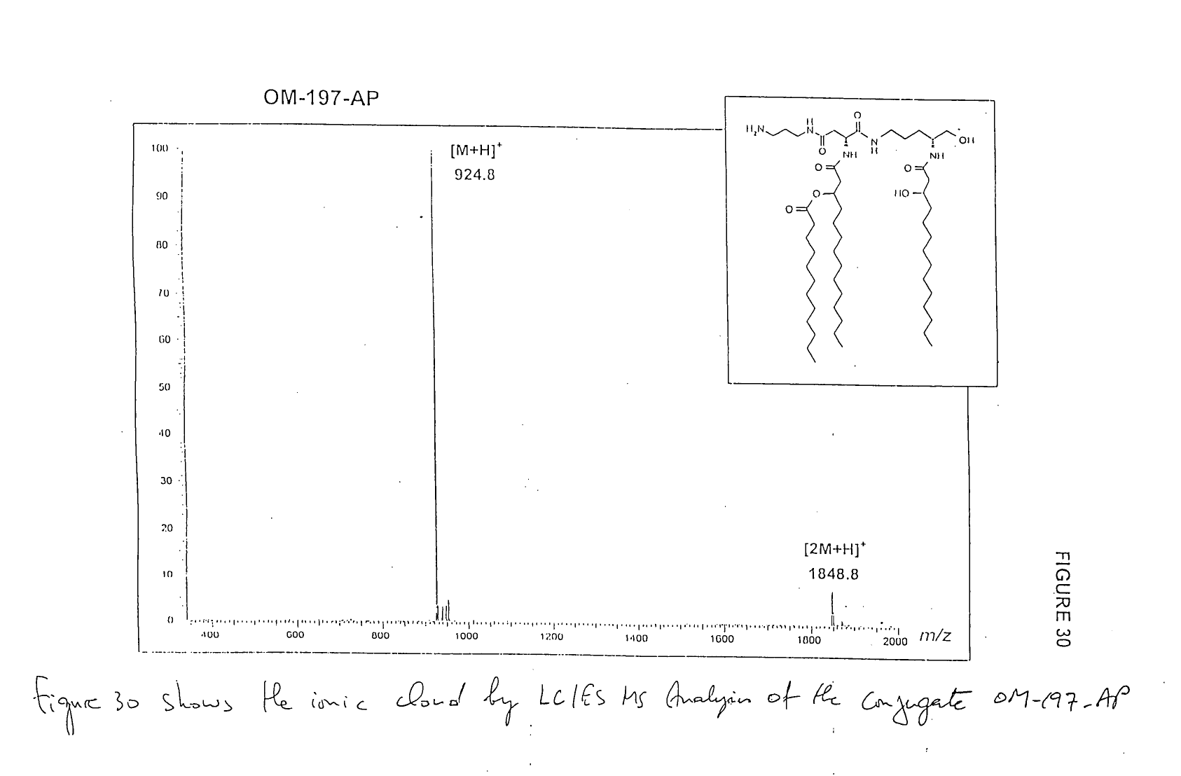

A solution of the compound prepared above (50 mg, 44 mmol.) in a 1/1 CH₂Cl_{2/}isopropanol mixture (8 ml) containing acetic acid (1 ml) is hydrogenated in presence of Pd on carbon containing 10% Pd (10 mg) at room temperature and under atmospheric pressure hydrogen for 6 to 8 hours. The catalyst is filtered off. The filtrate is evaporated to dryness and the residue is then dried by suction from a vacuum pump to obtain N-[(R)-3-dodecanoyloxytetradecanoyl]-D-aspartic acid, α-N-{(4R)-5-hydroxy-4-[(R)-3-hydroxytetradecanoylamino]-pentyl}amide β-N-(3-aminopropyl)amide (32 mg, 80%). C₅₂H₁₀₁N₅O₈: ES/MS: m/z ratio 924.8 ([M+H]⁺); 1848.8 ([2M+H]⁺);(FIG. 30).

### Example 2.21.

### N-[(R)-3-dodecanoyloxytetradecanoyl]-D-aspartic acid, α-N-{(4R)-5-hydroxy-4-[(R)-3-hydroxytetradecanoylamino]pentyl)-amide β-N-[(1S)-1-carboxy-5-aminopentylamide (=OM-197-Lys) (FIG. 31)

2.21.1. N-[(R)-3-dodecanoyloxytetradecanoyl]-D-aspartic acid, α-N-{(4R)-5-hydroxy-4-[(R)-3-benzyloxytetradecanoylamino]pentyl}-amide β-N-[(1S)-1-benzyloxycarbonyl-5-benzyloxycarbonylaminopentyl]amide

A solution of IIDQ (2-isobutoxy-1-isobutoxycarbonyl-1,2-dihydroquinoline) (114 mg; 0.38 mmol.) in anhydrous CH₂Cl₂ (5 ml) is added to a solution of N-[(R)-3-dodecanoyloxytetradecanoyl]-D-aspartic acid, α-N-{(4R)-5-hydroxy-4-[(R)-3-benzyloxytetradecanoylamino]-pentyl}amide (Section 2.20.1.) (300 mg, 0.31 mmol.) in anhydrous CH₂Cl₂ (20 ml) at room temperature and under argon flow. The reaction mixture is stirred for 15 minutes at RT then a solution of commercially available ε-N-benzyloxycarbonyl-L-lysine benzyl ester hydrochloride solution (140 mg, 0.31 mmol.) in anhydrous CH₂Cl₂ (5 ml) containing triethylamine (48 μl, 0.34 mmol.) is added. After stirring for 18 hours, the solution is evaporated to dryness. By running a flash chromatography purification on a silica gel (30/1 then 20/1 CH₂Cl₂/MeOH eluent), there is recovered the coupling reaction product (317 mg; 77%) as a white solid. ¹³C-NMR (62,89 MHz, CDCl₃), δ in ppm: 173.68, 172.35, 171.92, 170.74, 170.62, 170.54, 156.75, 156.52, 138.31, 136.54, 135.12, 128.54, 128.40, 128.28, 128.21, 127.96, 127.577, 127.59, 76.67, 71.40, 71.18, 67.20, 67.09, 66.48, 64.67, 52.30, 51.19, 41.64, 40.34, 39.24, 37.58, 34.40, 34.10, 31.83, 29.58, 29.27, 29.09, 25.16, 25.11, 24.93, 22.60, 14.04.

2.21.2. N-[(R)-3-dodecanoyloxytetradecanoyl]-D-aspartic acid, α-N-{(4R)-5-hydroxy-4-[(R)-3-hydroxytetradecanoylamino]pentyl}-amide β-N-[(1S)-1-carboxy-5-aminopentyl]amide

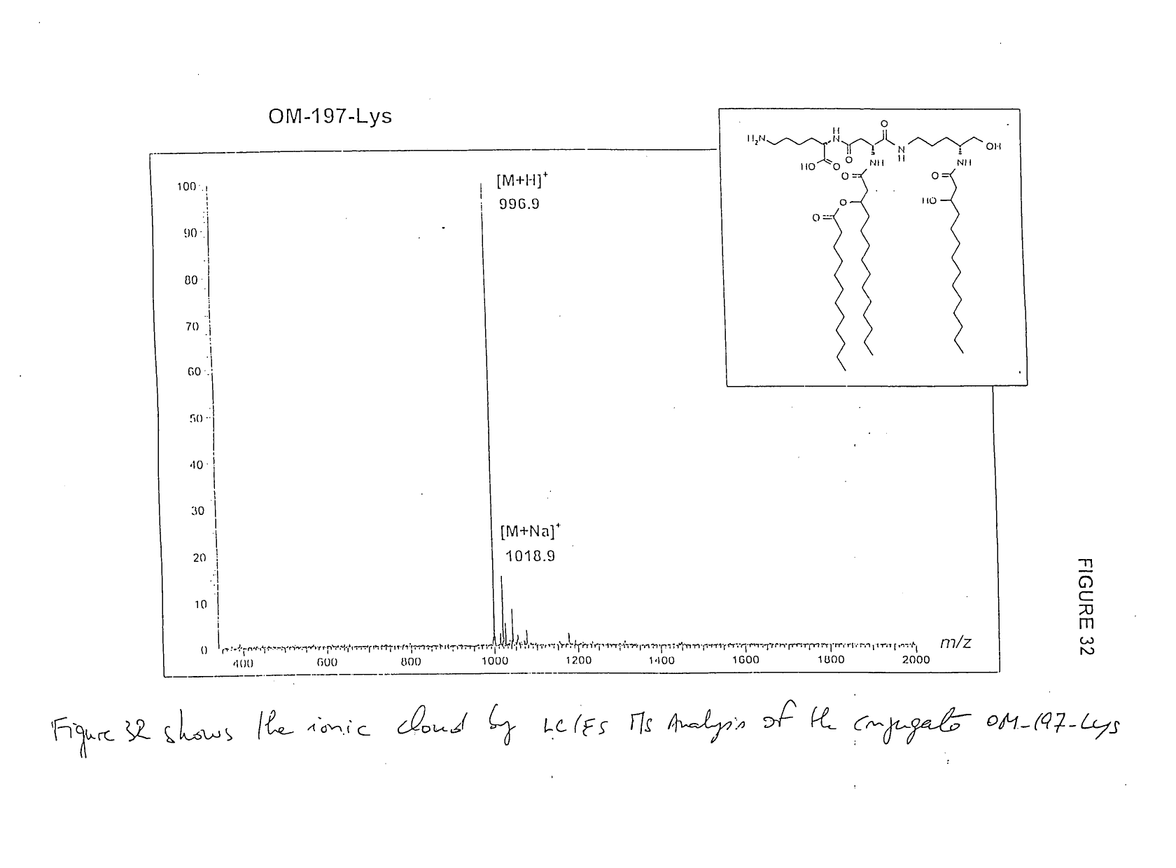

A solution of the above prepared compound (93 mg; 71 μmol.) in a 1/1 CH₂Cl₂/ethanol mixture (20 ml) containing glacial acetic acid (0.5 ml) is hydrogenated in presence of palladium on carbon containing 10% Pd (17 mg) at room temperature and under atmospheric pressure hydrogen for 12 to 24 hours. The catalyst is filtered off. The filtrate is evaporated to dryness and the residue is then dried by suction from a vacuum pump to provide N-[(R)-3-dodecanoyloxytetradecanoyl]-D-aspartic acid, α-N-{(4R)-5-hydroxy-4-[(R)-3-hydroxytetradecanoylamino]pentyl}-amide β-N-[(1S)-1-carboxy-5-aminopentyl]amide (71 mg, stoechiometric yield). C₅₅H₁₀₅N₅O₁₀ ES/MS: m/z ratio 996.9 ([M+H]⁺); 1018.9 ([M+Na]⁺) (FIG. 32).

### Example 2.22.

### N-[(R)-3-dodecanoyloxytetradecanoyl]-D-aspartic Acid, α-N-{(4R)-5-(6-aminohexanoyloxy)-4-[(R)-3-hydroxytetradecanoylamino]-pentyl}amide β-N-[(1S) 1-carboxy-5-aminopentylamide (=OM-197-Lys-AC) (FIG. 31)

2.22.1. N-[(R)-3-dodecanoyloxytetradecanoyl]-D-aspartic acid, β-N-{(4R)-5-(6-benzyloxycarbonylaminohexanoyloxy)-4-[(R)-3-benzyloxytetradecanoylamino]pentyl}-amide β-N-[(1S)-1-benzyloxycarbonyl-5-benzyloxycarbonylaminopentyl]-amide

To a solution of N-[(R)-3-dodecanoyloxytetradecanoyl]-D-aspartic acid, α-N-{(4R)-5-hydroxy-4-[(R)-3-benzyloxytetradecanoylamino]pentyl}-amide β-N-[(1S)-1-benzyloxycarbonyl-5-benzyloxycarbonylaminopentyl]-amide (Section 2.21.1.) (317 mg, 0.48 mmol.) and 6-(benzyloxycarbonylamino)hexanoic acid (128 mg, 0.48 mmol.) in dry CH₂Cl₂ (15 ml) at 0° C. and under argon flow, there are added in succession 1-(3-dimethylaminopropyl)-3-ethylcarbodiimide hydrochloride (93 mg, 0.48 mmol.) and 4-dimethylaminopyridine (12 mg, 0.98 μmol.). The reaction mixture is then stirred for 30 minutes at 0° C. and thereafter overnight at room temperature. The reaction medium is then washed with water and a solution of 1N HCl followed by layer separation. The organic layer is dried over MgSO₄, filtered and evaporated. By running a flash chromatography treatment on a silica gel (5/1 CH₂Cl₂/acetone eluent), there is recovered the coupling reaction product (226 mg; 71%) as a white solid.

2.22.2. N-[(R)-3-dodecanoyloxytetradecanoyl]-D-aspartic acid, α-N-{(4R)-5-(6-aminohexanoyloxy)-4-[(R)-3-hydroxytetradecanoylamino]pentyl}-amide β-N-[(1S)-1-carboxy-5-aminopentyl]amide (=OM-197-Lys-AC)

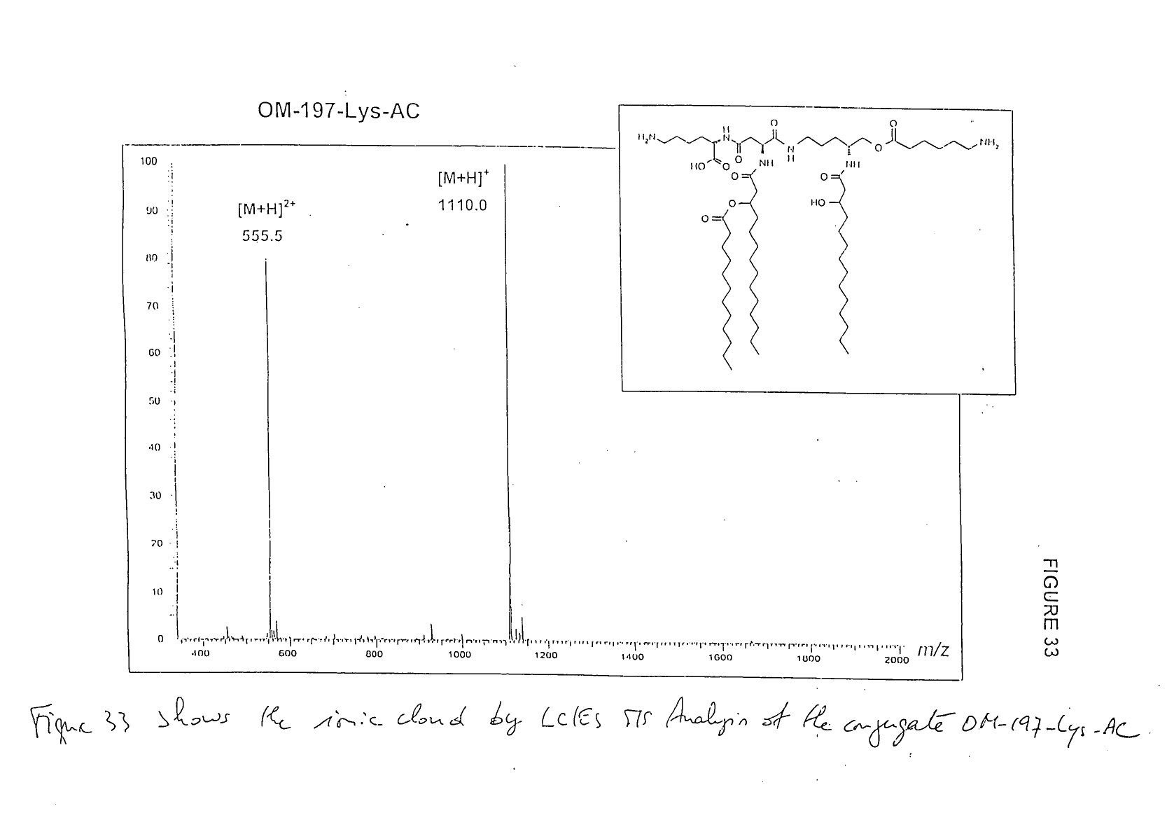

A solution of the above prepared compound (236 mg; 151 μmol.) in an 1/1 CH₂Cl₂/isopropanol mixture (20 ml) containing acetic acid (1 ml) is hydrogenated in presence of palladium on carbon containing 10% Pd (100 mg) at room temperature and under atmospheric pressure hydrogen for 12 to 24 hours. The catalyst is filtered off. The filtrate is evaporated to dryness and the residue is then dried by suction from a vacuum pump to provide N-[(R)-3-dodecanoyloxytetradecanoyl]-D-aspartic acid, α-N-{(4R)-5-(6-aminohexanoyloxy)-4-[(R)-3-hydroxytetradecanoylamino]-pentyl}amide β-N-[(1S)-1-carboxy-5-aminopentyl]amide (140 mg, 83%). C₆₁H₁₁₆N₆O₁₁: ES/MS: m/z ratio 555.5 ([M+H]⁺²); 1110.0 ([M+H]⁺) (FIG. 33).

### Example 2.23.

### N-[(R)-3-dodecanoyloxytetradecanoyl]-D-aspartic Acid, α-N-{(4R)-5-hydroxy-4-[(R)-3-hydroxytetradecanoylamino]-pentyl)amide β-N-[(1S)-1,2-dicarboxyethyl]amide (=OM-197-Asp) (FIG. 34)

2.23.1. N-[(R)-3-dodecanoyloxytetradecanoyl]-D-aspartic acid, α-N-{(4R)-5-hydroxy-4-[(R)-3-benzyloxytetradecanoylamino]pentyl}-amide β-N-[(1S)-1,2-bis(benzyloxycarbonyl)ethyl]amide

A solution of IIDQ (2-isobutoxy-1-isobutoxycarbonyl-1,2-dihydroquinoline) (96 g; 0.32 mmol.) in anhydrous CH₂Cl₂ (5 ml) is added to a solution of N-[(R)-3-dodecanoyloxytetradecanoyl]-D-aspartic acid, α-N-{(4R)-5-hydroxy-4-[(R)-3-benzyloxytetradecanoylamino]-pentyl}amide (Section 2.20.1.) (252 mg, 0.26 mmol.) in anhydrous CH₂Cl₂ (20 ml) at room temperature and under argon flow. The reaction mixture is stirred for 15 minutes at room temperature and a solution of commercially available L-aspartic acid dibenzyl ester paratoluenesulfonate salt (141 mg, 0.29 mmol.) in anhydrous CH₂Cl₂ (5 ml) containing triethylamine (40 μl, 0.29 mmol.) is added. After stirring for 18 hours, the solution is evaporated to dryness. By running a flash chromatography purification on a silica gel (20/1 CH₂Cl₂/MeOH eluent), there is recovered the coupling reaction product (260 mg; 78%).). ¹³C-NMR (62,89 MHz, CDCl₃), δ in ppm: 173.68, 171.92, 171.17, 170.60, 170.46, 170.37, 170.18, 170.06, 138.31, 138.19, 135.20, 135.12, 134.87, 128.48, 128.26, 128.18, 127.75, 127.63, 127.56, 76.66, 71.40, 71.19, 70.98, 68.79, 67.59, 66.85, 65.14, 64.73, 51.25, 50.17, 48.78, 48.67, 41.70, 39.37, 39.21, 37.50, 35.94, 34.48, 34.10, 31.81, 29.54, 29.25, 29.08, 27.93, 25.56, 25.14, 25.09, 24.91, 22.58, 14.02.

2.23.2. N-[(R)-3-dodecanoyloxytetradecanoyl]-D-aspartic Acid, α-N-{(4R)-5-hydroxy-4-[(R)-3-hydroxytetradecanoylamino]-pentyl}amide β-N-[(1S)-1,2-dicarboxyethyl]amide (=OM-197-Asp)

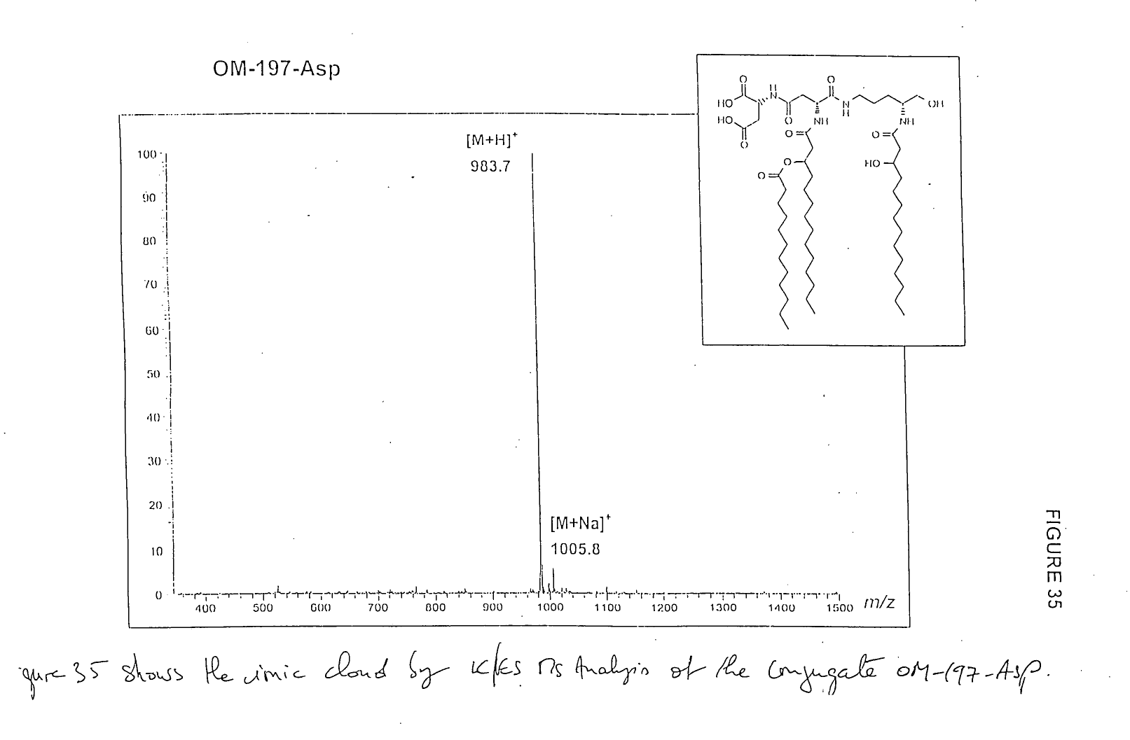

A solution of the above prepared compound (260 mg; 207 μmol.) in a 1/1 CH₂Cl₂/isopropanol mixture (20 ml) is hydrogenated in presence of palladium on carbon containing 10% Pd (50 mg) at room temperature and under atmospheric pressure hydrogen for 4 hours. The catalyst is filtered off. The filtrate is evaporated to dryness and the residue is then dried by suction from a vacuum pump to provide the diacid (108 mg, 88%). C₅₃H₉₈N₄O₁₂: ES/MS: m/z ratio 983.7 ([M+H]⁺); 1005.8 ([M+Na]⁺); (FIG. 35).

### Example 2.24.

### N-[(R)-3-dodecanoyloxytetradecanoyl]-D-aspartic Acid, α-N-{(4R)-5-(6-aminohexanoyloxy)-4-[(R)-3-hydroxytetradecanoyl-amino]-pentyl}amide β-N-[(1S)-1,2-dicarboxyethyl]amide (=OM-197-Asp-AC) (FIG. 34)

2.24.1. N-[(R)-3-dodecanoyloxytetradecanoyl]-D-aspartic Acid, α-N-{(4R)-5-(6-benzyloxyaminohexanoyloxy)-4-[(R)-3-benzyloxytetra-dodecanoylamino]pentyl}amide β-N-(1S)-1,2-bis(benzyloxycarbonyl)ethyl-amide

To a solution of N-[(R)-3-dodecanoyloxytetradecanoyl]-D-aspartic acid, α-N-{(4R)-5-hydroxy-4-[(R)-3-benzyloxytetradecanoyl-amino]pentyl}amide β-N-[(1S)-1,2-bis(benzyloxycarbonyl)ethylamide (Section 2.23.1.) (158 mg, 0.13 mmol.) and 6-(benzyloxycarbonylamino)hexanoic acid (84 mg, 0.32 mmol.) in dry CH₂Cl₂ (6 ml) at 0° C. and under argon flow, there are added in succession commercially available 1-(3-dimethylaminopropyl)-3-ethylcarbodiimide hydrochloride (61 mg, 0.32 mmol.) and 4-dimethylaminopyridine (4 mg, 33 μmol.). The reaction mixture is then stirred for 30 minutes at 0° C. and thereafter overnight at room temperature. The reaction medium is then washed with water and a solution of 1N HCl followed by layer separation. The organic layer is dried over MgSO₄, filtered and evaporated. By running a flash chromatography purification on a silica gel (6/1 CH₂Cl₂/acetone eluent), there is recovered the coupling reaction product (83 mg; 44%).¹³C-NMR (62,89 MHz, CDCl₃), δ in ppm: 173.69, 173.53, 173.26, 171.21, 171.11, 170.61, 170.38, 170.31, 170.22, 156.36, 138.26, 136.58, 135.20, 134.87, 128.48, 128.38, 128.31, 128.27, 128.19, 127.95, 127.55, 76.50, 71.24, 71.10, 67.58, 67.48, 66.79, 66.42, 65.69, 49.99, 48.77, 47.86, 41.70, 41.49, 40.70, 39.15, 37.50, 35.94, 34.44, 34.37, 33.97, 33.67, 31.80, 29.54, 29.24, 29.07, 28.37, 25.98, 25.55, 25.13, 25.08, 24.91, 24.28, 22.58, 14.02.

2.24.2. N-[(R)-3-dodecanoyloxytetradecanoyl]-D-aspartic acid, α-N-{(4R)-5-(6-aminohexanoyloxy)-4-[(R)-3-hydroxytetradecanoylamino]-pentyl}amide β-N-[(1S)-1,2-dicarboxyethyl]amide (=OM-197-Asp-AC)

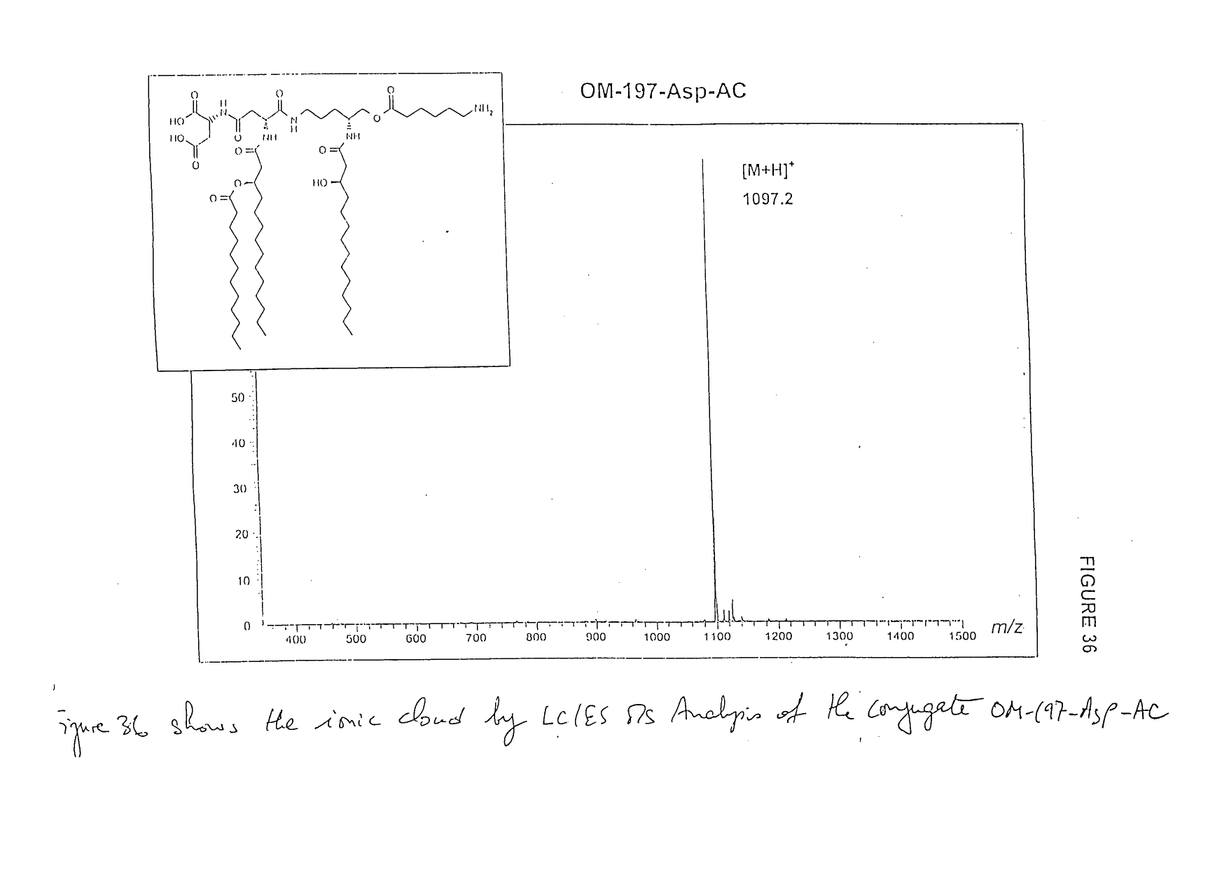

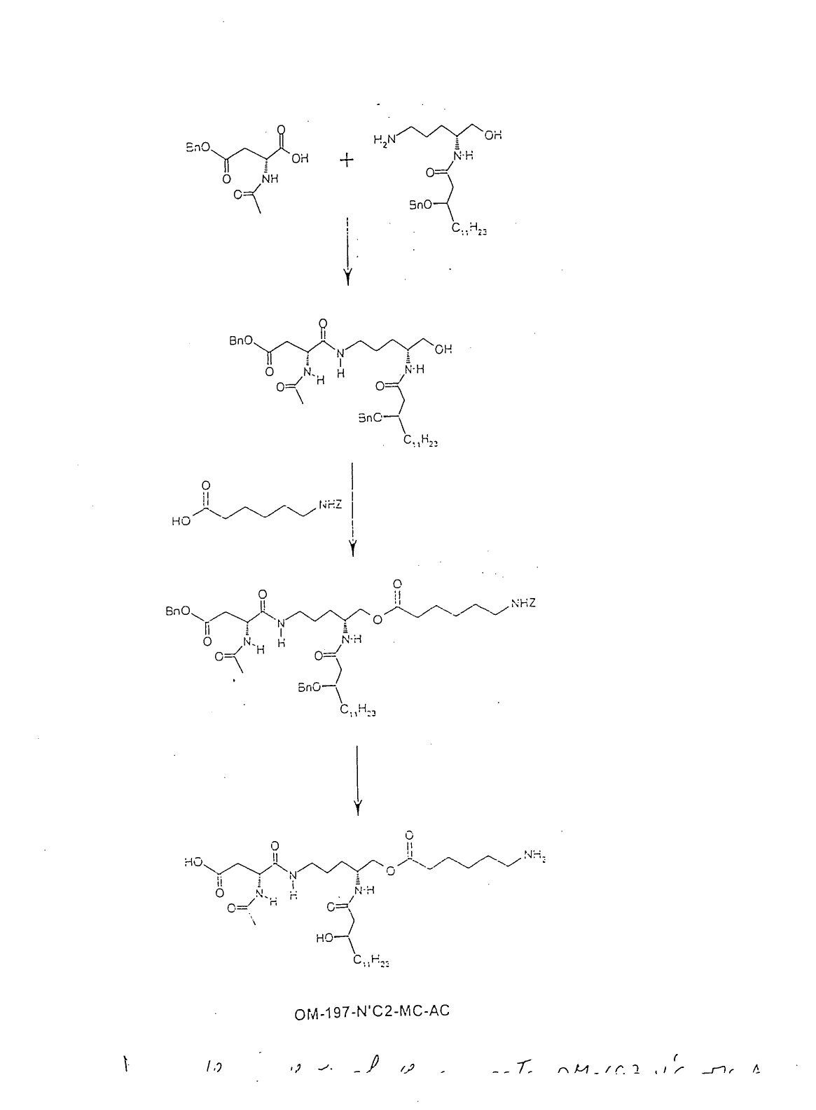

A solution of the above prepared compound (80 mg; 0.053 μmol.) in a 1/4 CH₂Cl₂/ethanol mixture (10 ml) containing acetic acid (1 ml) is hydrogenated in presence of palladium on carbon containing 10% Pd (50 mg) at room temperature and under atmospheric pressure hydrogen for 12 to 24 hours. The catalyst is filtered off. The filtrate is evaporated to dryness and the residue is then dried by suction from a vacuum pump to provide the diacid (61 mg, stoechiometric yield). Mass spectrometry MS: Caclulated for C₅₉H₁₀₉N₅O₁₃: 1095.8; Found ES/MS: m/z ratio 1097.0 ([M+H]⁺) (FIG. 36).

### Example 2.25.

### N-acetyl-D-aspartic acid, α-N-{(4R)-5-(6-aminohexanoyloxy)-4-[(R)-3-hydroxytetradecanoylamino]-pentyl}amide (=OM-197-N′C2-AC) (FIG. 37)

2.25.1. N-acetyl-D-aspartic acid, β-benzyl ester

To a solution of acetic anhydride (85 μl, 0.90 mmol.) in acetonitrile (1 ml), there is added a solution of commercially available H-D-Asp(OBn)-OH (Senn Chemicals, CH-Dielsdorf) (200 mg, 0.90 mmol.) in a CH₃CN.H₂O/Et₃N (3.5/1.0/0.4 ml) mixture. The reaction mixture is stirred for 18 hours at room temperature. The organic solvent is evaporated and the remaining aqueous phase is cooled down to 0° C., acidified with a 10% aqueous solution of citric acid till pH=3 and extracted with EtOAc (2×). The organic layers are pooled and dried over MgSO₄, filtered and evaporated. The -benzyl ester of N-acetyl-D-aspartic acid obtained as a white cristalline solid (192 mg, 81%) is used in the next step with no further purification. (Rf=0.45 in 20/1 CH₂Cl₂/MeOH containing 2% of acetic acid, phosphomolybdinium compound and U.V. color development agent).

2.25.2. N]-acetyl-D-aspartic Acid, α-N-{(4R)-5-hydroxy-4-[(R)-3-benzyloxytetradecanoylamino]pentyl}amide β-benzyl Ester

A solution of IIDQ (2-isobutoxy-1-isobutoxycarbonyl-1,2-dihydroquinoline) (254 g; 0.84 mmol., 1.2 eq.) in anhydrous CH₂Cl₂ (5 ml) is added to a solution of N-acetyl-D-aspartic acid β-benzyl ester prepared above (185 mg, 0.70 mmol.) in anhydrous CH₂Cl₂ (15 ml) at RT and under argon flow. The reaction mixture is stirred for 15 minutes and thereafter a solution of (2R)-5-amino-2-[(R)-3-benzyloxytetradecanoylamino]pentan-1-ol (Section 1.1.2.) (334 mg, 0.77 mmol., 1 eq.) in anhydrous CH₂Cl₂ (10 ml) is added. After stirring for 3 hours, the solution is evaporated to dryness. By subjecting the residual product to a flash chromatography purification on a silica gel (5/3 CH₂Cl₂/acetone then pure acetone eluent), there is recovered the coupling reaction product (369 mg; 78%). (Rf=0.22 in 5/2 CH₂Cl₂/acetone, phosphomolybdinium compound and U.V. color development agent). ¹³C-NMR (62,89 MHz, CD₃OD), δ in ppm: 171.92, 171.23, 171.07, 170.61, 170.35, 138.11, 135.24, 128.40, 128.24, 128.02, 127.69, 127.59, 71.25, 67.57, 64.56, 51.03, 49,41, 41.51, 39.21, 35.90, 33.88, 31.73, 29.46, 29.17, 28.32, 28.02, 25.35, 25.24, 24.97, 22.83, 22.51, 13.97, MS: Calc. for C₃₉H₅₉N₃O₇681.4, found: m/z 682.5 ([M+H]⁺), 704.5 ([M+Na]⁺).

2.25.3. N-acetyl-D-aspartic Acid, α-N-{(4R)-5-(6-aminohexanoyloxy)-4-[(R)-3-benzyloxytetradecanoylamino]-pentyl}amide β-benzyl Ester

To a solution of N-acetyl-D-aspartic acid, α-N-{(4R)-5-hydroxy-4-[(R)-3-benzyloxytetradecanoylamino]-pentyl}amide β-benzyl ester (200 mg 0.29 mmol.) and 6-(benzyloxycarbanoylamino)hexanoic acid (156 mg; 0.59 mmol.) in dry CH₂Cl₂ (8 ml) at 0° C. and under argon flow, there are added in succession commercially available 1-(3-dimethylaminopropyl)-3-ethylcarbodiimide hydrochloride (112 mg, 0.59 mmol.) and 4-dimethylaminopyridine (7 mg, 59 μmol.). The reaction mixture is then stirred for 30 minutes at 0° C. and thereafter overnight at room temperature. The reaction medium is then washed in succession with water and a solution of 1N HCl followed by layer separation. The organic layer is dried over MgSO₄, filtered and evaporated. By running a flash chromatography purification on a silica gel (6/1 CH₂Cl₂/acetone eluent), there is recovered the coupling reaction product (200 mg; 73%) as a white solid. ¹³C-NMR (62,89 MHz, CDCl₃), δ in ppm; 173.65, 171.71, 171.26, 170.30, 156.41, 138.16, 136.60, 135.33, 128.57, 128.46, 128.38, 128.20, 128.04, 127.76, 127.62, 71.21, 66.80, 66.53, 65.72, 65.63, 49.36, 47.80, 41.36, 40.76, 39.22, 39.13, 35.79, 33.75, 31.88, 29.61, 29.31, 28.58, 28.52, 26.04, 25.48, 25.11, 24.36, 23.13, 22.64, 14.09.

2.25.4. N-acetyl-D-aspartic acid, α-N-{(4R)-5-(6-aminohexanoyloxy)-4-[(R)-3-hydroxytetradecanoylamino]-pentyl}amide (OM-197-N′C2-AC)

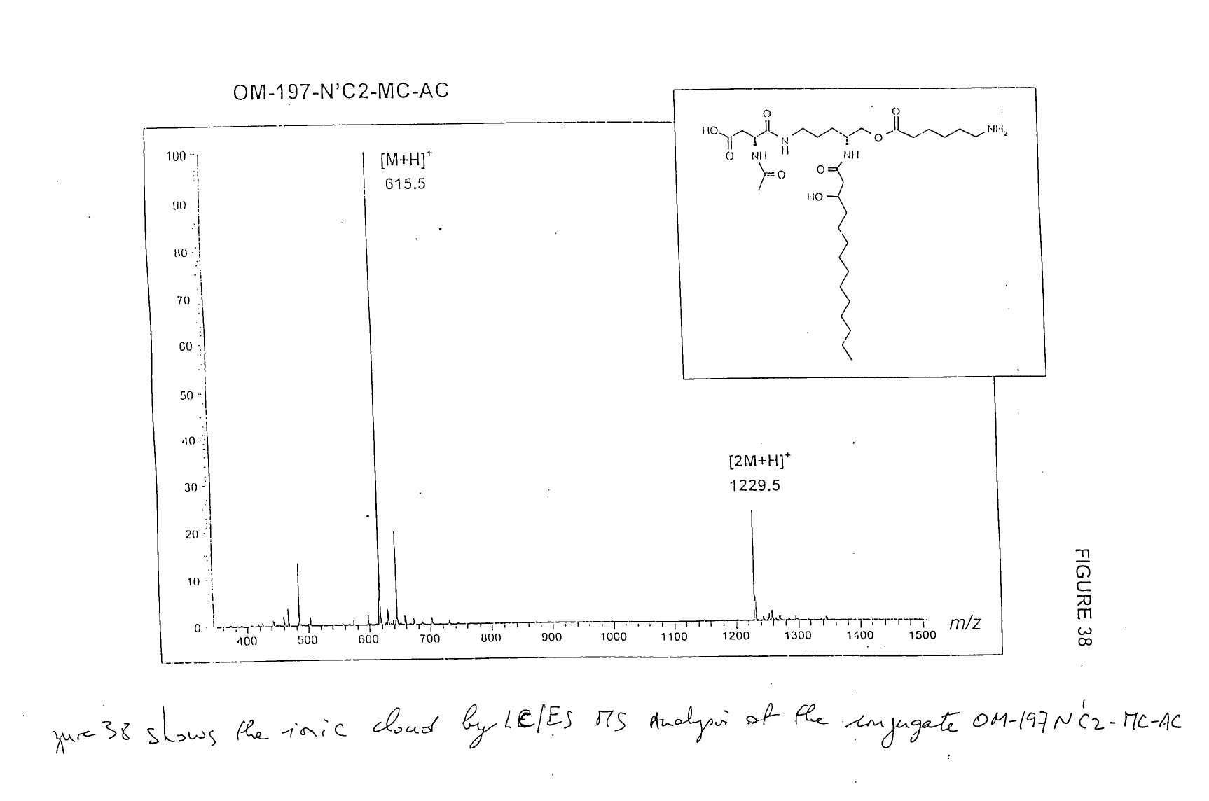

A solution of the compound obtained above (200 mg ; 0.22 mmol.) in a 1/1 CH₂Cl₂/ethanol mixture (16 ml) containing acetic acid (2 ml) is hydrogenated in presence of Pd on carbon containing 10% Pd (40 mg) at room temperature and under atmospheric pressure hydrogen for 12 to 24 hours. The catalyst is filtered off and the filtrate is evaporated to dryness and then dried by suction from a vacuum pump to provide N-acetyl-D-aspartic acid, α-N-{(4R)-5-(6-aminohexanoyloxy)-4-[(R)-3-hydroxytetra-decanoylamino]pentyl}amide (132 mg, stoechiometric yield); MS: Calc. For C₃₁H₅₈N₄O₈ 614.43; Found: m/z 615.5 ([M+H]⁺, 629.5 ([M+NH₄]⁺), 637.5 ([M+Na]⁺) (FIG. 38).

### Example 2.26.

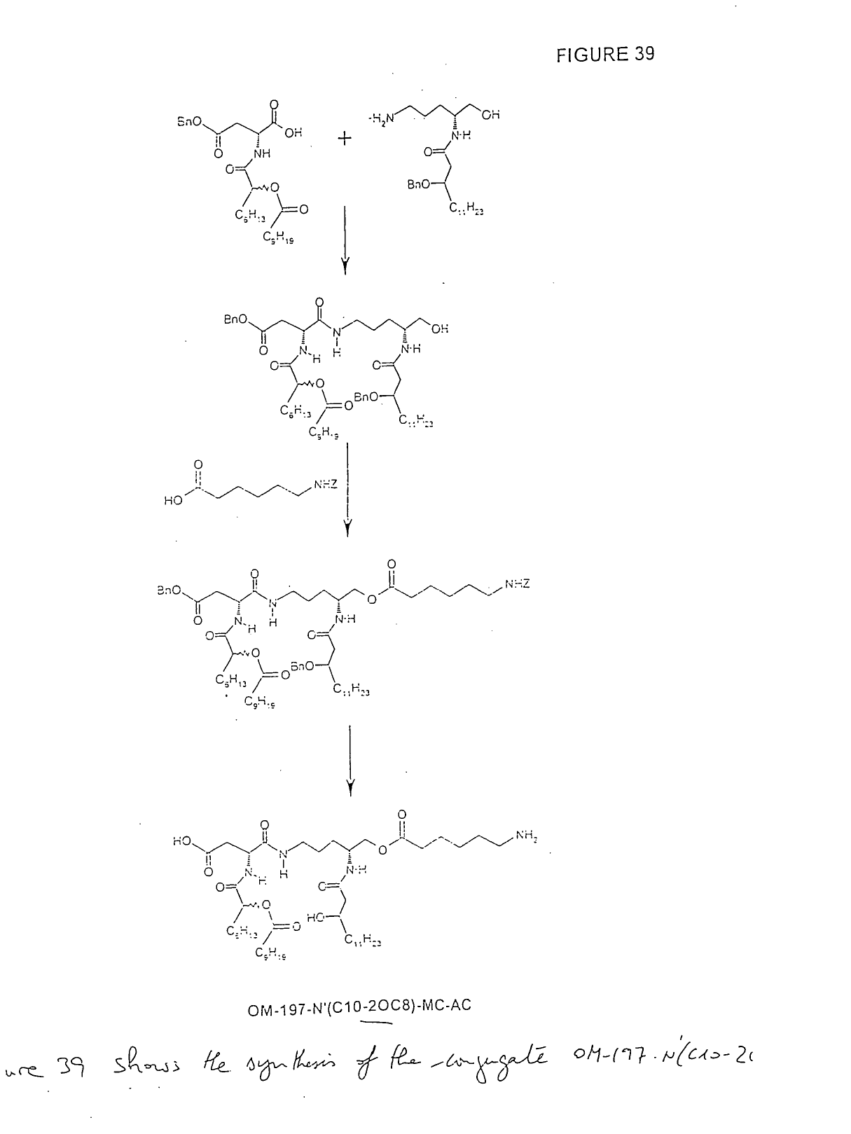

### N-(2-decanoyloxyoctanoyl)-D-aspartic acid, α-N-{(4R)-5-(6-aminohexanoyloxy)-4-[(R)-3-hydroxytetradecanoylamino]-pentyl}amide (=OM-197-N′(C10-2O8C)-AC) (FIG. 39)

2.26.1. Benzyl 2-hydroxyoctanoate

To a solution of a commercially available racemic 2-hydroxyoctanoic acid mixture (1.00 g, 6.2 mmol.) in HPLC-grade ethylacetate (30 ml), there are added in succession benzyl bromide (2.22 ml; 18.7 mmol.), triethylamine (2.6 ml, 18.7 mmol.) and tetrabutylammonium iodide (1.15 g; 3.12 mmol.). The solution is stirred for 18 hours at room temperature and the solvent is thereafter evaporated. The residue is taken up into ether and the organic layer is then washed with a saturated solution of NaHCO₃ and H₂O (2×). The organic layer is dried over MgSO₄, filtered and evaporated to yield crude benzyl 2-hydroxyoctanoate (Rf=0.57 in a 3/1 petroleum ether/EtOAc mixture; phosphomolybdinium compound and U.V. color development agent)

2.26.2. Benzyl 2-decanoyloxyooctanoate

To a solution of the ester as obtained above (780 mg; 3.12 mmol.) in dry CH₂Cl₂ (15 ml) at 0° C., there are added dropwise in succession pyridine (0.9 ml) and decanoyl chloride (654 mg; 3.43 mmol.). The mixture is stirred for 20 hours at room temperature and the reaction medium is then poured into ice-cold water containing (5%) NaHCO₃. The organic layer is separated, successively washed with a 1N HCl solution and water (2×). The organic layer is then dried over MgSO₄, filtered and evaporated. By running a flash chromatography purification on a silica gel (30/1 petroleum ether/EtOAc eluent), recovery of pure Benzyl 2-decanoyloxyooctanoate is achieved.

2.26.3. 2-decanovloxvoctanoic Acid

A solution of benzyl 2-hydroxyoctanoate as prepared above (515 mg; 1.27 mmol.) in HPLC-grade EtOH (40 ml) is hydrogenated in presence of Pd on carbon containing 10% Pd (100 mg) at room temperature and under atmospheric pressure hydrogen for 2 hours. The catalyst is filtered off on a millipore filter. The filtrate is evaporated to dryness and the residue is then dried under suction from a vacuum pump to obtain 2-decanoyloxyoctanoic acid (stoechiometric yield)

2.26.4. N-(2-decanoyloxyoctanoyl)-D-aspartic acid, β-benzyl Ester

To a solution of 2-decanoyloxyoctanoic acid (317 mg; 1.01 mmol.) in anhydrous THF (2 ml) at −15° C. and under argon flow, there are added in succession N-methylmorpholin (111 μl; 1.01 mmol.; 1 eq) and isobutyl chloroformate (131 μl; 1.01 mmol.; 1 eq.). Rapid formation of a N-methylmorpholin hydrochloride precipitate is observed. After stirring for 30 minutes at −15° C., a commercially available solution of H-D-Asp(OBn)-OH (Senn Chemicals AG, Switzerland-Dielsdorf) (225 mg; 1.01 mmol. ; 1 eq.) in a 3.5/1 CH₃CN/H₂O mixture (9 ml) containing Et₃N (0.4 ml) is added. The reaction mixture is then stirred overnight at room temperature. The organic layer is then evaporated and the aqueous layer is cooled down to 0° C., acidified with a 10% aqueous solution of citric acid down to pH=3 and extracted with EtOAc (2×). The organic layer is dried over MgSO₄, filtered and evaporated. By running a flash chromatography purification on a silica gel (2/1 petroleum ether/EtOAc eluent containing 2% acetic acid) followed by toluene coevaporation, there is recovered N-(2-decanoyloxyoctanoyl)-D-aspartic acid, β-benzyl ester (140 mg; 27%).

2.26.5. N-(2-decanoyloxyoctanoyl)-D-aspartic Acid, α-N-{(4R)-5-hydroxy-4-{(R)-3-benzyloxytetradecanoylamino]-pentyl}amide β-benzyl Ester

IIDQ (98 mg; 0.32 mmol.; 1.2 eq.) is added to a solution of N-(2-decanoyloxyoctanoyl)-D-aspartic acid, β-benzyl ester (140 g ; 0.27 mmol.) in anhydrous CH₂Cl₂ (15 ml) at room temperature and under argon flow. After stirring for 15 minutes, addition was made of a solution of (2R)-5-amino-2-[(R)-benzyloxytetradecanoylamino]pentan-1-ol (Section 1.1.2.) (129 mg ; 0.30 mmol.; 1.1 eq.) in anhydrous CH₂Cl₂ (5 ml). After stirring for 18 hours, the solution is evaporated to dryness. By running a flash chromatography purification on a silica gel (successively 5/1 CH₂Cl_{2/}acetone and 1/1 CH₂Cl_{2/}acetone eluent), there is recovered the coupling reaction product (197 mg; 78%).(Rf=0.30 in a 5/1 CH₂Cl₂/acetone mixture; phosphomolybdinium compound and U.V. color development agent).

2.26.6. N-(2-decanovloxyoctanoyl)-D-aspartic acid. α-N-{(4R)-5-[6-(benzyloxycarbonylamino)hexanoyloxy]-4-[(R)-3-benzyloxytetradecanoyl-amino]pentyl}amide β-benzyl Ester

To a solution of N-(2-decanoyloxyoctanoyl)-D-aspartic acid, α-N-{(4R)-5-hydroxy-4-[(R)-3-benzyloxytetradecanoylamino]-pentyl}amide β-benzyl ester (97 mg; 0.21 mmol.) and 6-(benzyloxycarbanoylamino)hexanoic acid (112 mg, 0.42 mmol.) in dry CH₂Cl₂ (8 ml) at 0° C. and under argon flow, there are added in succession 1-(3-dimethylaminopropyl)-3-ethylcarbodiimide hydrochloride (81 mg, 0.42 mmol) and 4-dimethylaminopyridine (6 mg, 42 μmol.). The reaction mixture is next stirred for 30 minutes at 0° C. and thereafter overnight at room temperature. The reaction medium is then washed in succession with water and a 1N HCl solution; followed by layer separation. The organic layer is dried over MgSO₄, filtered and evaporated. By running a flash chromatography purification on a silica gel (6/1 CH₂Cl₂/acetone eluent), there is recovered the coupling reaction product (170 mg; 68%).

2.26.7. N-(2-decanoyloxyoctanoyl)-D-aspartic Acid, α-N-{(4R)-5-[6-(aminohexanoyloxy]-4-[(R)-3-hydroxytetradecanoylamino]pentyl}amide

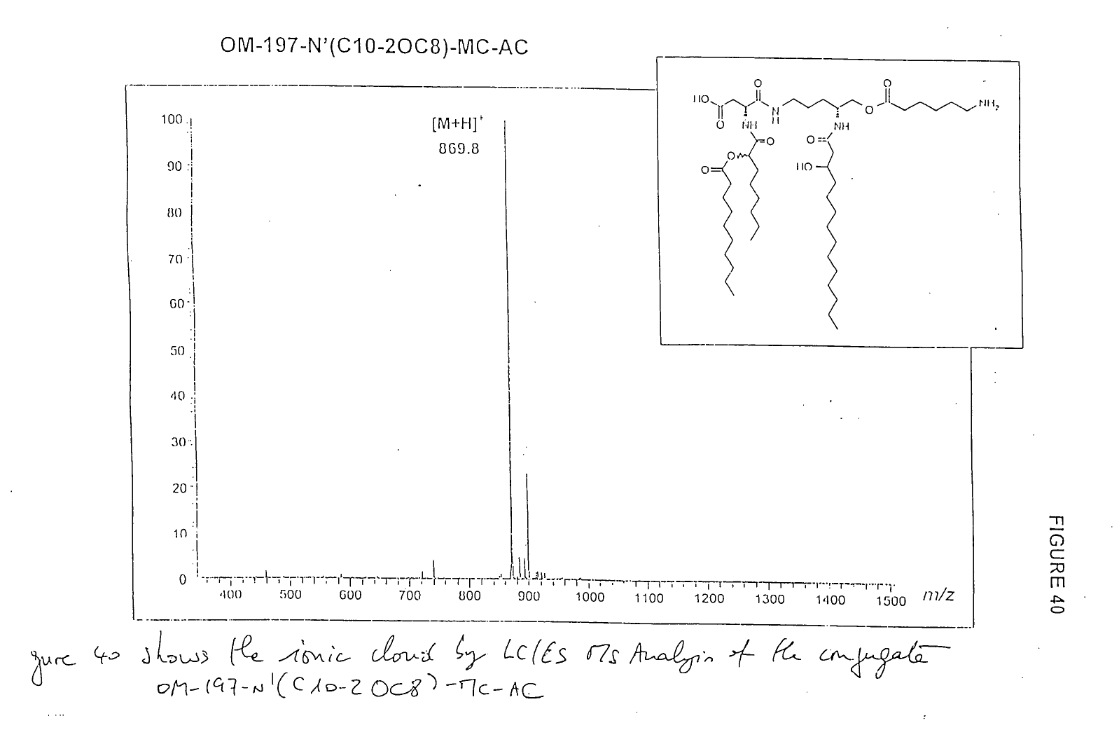

A solution of the compound as prepared above (150 mg ; 0.13 mmol.) in a 1/1 CH₂Cl_{2/}ethanol mixture (16 ml) containing acetic acid (2 ml) is hydrogenated in presence of Pd on carbon containing 10% Pd (40 mg) at room temperature and under atmospheric pressure hydrogen for 12 to 24 hours. The catalyst is filtered off. The filtrate is evaporated to dryness and the residue is then dried by suction from a vacuum pump to provide N-(2-decanoyloxyoctanoyl)-D-aspartic acid, α-N-{(4R)-5-[6-(aminohexanoyloxy]-4-[(R)-3-hydroxytetradecanoylamino]pentyl}amide (110 mg, stoechiometric yield) MS (mass spectrometry) Calc. For C₄₇H₈₈N₄O₁₀: 868.65 ; Found: m/z ratio 870.0 ([M+H]⁺); 884.0 ([M+NH₄]⁺), 891.5 ([M+Na]⁺) (FIG. 40).

### Example 2.27.

### Purification and Analysis of Compounds in Accordance with the Invention

The synthesis product and acyl-dipeptide-like compounds bearing an accessory functional side chain spacer are dissolved in a (1:1 v/v) water-isopropanol mixture The required quantity of 2 M ammonium bicarbonate is then added up to a concentration of 50 mM.

2.27.1. Purification

Purification process is performed by reverse phase preparative HPLC according to the following conditions:
Column:Bondapack C18 PrepPak, 40×200 mm, 15-20 μm, 300 Å, Waters
Mobile phase:
   A: (9:1 v/v)isopropanol-water, 50 mM ammonium bicarbonate
   B: (2:8 v/v) isopropanol-water, 50 mM ammonium bicarbonate
Flow rate: 40 ml/min.
Elution : Isocratic adsorption on column : 40% B (60% A), 10 min.
Gradient: A:B: 40-80% B within 10 min.
Isocratic elution : 80% B, 30 min.
Washing step: 100% B, 10 min.
Detection: UV, 210 nm.

Should any aromatic products be observed (incomplete deprotection step), a finer purification step must be carried out. This further purification process is performed according to the following conditions:
Column: Kromasil C18, 21×250 mm, 5 μm, 100 Å, Macherey-Nagel.
Mobile phase:
   A: (9:1 v/v) isopropanol-water 50 mM ammonium bicarbonate
   B : (2:8 v/v) isopropanol-water, 50 mM ammonium bicarbonate
Flow rate: 5 ml/min.
Elution : Isocratic adsorption on column : 40% B (60% A), 10 min.
Isocratic elution : 84% B, 30 min.
Washing step: 100% B, 10 min.
Detection: UV, 210 nm.
System : Waters 2000

The fractions containing the relevant compounds in the form of ammonium salts are pooled and concentrated by adsorption on C18 Bondapack, 15-20 μm, 300 Å, Waters. The counter-ion can then be exchanged by washing with an aqueous solution of an alkaline metal salt (such as NaCl or KCl, for instance) at a concentration of 10 g/l in a water-isopropanol mixture (9:1, v/v). Upon removal of excess salt by flowing 5 volumes of a (9:1, v/v) water-isopropanol mixture over the column, the compound is eluted with pure isopropanol.

2.27.2. Purification Process Monitoring

After each step, the fractions are analyzed by analytical reverse phase HPLC chromatography according to the following conditions:
Column : Supelcosil C18, 3 μm, 4.6×150 mm, 100 Å, Supelco.
Mobile phase:
   A: (1:1, v/v.) water-acetonitrile, 5 mM TBAP
   B: (1:9, v/v) water-isopropanol 5 mM, TBAP
TBAP : tetrabutylammonium phosphate
Flow rate: 1 ml/min.
Elution step: (75:25 to 0:100 range) A:B Gradient within 37.5 min.
Detection: UV, 210 nm and 254 nm.

Chromatography apparatus: For these assays, different HPLC chromatography apparatus were used (HP1050 Ti series, HP1090 series M, LabChromShimadzu). Retention times read by the particular system being used may vary by about 1 minute for a given compound.

2.27.3. Assay and Purity Analysis of Final Products

Assay and purity analysis of the obtained products are performed by an HPLC/UV technique according to the chromatography operating conditions specified above. Based on these analysis, purity of products ranges from 97 to 100%. In order to demonstrate that inactive impurities are present as judged by UV detection, LC/ES-MS analysis were carried out (electrospray type ionization, positive mode). In order to fulfill ionization conditions, chromatographic separation was carried out according to the following conditions:
Column : Vydac C4, 5 μm, 4.6×150 mm, 300 Å
Mobile phase:
   A: (1:1, v/v) water-acetonitrile, 0.05% TFA
   B: (1:9, v/v) water-isopropanol, 0.05% TFA
Flow rate: 1 ml/min.
Elution : (80:20-0:100 range) A/B gradient within 20 minutes.
Temperature: 40° C.

2.27.4. Spectroscopic Analysis

Mass Spectrometry

ES/MS spectra (positive and negative modes) were recorded on a variety of mass spectrometers (Finnigan LCQ, ion trap; Micromass Quattro II, triple stage quadrupole; Micromass Z-Bio-Q, triple stage quadrupole). Additional analysis based on MS/MS measurements were carried out.

Nuclear Magnetic Resonance

¹H-NMR and ¹³C-NMR spectra were recorded on a DPX Bruker type apparatus at 250.13 and 62.89 MHz.

3^{rd} Series of Examples: Preparation of Conjugates

### Example 3.1

### FGFG Peptide Conjugates

As shown by synthesis schemes set forth on FIGS. 5, 8, 15, 17, 19, 21 and 23, a vicinal dihydroxylation reaction is performed on dipeptide-like compounds bearing an olefin type accessory side chain spacer to finally result into a stable diol functional group. A periodic oxidation reaction is then conducted to form an aldehyde functional group on the accessory side chain spacer. A reductive amination reaction may then carried out using this functional group to couple, for instance, a small size peptide such as FGFG (Phenylalanine-Glycine-Phenylalanine-Glycine, Sigma). However, the aldehyde compound is preferably subjected to a purification step in order to remove salts present in the solution. This purification of the aldehyde compound is achieved on a C18 phase Bondapack (Waters), 15-20 μm, 300 Å, according to the following procedure:
sample adsorption after dilution in a (1:3) isopropanol-water mixture.
elution of salts with a (1:9) isopropanol-water mixture.
elution of the aldehyde compound with the least volume of isopropanol

The coupling reaction based on reductive amination was carried out in an aqueous solution at a 1:1 stoechiometric ratio of aldehyde (accessory side chain spacer of the acyl-dipeptide-like compound) and amine functional groups available on the peptide or the protein to be coupled.

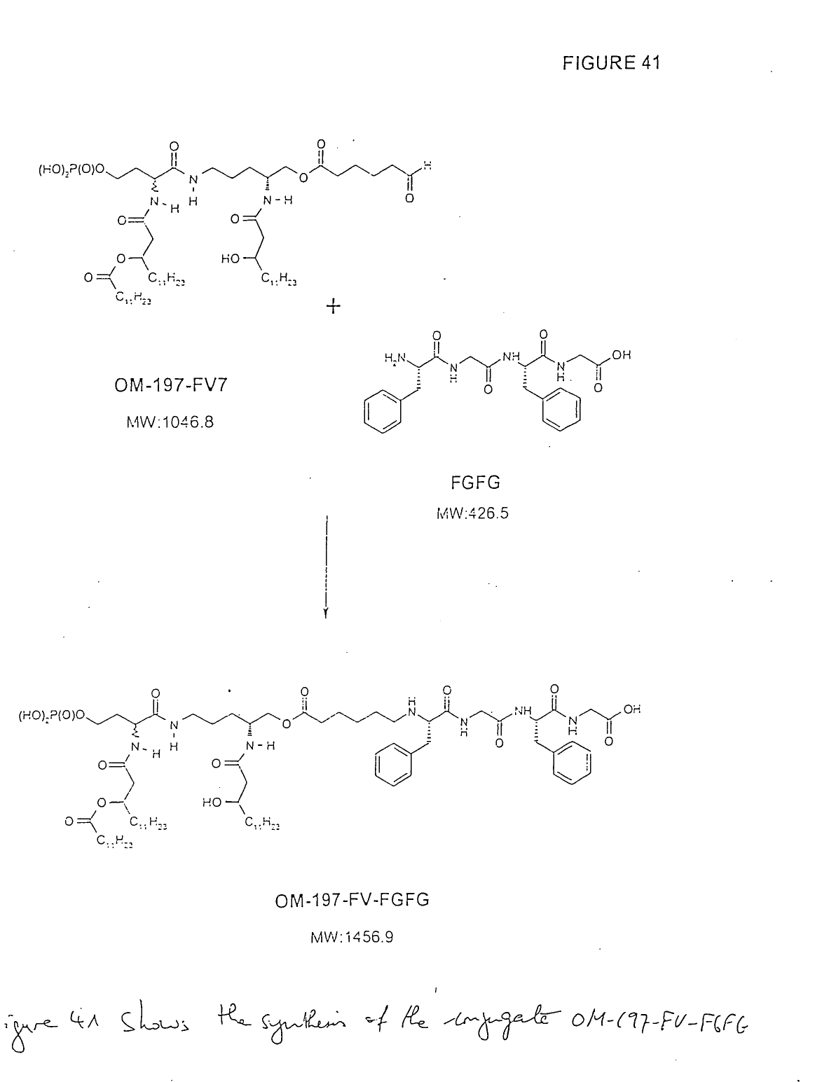

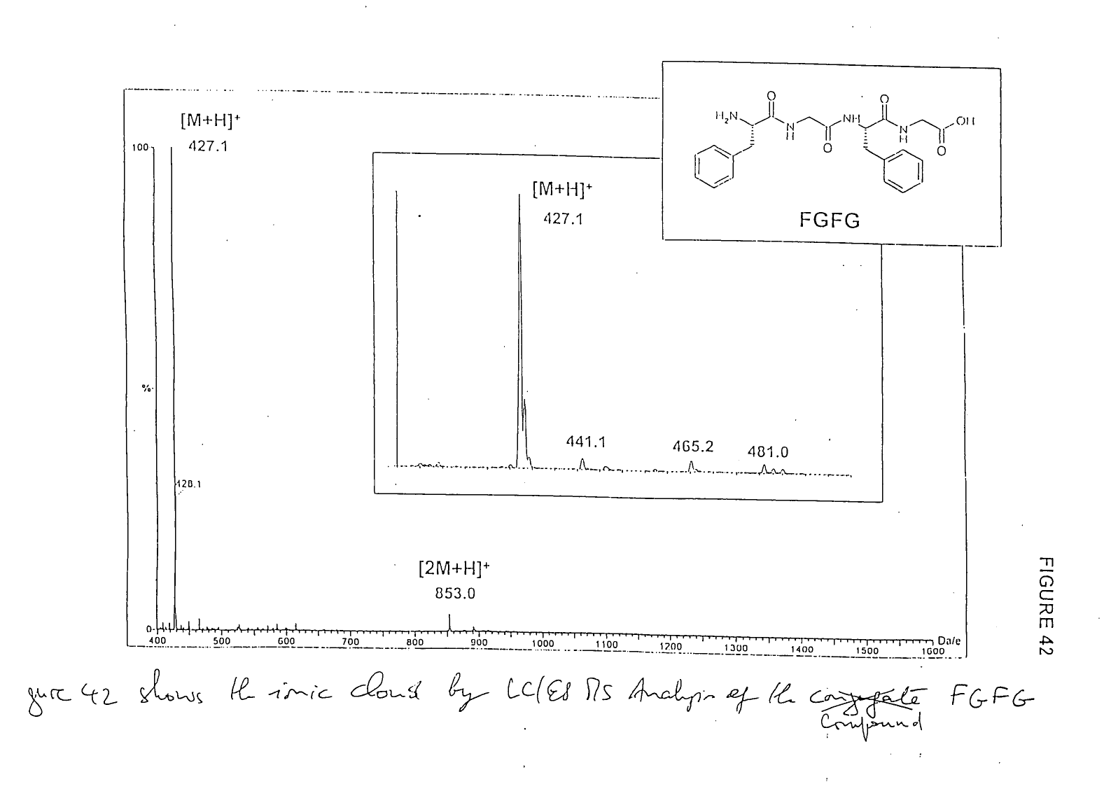

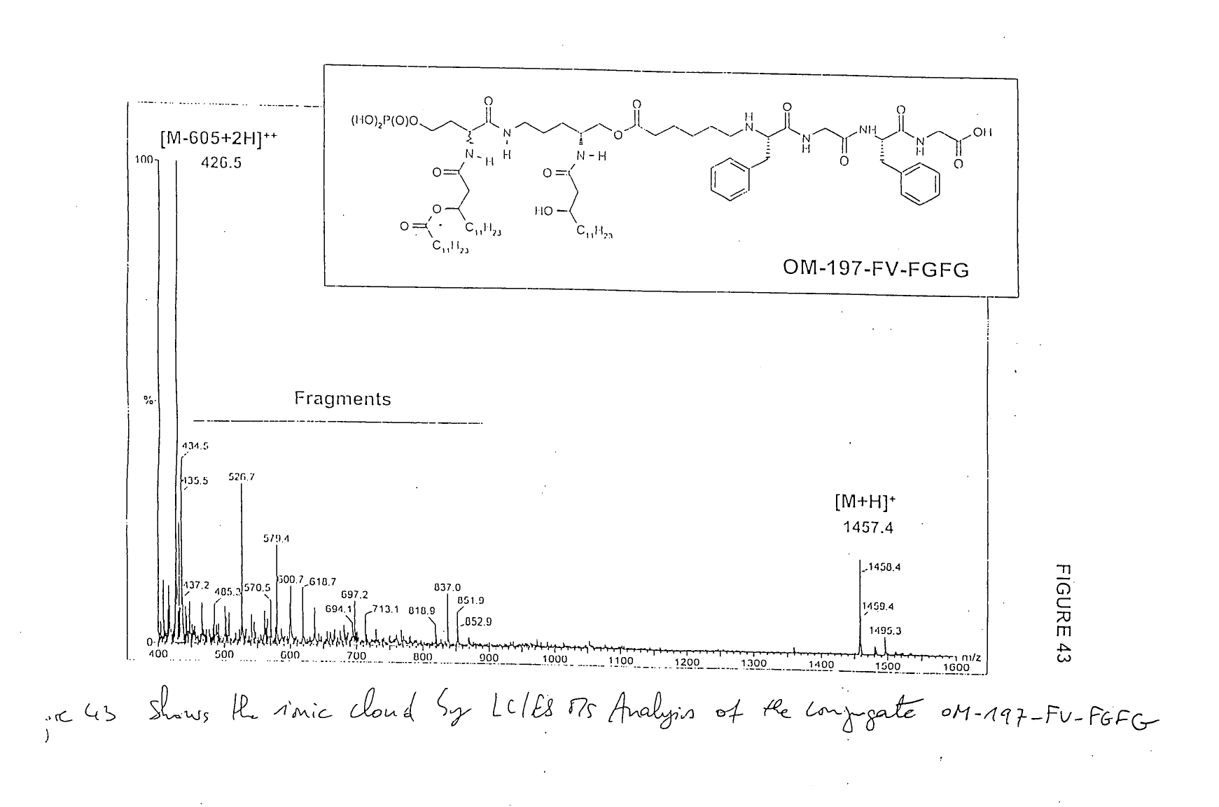

In case of an FGFG peptide, 2 mg of purified OM-197-FV7 (1.86 μmol., 1 eq.) were added to a solution of 0.8 mg FGFG (1.86 μmol., 1 eq.) dissolved in 1 ml of (1:1) H₂O-MeCN. The solution was stirred for a period of 30 minutes at room temperature to form an imine. The reduction step was subsequently carried out by adding 92 μl of 1 M NaBH₃CN (5.77 mg, 50 eq.) to form a highly stable carbon-nitrogen bond (FIG. 41). The conjugate was subjected to a preliminary purification step on a Vydac C4 column in order to remove the non reacted peptide, and thereafter on a Bondapack C18 phase (Waters) to obtain the sodium salt (see purification and analysis section of compounds according to the invention). Next, the conjugate was dissolved back in sterile H₂O/0.01% triethanolamine (TEOA) to meet the biological activity testing requirements. The ES/MS mass spectrum recorded after the purification process is shown on FIG. 43 and clearly demonstrates a [M+H]⁺ molecular ion at 1457.4 m/z ratio, which provides evidence that the required coupling did occur. Visible ions between 400 and 900 m/z ratio were identified by MS/MS analysis as fragments arising from a molecular peak of the conjugated peptide. Ions corresponding to the initial FGFG peptide (m/z ratio 427.1 [M+H]⁺, m/z ratio 853.0 [2M+H]⁺, FIG. 42) were not detected.

### Example 3.2.

### (NANP)₆P₂P₃₀ Peptide Conjugates

OM-197-FV7 compound obtained by the synthesis scheme set forth on FIG. 5 may be coupled, for instance, to a peptide of pharmaceutical interest such as (NANP)₆P₂P₃₀ [Valmori et al., 1992, J. Immunol., 149:717-721] having the following sequence:
(NANP)₆QYIKANSKFIGITEFNNFTVSFWLRVPKVSASHLE

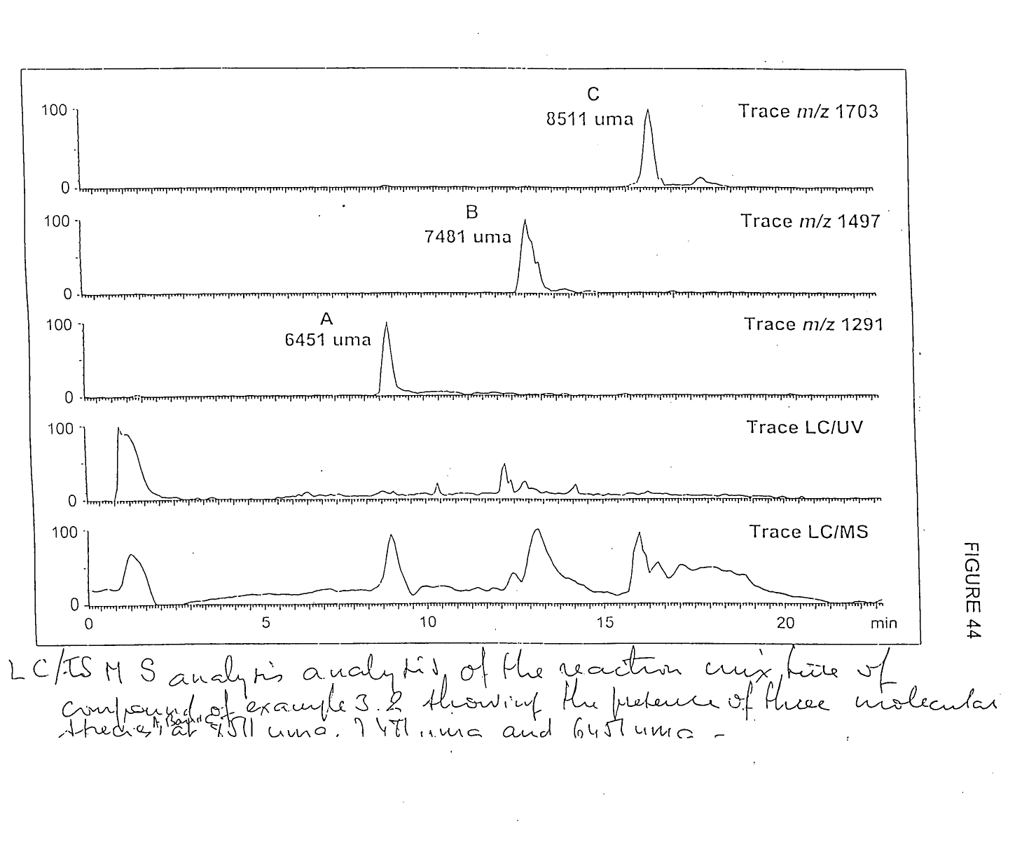

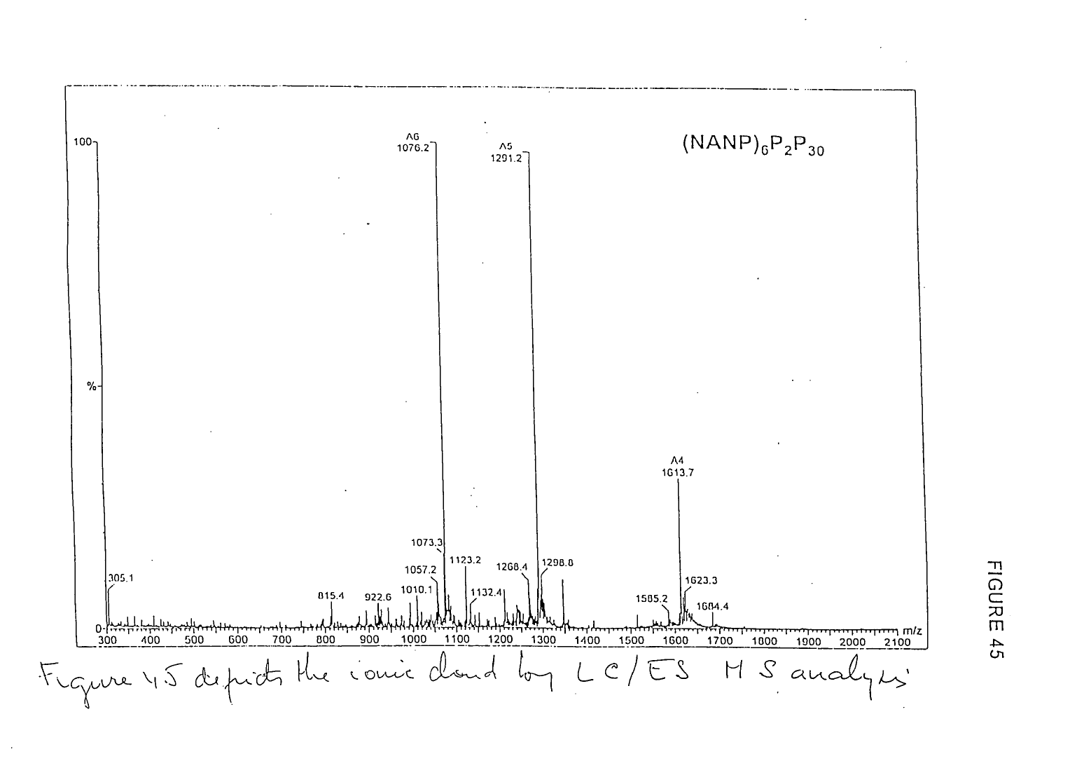

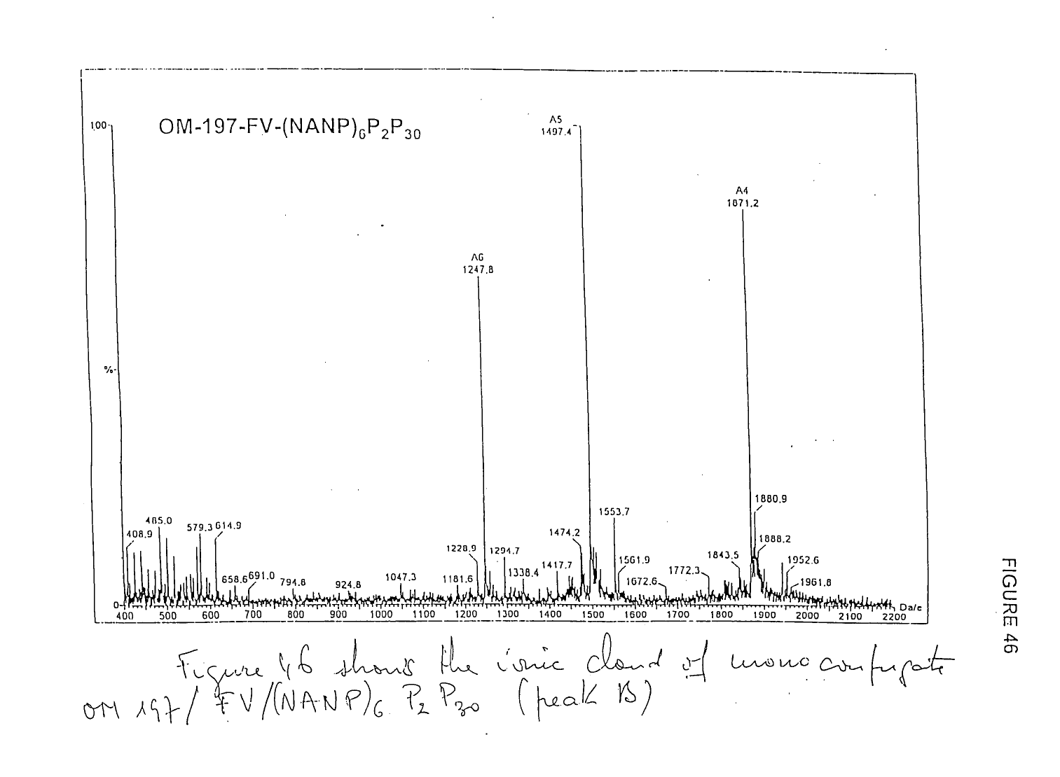

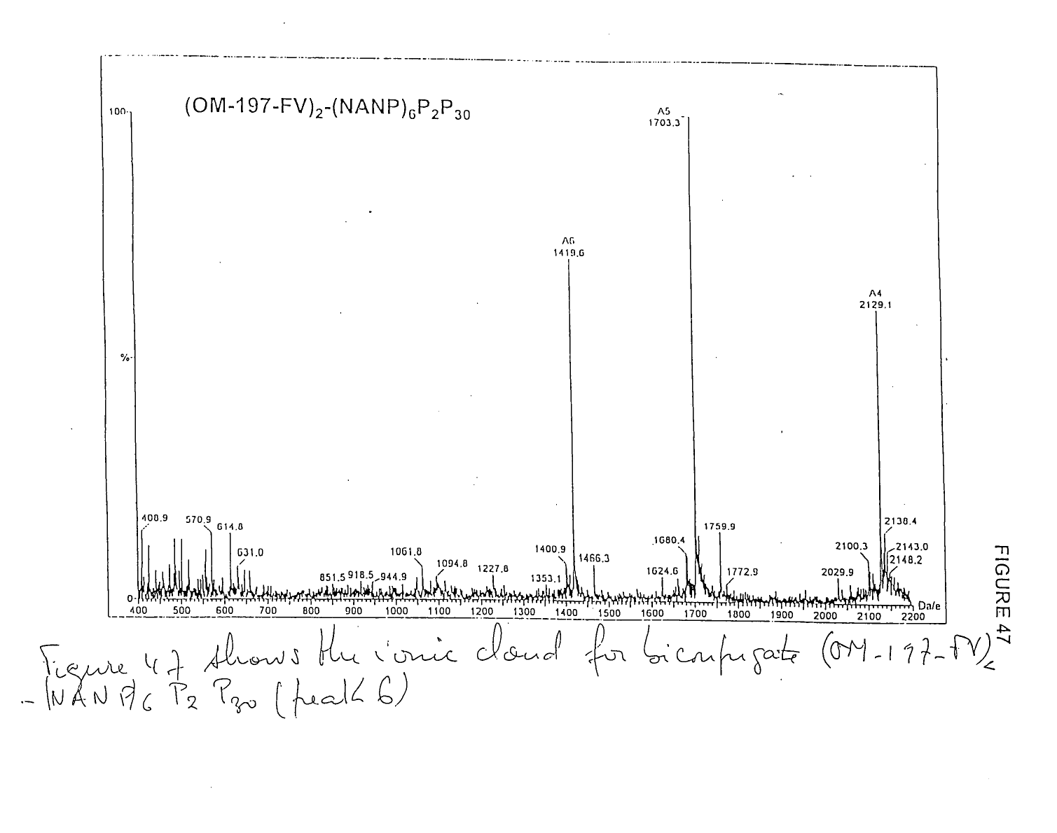

The (NANP)₆P₂P₃₀ peptide shows four potential conjuguation sites (terminal amine+three lysines). 5 mg of purified OM-197-FV7 (4.64 μmol. , 4 eq.) were added to a solution of 7.49 mg (NANP)₆P₂P₃₀ (1.16 μmol., 1 eq.) dissolved in 1 ml of a (1:1) H₂O-isopropanol mixture. The solution was stirred for 30 minutes at room temperature, and thereafter the reduction step was conducted by adding 230 μl of 1M NaBH₃CN (14.43 mg, 50 eq.). The solution was stirred for two hours at room temperature. The reaction mixture was then dialyzed against H₂O for 24 hours (3.5 kDa dialysis cassette, Slide-A-Lyzer, Pierce). LC/ES-MS analysis of the reaction mixture demonstrates the presence of three molecular species, as shown by the chromatograms depicted on FIG. 44: Peaks corresponding to the free peptide (peak A), to the monoconjugated peptide (peak B) and the biconjugated peptide (peak C) are clearly visible. The ionic cloud recorded for each of these are depicted on FIGS. 45, 46 and 47 and provide evidence that covalent conjugation to one or two molecules of OM-197-FV has taken place as for the monoconjugate and biconjugate, respectively,.
FIG. 45: Peptide (NANP)₆P₂P₃₀ (free peptide) MW: 6451. Ions at an m/z ratio of 1076.2 (A6), 1291.2 (A5), 1613.7 (A4)
FIG. 46: Monoconjugate OM-197-FV-(NANP)₆P₂P₃₀ MW: 7481 Ions at an m/z ratio of 1247.8 (B6), 1497.4 (B5), 1871.2 (B4)
FIG. 47: Biconjugate (OM-197-FV)₂-(NANP)₆P₂P₃₀ MW: 8511 Ions at an m/z ratio of 1419.6 (C6), 1703.3 (C5), 2129.1 (C4)

SDS-PAGE analysis of OM-197-FV-(NANP)₆P₂P₃₀ conjugates demonstrated various discrete bands corresponding to the unreacted peptide, the monoconjugate and the biconjugate. Under the electrophoretic conditions herein used (10-20% polyacrylamide gradient, tris-tricin buffer, Bio-Rad, # 161-1108), the difference between the relevant bands was estimated at 1000 amu based on standard markers, which provides evidence that coupling of one or two molecules of OM-197-FV to the peptide did occur.

### Example 3.3.

### P₂P₃₀ Peptide Conjugates

Other peptides can also be coupled by a reductive amination reaction to the compounds bearing an accessory aldehyde type side chain spacer. The example of coupling OM-197-FV with a P₂O₃₀ peptide is given for the purpose of illustration. The latter corresponds to the T epitope portion of the tetanus toxoid and has the following sequence:
KQYIKANSKFIGITEFNNFTVSFWLRVPKVSASHLE

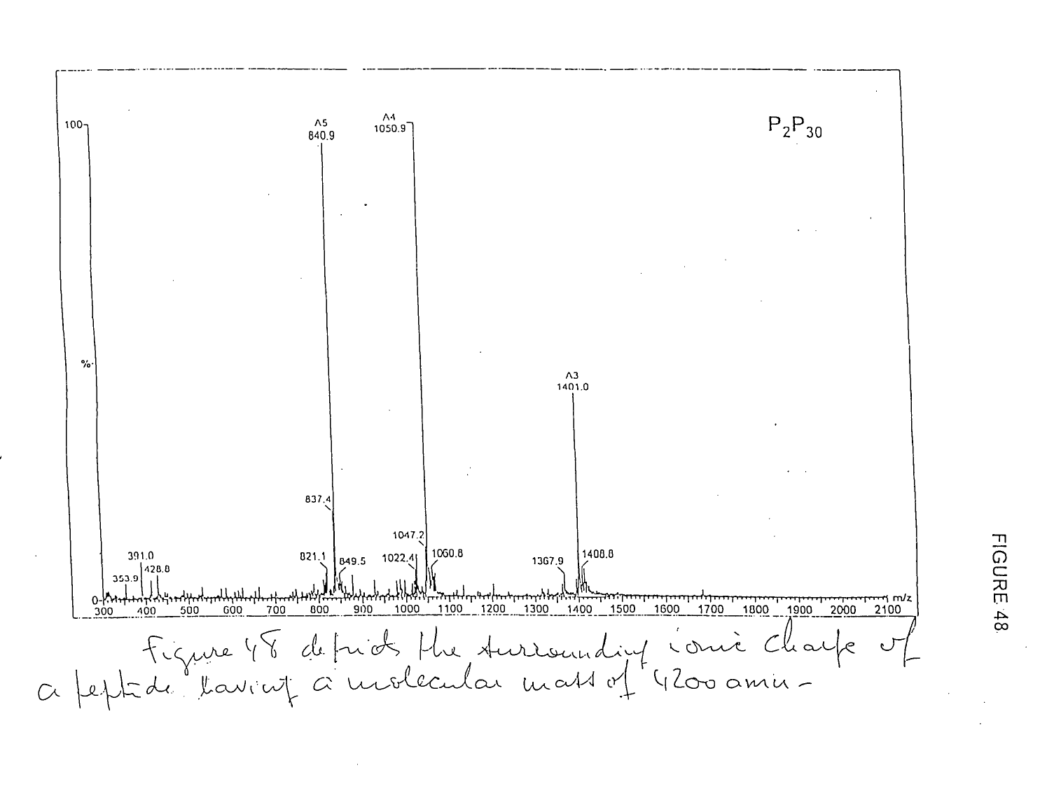

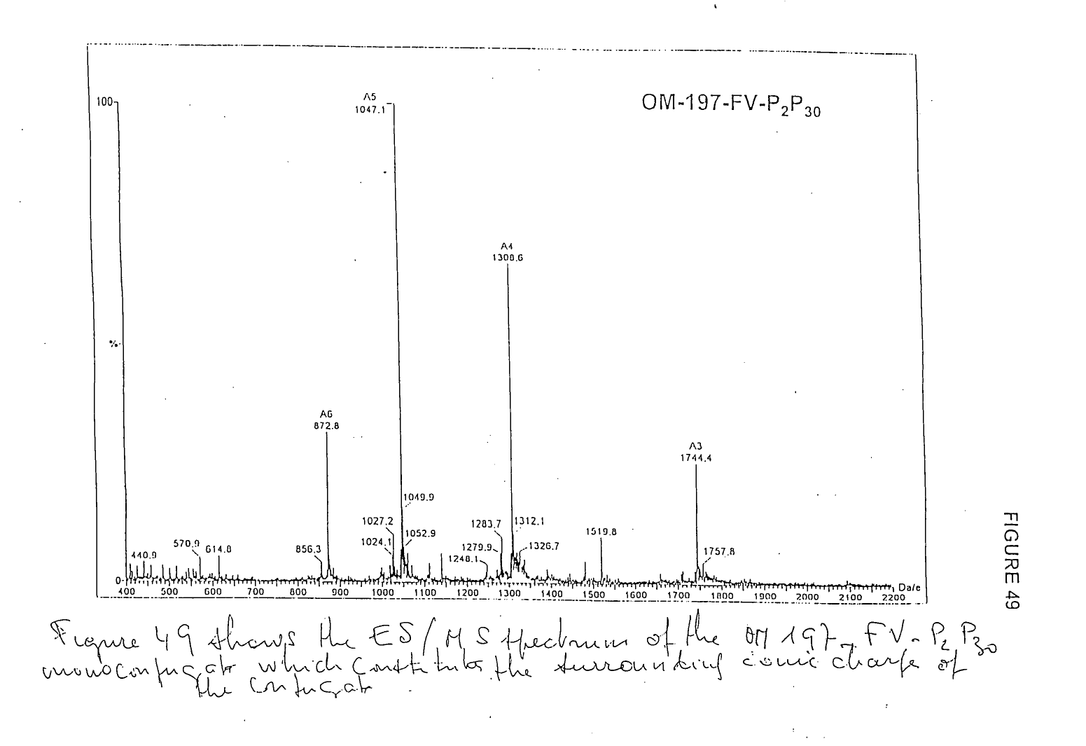

The P₂P₃₀ peptide displays five potential conjugation sites (terminal amine+four lysine residues) and a mass of 4200 amu. Hence, five equivalents of OM-197-FV7 were used up. Reaction conditions specified above were observed. After dialysis in H₂O (3.5 kDa dialysis cassette, Slide-A-lyzer, Pierce), the mass spectra of the obtained conjugates were measured by an LC/ES-MS technique and compared to spectra recorded for the free peptide P₂P₃₀ which displays polyvalent ions at an m/z ratio of 840.9 (A5) 1050.9 (A4) and 1401.0 (A3) which form the surrounding ionic charge (FIG. 48) of a peptide having a molecular mass of 4200 amu. The ES/MS spectrum of the OM-197-FV-P₂P₃₀ monoconjugate displays polyvalent ions at an m/z ratio of 872.8 (A6) 1047.1 (A5), 1308.6 (A4) and 1744.4 (A3) (FIG. 49) which constitute the surrounding ionic charges of the conjugate having a molecular mass of 5230 amu, which corresponds to the grafting of an OM-197-FV molecule on a P₂P₃₀ peptide molecule by reductive amination.

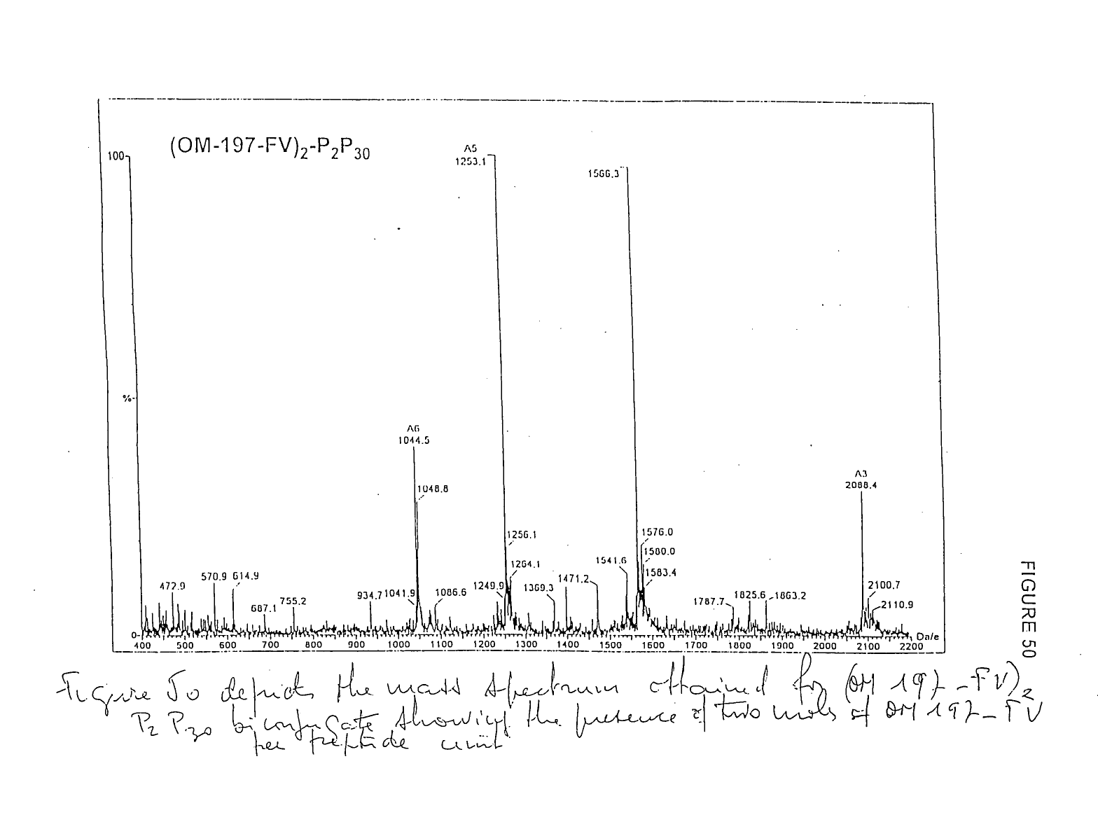

Likewise, the mass spectrum obtained for the (OM-197-FV)₂-P₂P₃₀ biconjugate ascertains the presence of two molecules of OM-197-FV per peptide unit. The surrounding ionic charge is formed by ions at an m/z ratio of 1044.5 (A6), 1253.1 (A5), 1566.3 (A4) and 2088.4 (A3) (FIG. 50), which after transform analysis verify with the expected molecular mass of the biconjugate (6261 amu).

### Example 3.4.

### (NANP)₃CS.T3 Peptide Conjugates

A similar coupling procedure can be conducted for the (NANP)₃CS.T3 peptide (TNO, MW: 3527 amu) having the following sequence:
NANPNANPNANPDIEKKIAKMEKASSVFNVVNS

Following H₂O dialysis, the observed ions on the ES/MS spectra reflect a molecular mass of 4557 and 5587 amu, corresponding to the expected values for the mono- and biconjugate compounds.

### Example 3.5.

### MR99B Peptide Conjugates

OM-197-MC-FV6 compound obtained through the reaction scheme shown on FIG. 8, can be coupled, for instance, to a peptide of pharamceutical interest such as PyCS 245-253. This peptide corresponding to the T-epitope portion of Plasmodium yoelli [Franke, E. D., et al., 1997, J. Immunol., 159:3424-3433] has the following sequence:
SYVPSAEQI (PyCS 245-253)

In order not to modify the T-epitope portion during the coupling step, amino acids SER were added to form a peptide referred to as MR99B, of the following sequence:
SERSYVPSAEQI (MR99B)

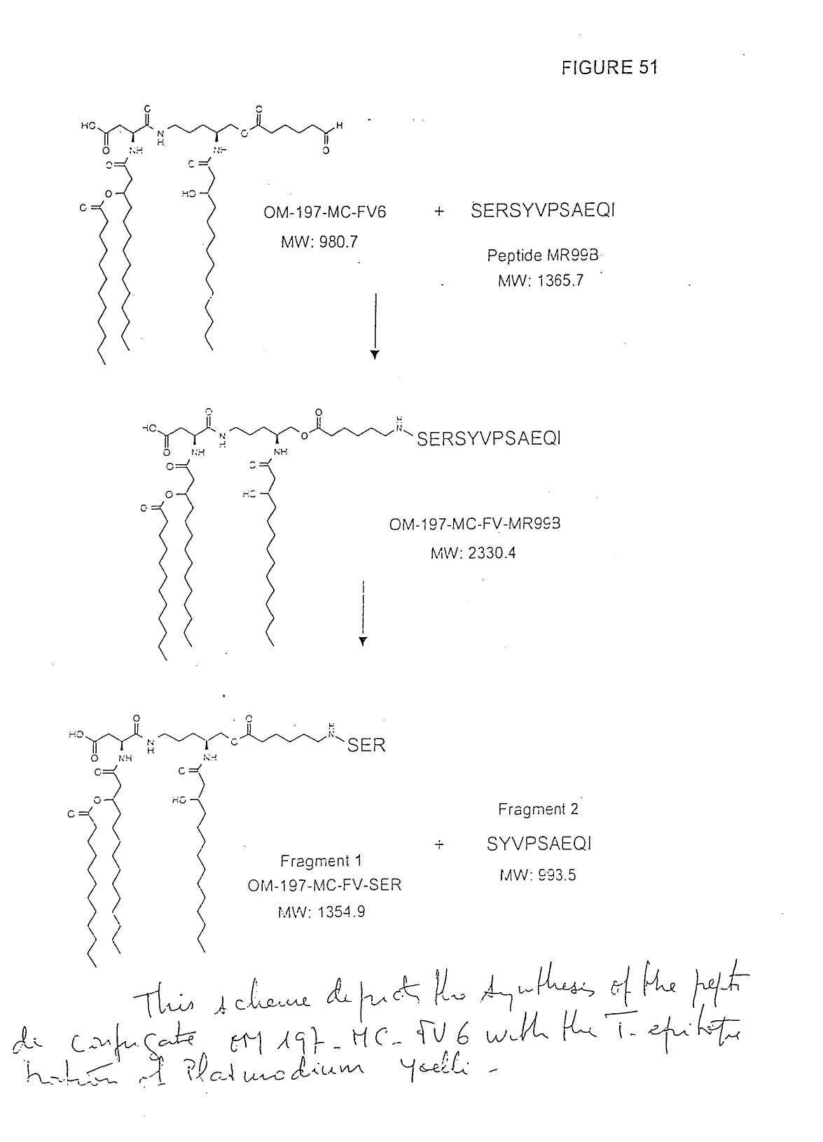

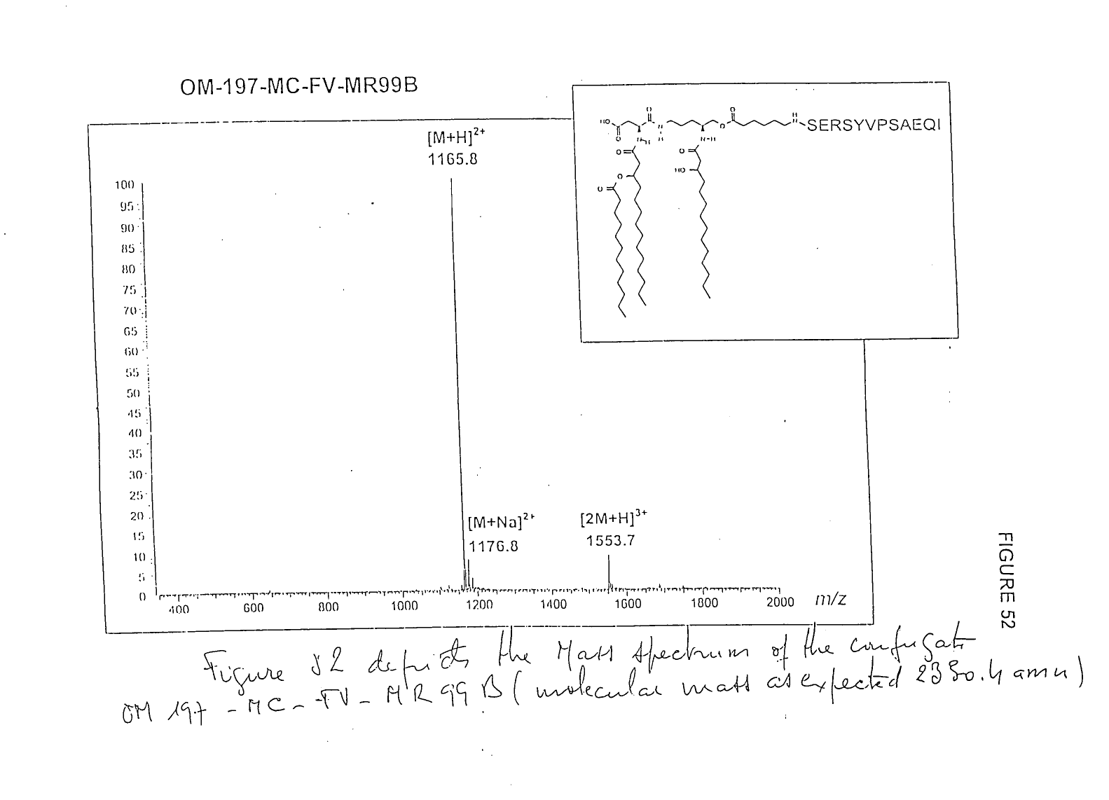

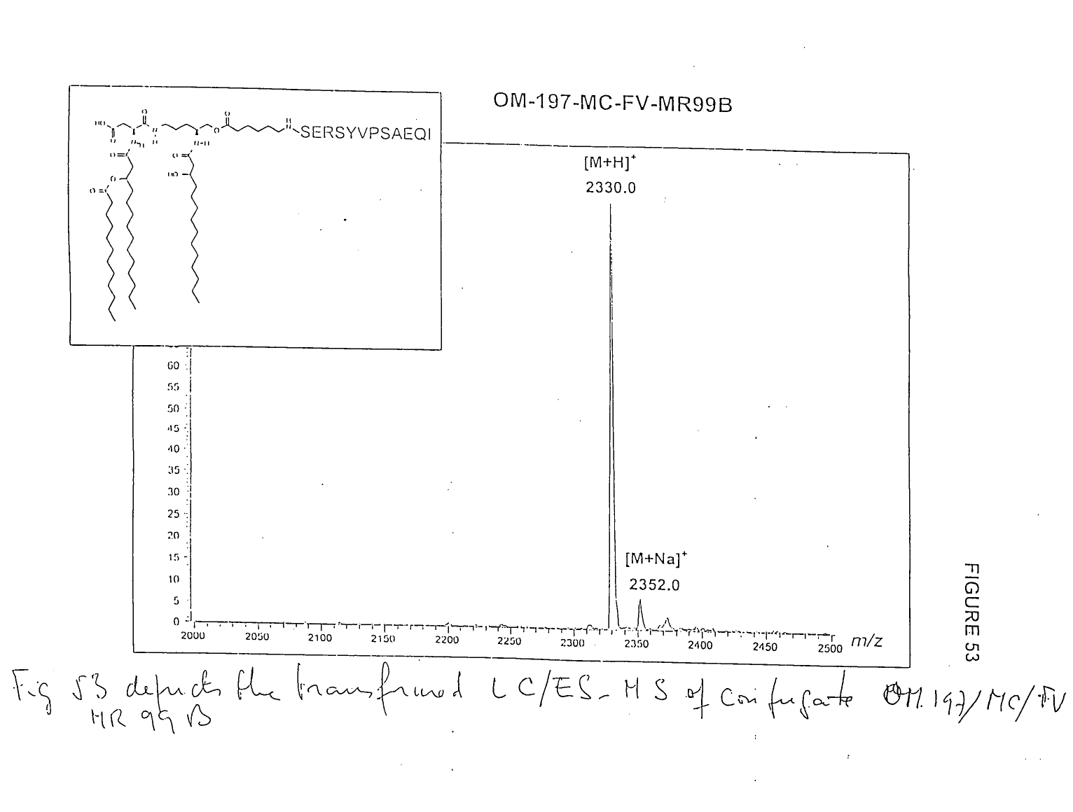

Since the present sequence is lysine-free, MR99B peptide has a unique reductive amination coupling site (at the terminal amine). Thus, 2 mg of purified OM-197-MC-FV6(2.04 μmol., 1.4 eq.) were added to a solution of 2 mg MR99B (1.47 μmol., 1 eq.) dissolved into 1 ml of a 1:1 H₂O-isopropanol mixture. The solution is stirred for 30 minutes at room temperature, then the reduction step is carried out by adding 29.4 μml. (1 M) NaBH₃CN (29.4 umol., 2 eq.) The solution is stirred for two hours at room temperature. LC/ES-MS analysis of the reaction mixture provides evidence of the formation of an OM-197-MC-FV-MR99B conjugate of an expected molecular mass (2330.4 amu, FIG. 51), as shown by spectra depicted on FIGS. 52 (surrounding ionic charge) and 53 (transformed spectrum).

Purification of OM-197-MC-FV-MR99B conjugate is carried out by semi-preparative liquid chromatography on a C4 phase according to the following conditions.
Column: Vydac C4, 250×10 mm, 5 μm, 300 Å (Vydac)
Mobile phase:
   A: (9:1, v/v) water—MeCN, 0.05% TFA
   B : (9:1, v/v) isopropanol—water, 0.05% TFA
Flow rate : 2.5 ml/min.
Elution : Isocratic elution : 65% B, 20 minutes
Washing: 100% B, 10 minutes
Detection : UV, 210 nm
System: HPLC 1050

In order to demonstrate that the peptide antigenic portion is not altered by conjugation, a trypsin digestion of the OM-197-MC-FV-MR99B is conducted. 0.5 mg of the conjugate is incubated for 24 hours with 83 μg of trypsin (Roche # 109819) (substrate-enzyme ratio (6:1)) in 1 ml of 50 mM Tris-HCl buffer, 2 mM CaCl₂, Ph 8.

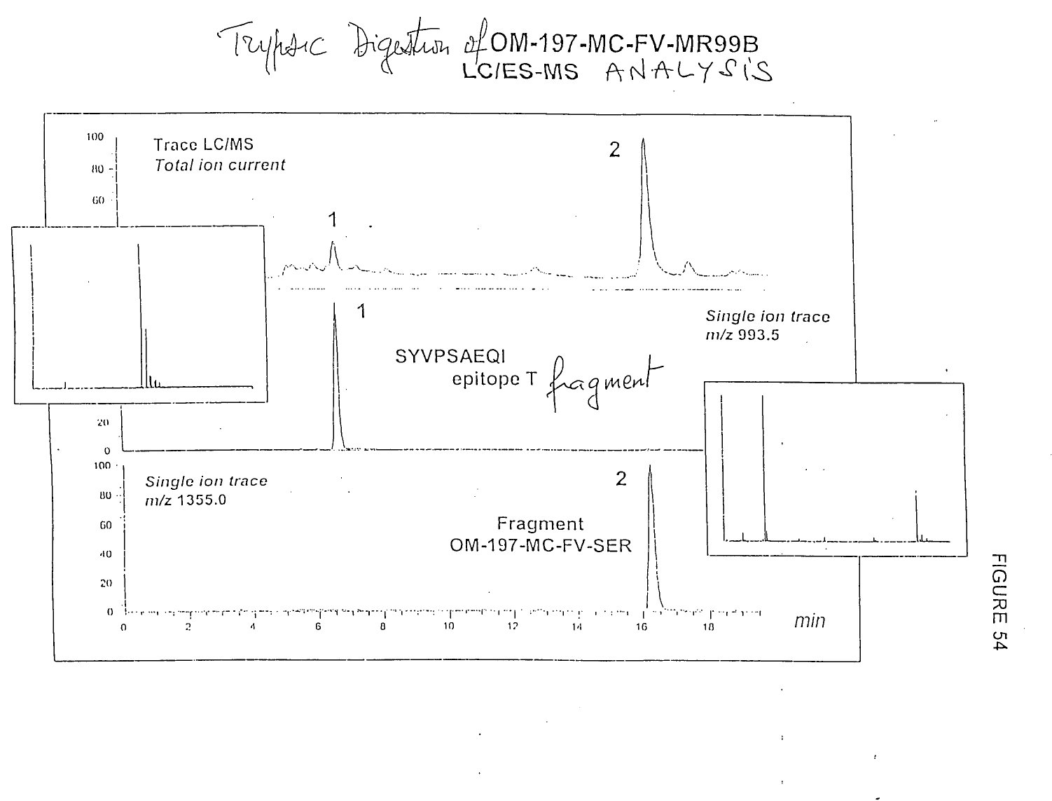

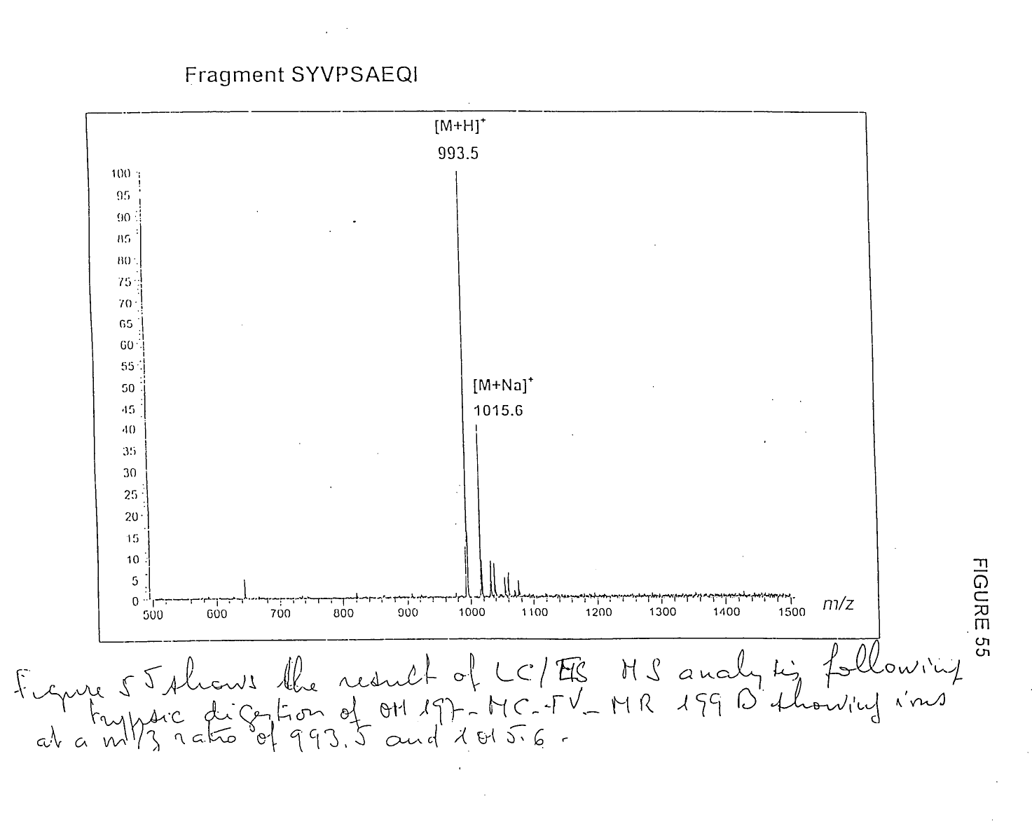

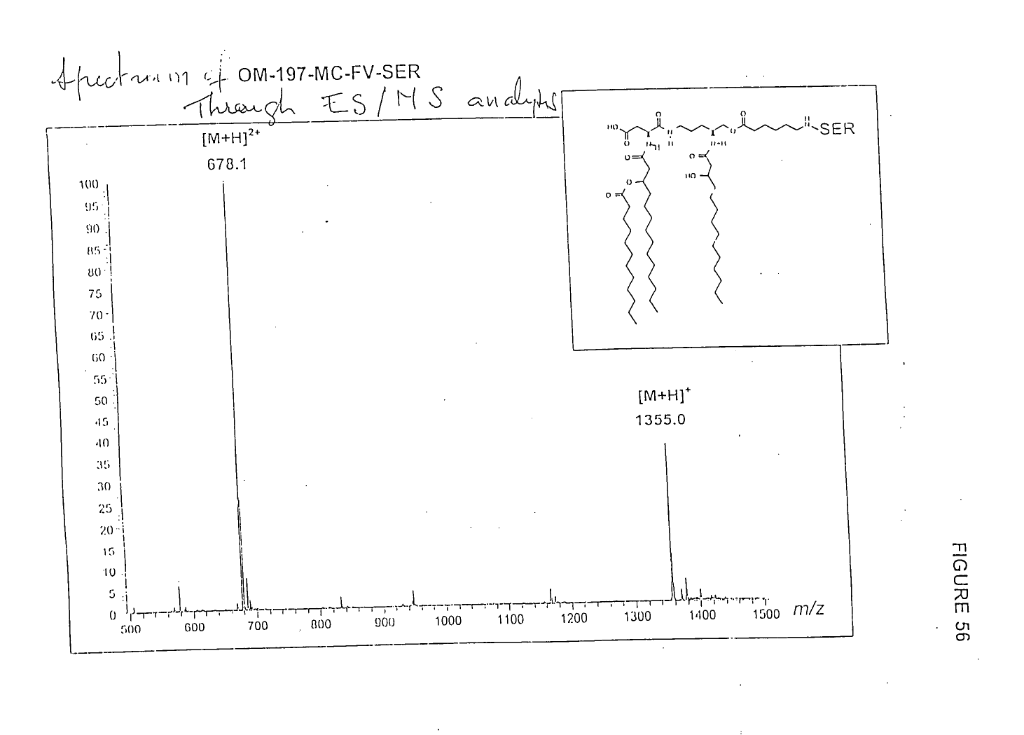

LC/ES-MS analysis following this reaction is shown on FIG. 54 and ascertains the presence of two fragments with the corresponding ES-MS spectra being shown on FIGS. 55 and 56. The first fragment clearly demonstrates ions at an m/z ration of 993.5 ([M+H]⁺) and 1015.6 ([M+Na]⁺) and corresponds to the CSPy 145-253 peptide (FIG. 55). The second fragment demonstrates ions at an m/z ration of 678.1 ([M+H]⁺) and 1355.0 ([M+H]⁺), demonstrating the presence of a OM-FV-SER type structure (FIG. 56). A third peak is also visible. This peak corresponds to both SYVPS and EQI fragments (coeluants according to chromatography separation conditions herein used). This reaction is also observed when MR99B is digested alone.

These results clearly demonstrate that conjugation does not alter the T-epitope portion of the MR99B peptide, as schematically depicted on FIG. 51.

### Example 3.6.

### MR99A Peptide Conjugates

In order to increase conjugation rate of OM-197-MR-FV to a given peptide and increase the adjuvant/antigen ration without altering the T-epitope portion, KGG type sequences can be grafted on the peptide to be grafted. Thus, a KGGKGGK sequence can be, for example, grafted to MR99B peptide, to yield the MR99A peptide as follows:
KGGKGGKSERSYVVPSAEQ (MR99A)

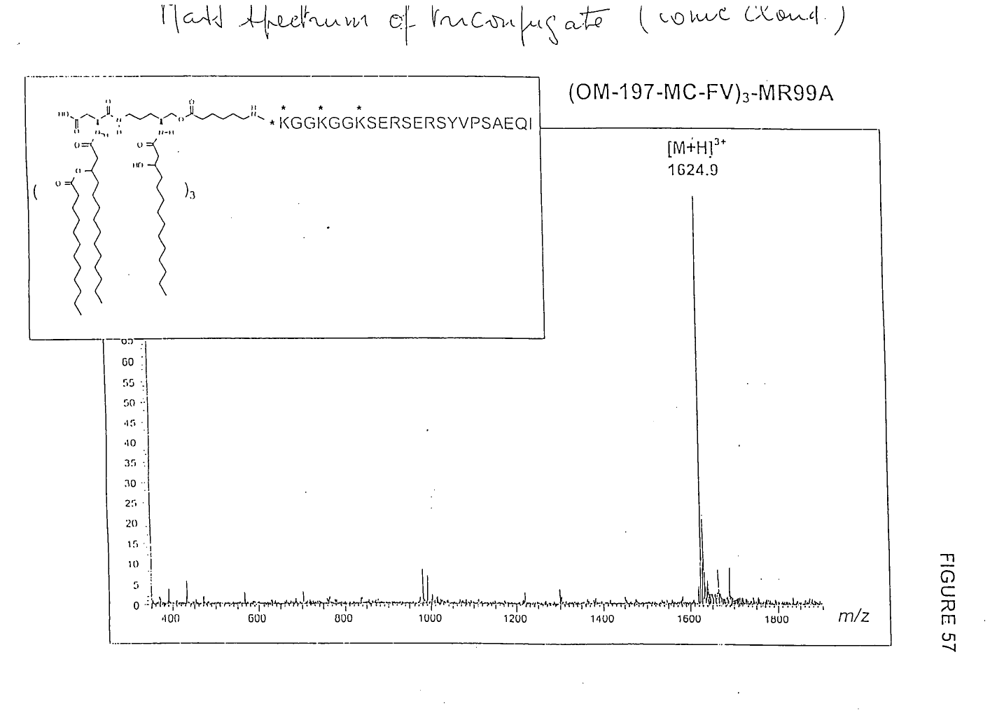

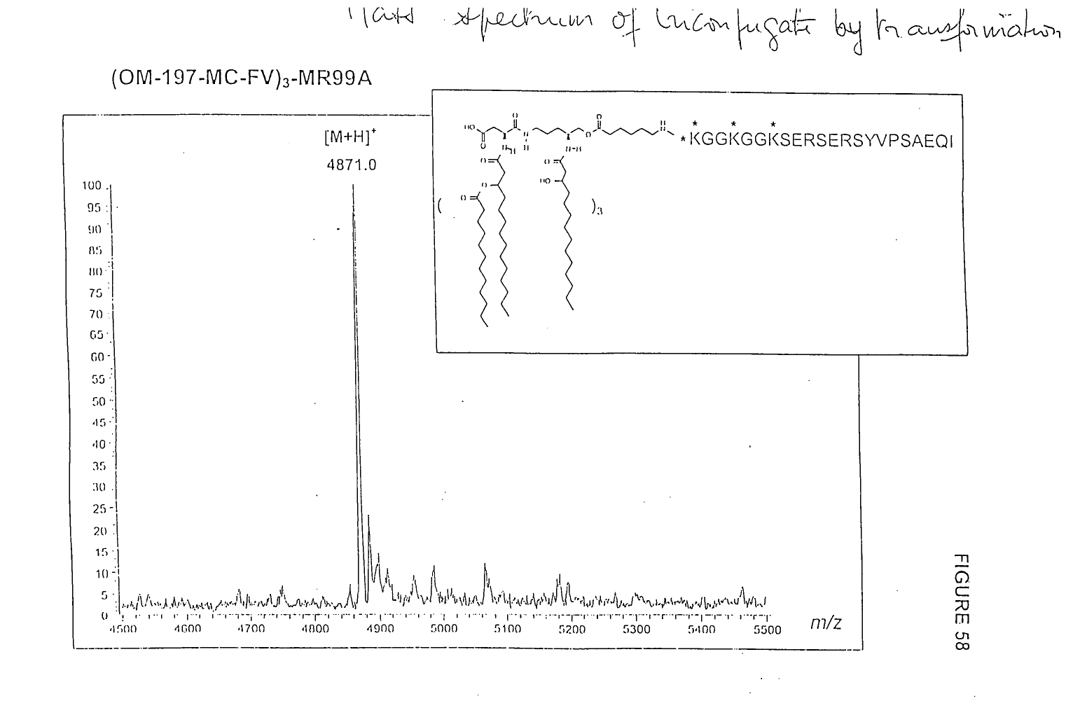

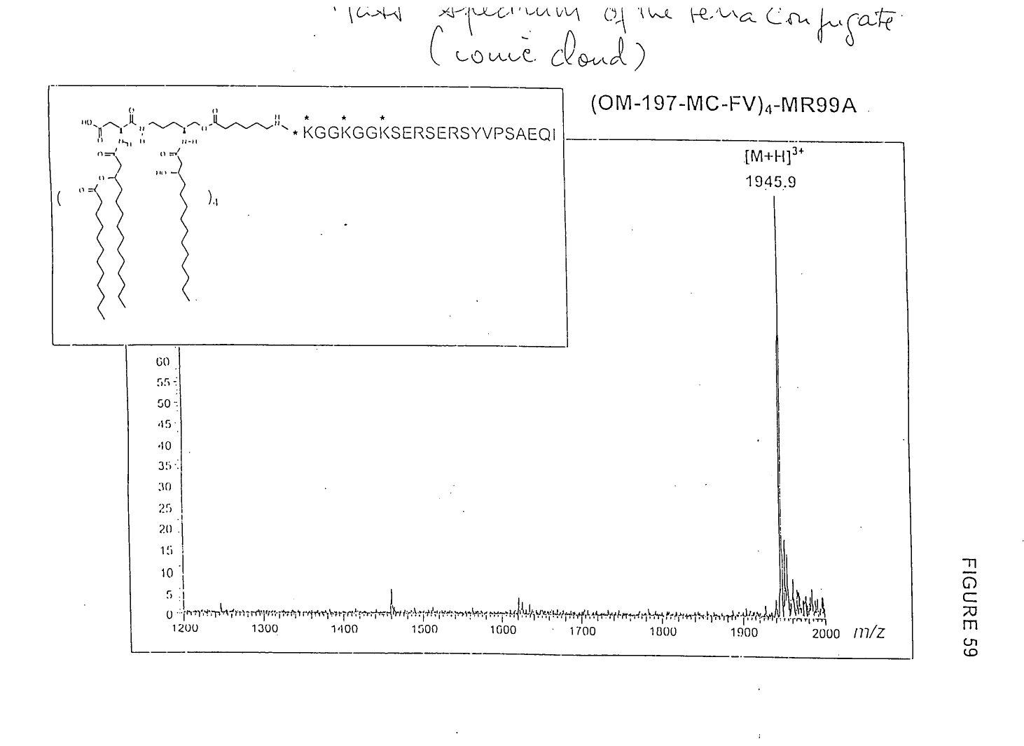

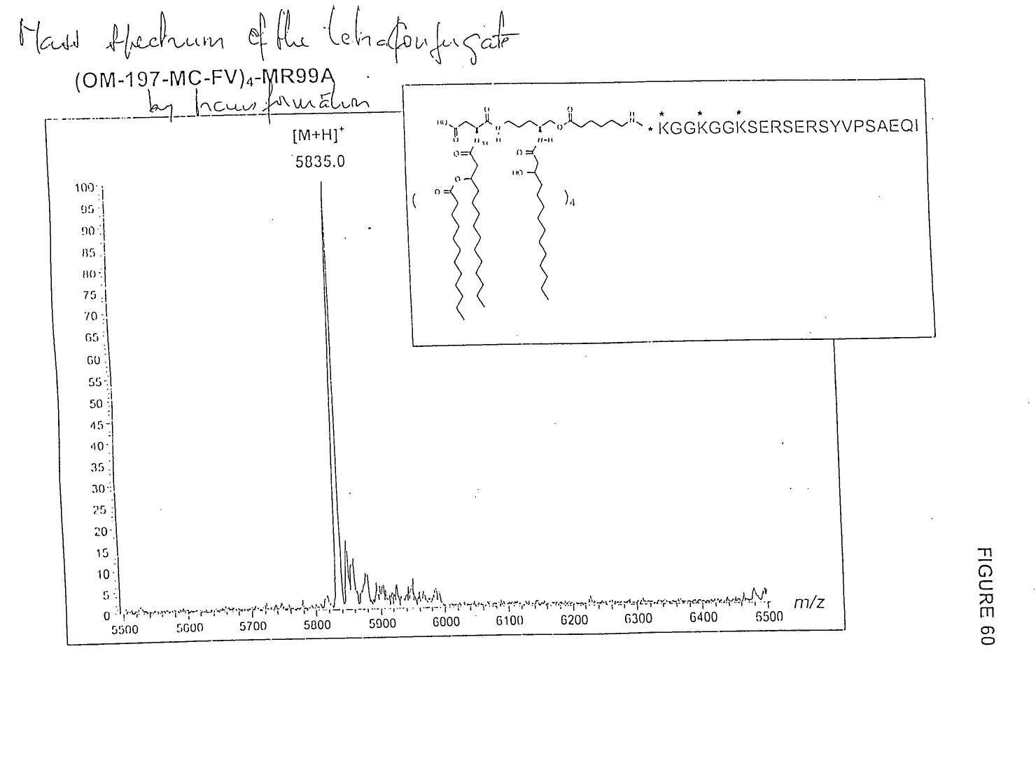

Accordingly, this peptide has four potentiel conjugation sites for a reductive amination reaction (three lysines and the terminal amine). Thus, 12 mg of purified OM-197-MC-FV6 (12.2 μmol., 12 eq.) are added to a solution of 2 mg of MR99A dissolved into 1 ml of 1:1 H₂O-isopropanol. The solution is stirred for 30 minutes at room temperature, then a reductive step is conducted by adding 242 μl of 1M NaBH₃CN (244 μmol., 242 eq.). The solution is stirred for 2 hours at room temperature. LC/ES-MS analysis of the reaction mixture demonstrates different peaks corresponding to variable conjugation rates of OM-197-MC-FV molecules to the MR99A peptide. Mass spectra corresponding to OM-197-MC-FV)₃-MR99A triconjugate (4870 amu) are shown on FIGS. 57 (ionic cloud) and 58 (transformed spectrum) whereas spectra shown on FIGS. 59 (ionic cloud) and 60 (transformed spectrum) demonstrate formation of (OM-197-MC-FV)4-MR99A tetraconjugate (5834 amu). Ions corresponding to a pentaconjuguate form are equally observed. This result shows that lysine, under certain reaction conditions (excess OM-197-MC-FV), is not the only reactive amino acid in a reductive amination reaction.

Purification of the tri and tetra-conjugates (OM-197-MC-FV)_{3, 4}-MR99A is conducted by semi-preparative liquide chromatography on a C4 phase according to the following conditions:
Column: Vydac C4, 250×10 mm, 5 μm, 300 Å (Vydac)
Mobile phase:
   A: (9:1, v/v) water—MeCN, 0.05% TFA
   B : (9:1, v/v) isopropanol—water, 0.05% TFA
Flow rate: 2.5 ml/min.
Elution : Isocratic elution : 65% B, 20 minutes
Washing: 100% B, 10 minutes
Detection: UV, 210 nm
System: HPLC 1050

As for an OM-197MC-FV-MR99B monoconjugate, (OM-197-MC-FV)n-MR99A polyconjugates are submitted to a trypsin digestion to dermine whether or not the antigenic portion (CSPy 245-253) is altered by conjugation. 1 mg of the polyconjugate is incubated for 24 hours at 25° C. with 166 μg of trypsin (Roche, #109819) (substrate-enzyme ratio: (6:1)) in 1 ml of 50 mM Tris HCl buffer, 2 mM CaCl, pH 8.

LC/ES-MS analysis carried out after the reaction ascertains the presence of a great number of cleavage products at variable locations of the MR99A peptide. Among such products, a fragment at an m/z ratio of 993.5 ([M+H]⁺) and 1015.6 ([M+Na]⁺) is noted which corresponds to a CSPy 245-253 peptide. Presence of this peptide is a proof of the integrity of the T-epitope portion of the peptide even after a mutiple conjugation reaction. Two fragments, detected through their triple charge ions at an m/z ratio of 1621.4 ([M+H]⁺) and 1942.8 ([M+H]⁺) are of special interest. In fact, such fragments correspond to fragments (OM-197-MC-FV)₄-KGGKGGKSER and (OM-197-MC-FV)₅-KGGKGGKSER, respectively. Dection thereof shows that different molecules of OM-197-MC-FV are actually grafted onto the sequence added to CSPy 245-253 even in case amino acids other than lysine have reacted in the reductive amination step. It should be noted that presence of several sterically relevant molecules of OM-197-MC-FV on a peptide does not affect protease activity as in case of trypsin. This result is of remarkable interest when considering conjugation to a prodrug where the active ingredient can be released after selective cleavage of a cleavable bond, for example.

### Example 3.7.

### Ag85c Peptide Conjugates

Structures bearing an accessory formylvaleryl type side chain spacer can be conjugated to a great number of peptides of pharmaceutical interest. Mention is made, for example, of grafting of OM-197-MC-FV molecules by reductive amination reaction to the terminal amine of synthetic peptides having sequences derived from an antigenic protein sequence of Mycobacterium tuberculosis. Peptides intially used were as follows

| | |
|---|---|
| MR 100 YLQVPSASMGR: | 1208.4 amu |
| MR 101 MVQIRPRLVANNTRIWVYC | 2176.6 amu |
| LR72 PYAASLSGFLNPSEGWWPTLIGLAM | 2676.1 amu |

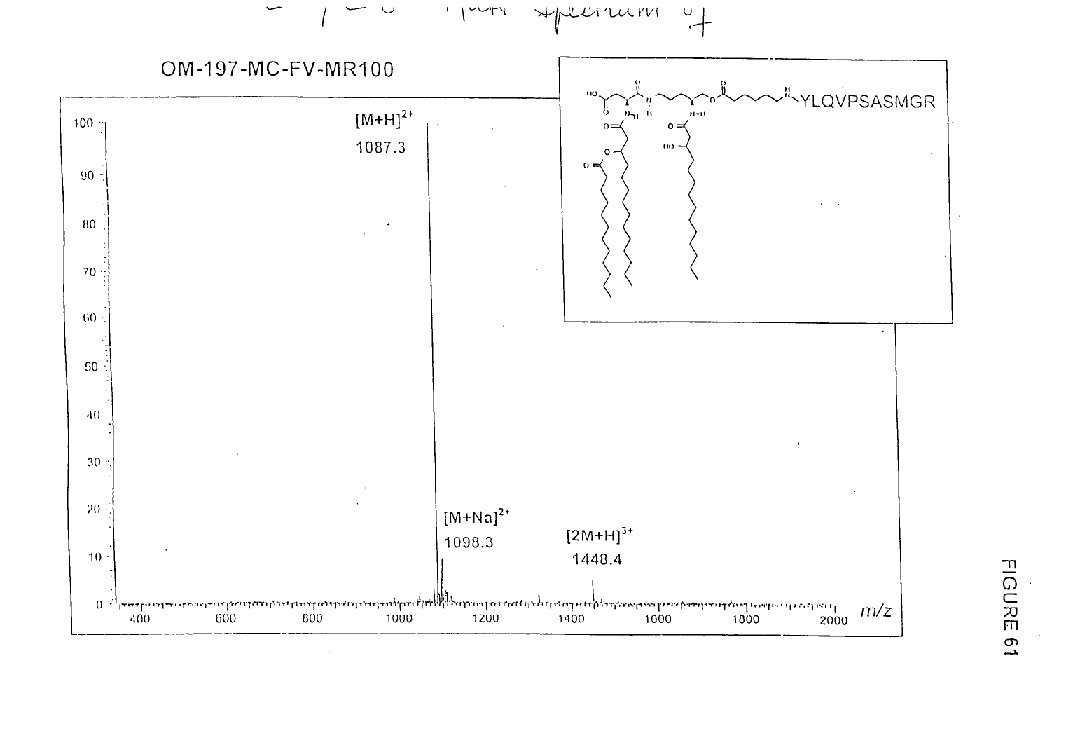

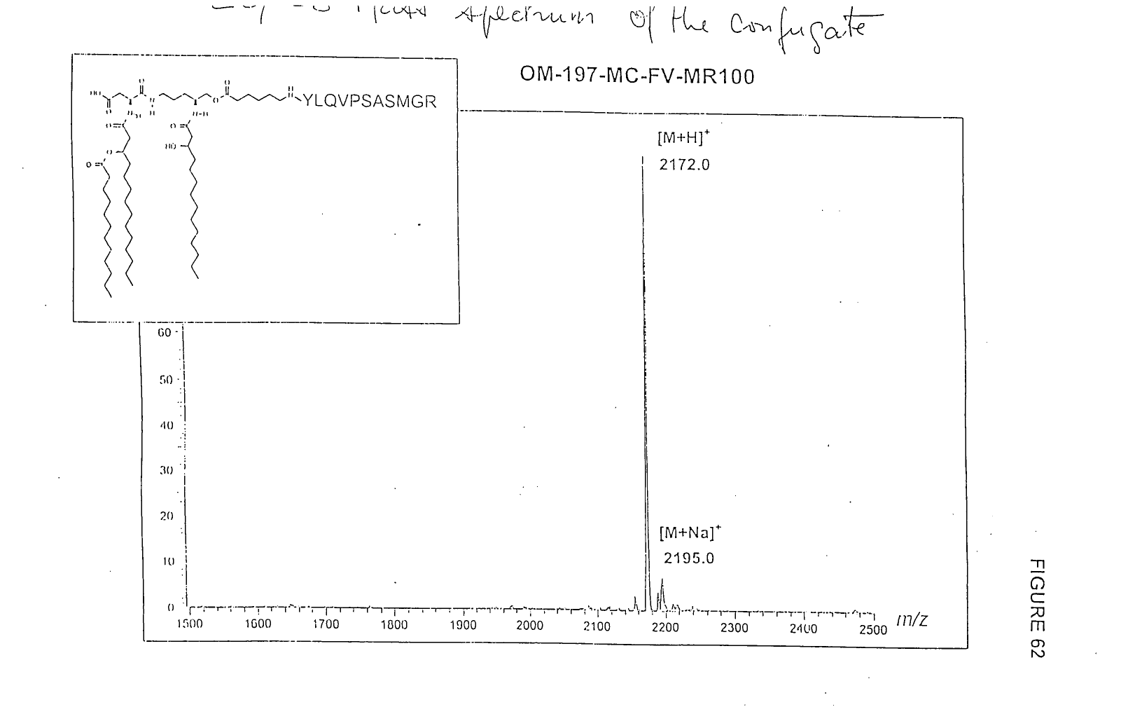

Following reaction with OM-197-MC-FV, the following conjugates were detected by LC/ES-MS analysis: for example, OM-197-FV-MR100 which displays. a molecular weight of 2171.0 amu (FIGS. 61 and 62). Alternatively, OM-197-MC-FV-MR101 of 3140.0 amu and OM-197-MC-FV-LR72 of 3643.0 amu were also obtained.

### Example 3.8.

### Dimers Having an OM-197-type Structure

The reductive amination method can be equally applied to the synthesis of dimers having an OM-197-type structure.

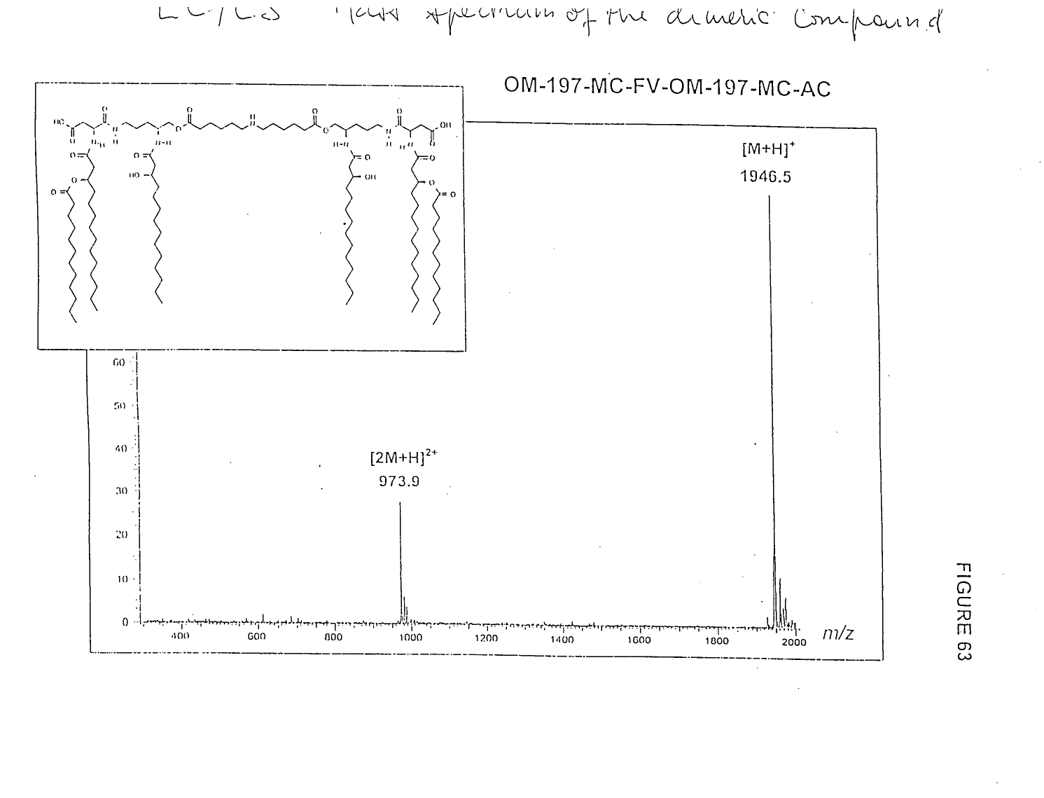

For example, a dimer can be formed starting from two compounds bearing an accessory functional side chain spacer such as OM-197-MC-FV and OM-197-MC-AC. Thus, 1 mg of purified OM-197-MC-FV6 (0.98 μmol., 1 eq.) is added to a solution of 1 mg of OM-197-MC-AC (0.98 μmol, 1 eq.) dissolved into 1 ml of (1:9) H₂O-isopropanol. The solution is stirred for 1 hour at room temperature, then the reductive step is carried out by adding 19.6 μl of 1M NaBH₃CN (19.6 μmol., 20 eq.). The solution is thereafter stirred for 12 hours at room temperature. LC/ES-MS analysis of the reaction medium demonstrates formation of an OM-197-MC-FV-OM-197-MC-AC dimer of the expected molecular weight (1945.5 amu) as evidenced by the spectrum shown on FIG. 63. This molecule hence shows two carboxylic acid functional groups at the end portions of the main chain bearing acyl chains.

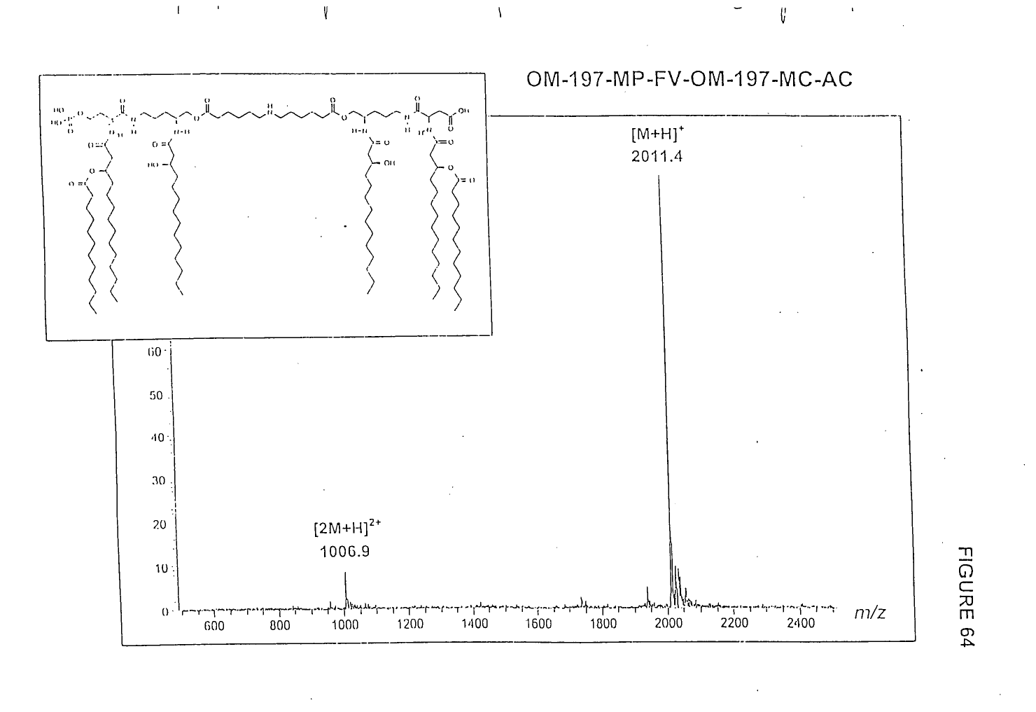

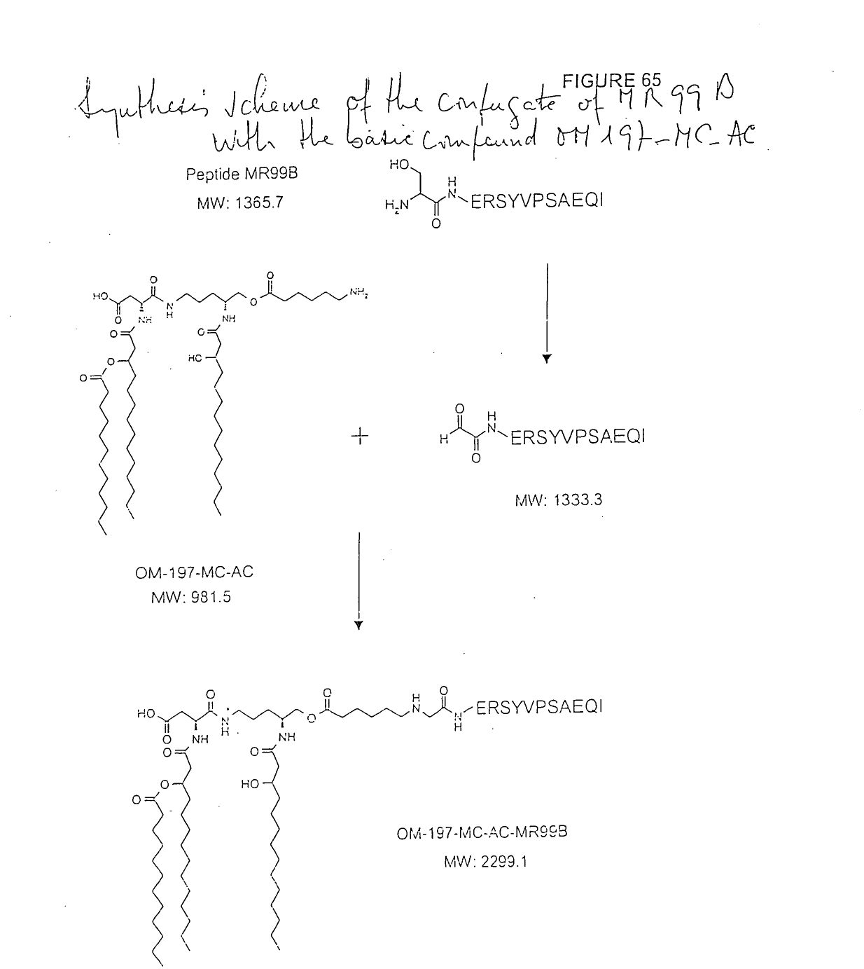

In structure shown on FIG. 63, one of the carboxylic groups can be replaced, for instance, by a phosphoryl group by conducting the above synthesis process starting from an OM-197-MP-FV or OM-197-MP-AC type compound. By performing a reductive amination similar to the one described above between two compounds bearing a functional side chain spacer (OM-197-MP-FV and OM-197-MC-AC), an OM-197-MP-FV-OM-197-MC-AC dimer can be obtained. The ES/MS spectrum depicted on FIG. 64 confirms the expected molecular mass for this compound. (2011.9 amu).

### Example 3.9.

### Conjugation of Compounds Bearing an Amino-Type Accessory Side Chain Spacer

Compounds bearing an amino type accessory side chain spacer can also be conjugated to peptide antigens through a reductive amination reaction. It is further possible to make use of the presence of a terminal serine, for example, in the sequence of petides to be conjugated. In a first step, a periodic oxidation reaction is conducted upon the peptide, leading to the formation of a highly reactive glyoxylyl group (—CO—CHO). This group can then react through a reductive amination reaction with a primary amine born on the accessory functional side chain spacer of OM-197 type compounds.

For example, the synthetic peptide MR99B can be conjugated to an OM-197-MC-AC compound, as shown by the synthesis scheme of FIG. 65. To this end, 147 μl of 0.1 M NaIO₄ (14.6 μmol., 20 eq.) are added to a solution of 1 mg of MR99B peptide (0.73 μmol., 1 eq.) dissolved into 1 ml of water. The solution is stirred for 2 hours at RT. The aldehyde compound thus formed is purified on a C18 phase and recovered into 1 ml of a (1:1) isopropanol-water mixture. 0.72 mg of the OM-197-MC-AC compound (0.73 μmol., 1 eq.) are then added, as well as 1 ml of a 0.2 M borate buffer, pH 9.2. After stirring at room temperature for 30 minutes, the reduction step is carried out by adding 14.6 μl of 1 M NaBH₃CN (14.6 μmol., 20 eq.). The solution is stirred for 24 hours at room temperature.

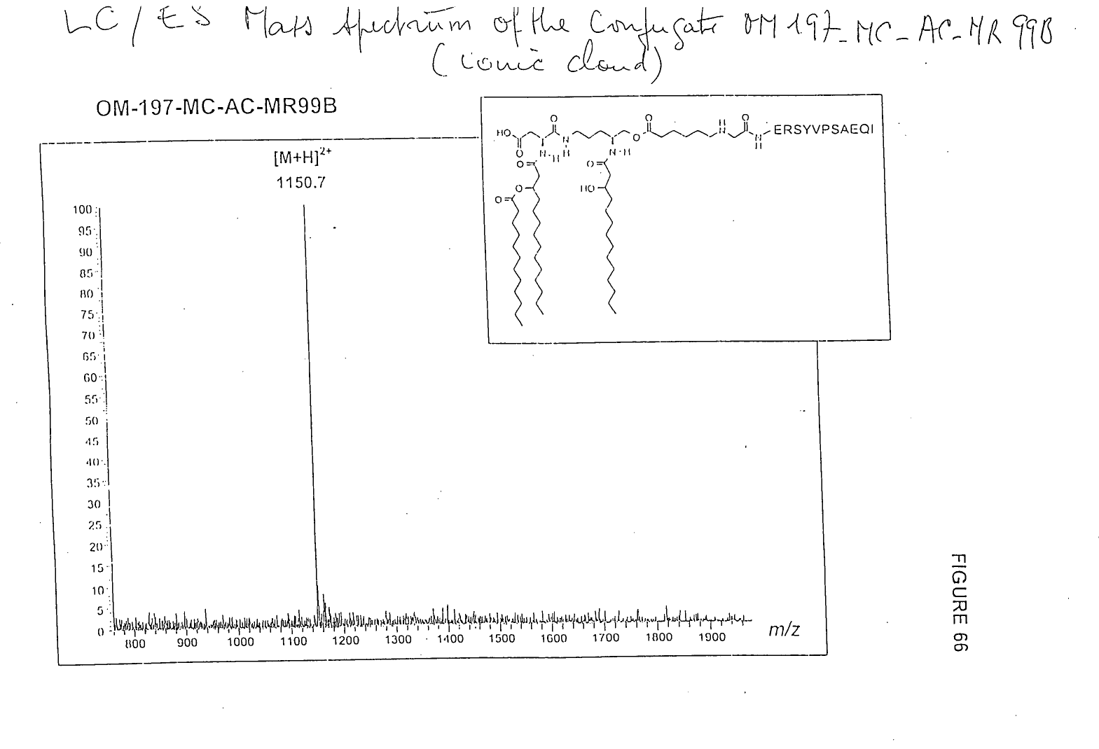

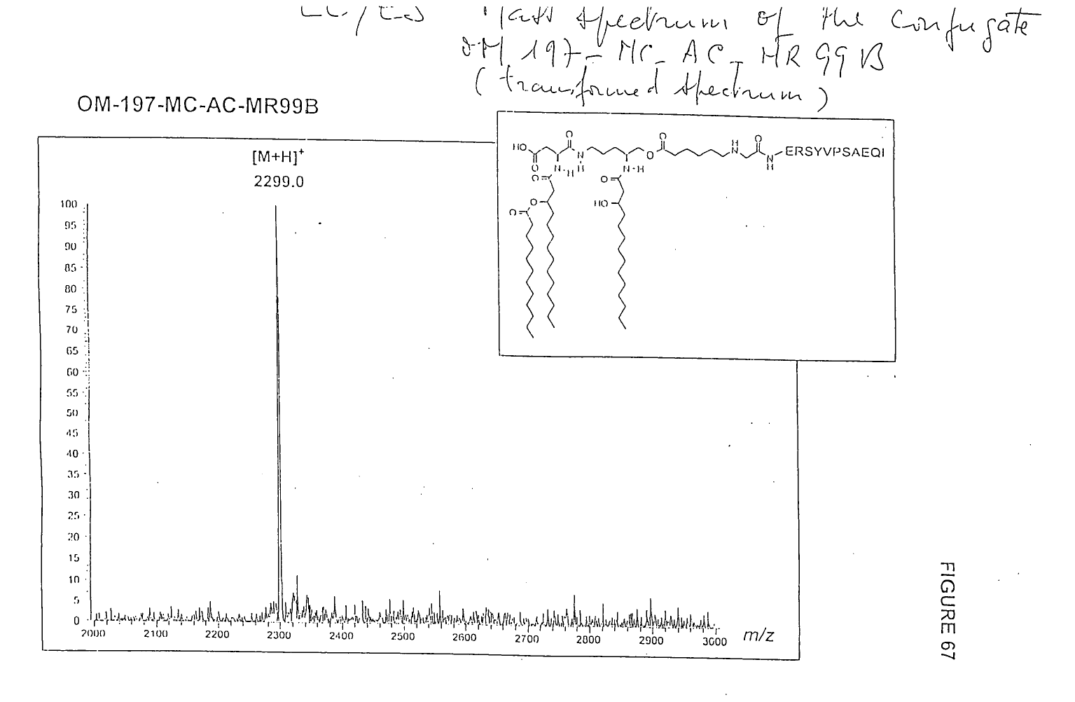

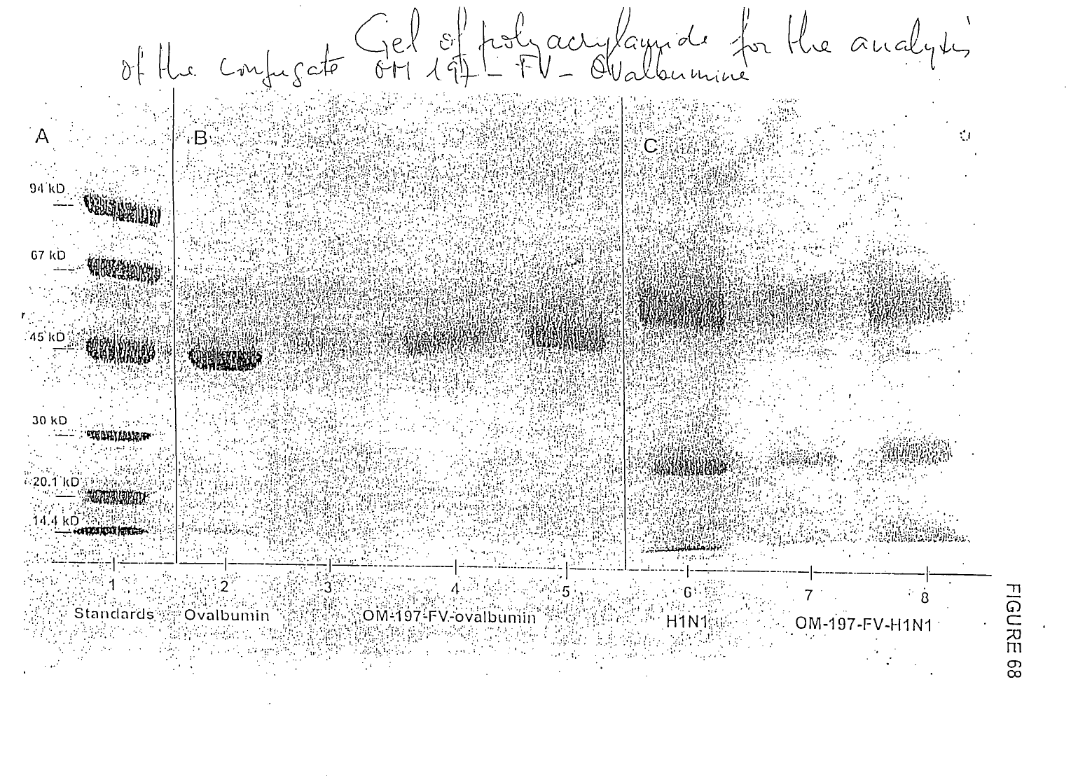

LC/ES-MS analysis of the reaction mixture demonstrates the formation of an OM-197-MC-AC-MR99B conjugate of the expected molecular mass (2299.1 amu, FIG. 65) as evidenced by spectra depicted on FIGS. 66 (ionic cloud) and 67 (transformed spectrum).

Other compounds bearing an amino-type accessory functional side chain spacer can be conjugated in a similar fashion. Mention is made, for example, of OM-197-MC-AP, OM-197-MP-AC,, OM-212-AH1, OM-197-MC-gly, OM-197-MC-Lys, OM-197-Lys-AC or OM-197-Asp-AC.

### Example 3.10.

### Ovalbumin Conjugates

Protein, such as ovalbumin for instance, are particularily adapted to reductive amination coupling. The great number of lysine residues contained in the sequence (20 lysine residues for ovalbumin) are all potential conjugation sites for the dipeptide-like compounds bearing an accessory functional side chain spacer with an aldehyde functional group. By varying the stoechiometric ratio of the reductive amination reaction (acyl-dipeptide-like compound/protein), conjugation to a variable extent can be achieved.

1 mg of purified OM-197-FV7 (0.928 μmol., 10 eq.) was added to a solution of 4 mg of ovalbumin (0.09 μmol., 1 eq.) dissolved in 5 ml of H₂O. The solution was stirred for 30 minutes at room temperature, before proceeding with a reduction step by adding 46 μl of 1M NaBH₃CN (2.89 mg, 50 eq.) The solution was stirred for two hours at room temperature. The reaction mixture was subsequently dialyzed against H₂O for a period of 24 hours (3.5 kDa dialysis cassette, Slide-A-Lyzer, Pierce).

Owing to its size and its inherent heterogenous character, ovalbumin raises a great number of analytical problems. As a result, characterization of an OM-1976FV-ovalbumin conjugate by mass spectrometry may prove a very difficult task. To show that a conjugation reaction did occur, SDS-PAGE analysis were conducted on different batches of OM-197-FV-ovalbumin conjugates. As is apparent from the gel shown on FIG. 68 (B) (4-20% polyacrylamide gel gradient), a mass exceeding the initial mass of ovalbumin by about 3000 amu (lane 2) is observed for OM-1976FV-ovalbumin conjugates (lanes 3, 4 and 5). This mass difference suggests that several molecules of OM-197-FV are present on each molecule of ovalbumin. LC/UV analysis conducted on a C₄ phase confirm this result. As a matter of fact, in these conditions, the initially used ovalbumin clearly appears at R_{T}=11.3 min., whereas following the conjugation reaction, the peak corresponding to initially used ovalbumin is no longer visible, a fact which shows that reaction with ovalbumin was stoechiometric. The OM-197-FV-ovalbumin conjugate is however not eluted. This chromatographic profile also indicates that more than one single OM-197-FV molecule is present on ovalbumin, thus preventing the elution of the conjugate in such conditions.

### EXAMPLE 3.11.

### 3.6. H1N1 Hemagglutinin Conjugates

H1N1 hemagglutinin protein also provides a fine example intended to illustrate coupling between OM-197 type molecules and a protein antigen.

1 mg of purified OM-197-FV7 (0.928 μmol., 15 eq.) is added to a io solution of 5 mg of H1 N1 (hemagglutinin A/Beijig 262/95, Solvay Duphar, Weesp, NL) (0.06 μmol., 1 eq.) dissolved in 8 ml of H₂O. The solution is stirred for 30 minutes at room temperature, then the reduction step is performed by adding 46 μl of 1 M NaBH₃CN (2.89 mg, 50 eq.). The solution is stirred for 2 hours at room temperature. The reaction mixture is then dialyzed against H₂O for 24 hours (3.5 kDa dialysis cassette).

The OM-197-FV-H1N1 conjugate is analyzed by SDS-PAGE in the same conditions used for OM-197-FV-ovalbumin conjugate (4-20% polyacrylamide gradient). The electrophoretic profiles obtained for the starting protein (lane 6) and the conjugate before and after dialysis (lanes 8 and 7, respectively) are shown on FIG. 68(C). H1N1 protein appears as three bands, two of which correspond to HA1 and HA2 subunits reported in the litterature (Swiss-Prot, Swiss Institute of Biological Computer Science, Geneva). In case of OM-197-FV-H1N1 conjugates, a mass difference is observed for each band, demonstrating that coupling of OM-197-FV molecules with H1N1 protein was achieved.

### Example 3.12.

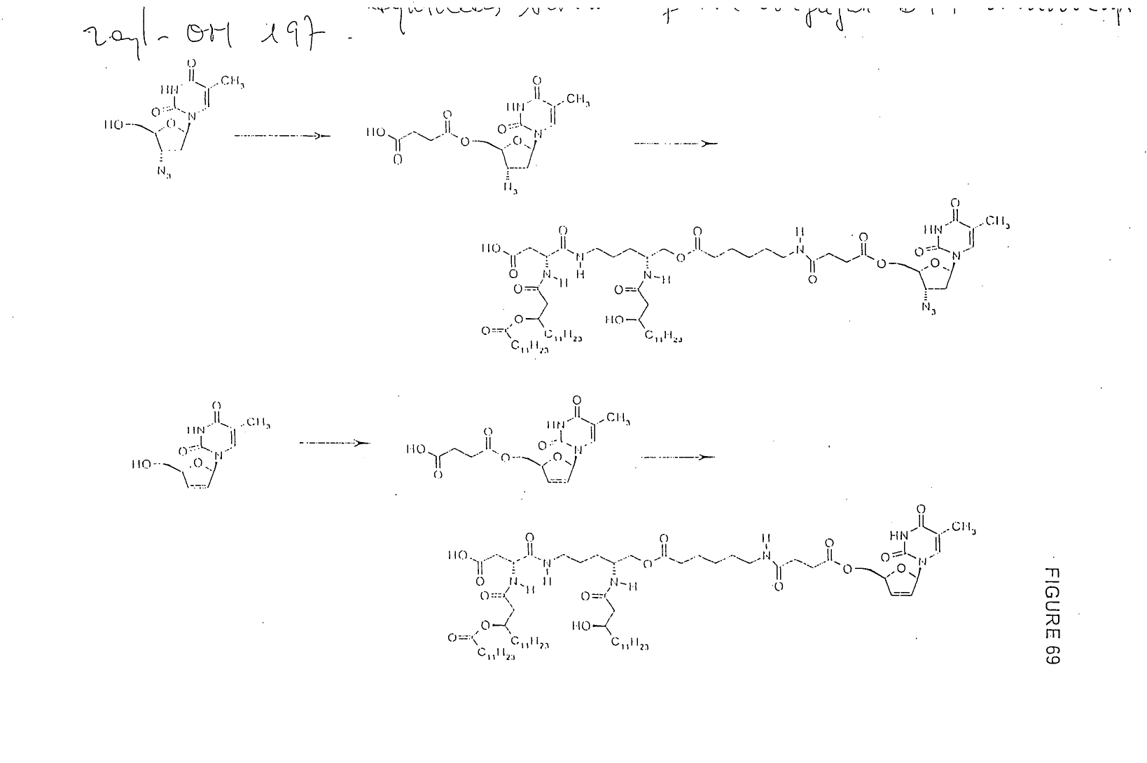

### AZT Conjugates (FIG. 69)

To a solution of AZT (200 mg; 0.749 mmol.) in pyridine (4 ml), there are added in succession succinic anhydride (150 mg; 0.5 mmol.) and 4-dimethylaminopyridine (50 mg ; 0.41 mmol.) After being allowed to stand for 12 hours at 50° C., methanol (2 ml) is added and the reaction medium is stirred for further 15 minutes at room temperature. The reaction medium is then concentrated and the product is purified by flash chromatography on a silica gel using the following eluent: 5/1 CH₂Cl₂/MeOH. As a result, the product is obtained as a white solid (266 mg; 97%). C₁₄H₁₇N₅O₇ (M=367 g/mol.); Rf=0.25 (4/1 CH₂Cl₂/MeOH)

To a solution of a succinyl-AZT derivative thus obtained (60 mg 0.163 mmol.) in THF (3 ml) at 0° C. and under argon flow, there are added in succession triethylamine (24 μl ; 0.172 mmol.) and ethyl chloroformate (14 μl; 0.172 mmol.). Rapid formation of a triethymamine hydrochloride precipitate is observed. After stirring for 40 minutes, addition is made of an aminocaproyl derivative solution (Ex. 2.16.) (160 mg ; 0.163 mmol.) in a 9/1 DMF:water mixture (10 ml) containing triethylamine (24 μl ; 0.172 mmol.). The reaction mixture is then stirred for 15° C. at 0° C. and thereafter overnight at room temperature before being evaporated under parial vacuum. The crude product is then taken up into dichloromethane (15 ml) and water (5 ml). The organic layer is separated. The aqueous layer is acidified with a 10% citric acid aqueous solution then extracted by dichloromethane (2×). The organic layers are then pooled, dried over MgSO₄, filtered and concentrated. By running a flash chromatography purification on a silica gel with the following eluent (9/1 then 7/1 CH₂Cl₂/MeOH), there is recovered the AZT-conjugated compound (123 mg ; 57%) as a white solid. Rf=0.2 (4/1 CH₂Cl₂/MeOH); C₆₉H₁₁₉N₉O₁₆ (M=1329 g/mol); Found: m/z 1330.9 ([M+H]⁺).

Analytic HPLC : Retention time Tr=24.437 min., C18 Supelcosil Column (15 cm×4.6 mm, 3 μm, 100 Å); Solvent A=50% MeCN, 5 mM TBAP ; Solvent B =90% 2-propanol, 5 mM TBAP. Gradient: 25-100% B within 37.5 min., UV detection at 210 nm.

Preparative HPLC : C18 Kromasil column (25 cm×21 mm, 5 μm, 100 Å), solvent A=50% MeCN, 5 mM TBAP ; Solvent B=90% 2-propanol, 5 mM TBAP.

Product elution with 80% B.

Final phase : flowing a Na⁺ salt by SPE, C18 Bondapack phase 15 μm, elution by 90% 2-propanol.

d4T Conjugates (FIG. 69)

To a solution of d4T (200 mg ; 0.89 mmol.) in pyridine (4 ml), there are added in succession succinic anhydride (179 mg ; 0.786 mmol.) and 4-dimethylaminopyridine (109 mg ; 0.89 mmol.) After stirring for 12 hours at room temperature, methanol (2 ml) is added and the reaction medium is stirred for further 15 minutes at room temperature and finally evaporated. The crude product is then purified by flash chromatography on a silica gel using the following eluent: 5/1 CH₂Cl₂/MeOH. The resulting product is obtained as a white solid (280 mg; 97%). C₁₄H₁₆N₂O₇ (M=324 g/mol.); Rf=0.25 (4/1 CH₂Cl₂/MeOH)

To a solution of a succinyl-d4T derivative thus obtained (60 mg 0.185 mmol.) in THF (3 ml) at 0° C. and under argon flow, there are added in succession triethylamine (27 μl; 0.194 mmol.) and ethyl chloroformate (16 μl ; 0.194 mmol.). Rapid formation of a triethylamine hydrochloride precipitate is observed. After stirring for 40 minutes, addition is made at 0° C. of an aminocaproyl derivative (Ex. 2.16.) (181 mg ; 0.185 mmol.) in a 9/1 DMF:water mixture (10 ml) containing triethylamine (27 μl ; 0.194 mmol.). The reaction mixture is then stirred for 15 min. at 0° C. and thereafter at room temperature before being evaporated under parial vacuum. The crude product is then taken up into dichloromethane (15 ml) and water (5 ml). The organic layer is separated. The aqueous layer is acidified with a 10% citric acid aqueous solution then extracted by dichloromethane (2×). The organic layers are then pooled, dried over MgSO₄, filtered and concentrated. By running a flash chromatography purification on a silica gel with the following eluent (9/1 then 7/1 CH₂Cl₂/MeOH), there is recovered the d4T-conjugated compound (107 mg ; 45%) as a white solid. Rf=0.2 (4/1 CH₂Cl₂/MeOH); C₆₉H₁₁₈N₆O₁₆ (M=1329 g/mol); Found: m/z 1288.9 (MH⁺); 1310 (Na).

Analytic HPLC : Retention time Tr=24.455 min., C18 Supelcosil Column (15 cm×4.6 mm, 3 μm, 100 Å); Solvent A=50% MeCN, 5 mM TBAP ; Solvent B=90% 2-propanol, 5 mM TBAP. Gradient : 25-100% B within 37.5 min., UV detection at 210 nm.

Preparative HPLC : C18 Kromasil column (25 cm×21 mm, 5μm, 100 Å), solvent A=50% MeCN, 5 mM TBAP; Solvent B=90% 2-propanol, 5 mM TBAP.

Product elution with 80% B.

Final phase : flowing a Na⁺ salt by SPE, C18 Bondapack phase 15 μm, elution by 90% 2-propanol.

### Example 4

### 4^{th} Series of Examples

### Pharmacological Study of Compounds in Accordance with the Invention (In Vitro)

### Example 4.1.

### Endotoxicity Determination Assay

Endotoxicity levels were determined by a time-course chromogenic Limulus Amoebocyte Lysate test (Charles River Endosafe, product R1708K, batch EK2251E).

This LAL test was used to demonstrate that antigens coupled to the inventive compounds have low endotoxicity.

The biological principle on which this test is based consists in priming of a proenzyme present in Limulus amoeboycte lysate (LAL) by bacterial endotoxins, which newly formed enzyme activates at its turn a serine-protease cascade. In presence of a colorless substrate (S-2834 peptide coupled with p-nitroaniline), cleavage of a chromophore finally is results in the liberation of p-nitroaniline (pNA) and development of a yellow color which can be monitored with a spectrophotometer at 405 nm.

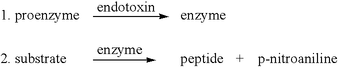

This time-course chromogenic test is based on the fact that endotoxin quantity is proportional to the reciprocal of the time period required to reach an optical density (O.D.) of 0.2. The concentration is determined in relation to a standard curve covering the 0.005-50 EU/ml range.

Products are tested using a (5-, 10-, 50-, 100-, 500- or 1000-fold) dilution of a 0.1 mg/ml product solution. LAL result corresponds to the lowest dilution which does no longer inhibit recovery of an LPS overload.

Results are expressed in EU (endotoxin unit) in relation to an international standard solution (EC-6). For this series of assays, 1 EU stands for 0.08 ng of E. coli strain 055:B5 LPS.

LAL values obtained for various acyl-dipeptide-like compounds bearing
an accessory functional side chain spacer having the general formula I
(table listing)

| | | | ng of LPS |
|---|---|---|---|
| | | | equivalent |
| | | | per mg |
| Tested Compounds | dilution | EU/mg of product | of product |
| OM-197-MC | 50 | 88.6 | 7.4 |
| OM-197-MC-MP | 50 | 210.5 | 16.8 |
| OM-197-FV6 | 50 | 540 | 43 |
| OM-197-FV8 | 50 | 1820 | 145.6 |
| OM-197-MC-FV5 | 50 | 49.3 | 3.9 |
| OM-197-MC-FV7 | 10 | <0.05 | <0.004 |
| OM-197-MP-AC(R/S, R) | 25 | 0.14 | 0.011 |
| OM-197-MP-AC (R, R) | 25 | 0.15 | 0.012 |
| OM-197-MP-AC (S, R) | 25 | <0.125 | <0.01 |
| OM-197-MC-AC | 25 | 6.0 | 0.48 |
| OM-197-N′C2-MC-AC | 10 | <0.5 | <0.04 |
| OM-197-N′(C10-20C8)-MC-AC | 10 | <0.5 | <0.04 |
| OM-197-MC-Succ | 25 | 5.3 | 0.42 |
| OM-197-Lys | 25 | 3.0 | 0.24 |
| OM-197-AP | 10 | <0.5 | <0.04 |
| OM-197-Asp | 50 | <2.5 | <0.2 |
| OM-197-Asp-AC | 50 | 2307 | 184.6 |
| OM-197-Lys-AC | 50 | <2.5 | <0.2 |
| OM-197-MC-AC-Succ-AZT | 10 | 1.34 | 0.11 |
| OM-197-MC-AC-Succ-d4T | 10 | 1.05 | 0.08 |
| OM-197-MC-AC-Succ | 10 | <0.05 | <0.004 |

All tested dipeptide-like compounds show low endotoxicity levels. For many such products, this endotoxicity is even lower than assay sensitivity. Quantitative determination of endotoxicity is limited due to the fact that samples must be diluted in order to recover overload. Even the most active compounds in this LAL test display an endotoxicity level 5000-fold less than LPS.

Dipeptide-like compounds of the invention are interesting in view of their biological activities and low endotoxicity.

TABLE
LAL results for different products used in immunization experiments
with ovalbumin-conjugated products

| Tested compound or | | ng of LPS equivalent |
|---|---|---|
| admixture | EU/mg | per mg of product |
| Ovalbumin | <0.8 | <0.064 |
| Ovalbumin + OM-197-MP | 5.8 | 0.48 |
| OM-197-FV-ovalbumin | 99 | 8.21 |

It is believed that coupling OM-197-FV to ovalbumin results in an increase in LAL activity actually detected. This increase may be ascribed to a change in how the OM-197-FV molecule is presented. The other products of the ovalbumin series all have equivalent LAL activities. LAL results are highly variable and a 3-fold to 4-fold difference between groups is non significant. Peak LAL activity is equal to 2.4 ng of LPS equivalent per injection (25 μg/shot) for the coupling reaction products, as for other groups, the peak LAL activity is equal to 0.012 ng of LPS equivalent per injection.

TABLE
LAL results for different products used in immunization experiments
with H1N1 hemagglutinin-conjugated products

| Tested compound or | | ng of LPS equivalent |
|---|---|---|
| admixture | EU/mg | per mg of product |
| H1N1 | 2 | 0.17 |
| H1N1 + OM-197-MP | 20 | 1.67 |
| OM-197-FV-H1N1 | 15.6 | 1.30 |

Products of the H1N1 series all have comparable LAL activities. LAL results are highly variable and a 3-fold to 4-fold difference between groups is non significant. Coupling has no effect on LAL activity. Peak LAL activity is equal to 0.008 ng of LPS equivalent per injection (5 μg/shot).

TABLE
LAL results for different products used in immunization experiments
with (NANP)₆P₂P₃₀ peptide-conjugated products

| | | ng. of |
|---|---|---|
| | | LPS equivalent |
| Tested compound or admixture | EU/mg | per mg of product |
| (NANP)₆P₂P₃₀ | 16 | 1.33 |
| (NANP)₆P₂P₃₀ + IFA | n.d.* | n.d.* |
| (NANP)₆P₂P₃₀ + OM-197-FV6 | 13 | 1 |
| (NANP)₆P₂P₃₀ + OM-197-MC | 12 | 1 |
| (OM-197-FV)_{1,2,3}-(NANP)₆P₂P₃₀ | 2 | 0.17 |
| OM-197-FV-(NANP)₆P₂P₃₀ | 9.4 | 0.78 |
| OM-197-FV-(NANP)₆P₂P₃₀ + OM-197-MP | 17.4 | 1.45 |

*cannot be assayed by LAL as this test is unsuitable for emulsion samples

Products of the (NANP)₆P₂P₃₀ series all have comparable LAL activities. LAL results are highly variable and a 3-fold to 4-fold difference between groups is non significant. Coupling has no effect on LAL activity. Maximum LAL activity is equal 0.03 ng of LPS equivalent per injection (20 μg/shot).

### Example 4.2.

### Determination of the Bone Marrow Stem Cell Proliferation in Mice Challenged with the Inventive Products

4.2.1. Proliferation Experiment

6-week old male C57/BL6 mice are killed by CO₂ inhalation. The hip, femur, tibia and hind leg bones are removed. The marrrow is extracted from the bone lumen by injecting Dulbecco's Modified Eagle Medium (DH medium) from the end portions which had been excised. The stem cells are washed and suspended again in DH medium supplemented with 20% foetal calf serum (FCS) The cell concentration is adjusted to 500000 cells/ml.

Products previously dissolved in DH medium supplemented with 20% FCS, amino acids and antibiotics are serially diluted directly into a microtiter plate. The products are tested in triplicate and each microtiter plate includes a negative control containing plain medium. The final volume in each well is 100 μl.

100 μl of the cell suspension are added to diluted solutions of products and the cells are incubated for 7 days in an incubator at 37° C., under 8% CO₂ and a moisture saturated atmosphere. Cell proliferation is determined by measuring the oxidation of a chromogenic substrate XTT (2, 3-bis[2-methoxy-4-nitro-5-sulphophenyl]-2H-tetrazolium-5-carboxanilide) in mitochondria of viable cells.

At the end of the incubation period, 50 μl of the XTT substrate solution (1 mg/ml XTT+0.008 mg/ml phenazine methosulfate) are added to each well. After an 8 hour incubation period at 37° C. under 8% CO₂ in a moisture saturated incubator, the microtiter plates are read with a spectrophotometer at 492 nm against a reference sample at 690 nm.

Results are expressed as mean value±standard deviation and plotted as a dose versus response curve. Values for a negative control composed of DH medium (mean±standard deviation) are also graphically shown.

From this dose versus response curve, 3 curve variables are computed:
Max: maximum curve amplitutde and corresponding concentration
EC₅₀: concentration corresponding to 50% of maximum amplitude
Min : lowest concentration inducing a significant proliferation corresponding to the blank±3× standard deviation value

Concentration values corresponding to EC₅₀ and Min are determined from line segments joining differents points on the curve.

If the curve has no plateau phase but an ascending configuration for the highest concentration being tested, a “>” sign is shown before Max and EC₅₀ values.

If the curve does not drop below the blank±3× standard deviation value, minimum concentration value is shown as “ ” lowest concentration being tested.

4.2.2. Results

Proliferation of Bone Marow Stem Cells Induced by acyl-dipeptide-like Compounds Bearing an Accessory Functional Side Chain Spacer

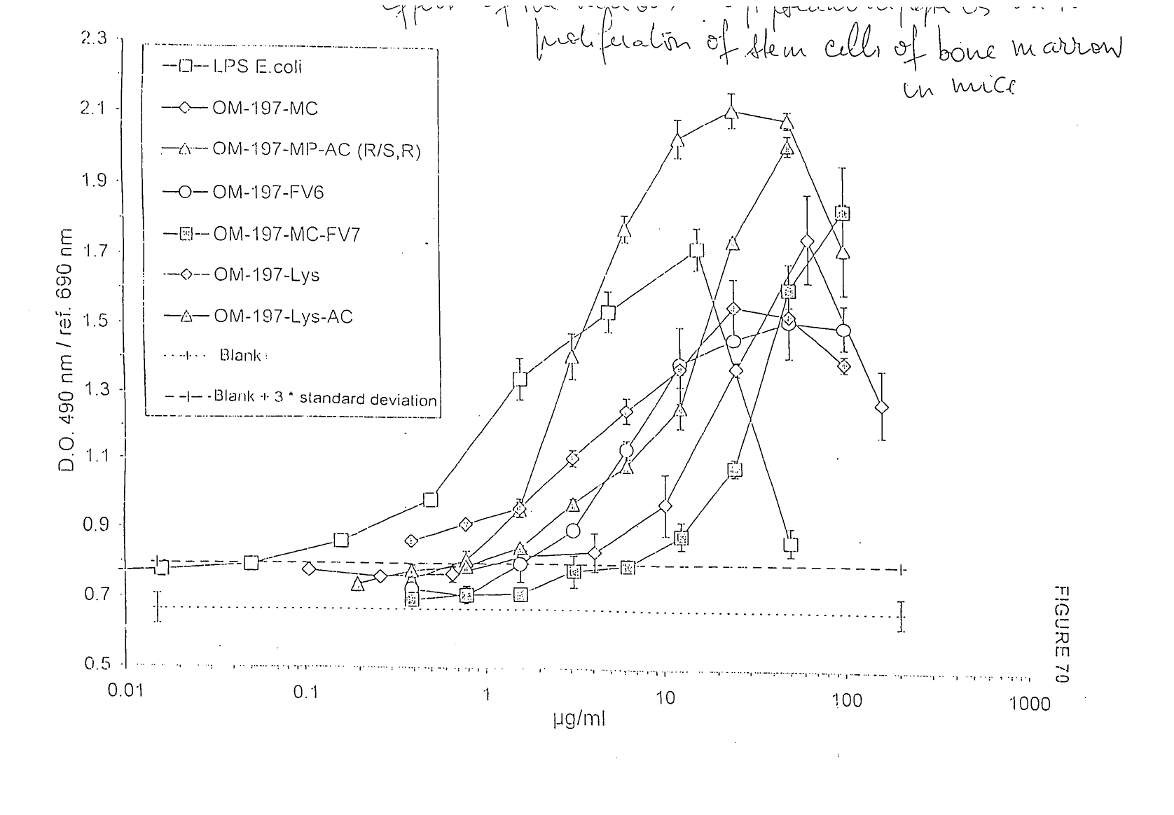

FIG. 70 displays the activity of different acyl dipeptide-like compounds and clearly shows the influence of accessory functional side chain spacer on the ability to induce bone marrow stem cell proliferation in mice.

Certain acyl- dipeptide-like compounds are able to induce profilferation to a greater extent than positive control, made up of E. coli LPS. Minimal product concentration capable of inducing a significant proliferation is higher than concentration used in positive control. This minimal product concentration is greatly affected by the particular accessory functional side chain spacer being used.

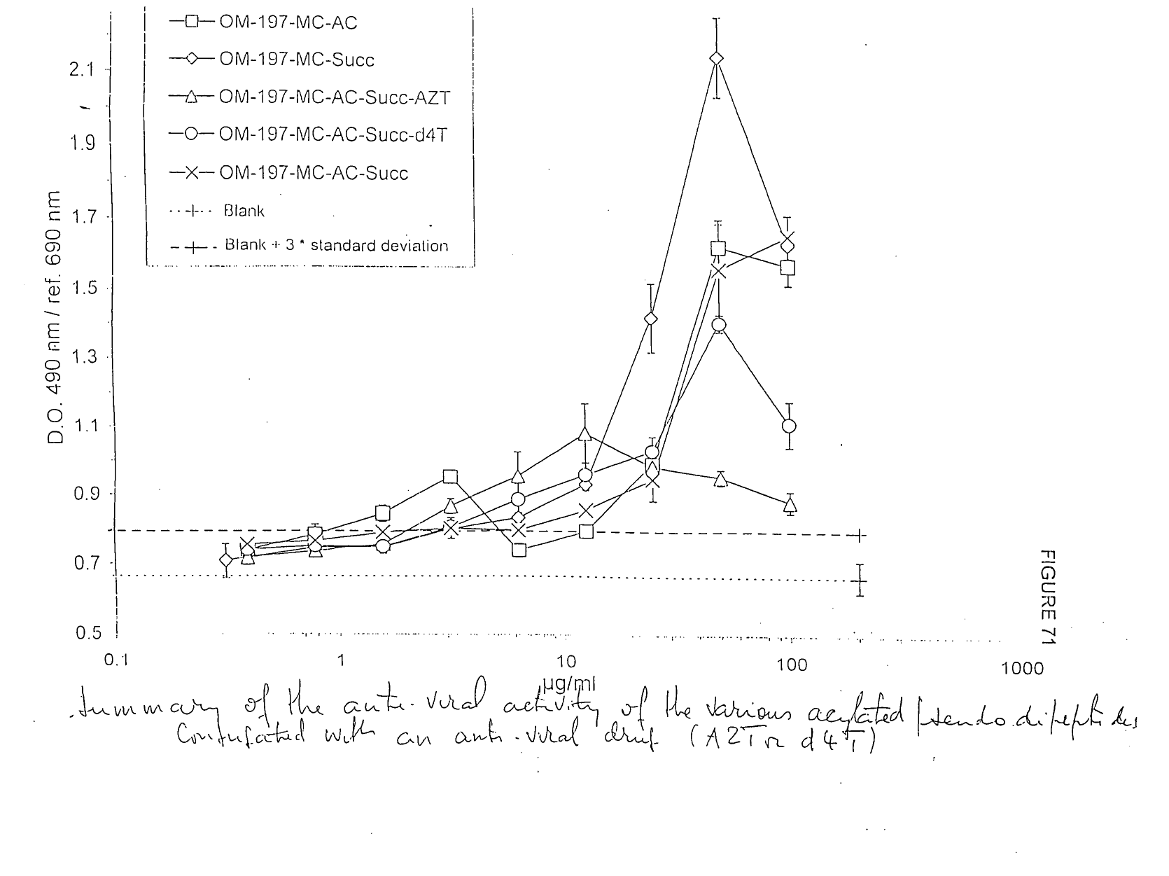

FIG. 71 shows the activity of acyl dipeptide-like compounds coupled to a given drug. This activity provides evidence that compounds remain partially active inspite of the coupling process. It has not been possible to discriminate between intrinsic activity and activity resulting from coupling of the ester bond by intracellular esterases. Antiviral activity of these products strongly suggests that ester bonds undergo at least partial cleavage. Whatever the underlying mechanism, retention of biological activity underscores the full potential of combining antiviral activity and product activity.

Bone marrow stem cell proliferation induction results by various acyl-
dipeptide-like compounds bearing an accessory functional side chain
spacer having the general formula I (table listing)

| | Max | EC₅₀ | Min. |
|---|---|---|---|
| | amplitude/ | amplitude/ | amplitude/ |
| | conc. | conc. | conc. |
| Products | O.D./μg/ml | O.D./μg/ml | O.D./μg/ml |
| DH Medium (negative | 0.67 ± 0.04 | | |
| control) | | | |
| E. coli LPS (positive control) | 1.75/16 | 1.19/1.2 | 0.80/0.06 |
| OM-197-MC | 1.75/64 | 1.21/19.4 | 0.80/1.25 |
| OM-197-MC-MP | >1.60/>160 | >1.13/>6.5 | 0.80/0.77 |
| OM-197-FV6 | 1.52/50 | 1.10/5.7 | 0.80/1.60 |
| OM-197-FV8 | 1.07/25 | 0.87/1.8 | 0.80/1.24 |
| OM-197-MC-FV5 | 1.75/50 | 1.21/32.0 | 0.80/8.20 |
| OM-197-MC-FV7 | >1.83/>100 | >1.25/>33.1 | 0.80/6.58 |
| OM-197-MP-AC(R/S, R) | 2.12/25 | 1.39/3.1 | 0.80/0.71 |
| OM-197-MP-AC (S, R) | 1.22/6.25 | 0.94/2.62 | 0.80/1.17 |
| OM-197-MC-AC | 1.62/50 | 1.15/31.0 | 0.80/0.92 |
| OM-197-N′C2-MC-AC | 0.75/1.56 | 0.71/nd | 0.80/nd |
| OM-197-N′(C10-20C8)-MC- | 0.75/6.25 | 0.71/nd | 0.80/nd |
| AC | | | |
| OM-197-MC-Succ | 2.16/50 | 1.41/24.7 | 0.80/2.95 |
| OM-197-Lys | 1.56/25 | 1.11/3.3 | <0.86/<0.39 |
| OM-197-AP | 1.49/25 | 1.08/2.2 | 0.80/0.41 |
| OM-197-Asp | 1.22/25 | 0.94/5.0 | 0.80/1.89 |
| OM-197-Asp-AC | 1.33/50 | 0.10/2.8 | 0.80/1.27 |
| OM-197-Lys-AC | >2.02/>50 | >1.34/>14.6 | 0.80/0.90 |
| OM-197-MC-AC-Succ-AZT | 1.08/12.5 | 0.87/3.3 | 0.80/2.11 |
| OM-197-MC-AC-Succ-d4T | 1.40/50 | 1.03/25.4 | 0.80/2.81 |
| OM-197-MC-AC-Succ | >1.65/>100 | >1.16/33.7 | 0.80/2.04 |

nd = undetermined

All acyl-dipeptide-like compounds of the invention except compounds with short chains are capable of inducing significant bone marrow stem cell proliferation.

### Example 4.3.

### Determining the Production of Nitric Oxide by Murine Macrophage Cells Challenged with Products of the Invention

4.3.1. Experimental Assay of Nitric Oxide Production

Six-week old male C57/BL6 mice are killed by CO₂ inhalation. The hip, femur, tibia and posterior appendage bones are removed. The bone marrrow is extracted from the bone lumen by injecting Dulbecco's Modified Eagle Medium (DH medium) from the. end portions which had been excised. The stem cells are washed and resuspended (approximate cell concentration 4000 cells/ml) in DH medium supplemented with 20% equine serum (SH) and 30% L929 cell supernatant. L929 is a murine fibroblast cell line the supernatant fluid of which is rich in growth factor for macrophage cells (M-CSF). The cell suspension is divided into Petri dishes which are incubated for 8 days in an incubator at 37° C. under 8% CO₂ and a moisture saturated atmosphere.

After 8 days, the stem cells have differenciated into mature macrophage cells. The macrophage cells are scraped off by rapid cooling, washed and resuspended in DH medium supplemented with 5% foetal calf serum (FCS), amino acids and antibiotics. The cell density is adjusted to 700 000 cells/mi.

Products previously dissolved in DH medium supplemented with 5% FCS, amino acids and antibiotics are serially diluted directly in microtiter plates. The products are tested in triplicates and each microtiter plate comprises a negative control containing plain medium. The final volume in each well is 100 μl.

100 μl of cell suspension are added to diluted solutions of such products and the cells are incubated for 22 hours in an incubator at 37° C., under 8% CO₂ and a moisture saturated atmosphere. At the end of the incubation period with the products, 100 μl of supernatant are withdrawn and the nitrite concentration is determined by running a Griess reaction.

100 μl of Griess reagent (5 mg/ml of sulfanilamide ±0.5 mg/ml of N-(1-naphtylethylene diamine hydrochloride)) in 2.5% aq. phosphoric acid, are added to each well. The microtiter plates are read with a spectrophotometer at 562 nm wavelength against a reference sample at 690 nm. The nitrite concentration is proportional to nitric oxide content being formed. The nitrite content is determined based on a standard curve.

The results are given as mean value±standard deviation and plotted as a dose versus response curve.

From this dose versus response curve, 3 curve variables are computed:
Max: maximum curve amplitutde and corresponding concentration
EC₅₀: concentration corresponding to 50% of maximum amplitude
Min: lowest concentration inducing a significant proliferation corresponding to the blank±3× standard deviation value

Concentration values corresponding to EC₅₀ and Min are determined from line segments joining differents points on the curve.

If the curve has no plateau phase but an ascending configuration for the highest concentration being tested, a “>” sign is shown before “Max ” and “EC₅₀” values.

If the curve does not drop below the blank±3× standard deviation, minimum concentration value is shown as “<” lowest concentration being tested.

4.3.2. Results

Nitric Oxide Production by Murine Macrophage Cells induced by Acyl Dipeptide-like Compounds bearing an Accessory Functional Side Chain Spacer

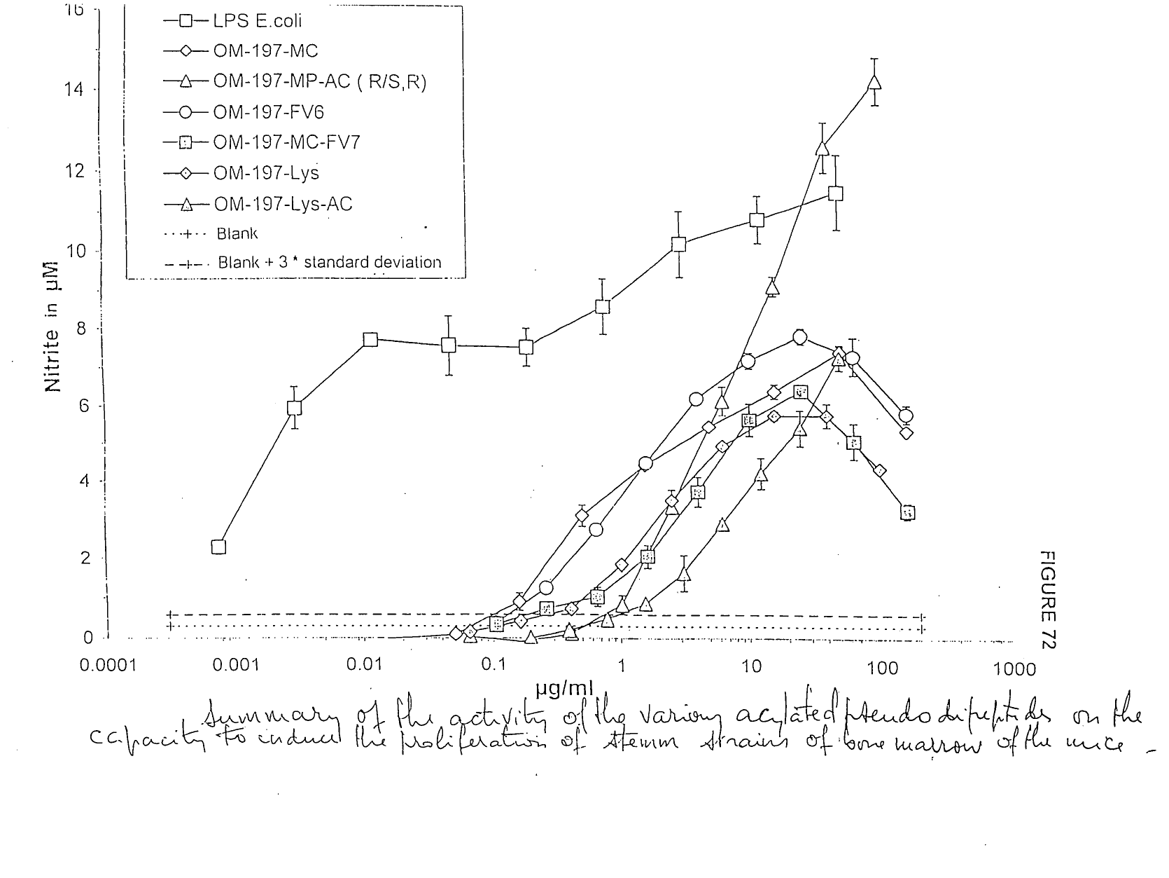

FIG. 72 shows the activity of different acyl- dipeptide-like compounds and demonstrates the influence of the accessory functional side chain spacer on the ability to induce murine bone marrow stem cell proliferation.

OM-197-MP-AC (R/S, R) is capable of inducing a higher proliferation response as compared to positive control made up of E. coli LPS. Minimal product concentration capable of inducing a significant proliferation is nonetheless far lower than positive control concentration. NO production by murine macrophage cells challenged by acyl dipeptide-like compounds is greatly affected by the particular accessory functional side chain spacer being used.

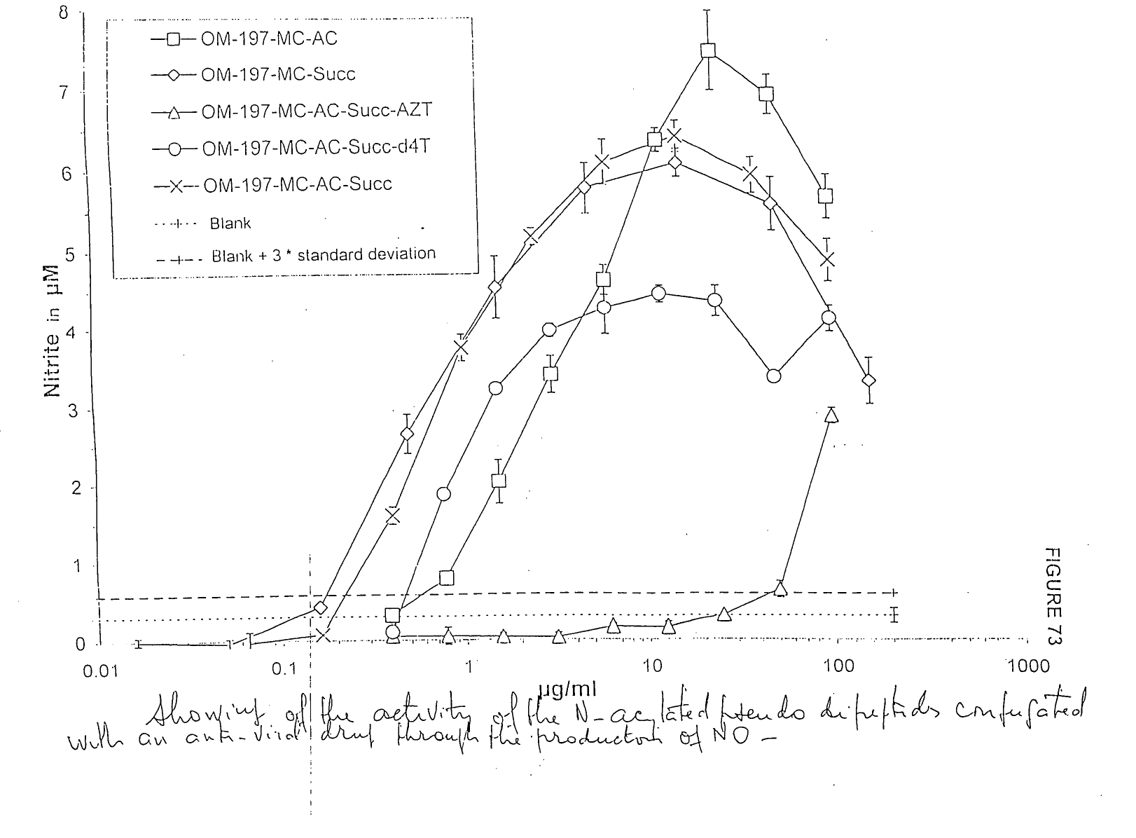

FIG. 73 shows the activity of acyl dipeptide-like compounds coupled to a given drug. This activity is regarded as evidence that compounds remain partially active inspite of the coupling step. It has not been possible to discriminate between intrinsic activity and activity resulting from coupling of the ester bond by intracellular esterases. Antiviral activity of these products strongly suggests that ester bonds undergo at least partial cleavage. Whatever the underlying mechanism, retention of biological activity demonstrates the full potential of combining antiviral activity and product activity.

Nitric oxide induced production results in murine macrophage cells by various acyl-
dipeptide-like compounds bearing an accessory functional side chain spacer
having the general formula I (table listing)

| | Max amplitude/ | EC₅₀ amplitude/ | Min. amplitude/ |
|---|---|---|---|
| | conc. | conc. | Conc. |
| | μM nitrite/ | μM nitrite/ | μM nitrite/ |
| Products | μg/ml) | (μg/ml) | (μg/ml) |
| Medium DH (negative control) | 0.29 ± 0.09 | | |
| E. coli LPS (positive control) | >10.37/>50 | >5.33/>0.01 | <1.41/<0.001 |
| OM-197-MC | 7.16/51 | 3.73/1.23 | 0.57/0.15 |
| OM-197-MC-MP | 7.94/51 | 4.12/1.18 | 0.57/0.11 |
| OM-197-FV6 | 7.48/25.6 | 3.89/1.49 | 0.57/0.20 |
| OM-197-FV8 | 8.00/51 | 4.15/7.89 | 0.57/1.45 |
| OM-197-MC-FV5 | 5.64/16 | 2.97/2.56 | 0.57/0.67 |
| OM-197-MC-FV7 | 6.17/>25.6 | 3.23/>3.70 | 0.57/0.36 |
| OM-197-MP-AC(R/S, R) | >14.09/>100 | >7.19/>10.69 | 0.57/1.04 |
| OM-197-MP-AC (S, R) | 7.56/40 | 3.93/3.28 | 0.57/0.59 |
| OM-197-MP-AC (R, R) | >8.31/>100 | >4.3/>15.32 | 0.57/3.35 |
| OM-197-MC-AC | 7.08/25 | 3.69/4.91 | 0.57/0.91 |
| OM-197-N′C2-MC-AC | >0.24/200 | nd | nd |
| OM-197-N′(C10-20C8)-MC- | >0.96/>100 | 0.63/61.05 | 0.57/54.3 |
| AC | | | |
| OM-197-MC-Succ | 5.84/16 | 3.07/0.89 | 0.57/0.22 |
| OM-197-Lys | 5.50/40 | 2.90/2.23 | 0.57/<0.47 |
| OM-197-AP | >9.64/>100 | >4.97/>13.69 | 0.57/1.87 |
| OM-197-Asp | 8.26/51 | 4.28/0.95 | 0.57/0.086 |
| OM-197-Asp-AC | >7.97/>100 | >4.13/>7.04 | 0.57/2.00 |
| OM-197-Lys-AC | >7.04/>50 | >3.67/>11.06 | 0.57/1.46 |
| OM-197-MC-AC-Succ-AZT | >2.53/>100 | >1.41/>74.89 | 0.57/55.98 |
| OM-197-MC-AC-Succ-d4T | 4.17/12.5 | 2.23/1.15 | 0.57/0.56 |
| OM-197-MC-AC-Succ | 6.17/>16 | 3.23/0.72 | 0.57/0.28 |

nd = undetermined

All acyl-dipeptide-like compounds of the invention except compounds with short chains (minor production for OM-1 97-N′(C10-20C8)-MC-AC) are capable of inducing a significant nitric oxide production by murine macrophage cells.

### Example 4.4.

### Differenciation Test of Dendritic Cells

The ability of products of the invention to induce maturation of predendritic cells into dendritic cells was assessed. The following parameters were measured: FITC-Dextran conjugate take-up and expression of CD83, CD86 surface markers.

4.4.1. Experimental Procedure

Mononucleated cells of peripheral blood are isolated from buffy coats of healthy donors. Purified monocytes by adherence selection are resuspended in RPMI-1640 medium containing 10% of foetal calf serum (FCS), GM-CSF and IL-4 (10 ng/ml) at a density of 1×10⁶ cells/ml. Cells are divided into Petri Dishes (10×10⁶ cells per dish) and cultured for 6 days with a change to fresh medium after 3 days. Cells thus obtained are called predendritic cells (DC-6). Maturation of predendritic cells into mature dendritic cells is achieved by incubating cells with diluted solutions of the products or LPS (positive control) for 3 more days (see product section below). At day 9 (DC-9), cells are harvested and analyzed for different indicators of dendritic cell maturation : assessment of CD83, CD86 surface marker expression as well as of their ability to take up FITC-Dextran conjugate. All these parameters are analyzed by an EPICS-XL-MCL model FACS (Coulter Immunology, Hialeah, Finland).

Expression of surface markers is given as % of mean fluorescence of LPS-activated cells (positive control); FITC-Dextran conjugate take-up rate is calculated based on take-up of cells maintained in basic medium and is expressed in %.

Products:

Stock solutions of OM-197-MC, OM-197-FV6 and OM-197-MP-AC (R/S, R) are prepared at a concentration of 0.5 mg/ml in 0.9% NaCl/water, with addition of 0.1% triethanolamine. Solutions are incubated at 37° C. for 20 minutes, subjected to vigorous stirring during 3 minutes and then diluted to 100 μg/ml in RPMI-1640 culture medium and used in diluted state at concentrations ranging from 10 μg/ml to 0.03 μg/ml.

Reference Product:

E. coli lipopolysaccharide (LPS, DIFCO, Detroit, Mich., USA), as a 5 mg/ml stock solution in PBS. An intermediate 100 μg/ml solution is prepared in RPMI 1640 culture medium. Concentrations of dilute solutions being tested range from 1 μg/ml to 0.03 μg/ml.

In another series of experiments, all acyl dipeptide-like compounds were freeze-dried and directly redissolved into pyrogen-free water. Different products as well as LPS control are tested at a concentration of 10 μg/ml. Results are given as % of dendritic cells and mean fluorescence value for dextran phagocytosis or CD86 surface marker expression. These results stand for the mean value of 2 to 6 experiments depending on the product being used.

4.4.2. Result Analysis

Immature dendritic cells (DC-6) resulting from monocyte differenciation, through the joint action of GM-CSF and IL-4, are able to incorporate FITC-Dextran conjugate. During the maturation process, cells lose their ability to incorporate the FITC-Dextran conjugate. Assays are conducted upon reaching the DC-9 differenciation stage.

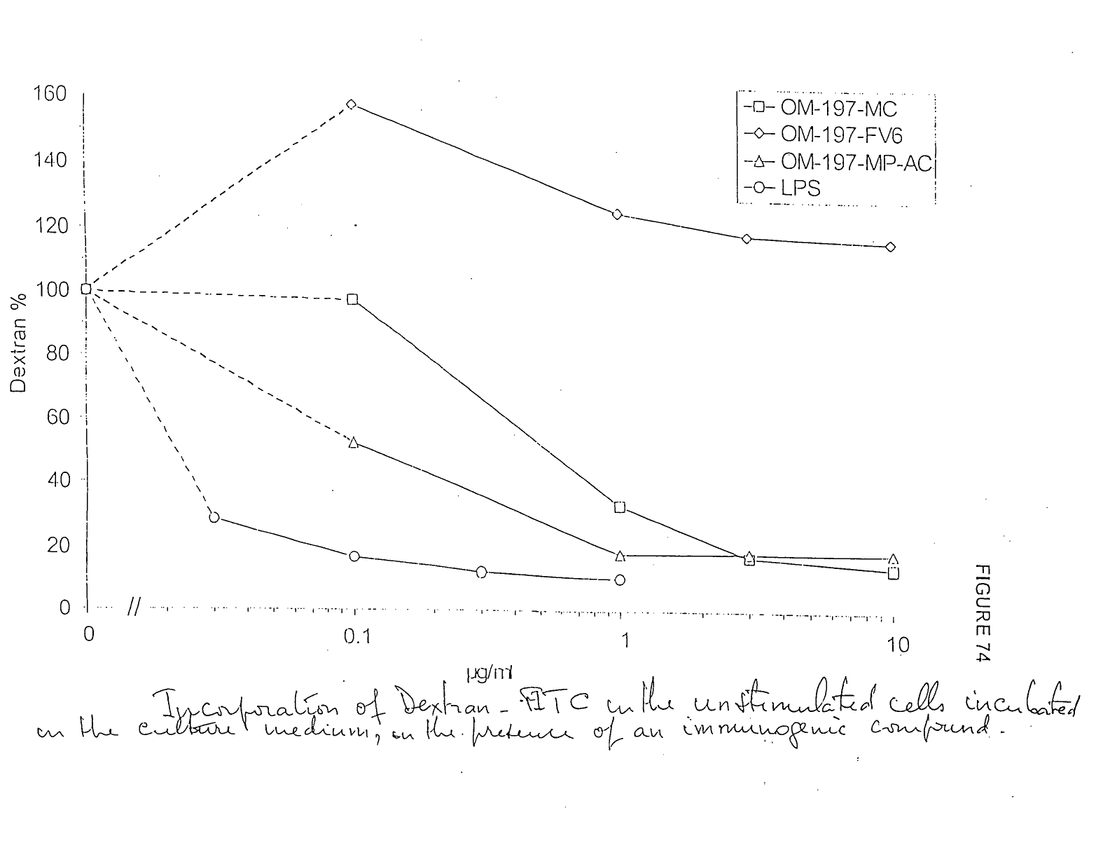

Results (FIG. 74) are expressed in terms of % incorporation of FITC-Dextran conjugate observed in unchallenged cells incubated in basic medium. Cells treated with LPS or OM-197-AC (R/S, R) retain only 15% and 22% of their phagocytic capacity, respectively. It should be noted that at least 1 μg/ml of OM-197-MP-AC (R/S, R) is required to induce full decrease of Dextran take-up whereas 0.1 μg/ml of LPS results in a similar decrease. 10 μg/ml of OM-197-MC are required to induce a 22% decrease of phagocytic capacity. Furthermore, for concentrations lower than 3 μg/ml, OM-197-MC has no effect on phagocytosis. OM-197-FV6 has apparently no effect on maturation of dendritic cells.

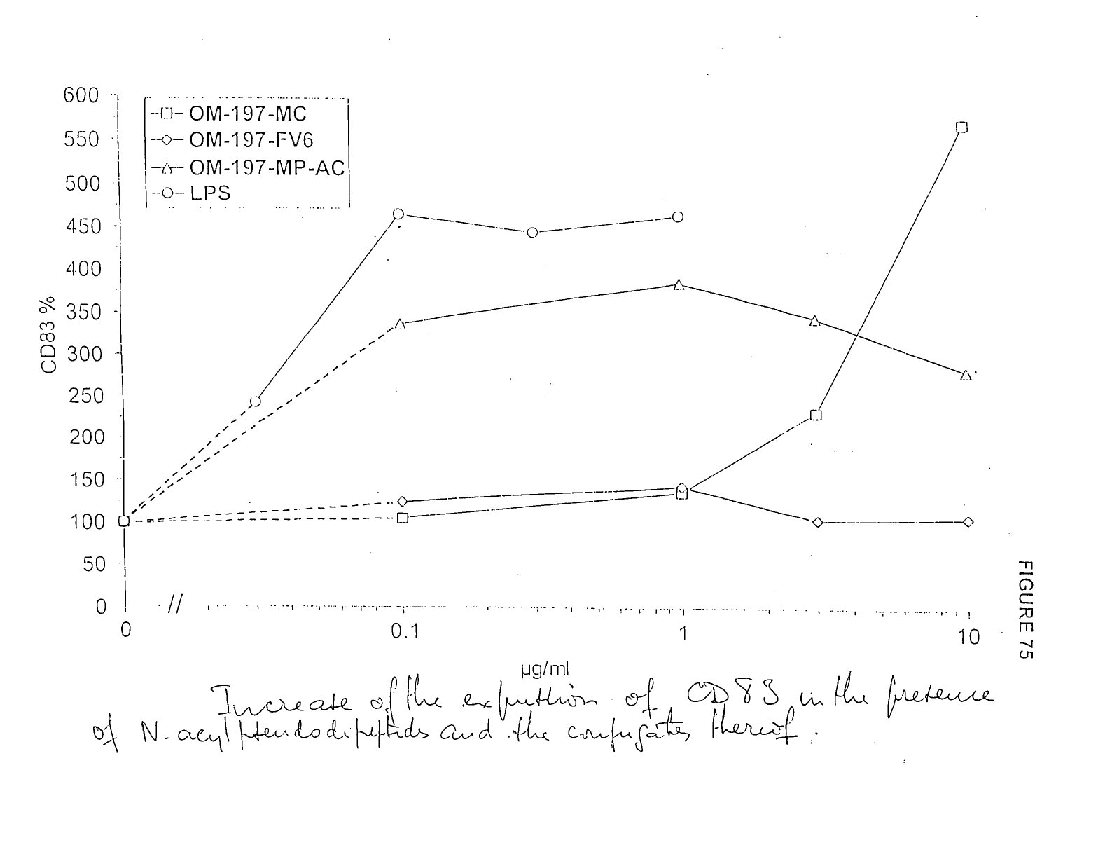

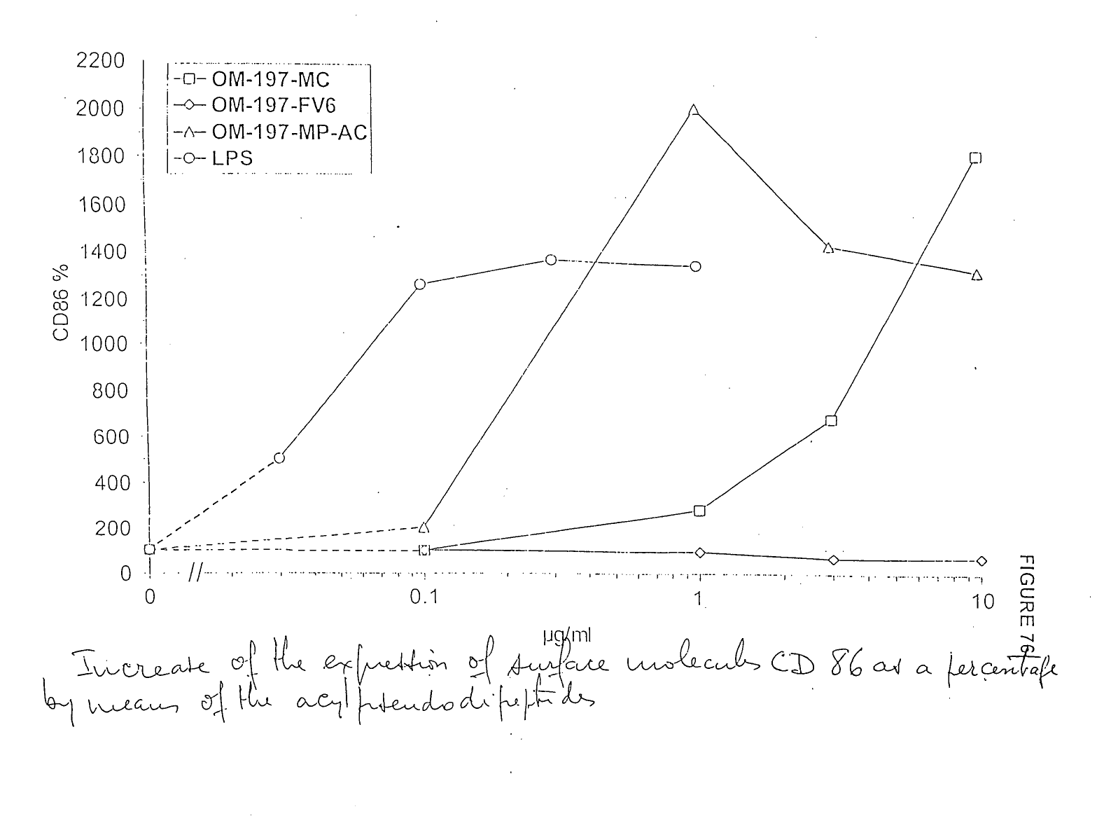

Expression of co-stimulation surface markers is another criterion to assess DC maturation. Rise in expression of CD83, CD86 (FIGS. 75 & 76, respectively) is tested. Results are expressed in terms of % of mean fluorescence based on LPS-induced expression of these markers. These results are fully consistent with those obtained from phagocytosis studies. OM-197-MP-AC (R/S, R) induces an increase in expression of surface markers at concentrations as low as 0.1 μg/ml, while at least 1 μg/ml is needed in case of OM-197-MC to initiate a rise in expression of CD83 and CD86 markers. OM-197-FV6 has no effect at all on expression of surface markers which are typical of dendritic cells.

The aforegoing data show that acyl- dipeptide-like compounds act very differently depending on the selected accessory side chain spacer. OM-197-MP-AC (R/S, R) displays an outstanding ability to induce differenciation of DC-6 cells into DC-9 cells in concentrations as low as 0.1 μg/ml, whereas concentrations greater than 1 μg/ml are required for OM-197-MC to initiate differenciation, and OM-197-FV6 does totally lack such an activity.

Fluorescein-labelled dextran phagocytosis and CD86 expression
after predendritic cell (CD6) stimulation by acyl dipeptide-like
compounds bearing an accessory functional side chain spacer
having the general formula I
Differentiation of predendritic cells into mature dendritic cells

| | Dextran | CD86 |
|---|---|---|
| Products | % CD/Fluorescence | % CD/Fluorescence |
| 37° C. | 18.3/0.9 | 33.5/2.9 |
| LPS E. coli | 100/7.3 | 100/7.3 |
| OM-197-FV6 | 84.5/6.3 | 62.9/4.9 |
| OM-197-MC-FV5 | 86.5/6.4 | 74.5/5.6 |
| OM-197-MP-AC (R/S, R) | 84.6/6.3 | 80.0/6.0 |
| OM-197-MC-AC | 68.3/5.2 | 67.4/5.2 |
| OM-197-MC-MC-Succ | 35.5/3.0 | 34.9/3.0 |
| OM-197-Lys | 12.1/0.5 | 21.2/2.1 |
| OM-197-AP | 56.1/4.4 | 62.0/4.8 |
| OM-197-Asp | 26.4/2.4 | 29.0/2.6 |
| OM-197-Asp-AC | 22.0/22.1 | 26.7/2.4 |
| OM-197-Lys-AC | 92.2/6.8 | 93.5/6.9 |
| OM-197-MC-AC-Succ-AZT | 62.2/4.8 | 81.4/6.1 |
| OM-197-MC-AC-Succ-d4T | 11.6/0.5 | 25.9/2.4 |

At a concentration of 10 μg/ml, a great number of acyl dipeptide-like compounds are able to induce differenciation of predendritic cells into dendritic cells. The functional group born by the accessory side chain spacer regulates this ability to activate predendritic cells.

Acyl-dipeptide-like compound OM-197-MC-AC-Succ-AZT coupled to a drug has a significant ability to induce differenciation into dendritic cells. The way acyl-dipeptide-like compounds are formulated has a determining effect on their ability to induce predendritic cell differentiation into dendritic cells. The activity of certain products varies to a great extent depending on whether said products are dissolved in presence or absence of 0.1% TEOA.

### Example 4.5.

### Analysis of MxA Protein Induction in Dendritic Cells

The ability of products of the invention to induce production of antiviral MxA protein by predendritic cells has been assessed. MxA production by cells was measured after an SDS-PAGE electrophoretic separation by a Western Blot assay

4.5.1. Experimental Procedure

DC-6 Staged dendritic cells obtained as described above were either activated or not with α IFN (500 U/ml), LPS (1 μg/ml), TRANCE (100 ng/ml), CD40L (1 μg/ml), OM-197-MP-AC (1 μg/ml) or OM-197-FV6 (1 μg/ml) for 48 hours. Extraction and analysis of protein was conducted as follows: cells were harvested, then washed 3 times with PBS (phosphate buffered saline) and lyzed with a 100 Mm Tris-HCl buffer, 150 mM NaCl, 5 mM EDTA, 1% Triton-100 containing 1 mM PMSF (phenylmethylsulphonyl fluoride) and 1 μ/ml of pepstatin as protease inhibitors. Lysis was carried out at a density of 10⁶ cells for 100 μl of (5×) buffer containing 60 mM Tris-HCl (pH 6.8), 10% glycerol, 2% SDS, 14.4 mM 2-mercaptoethanol and 0.1% bromophenol). Samples were heated up to 95° C. for 5 min. in sealed Eppendorf tubes. Electrophoresis was performed on a 10 cm×10 cm 12% acrylamide minigel by running a sample having the same number of cells. Migration was effected with the following buffer: 25 mM Tris, 192 mM glycine, 0.1% SDS, pH 8.3.

Blotting : Protein are blotted from an SDS-PAGE gel to a nitrocellulose membrane (blotting buffer: 25 mM Tris, 192 mM glycine, methanol pH 8.3)

Incubation with antibodies : Blotting was demonstrated by dyeing the membrane with Ponceau Red. Non specific bonding sites on the membrane were blocked with 5% skimmed milk in TBS (10 mM Tris-HCl, 150 Mm NaCl, pH 7.4) and allowing to stand for 1 hour at room temperature. The membrane was washed 3 times with TBS-0.1% Tween 20. The membrane was incubated with an anti-MxA monoclonal antibody solution (clone 143) previously diluted 100-fold (vol./vol.) in TBS-milk. The membrane was incubated with a second peroxidase coupled anti-mouse antibody (HRP, Sigma) previously diluted 1000-fold (vol./vol.). The membrane was washed three times with TBS-Tween. Bands containing MxA were revealed by incubation of the membrane with chemiluminescent reagents (ECL, Amersham Pharmacia). In this system, HRP catalyzes a luminol-mediated chemiluminescent reaction which can be detected on a photographic film.

4.5.2. Results

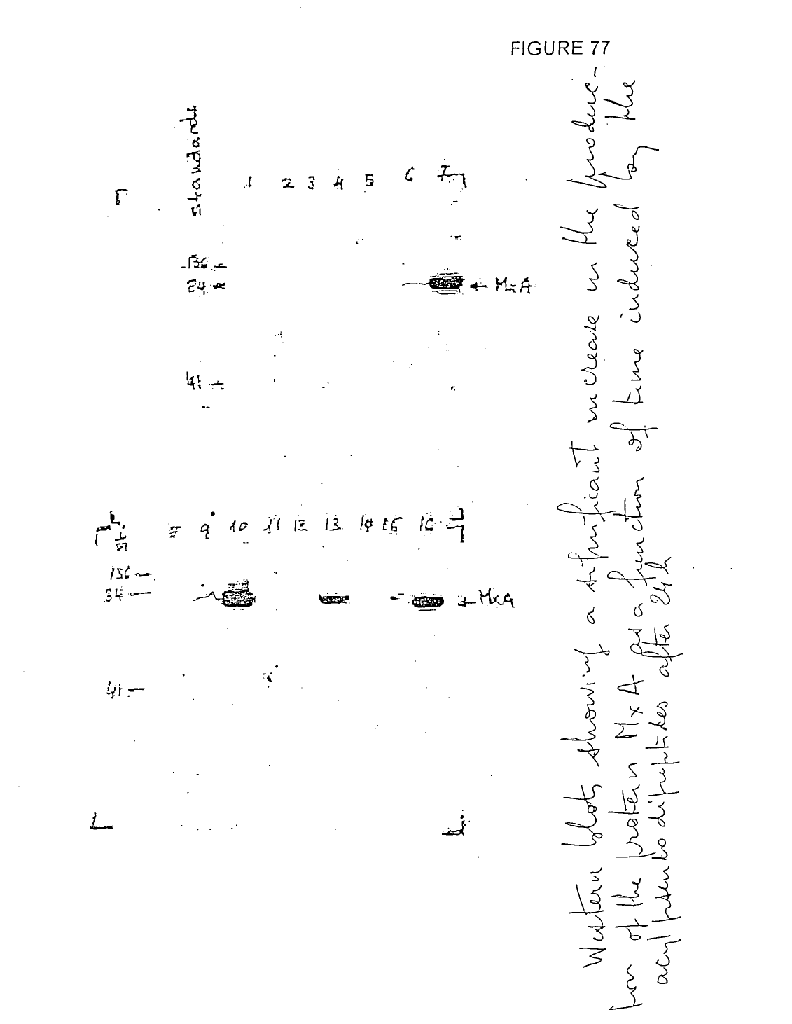

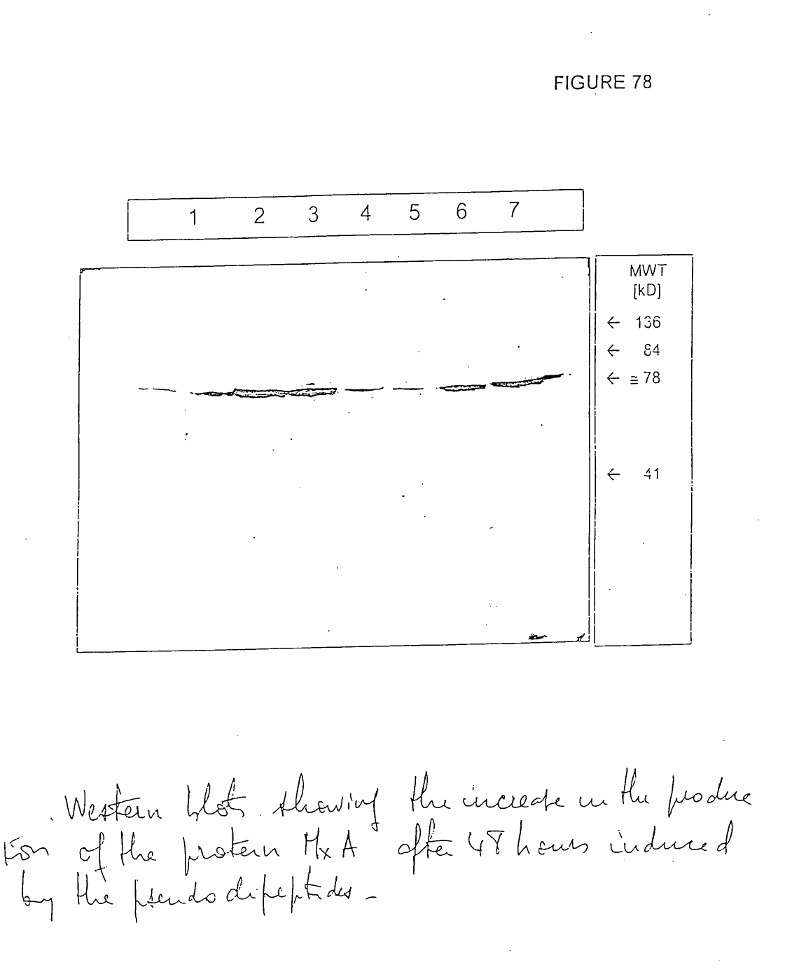

DC-9 cells were activated for 0, 2 hr, 4 hr, 24 hr (FIG. 77) or 48 hr. (FIG. 78) as follows:

| | | | | | | | location |
|---|---|---|---|---|---|---|---|
| | | | | | | | on |
| Sam- | Assay: culture medium | FIG. 77 | | | | | Western |
| ple | GMCSF/IL-4 | 0 h | 2 h | 4 h | 24 h | 48 h | Blot |
| 1. | +NaCl (negative control) | 1 | 2 | 3 | 4 | 1 | ⇓ |
| 2. | +LPS (1 μg/ml) | | 5 | 6 | 7 | 2 | ⇓ |
| 3. | +IFN-α (500 U/ml) | | 8 | 9 | 10 | 3 | ⇓ |
| | (positive control) for | | | | | | |
| | MxA proteine induction | | | | | | |
| 4 | +TRANCE (1 ng/ml) | | | | | 4 | ⇓ |
| 5 | +CD40 (1 μg/ml) | | | | | 5 | ⇓ |
| 6 | +OM-197-MP-AC | | 11 | 12 | 13 | 6 | ⇓ |
| | (1 μg/ml) | | | | | | |
| 7. | +OM-197-FV6 (1 μg/ml) | | 14 | 15 | 16 | 7 | ⇓ |

Western Blots shown on FIGS. 77 and 78 illustrate the time-course production of MxA: induction of a significant rise of MxA protein by IL-197-AC5 and OM-197-FV6 products after 24 hr. (FIG. 77) and after 48 hours, respectively (FIG. 78). MxA production noted herein is comparable to the one induced by LPS positive control or a IFN with production level being higher than the level induced by negative control, by TRANCE (a cytokine belonging to the.family of α TNF which induces maturation of dendritic cells) or by CD40L (another agent promoting maturation of dendritic cells), respectively.

Compounds according to the invention OM-197-MP-AC and OM-197-FV6 show antiviral properties by virtue of their capacity to induce MxA-protein production

### Example 4.6.

### Determining the Ability of Compounds according to the Invention to Elicit α TNF Production by Human Peripheral Blood Mononuclear Cells

4.6.1. Procedure

Mononuclear cells of peripheral blood are recovered from buffy coats of 6 healthy donors. Such buffy coat cells are resuspended and mononuclear cells are isolated by centrifugation on a Ficoll-Paque gradient (Amersham Pharmacia). Purified mononuclear cells are resuspended into RPMI -1640 medium supplemented with 10% FCS, antibiotics, mercaptoethanol and L-glutamine. Cell concentration is adjusted to 2×10⁶ viable cells/ml

100 μl of each one of these solutions of acyl dipeptide-like compounds bearing an accessory functional side chain spacer, diluted 100-, 10- or 1-fold in RPMI medium 1640, are dispensed into a microtiter plate. Samples are tested in triplicates, and each microtiter plate includes a negative control made up of plain medium,. Dilutions of E. coli LPS (ranging grom 1 to 0.00001 μg/ml) are used as a positive control.

100 μl of the cell suspension are added to each well containing product dilute solutions or control. Cells are incubated with the products for 18 hours in an incubator at 37° C., under 5% CO₂ and a moisture saturated atmosphere.

At the end of the incubation period, microtiter plates are centrifuged, and the supernatant fluids are pooled and frozen at −80° C. as aliquots until time of ELISA assay.

Concentration of α TNF secreted by mononuclear cells is determined by chemiluminescent ELISA (QuantiGlo #QTAOO R&D kit). 4-fold supernatant fluid dilutions are dispensed into each well of the microtiter plate to which anti-αTNF antibodies are bound. The microtiter plate is incubated for 4 hours at room temperature. After washing, peroxidase-conjugated anti-human α TNF antibody is added. Following a 2 hour incubation period and thorough washing, a chemiluminescent substrate is added and chemilunescence is read after 20 minutes. α TNF content in the supernatant fluid is determined with respect to a standard curve covering the 7000 to 0.7 μg/ml range.

Results are expressed in μg/ml of α TNF produced. Results derive from an experiment representative of acyl-dipeptide-like compound activity

4.6.2. Results

Induction of α TNF production by acyl dipeptide-like compounds bearing an accessory functional side chain spacer having the general formula I in peripheral blood mononuclear cells

| Product | 100 μg/ml | 10 μg/ml | 1 μg/ml |
|---|---|---|---|
| OM-197-MC | 12 | 0 | 0 |
| OM-197-MC-MP | 72 | 0 | 0 |
| OM-197-FV6 | 88 | 104 | 0 |
| OM-197-FV8 | 252 | 100 | 12 |
| OM-197-MC-FV5 | 116 | 104 | 0 |
| OM-197-MC-FV7 | 76 | 80 | 0 |
| OM-197-MP-AC(R/S, R) | 160 | 156 | 244 |
| OM-197-MP-AC (R, R) | 84 | 88 | 0 |
| OM-197-MC-AC (S, R) | 80 | 124 | 488 |
| OM-197-MC-AC | 144 | 140 | 12 |
| OM-197-N′C2-MC-AC | 0 | 0 | 0 |
| OM-197-N′(C10-20C8)-MC-AC | 0 | 0 | 0 |
| OM-197-MC-Succ | 176 | 96 | 0 |
| OM-197-Lys | 20 | 164 | 284 |
| OM-197-AP | 84 | 116 | 428 |
| OM-197-Asp | 80 | 28 | 0 |
| OM-197-Asp-AC | 132 | 172 | 20 |
| OM-197-Lys-AC | 256 | 184 | 232 |
| OM-197-MC-AC-Succ-AZT | 64 | 224 | 108 |
| OM-197-MC-AC-Succ-d4T | 52 | 40 | 0 |
| OM-197-MC-AC-Succ | 40 | 32 | 0 |

Negative control comprised of RPMI medium induced a baseline α TNF production below test sensitiviy level of 0.7 mg/ml.

Induction of TNF production by E. coli. LPS in peripheral blood mononuclear cells.

| | E. coli LPS in μg/ml | | | | | |
|---|---|---|---|---|---|---|
| Product | 1 | 0.1 | 0.01 | 0.001 | 0.0001 | 0.00001 |
| TNF-α | 404 | 436 | 356 | 276 | 132 | 0 |
| (pg/ml) | | | | | | |

Positive control made up of E. coli LPS elicits high production of αTNF even for the lowest concentrations tested.

Most acyl- dipeptide-like compounds induce substantial α TNF-production even though production level is less than what is seen for the positive control. Greater concentrations are required with respect to LPS to induce a signficant production of α TNF.

Certain compounds like OM-197-MC and OM-197-MC-MP induce a significant production level only at a concentration of 100 μg/ml.

Amine functional group is of particular interest for inducing αTNF production as seen on one hand, by increasing amplitude and decreasing minimal product concentration required to induce a significant production on the other.

Short chain acyl-dipeptide-like compounds OM-197-N′C2-MC-AC and OM-197-N′(C10-20C8)-MC-AC do not induce any αTNF production even at a concentration of 100 μg/ml.

### EXAMPLE 4.7.

### Determining the Capacity of Compounds in Accordance with the Invention to Inhibit α TNF Production in Human Peripheral Blood Mononuclear Cells in Response to E. coli Lipopolysaccharide (LPS)

4.7.1. Procedure

Mononuclear cells of peripheral blood are recovered from buffy coats of 6 healthy donors. Such buffy coat cells are resuspended and mononuclear cells are isolated by centrifugation on a Ficoll-Paque gradient (Amersham Pharmacia). Purified mononuclear cells are resuspended into RPMI -1640 medium supplemented with 10% FCS, antibiotics, mercaptoethanol and.L-glutamine. Cell concentration is adjusted to 2×10⁶ viable cells/ml.

Product mediated Inhibition is determined by preincubating those products with mononuclear cells, and adding, 1 hour later, serially diluted solutions of LPS capable of inducing α TFN production. Inhibitory concentration of different acyl dipeptide-like products bearing an accessory functional side chain spacer was found to be 10 μg/ml. E. coli LPS is serially diluted (10-fold dilution) to be in the range of 1 to 0.00001 μg/ml 50 μl of 10 μg/ml dilute product solutions in RPMI 1640 medium are dispensed into a microtiter plate. Each assay (10 μg/ml product concentration +dilute E. coli LPS) is run in triplicates and each microtiter plate includes a negative control comprised of plain medium. LPS-induced αTNF production (without inhibitor) is determined by adding E. coli LPS dilutions to RPMI medium.

100 μl of the cell suspension are added to each well containing dilutions of either product or control. Cells are incubated with the products for 1 hour in an incubator at 37° C., under 5% CO₂ and a moisture saturated atmosphere.

At the end of the pre-incubation period, 50 μl of serially diluted solutions of E. coli LPS are added. Incubation of mononuclear cells with the product and LPS is extended for −18 hours. At the end of the cell activation period, the microtiter plates are centrifuged, and the supernatants are pooled and frozen at −80° C. as aliquots until time of ELISA assay.

Concentration of α TNF secreted by mononuclear cells is determined by chemiluminescent ELISA (QuantiGlo #QTAOO R&D kit). 4-fold dilutions of supernatant fluid are dispensed into each well of the microtiter plate to which anti-human αTNF antibodies are bound. The microtiter plate is incubated for 4 hours at room temperature. After washing, peroxidase-conjugated anti-human a TNF antibody is added. Following a 2 hour incubation period and a thorough washing, a chemiluminescent substrate is added and chemilunescence is read after 20 minutes. α TNF content in the supernatant fluid is determined with respect to a standard curve covering the 7000 to 0.7 μg/ml range.

Inhibition is characterized by the E. coli LPS concentration which elicits 50% production of a TNF during co-incubation in comparison to a control including only RPMI. This concentration increases as product inhibition activity becomes stronger. Knowing inhibition values of αTNF production, this concentration is given by calculating 100-αTNF production induced by product and LPS)/(αTNF production induced by LPS alone)*100. From these inhibition data, concentration is determined by linear regression along the line segment which joins 2 LPS dilutions giving approx. 50% inhibition.

Product ability to inhibit α TNF production in mononuclear cells in response to LPS is further characterized by maximum inhibition. LPS concentration corresponding to this maximum inhibition level (inhibition>95%) is also representative of product inhibitory potency. Results are calculated from a representative experience using all products.

4.7.2. Results

Inhibition of α TNF production in response to LPS in human mononuclear cells by acyl dipeptide-like compounds bearing an accessory functional side chain spacer having the general formula I

| | 50% | max. | max. |
|---|---|---|---|
| | Inhibition | Inhibition in | Inhibition |
| Products | LPS in μg/ml | (%) | LPS in μg/ml |
| OM-197-MC | 1 | 100 | 0.0043 |
| OM-197-MC-MP | 0.19 | 100 | 0.001 |
| OM-197-FV6 | >1 | 100 | 0.014 |
| OM-197-FV8 | >1 | 100 | 0.0033 |
| OM-197-MC-FV5 | >1 | 100 | 0.016 |
| OM-197-MC-FV7 | >1 | 100 | 0.00035 |
| OM-197-MP-AC (R/S, R) | >1 | 100 | 0.00002 |
| OM-197-MP-AC (R, R) | 0.68 | 100 | 0.0013 |
| OM-197-MP-AC (S, R) | >1 | 100 | 0.00002 |
| OM-197-MC-AC | 0.88 | 100 | 0.00003 |
| OM-197-N′C2-MC-AC | 0.0003 | 100 | 0.00001 |
| OM-197-N′(C10-20C8)-MC- | 0.0009 | 85 | <0.00001 |
| AC | | | |
| OM-197-MC-Succ | 1 | 100 | 0.018 |
| OM-197-Lys | 0.70 | 100 | 0.0055 |
| OM-197-AP | >1 | 100 | 0.016 |
| OM-197-Asp | >1 | 100 | 0.00036 |
| OM-197-Asp-AC | 0.29 | 100 | 0.0058 |
| OM-197-Lys-AC | >1 | 100 | 0.001 |
| OM-197-MC-AC-Succ-AZT | 0.45 | 100 | 0.0005 |
| OM-197-MC-AC-Succ-d4T | 0.64 | 100 | 0.00016 |
| OM-197-MC-AC-Succ | >1 | 100 | 0.044 |

Negative control comprised of RPMI medium induced in a baseline α TNF production below test sensitivity level of 0.7 mg/ml.

All acyl-dipeptide-like compounds are able to induce inhibition of TNF production in response to E. coli LPS. Product concentration required to achieve this inhibition depends on the particular accessory functional side chain spacer and length of fatty acid chains. Even acyl- dipeptide-like compounds coupled to a drug by an ester bond are able to induce such an inhibition.

### Example 4.8.

### Determining Capacity of Compounds in Accordance with the Invention to Activate of NK Cell Cytotoxicity against Target Cells of K562 Cell Line

4.8.1. Procedure

PBMC were recovered from buffy coats according to the procedure peviously described in example 4.6. Briefly stated, after centrifugation over a Ficoll gradient and 3 washing steps, PBMC were divided into a 6-well plate at a density of 3×10⁶ cells/ml in 3 ml of RPMI containing 10% FCS and incubated either in medium alone or stimulated with y IFN (1000 U/ml), LPS (1 μg/ml), OM-197-AC5 (1 μg/ml), or OM-197-FV6 (1 μg/ml). Cells were incubated under such conditions for 24 hours. K562 cells (human leukemia cell line, ATCC #CCL 243, 20110-2209 Manassas (Va.), U.S.A.) were maintained in culture using RPMI medium containing 10% FCS and subjected to 2 passages per week.

In order to make cytotoxicity measurement, PBMC and K562 cells were washed twice in HBSS and resuspended in Phenol Red-free RPMI medium containing 5% FCS. PBMC were divided into a round bottom 96-well plate so as to achieve a PBMC/target ratio of 20/1, 10/1 and 5/1 in 50 μl volume. 5000 K562 cells (target) were added in each well using 50 μl of Phenol Red-free RPMI. Final volume was 100 μl. The plate was centrifuged to promote contact then incubated at 37° C. for 4 hours.

Cytotoxicity was assayed using a non radioactive CytoTox 96 kit based on measuring LDH release during cell lysis (kit #G1780, batch # 124100, Promega, Madison, Wis.) according to the supplier's instructions.

The percentage of cytotoxicity was calculated according to the following equation

$$\% cytotoxicity = \frac{O . D . ( K562 + PBMC ) - O . D . PBMC + O . D . K562 . alone}{O . D . ( K562 . total . lysis ) - O . D . K562 . alone} \times 100$$

4.8.2. Results po Compounds OM-197-AC5 and OM-197-FV6 induce an NK activity level which is comparable or slightly inferior to that induced by LPS when tested at the same concentration (1 μg/ml).

| | | Optical density | | |
|---|---|---|---|---|
| | | at 490 nm | | |
| | Ratio | | K562 + | % |
| stimulus | PBMC:K562 | PBMC alone | PBMC | cytotoxicity |
| Medium | 20:1 | 0.141 | 1.5 | 0.5 |
| | 10:1 | 0.073 | 1.55 | 7.7 |
| | 5:1 | 0.055 | 1.4 | 0 |
| IFN-γ | 20:1 | 0.101 | 2.1 | 39.3 |
| | 10:1 | 0.064 | 1.9 | 29.4 |
| | 5:1 | 0.035 | 1.68 | 17.9 |
| LPS | 20:1 | 0.17 | 2.2 | 42 |
| | 10:1 | 0.122 | 1.9 | 26 |
| | 5:1 | 0.035 | 1.7 | 19 |
| OM-197-MP-AC | 20:1 | 0.084 | 1.8 | 22 |
| | 10:1 | 0.084 | 1.66 | 13.7 |
| | 5:1 | 0.027 | 1.5 | 7.4 |
| OM-197-MC-FV6 | 20:1 | 0.11 | 2.1 | 38.8 |
| | 10:1 | 0.087 | 1.65 | 12.9 |
| | 5:1 | 0.079 | 1.4 | 0 |

| | plain culture | | |
|---|---|---|---|
| Controls | medium | K562 alone | K562 full lysis |
| Optical density at 490 nm | 0.408 | 1.35 | 3 |

### Example 4.9

### Determining the Capacity of Compounds in Accordance with the Invention to Induce Lymphocyte Proliferation in Mice

5 4.9.1. Experiment Outline

Spleenic cells derived from a group of 4 naive CBA mice are pooled, cultured in quadriplicate either in absence or presence of OM-197-MP-AC adjuvant (0.1 to 10 μg/ml). Cell proliferation response is assessed by measuring tritiated thymidine (³H-TdR) take-up after 1 or 2 days of culture. Values reported in the table stand for the arithmetic mean +standard deviation of 4 cultures.

Adjuvants:

OM-197-MP-AC stock solution is prepared at a concentration of 0.9 mg/ml in water for injection to which 0.1% triethanolamine is added.

Results

OM-197-MP-AC product triggers spleenic cell proliferation in vitro. This effect which is seen to be at a peak level after one day of culture, results in a 5-fold increase of thymidine take-up at a concentration of 1 μg/ml and a 12-fold increase of thymidine take-up at a concentration of 10 μg/ml After one day of culture, OM-197-MP-AC product, at a concentration of 1 μg/ml, exerts a powerful mitogenic effect of the same magnitude as Concanavalin A at 5 μg/ml. (see Table 1).

TABLE 1
Measuring proliferation response of murine spleenic cells
to OM-197-MP-AC adjuvant

³H-TdR take-up
(CPM × 10⁻³)
Incubation time
(days)

| Adjuvant | | 1 | 2 |
|---|---|---|---|
| No adjuvant | | 6.4 ± 1.0 | 14.3 ± 0.8 |
| OM-197-MP-AC (μg/ml) | 0.1 | 8.7 ± 0.7 | 21.1 ± 1.3 |
| | 1.0 | 34.3 ± 2.6 | 55.6 ± 5.2 |
| | 10 | 79.0 ± 12.0 | 92.7 ± 8.8 |

Data reported in the above table stand for mean ±SD of take-measurements for quadriplicate cultures. (5 μg/ml) concanavaline A used as a positive control results in a thymidine take-up of 37.5±6.2×10³ cpm after one day of culture.

### Example 4.10

### Determining Antiviral Activity of Compound Conjugates in Accordance with the Invention Obtained Through Coupling to AZT and d4T as a Prodrug

4.10.1. Procedure

T MT-4 lymphocyte cekk line which had been transformed into a continuous cell line by an HTLV infection, a virus associated with one form of leukemia, was used to test the antiviral activity (anti-HIV) of AZT- and d4T-conjugated compounds according to the invention, respectively. A characteristic feature of such cells is lack of survival following an HIV infection. The test is based on protection provided by the product (like AZT) against this viral destructive effect.

Products were tested in serially diluted solutions in a 96-well plate. In the upper portion of the microplate, products were tested by incubation at various concentrations with cells and virus (in triplicate), so as to determine the concentration of product which affords cell protection against the viral destructive effect. IC₅₀ of the product is taken as the concentration where a 50% loss of viable cell count is recorded. Viable cell count was done with MTT, a yellow tetrazolium compound which undergoes enzymatic reduction (at the mitochondrial level in viable cells) into a purple colored formazan compound. Wells containing viable cells are purple in colour, whereas dead cells give a persistent yellow color. Measurement of optical density (O.D.) allows this effect to be quantified.

Cytotoxicity of a given product is assessed in the same manner in the lower portion of the plate where the test is conducted by incubating serially diluted products over virus-free cells, to thereby determine the toxic effect of products on cells. The result is given as CC₅₀, i.e. the concentration at which 50% of cells are dead due to product toxicity. Selectivity (selectivity index, SI) of a given product is defined as a CC₅₀/IC₅₀ ratio. This parameter is of great importance since the selectivity index of a fairly efficient product is high.

4.10.2. Results

Conjugates obtained by coupling products according to the invention to AZT (OM-197-LC-succ-AZT) or d4T have antiviral activities directed against HIV 1 IIIB and HIV 2 ROD. Results of two experiments (run in triplicates) on MT-4 cells are given below:

| Product | HIV strain | IC₅₀ (μM) | CC₅₀ (μM) |
|---|---|---|---|
| AZT | HIV-1 IIIB | 0.0029 | 187 |
| OM-197-MC-Succ-AZT | HIV-1 IIIB | 0.0519 | >94 |
| D4T | HIV-1 IIIB | 0.107 | 321 |
| OM-197-MC-Succ-d4T | HIV-1 IIIB | 0.963 | +/−97 |
| AZT | HIV-2 ROD | 0.0018 | 187 |
| OM-197-MC-Succ-AZT | HIV-2 ROD | 0.061 | >94 |
| D4T | HIV-2 ROD | 0.22 | 321 |
| OM-197-MC-Succ-d4T | HIV-2 ROD | 0.51 | +/−97 |

AZT or d4T-conjugated inventive compounds display antiviral activities in this MT-4 cell model. One advantage of such prodrugs lies on one hand in that conjugates have a lipid structure which makes it possible to target compounds to the infected cells by releasing antiviral substances into the virus reservoir and in their ability to regulate the immunological activity of target cells on the other.

### Example 4.11

### Determining Ability of Compounds in Accordance with the Invention to Activate Maturation of Murine Predendritic Cells into Dendritic Cells

4.1 1.1. Procedure

Preparation of Dendritic Cells

The marrrow was aspirated from the femur and tibia. bones of (C57BI/6, 6 to 8 week-old) mice. The marrow suspension was screened through a 70 μm cell sieve in order to obtain a single cell suspension. Marrow cells were resuspended into Isocove's modified Dulbecco medium (IMDM) supplemented with 10% of heat inactivated foetal calf serum (iFCS) and antibiotics. Cells were incubated overnight at 37° C., under CO₂ and a moisture saturated atmosphere, inside 6-well microtiter plates. Next day (day 0), non adherent cells were collected and washed. Viable cells were counted by trypan blue dye exclusion test. Cell concentration was adjusted to 2×10⁵ cells/ml in IMDM containing 20 ng/ml of murine recombinant GM-CSF and 20 ng/ml of murine recombinant IL-4 (rmIL-4) and plates were further incubated. On the 3^{rd} day, there were added freshly prepared mGM-CSF and rmIL-4 at a concentration of 10 ng/ml. On the 6^{th} day, non adherent cells were collected, washed and counted by trypan blue dye exclusion. Cell concentration was adjusted to 2×10⁵ cells/ml in IMDM containing 10 ng/ml of rmGM-CSF and rmIL-4, and plates were further incubated. On the 8^{th} day, non adherent cells made up of both mature and immature dendritic cells (CD-DM) were collected and used in product co-incubation experiments.

Incubation with Products According to the Invention:

CD-DM were incubated at a concentration of 5×10⁵ cells/ml in 24-well microtiter plates in presence of the following activators: Esherichia coli strain 0111:B4 LPS as positive control, IMDM as negative control; as well as 2 compounds in accordance with the invention: OM-197-MC-FV5 and OM-197-PM-AC at a concentration of 0.1, 1 and 10 μg/ml. After a 48 hour incubation period, cells were collected and stored for flow cytometry analysis

Flow Cytometry Analysis

CD-DM were washed with PBS containing 2% iFCS and 0.01% sodium azide (FACS buffer). Then, cells were incubated on ice for 30 minutes with optimal concentrations of anti-CD86 antibody; anti-CD40 antibody, FITC conjugated anti-MCHII antibody, and peroxidase-conjugated anti-CD80 antibody. Then, cells were washed 3 times. Labelling of anti-CD86 and anti-CD40 antibodies was performed by incubating cells on ice for 30 minutes with peroxidase-conjugated donkey anti-rat immunoglobulins. Following incubation, cells were washed and resuspended into FACS medium. Fluorescence was measured with the aid of FACScan™ flow cytometry. 15000 pulse events were analyzed per sample. Cells which had been incubated with the conjugate alone were used as a negative control.

Cell Expression

| | | Surface Markers | | | | | | | |
|---|---|---|---|---|---|---|---|---|---|
| | | CD40 | | CD80 | | CD86 | | MHCII | |
| | μg/ml | MFI | % Cells | MFI | % Cells | MFI | % Cells | MFI | % Cells |
| OM-197-MC-FV5 | 10 | 630 | 54 | 97 | 48 | 4097 | 51 | 1681 | 48 |
| | 1 | 517 | 44 | 84 | 45 | 3888 | 43 | 1691 | 43 |
| | 0.1 | 442 | 33 | 66 | 42 | 3437 | 37 | 1689 | 40 |
| OM-197-MC-AC | 10 | 605 | 55 | 97 | 53 | 4162 | 52 | 1622 | 49 |
| | 1 | 493 | 48 | 73 | 47 | 3791 | 45 | 1607 | 45 |
| | 0.1 | 446 | 34 | 66 | 41 | 3504 | 37 | 1597 | 38 |
| LPS | 0.1 | 539 | 51 | 95 | 51 | 3445 | 50 | 1546 | 51 |
| Negative control | 0 | 431 | 29 | 74 | 41 | 3011 | 34 | 1553 | 41 |

MFI: mean fluorescence intensity

Products in accordance with the invention are capable of inducing maturation of predendritic cells into dendritic cells. These results obtained in vitro are consistent with observations made in mice in vivo.

In vivo, i.v. and s.c. administration of OM-197-MP-AC at a concentration of 20 μg and 5 μg per mouse, respectively, induce maturation and migration of dendritic cells into the spleen, from the marginal zone (located between red and white pulp) to the white pulp where T cells are found. Immunostaining of histological slides taken from spleen of animals treated with products of the invention reveal colocalization of mature dendritic cells and T cells in the white cortex, as is seen with LPS treatment, whereas in untreated animals, no migration and maturation are observed.

### 5^{th} Series of Examples

### Pharmacological Studu of Compounds in Accordance with the Invention (In Vivo)

### Example 5.1

### Assessing Properties of Dipeptide-Like Compounds Bearing an Accessory Functional Side Chain Spacer in Admixture or Coupled with (NANP)₆P₂P₃₀ in a Murine Immunization Model

5.1.1. Experimental Procedure

Antigen: (NANP)₆P₂P₃₀ peptide comprises a recurrent (NANP) sequence of Plasmodium falciparum and two sequences of tetanus toxin (P₂: 830-843 and P₃₀: 947-967). This peptide is known as a T helper epitope and has shown its capacity to induce a strong prolonged immune reponse in presence of common adjuvants. Such a peptide has been obtained by synthesis means according to a method disclosed in the litterature (Valmori et al., 1992, J. Immunol., 149: 717-721). Antigen stock solution is prepared at a concentration of 0.4 mg/ml in a 0.9% NaCl/water mixture at pH 8.0.

Adjuvants:

Stock solutions of dipeptide-like compounds (OM-197-MP, OM-197-FV6 and OM-197-MC) are prepared at a concentration of 1 mg/ml in 0.9% NaCl-water, to which 0.1% triethanolamine is added. The positive control is comprised of Incomplete Freund's Adjuvant (IFA from Difco, Detroit, Mich., USA) with the negative control being a 0.9% NaCl/water solution.

Throughout this experimental procedure, the antigen and adjuvant are formulated in the form of a mixture or conjugates as previously described. Two types of conjugates (OM-197-FV)ₙ-(NANP)₆P₂P₃₀ were tested. The extent of coupling of such products is variable, i.e. 1, 2 or 3 molecules of OM-197-FV may be found on a single peptide molecule (mixture of mono-, bi- and triconjugates, n=1, 2, and 3, respectively), i.e. 1 molecule of OM-197-FV per one peptide molecule (monoconjugate, n=1).

Immunization schedule:

6-week old female BALB/c mice (6 mice per group) are immunized twice, with a subcutaneous shot in the tail end containing 0.1 ml. Amounts administered are 20 μg of antigen plus 50 μg of adjuvant during the first shot and 10 μg of antigen plus 50 μg of adjuvant during the second shot. In case of conjugates, the antigen portion is critical in determining the injected dose.

Table listing of experimental groups:

| Group | Adjuvant | Antigen | Number of mice |
|---|---|---|---|
| 1 | | NaCl | 6 |
| 2 | — | (NANP)₆P₂P₃₀ | 6 |

| | | |
|---|---|---|
| 3 | IFA + (NANP)₆P₂P₃₀ | 6 |
| 4 | OM-197-FV6 + (NANP)₆P₂P₃₀ | 6 |
| 5 | OM-197-MC + (NANP)₆P₂P₃₀ | 6 |
| 6 | (OM-197-FV)_{1,2,3}-(NANP)₆P₂P₃₀ | 6 |
| 7 | OM-197-FV-(NANP)₆P₂P₃₀ | 6 |
| 8 | OM-197-MP + OM-197-FV(NANP)₆P₂P₃₀+ | 6 |

Immunization and sampling schedule:

| | Weeks | | |
|---|---|---|---|
| | O | 3 | 5 |
| Immunization shots | ↑ | ↑ | |
| (N^{b}) | (1) | (2) | |
| specific antibody response | ↑ | | ↑ |

Blood Sampling:

Serum Preparation:

Blood sampling is conducted at weeks 0 & 5. Blood is allowed to stand for 60 min. at 37° C., and then is kept overnight at 4° C. Serum is subsequently frozen at −80° C. until time of antibody assay.

5.1.2. Determination of anti-(NANP)₆P₂P₃₀ IgG Antibody Titer

Assay of IgG antibodies specifically raised against (NANP)₆P₂P₃₀ antigen is performed by an ELISA technique. Binding of antigen is done in 96-well microtiter plates (Maxisorp F96, Nunc, DK) by conducting an overnight incubation in a moist chamber at 4° C. with each well containing 1.1 ml of PBS (phosphate buffered saline) containing 0.001 mg/ml (NANP)₆P₂P₃₀ antigen. Blocking of the microtiter plate is performed with PBS containing 1% of bovine serum albumin (BSA, Fluka, Switzerland). Plates are washed with PBS containing 0.05% Tween 20 (Sigma, St. Louis, Mo., USA). Serum samples collected at 5 weeks are serially diluted with dilution buffer (PBS containing 2.5% of skimmed milk powder and 0.05% of Tween 20), then transferred into a microtiter plate and allowed to stand for 1 hr. at room temperature (RT). Plates are then washed with PBS. A dilute solution containing mouse polyclonal anti-immunoglobulin G antibody coupled to alkaline phosphatase (Sigma, St. Louis, Mo., USA) is then dispensed into those plates and incubated for 1 hr. at RT. Plates are washed with PBS and specific antibodies are revealed by a color reaction involving addition of an alkaline phosphatase substrate, p-nitrophenylphosphate (Sigma, St. Louis, Mo., USA). Absorbance at 405 nm is read with a microtiter plate reader (Dynatech 25000 ELISA reader, Ashford, Middlesex, UK), each serum sample is measured in duplicate. Results stand for the mean of all absorbance measurements at 405 nm relating to mice in each group.

5.1.3. Results : Specific Response

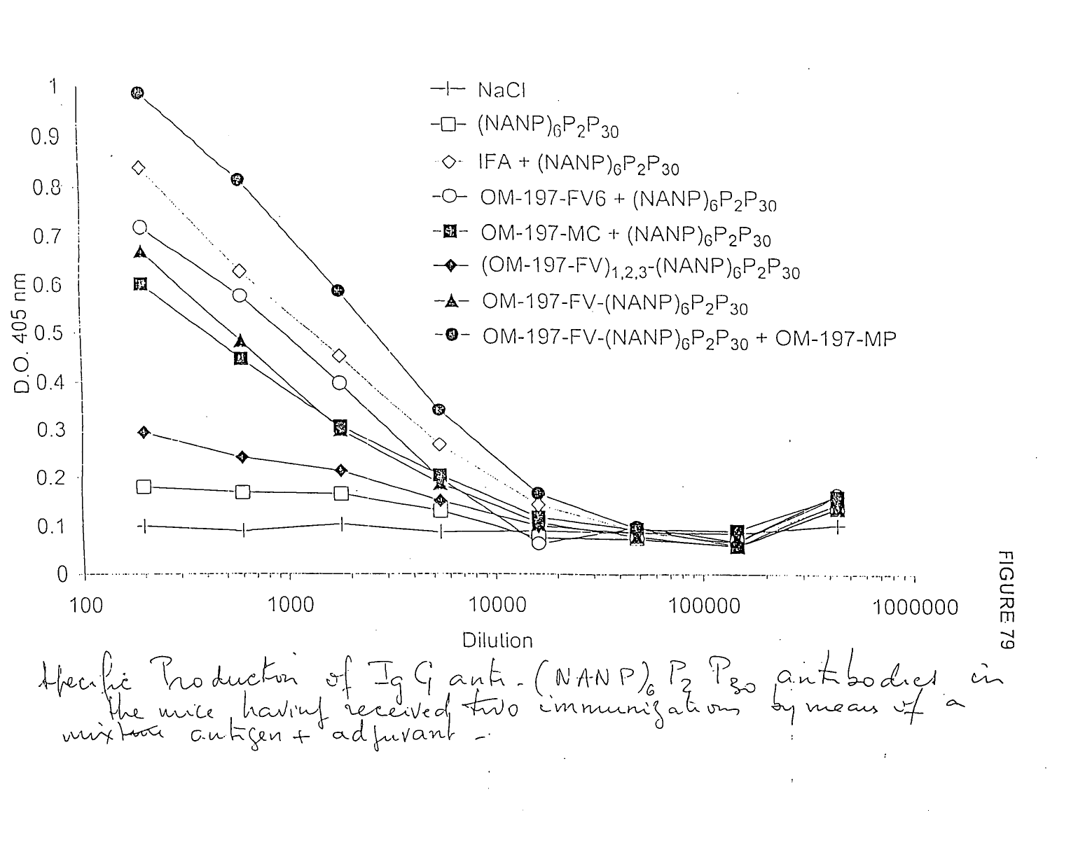

Production of IgG antibodies specifically directed to (NANP)₆P₂P₃₀ antigen, as determined by ELISA, is graphically shown for mice administered one, two and three immunization shots (FIG. 79)

Two shots of an antigen+adjuvant mixture (OM-197-FV or OM-197-MC) induce a serologic response which is greatly superior to the one observed when injecting antigen alone. Immunization with the OM-197-FV-(NANP)₆P₂P₃₀ monoconjugate induces an IgG response which is slightly greater than the one obtained with the antigen+OM-197-FV6 mixture. Higher coupling rate for (OM-197-FV)_{1, 2, 3}-(NANP)₆P₂P₃₀ does not improve the serologic response directed to this antigen. The mixture containing OM-197-MP adjuvant and OM-197-FV-(NANP)₆P₂P₃₀ monoconjugate does result in a significantly higher serologic response against this antigen that exceeds values obtained for the positive control consisting of (NANP)₆P₂P₃₀ +IFA.

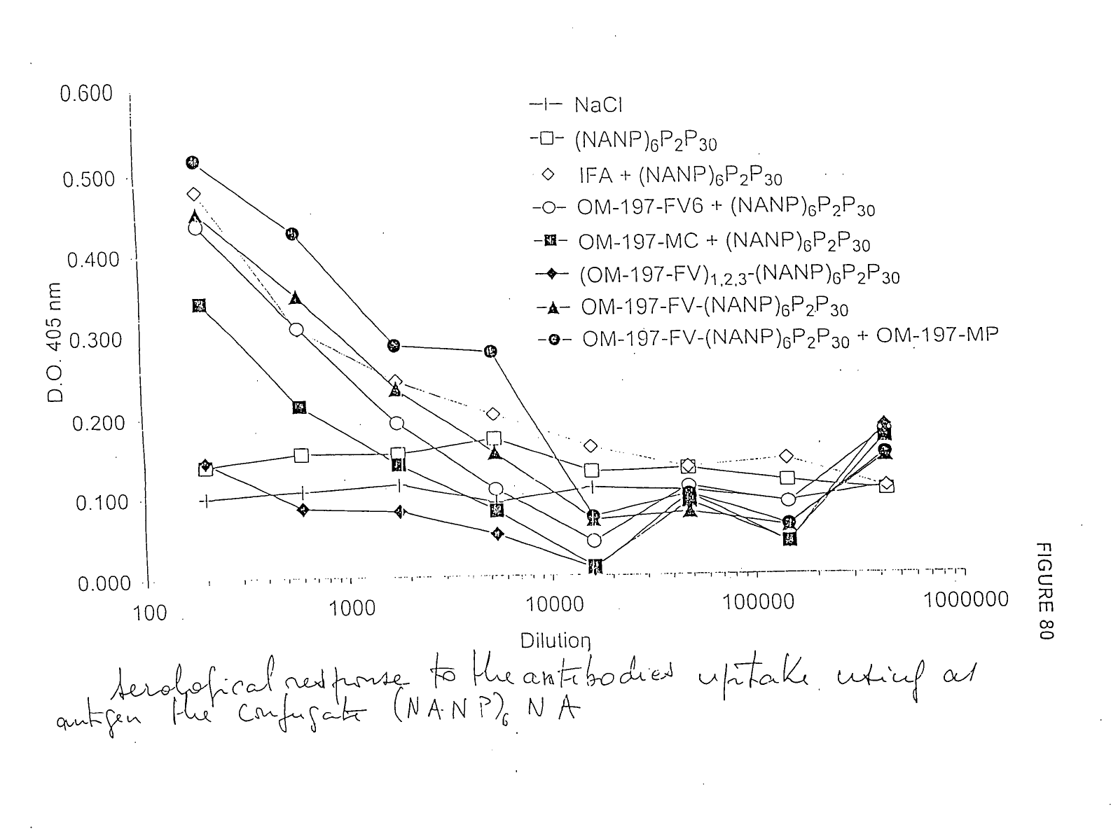

Similar results are obtained when the antibody is captured by an ELISA technique using (NANP)₆NA peptide as antigen (FIG. 80).

### Example 5.2

### Ovalbumin Immunization Group: Determining Specific Antibodies Raised in Mice Following 1 and 2 Subcutaneous Injections

5.2.1. Experiment Outline

This study is aimed at comparing the serologic response induced by injecting an adjuvant+antigen mixture (OM-197-MP+ovalbumin) with the one elicited by a conjugate based on the same antigen (OM-197-FV-ovalbumin). To this end, 30 BALB/c mice (females, aged 8 weeks at the beginning of treatment) were divided into 3 groups as follows:

| | | | Injected |
|---|---|---|---|
| | Adjuvant - antigen | Adjuvant (unbound) | volume |
| Groups | 25 μg prot/animal/injection | 50 μg/animal/injection | μl |
| A | ovalbumin | — | 200 |
| B | ovalbumin | OM-197-MP | 200 |
| C | OM-197-FV-ovalbumin | — | 200 |

Solutions:
Group A: A stock solution of antigen alone (ovalbumin, Fluka AG, Buchs, Switzerland) was prepared at a concentration of 125 μg/ml in H₂O+0.01% thiomersal.
Group B: Stock solution of an antigen+adjuvant mixture (ovalbumin+OM-197-MP) contains 125 μg/ml of ovalbumin and 250 μg/ml of OM-197-MP in H₂O+0.01% thiomersal.
Group C: Stock solution of OM-197-FV-ovalbumin conjugate at a concentration of 125 μg/ml of protein in H₂O+0.01% thiomersal.

Preparation of Injection Solutions:

Each stock solution is incubated for 15 minutes at 37° C., then an adequate volume of NaCl (14%) is systematically added to each solution to obtain an isotonic solution (0.9% NaCl). The whole mixture is vortexed for 3 minutes.

Immunization Schedule:

Injections are schedulled at days 0 and 14. The solutions are administered subcutaneously (100 μl at 2 different sites, with a total of 200 μl per animal). Blood sampling occurred at days 14 and 28 (retro-orbital puncture).

Assay for Anti-Ovalbumin Immunoglobulins:

The following serum Immunolglobulins which are specifically directed against ovalbumin were assayed in duplicate by ELISA: IgG1, IgG2a, and IgM. Briefly stated, microtiter plates (NUNC Immunoplate, Roskilde, DK) were incubated (overnight coating) at 4° C. together with 100 μl ovalbumin (0.5 μg) in bicarbonate buffer pH 9.6). After washing with 0.5% Tween-20 (Merck Hohenbrunn, D), sera were diluted 50-, 200- and 800-fold (diluting solution : phosphate buffered saline (PBS)+1% bovine serum albumin (BSA, Sigma, St; Louis, Mo., USA)+0.02% Tween-20)). 100 μl of each dilute serum sample were added to the wells. Incubation lasted 45 minutes at 37° C.

After a second washing step, IgG1, IgG2a and IgM specifically directed to ovalbumin were incubated for 30 minutes at 37° C. together with 100 μl of anti-IgG1 antibodies (anti-mouse rat antibody)-peroxidase conjugate (Serotec, Oxford, UK), IgG2a-peroxidase conjugate (Pharmingen, San Diego, Calif., USA) and IgM-biotin conjugate (Pharmingen, San Diego, Calif., USA), diluted beforehand in PBS/BSA/Tween buffer (250-, 1000-, 500-fold dilutions, respectively). For IgM, after an extra washing step, a ₃^{rd} incubation step was required (30 min. at 37° C.) with a 1:100 dilute solution of streptavidin-peroxidase conjugate (Dako, Glostrup, DK).

After the washing step, 100 μl of a phenylene 1,2-diamine solution (OPD, Merck, Darmstadt, GFR) were added to detect peroxidase-coupled anti-IgG1 secondary antibodies whereas for IgG2a and IgM, the reagent used was 3′, 3′, 5′, 5′-tetramethylbenzidine (TMB, Sigma, St. Louis, Mo., USA). After a 20 minute incubation period at room temperature, the reaction was stopped by adding 100 μl of 2N H₂SO₄. Absorbance values were read at 490 nm with a Bio-Rad 3550 model plate reader

5.2.2. Results

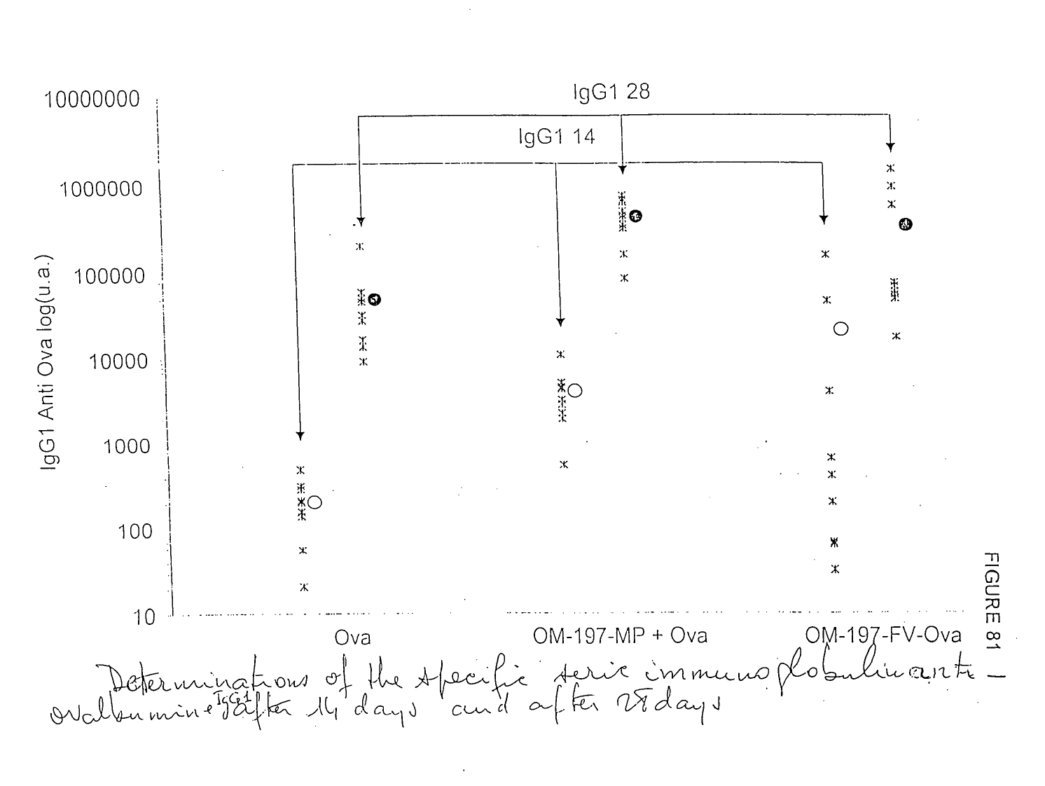

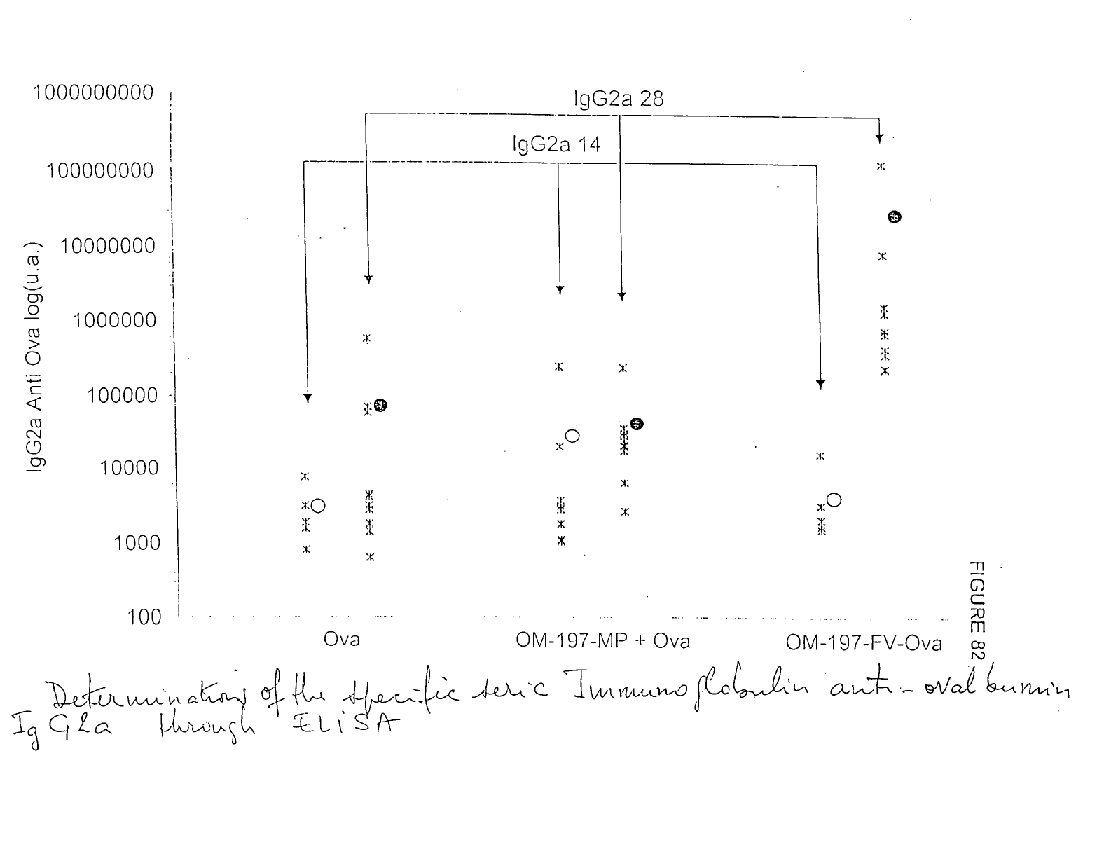

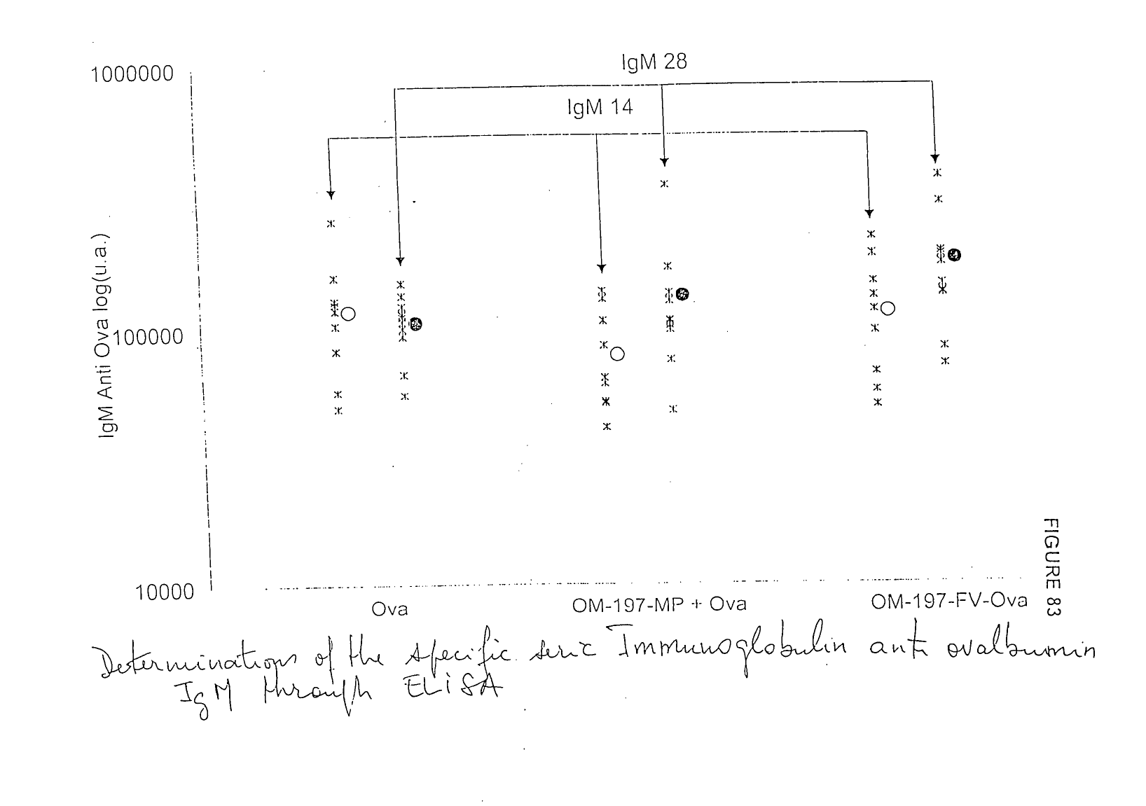

Results of each 490 nm reading are given in arbitrary units (A.U.) per ml. This is achieved by comparing each sample with a standard sample prepared from different dilutions of a sample pool collected from group A at 28 days (animals injected with ovalbumin alone). As clearly understood by this term, the sample pool diluted 50-fold is at a concentration of 1000. A.U./ml. Individual results are then corrected for the corresponding dilution factor (50-, 200-, or 800-fold) and are shown at FIGS. 81, 82 and 83. Void circles correspond to the mean value at 14 days while black circles correspond to the mean value at 28 days; Ova stands for ovalbumin in short.

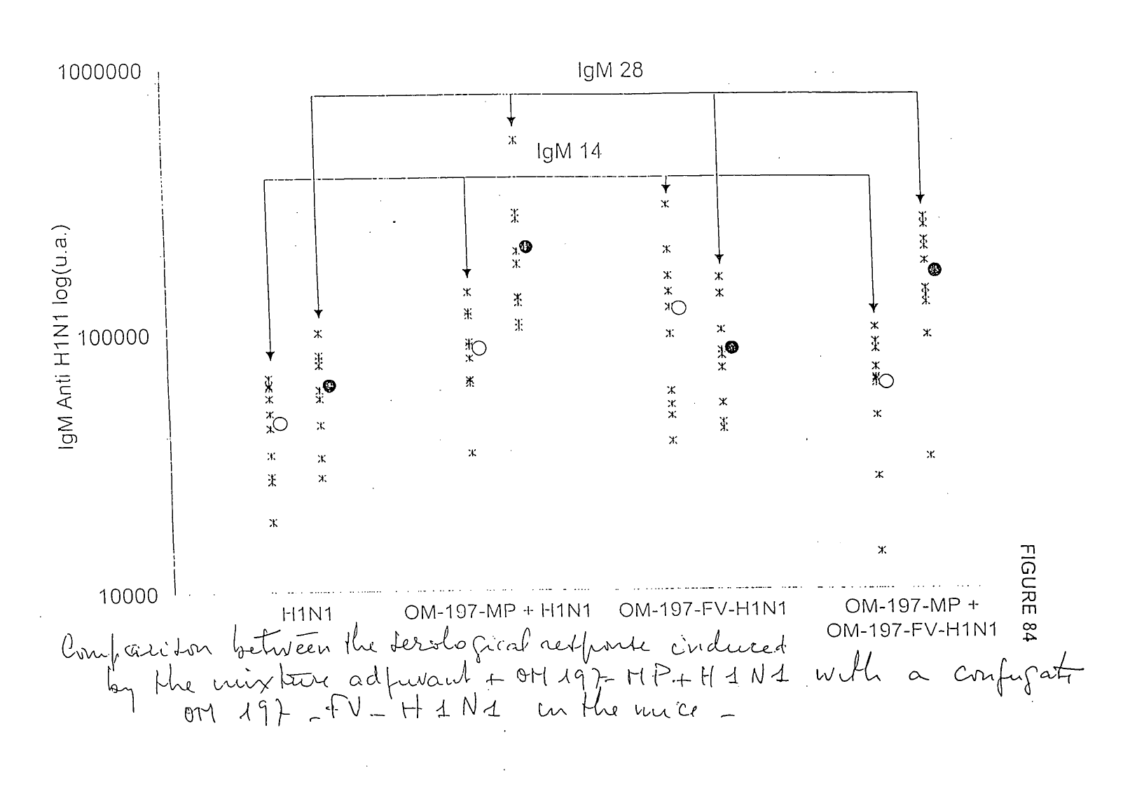

These results indicate that the OM-137-FV-ovalbumin conjugate is a particulary effective immunogen in the model under study, since it increases significantly the titer of specific antibody to ovalbumin in mice after the second injection, irrespective of the immunoglobulin subclass being analyzed (IgG1, IgG2a and IgM). In particular, there is a striking increase of anti-ovalbumin IgG2a (FIG. 84) which suggests that a TH1 immune reaction is favoured.

Ovalbumin+OM-197-MP non covalent mixture gives rise to an IgG2a response lower than what is seen with the conjugate.

### Example 5.3

### H1N1 Hemagglutinin Immunization Group: Determining Specific Antibodies Raised in Mice Following 1 and 2 Subcutaneous Injections

5.3.1. Experiment Outline

This study is designed to compare the serologic response induced by injecting an adjuvant+antigen mixture (OM-197+H1N1) to that elicited by a conjugate based on the same antigen (OM-197-FV-H1N1). To this end, 30 BALB/c mice (females, 8 weeks-old at the beginning of treatment) are divided into 3 groups as follows:

| | | Adjuvant (unbound) | Injected |
|---|---|---|---|
| | Adjuvant - antigen | 50 μg/ | volume |
| Groups | H1N1 2.5 μg/animal/injection | animal/injection | μl |
| A | H1N1 | — | 100 |
| B | H1N1 | OM-197-MP (50 μg) | 100 |
| C | OM-197-FV-H1N1 | — | 100 |

5.3.2. Preparation of Injection Solutions
Group A: The stock solution of H1N1 (A/Beijing 262/95 hemagglutinin, Solvay Duphar, Weesp, NL) is prepared at a concentration of 25 μg/ml in H₂O.
Group B: The stock solution of H1N1+OM-197-MP contains 25 μg/ml of H1N1 and 500 μg/ml of OM-197-MP in H₂O.
Group C: The stock solution. of OM-197-FV-H1N1 (H1N1 covalently bound to OM-197-FV) is at a concentration of 25 μg/ml in H₂O).

Each stock solution is incubated for 15 minutes at 37° C. and 135 μl of (14%) NaCl are then added to 2 ml of each solution. The whole mixture is vortexed for 3 minutes.

5.2.3. Injection Schedule and anti-H1N1 Immunoglobulin Assay:

Injections are schedulled on days 0 and 14. Solutions are administered subcutaneously (50 μl at 2 different sites, with a total of 100 μl per animal). Blood sampling is schedulled on day 14 (retro-orbital puncture).

IgM antibodies specifically directed to H1N1 were assayed in duplicate by ELISA. Briefly stated, microtiter plates (NUNC Immunoplate, Roskilde, DK) were incubated (overnight coating) at 4° C. with 100 μl H1 N1 (0.5 μg) in bicarbonate buffer (pH 9.6). After washing with 0.5% Tween-20 (Merck Hohenbrunn, D), sera were diluted 50-, 200- and 800-fold (diluting solution: phosphate buffered saline (PBS)+1% bovine serum albumin (BSA, Sigma, St. Louis, Mo., USA)+0.02% Tween-20)). 100 μl of each dilute serum sample were added to the wells. Incubation lasted 45 minutes at 37° C.

After a second washing step, IgM specifically directed to H1N1 are incubated for 30 minutes at 37° C. together with 100 μl, of anti-IgM antibody (rat anti-mouse antibody)-biotin conjugate (Pharmingen, San Diego, Calif., USA), diluted beforehand in a Tween buffer (500-fold dilution). After an extra washing step, a 3^{rd} incubation period is required (30 min. at 37° C.) with a 1:100 dilute solution of streptavidin-peroxidase conjugate (Dako, Glostrup, DK).

After the washing step, 100 μl of a 3′, 3′, 5′, 5′-tetramethylbenzidine solution (TMB, Sigma, St. Louis, Mo., USA) are added to detect peroxidase-coupled anti-IgM secondary antibodies. After a 20 minute incubation period at room temperature, the reaction is stopped by adding 100 μl of 2N H₂SO₄. Absorbance values are read at 490 nm with a Bio-Rad 3550 model plate reader.

5.3.4. Results

Results of each 490 nm reading are given in arbitrary units (A.U.) per ml. This is achieved by comparing each sample with a standard prepared from variable dilutions of a sample pool collected from group A (animals injected with H1N1 alone) at 28 days. As the term implies, the sample pool diluted 50-fold is at a concentration of 1000 A.U./ml. Individual results are then corrected for the corresponding dilution factor (50, 200, or 800-fold) and are shown on FIG. 84: black circles correspond to the mean value at 28 days.

Mice immunized with an OM-197-FV-H1N1 conjugate show an anti-H1N1 IgM antibody titer greater than control consisting either of H1N1 antigen alone, or of an OM-197-MP+H1N1 mixture.

### Example 5.4

### Evaluation of the Adjuvant Properties of OM-294-MP-AC in a Mouse Immunization Model by Subcutaneous Administration of Leishmania LmCPb Antigen.

5.4.1. Experiment Outline

CBA mice were administered through the tail a single subcutaneous injection (100 μl) of Leishmania parasite LmCPb antigen (3 μg per mouse) ±adjuvant. Two groups of 6 mice each were formed and received the following treatments: antigen alone (control group) and antigen+50 μg OM-197-MP-AC respectively.

Eleven days after injection, inguinal and periaortic lymph node cells (2 groups of 3 mice each) were cultured in triplicate in presence of purified LmCPb antigen or whole extract of the parasite (amastigote). At day 4, a supernatant sample is withdrawn and stored at −20° C. for IL-4 and γ IFN assay. Finally, tritiated thymidine (³H-TdR) is added to the cultures to measure the proliferation reaction.

Cytokine assay is carried out by a sandwich ELISA assay (OptEIA™ kits for γ IFN and IL-4, BD, PharMingen, Basel, Switzerland) and the proliferation response is determined by measuring thymidine take-up (³H-TdR). ³H-TdR take-up given in cpm as the arithmetic mean value±standard deviation (8 pools of 3 mice each) and IL-4 concentration given in pg/ml are individually reported for each pool of 3 mice as the arithmetic mean of two measurements (cytokine assay).

Antigen:

An LmCPb stock solution is prepared at a concentration of 150 μg/ml in PBS

Adjuvants:

The stock solution of OM-294-MP-AC is prepared at a concentration of 0.9 mg/ml in water for injection, with addition of 0.1% triethanolamine.

Antigen-Adjuvant Mixture:

Adjuvant preparation (4 volumes), which had been adjusted to an adequate final concentration in sterile PBS, is mixed just prior to injection with the antigen stock solution (1 volume).

5.4.2. Results

In CBA mice immunized with Leishmania LmCPb antigen, a single 50 μg dose of OM-197-MP-AC is sufficient to induce an increase (2 to 6-fold) in lymph node cell proliferation as measured in vitro in response to purified antigen (in the range of 0.6 to 15 μg/ml) or a whole extract of amastigote stage parasite (in the range of 2 to 50×10⁻⁶/ml). These results are reported in Table 1. Furthermore, treatment of mice by OM-197-MC-AC reveals substantial quantities of IL-4 in the supernatant fluid of lymph node cell cultures challenged with the parasite antigen (see Table 2) whereas γ IFN production was undetected (<100 pg/ml).

TABLE 1
In vitro Proliferation response of lymph node lymphocytes in
CBA mice immunized with LmCPb: Effect of OM-197-MP-AC
adjuvant alone or in combination with CpG-DNA

| | | tritiated thymidine take-up (CPM) | |
|---|---|---|---|
| In vitro challenge | | Without adjuvant | OM-197-MP-AC |
| No challenge | | 0.7 ± 0.2 | 0.7 ± 0.1 |
| LmCPb (μg/ml) | 15 | 2.8 ± 0.8 | 7.0 ± 0.7 |
| | 5 | 1.6 ± 0.4 | 3.0 ± 1.1 |
| | 1.7 | 1.1 ± 0.3 | 1.9 ± 0.5 |
| | 0.6 | 0.9 ± 0.2 | 1.3 ± 0.5 |
| Amastigotes (×10⁻⁶/ml) | 50 | 5.1 ± 1.4 | 32.6 ± 5.2 |
| | 17 | 2.8 ± 1.0 | 12.6 ± 2.5 |
| | 6 | 1.1 ± 0.6 | 5.3 ± 1.5 |
| | 2 | 0.9 ± 0.4 | 1.6 ± 0.3 |
| Con A (μg/ml) | 5 | 124.3 ± 48.9 | 208.5 ± 56.0 |

Values reported in this Table stand for the arithmetic mean ± standard deviation of take-up measurements conducted on two pools of lymph nodes (3 mice per pool; triplicate cultures of each pool)

TABLE 2
In vitro IL-4 production by CBA mouse lymphocytes challenged
with LmCPb: Effect of OM-197-MP-AC adjuvant alone or in
combination with CpG-DNA

| | | | IL-4 production (pg/ml) | |
|---|---|---|---|---|
| In vitro challenge | | | without adjuvant | OM-197-MP-AC |
| No challenge | | a | <8 | <8 |
| | | b | <8 | <8 |
| LmCPb (μg/ml) | 15 | a | <8 | 12 |
| | | b | <8 | 13 |
| Amastigotes (×10⁻⁶/ml) | 50 | a | <8 | 55 |
| | | b | <8 | 70 |
| | 17 | a | <8 | 29 |
| | | b | <8 | 28 |
| Con A (μg/ml) | 5 | a | 117 | 103 |
| | | b | 168 | 102 |

Values reported in this Table stand for the arithmetic mean ± standard deviation of 2 cytokine measurements conducted on two pools of lymph node cell supernatant fluids (a & b; 3 mice per pool) cultured in triplicates

Conclusion

OM-294-MP-AC adjuvant potentiates specific T response in vitro in CBA mice which have been immunized with LmCPb (an abundant protease during the amastigote stage of the Leishmania parasite) as assayed by lymphocyte proliferation in vitro. Moreover, this adjuvant promotes development of anti-LmCPb lymphocytes which secrete substantial quantities of IL-4.

### EXAMPLE 5.5

### Evaluation of the Properties of acyl-dipeptide-like Compounds Bearing an Accessory Functional Side Chain Spacer Either Coupled or in Admixture with Synthetic Peptides Derived from the Circumsporozoite Protein of Plasmodium yoelii (PyCS-252-260) in a Murine Immunization Model

This experiment is aimed at evaluating the capacity of acyl dipeptide-like compounds bearing an accessory side chain spacer when grafted or in admixture with a PyCS 252-260 synthetic peptide derived from the circumsporozoite protein of Plasmodium yoelli, to induce a humoral or cell mediated immune response specifically directed against this peptide

5.5.1. Experimental Procedure

Formulation of Antigens with Adjuvants:

Synthetic peptide PyCS 252-260 corresponding to the T cell epitope of circumsporozoite protein of Plasmodium yoelli has the following sequence SYVPSAEQI (Table 1). Peptide 2 MR99B (SERSYVPSAEQI) was obtained by adding SER amino acids to the N-terminal end and peptide 1 MR99A (KGGKGGKSERSYVPSAEQI) was obtained by adding lysine-glycine-glycine amino acis to the N-terminal end of the previously disclosed MR99B peptide. Peptides were obtined by solid phase synthesis (F-moc, according to the Merrifield and Atherton synthesis method (Atherton et al., Bioorg Chem., 8: 350-351, 1979) using reagents and solvents purchased from Bachem Feinchemikallien (Buddendorf, Switzerland), Novabiochem (Laufelfingen, Switzerland) and Fluka (Buchs, Switzerland). Peptides were purified by reverse phase HPLC.

Mono- and polypeptide conjugates with acyl dipeptide-like compounds bearing an accessory side chain spacer (OM-197-MC-FV)₄ peptides were obtained as described above in Example 3.6. ([OM-197-MC-FV) and Example 3.5. (OM-197-MC-FV-peptide 2). Final sterile solutions of the peptide conjugates were freeze-dried.

Mouse Immunization Regimen:

Antigen dose corresponds to 20 μg of peptide 2 per mouse and per injection (Table 2). Before injection, freeze-dried preparations are taken up into 0.9% NaCl, vortexed for 3 min. and sonicated for 10 min. at 50° C. Formulations incorporating IFA are prepared by mixing one volume of antigen (14.5 μg of peptide PyCS 252-260 plus 50 μg of P30 (universal T cell epitope) in PBS) and one volume of adjuvant.

4-week old BALB/c mice (Harlan, Zeist, NL) received one subcutaneous shot in the tail end. Formulations are injected with a 23″ gauge needle using a final volume of 50 μl. Animals received a second shot of antigen at the beginning of the 5^{th} week and a third shot during the 9^{th} week.

TABLE 1
Antigens

| | | Molecular | Dose/shot |
|---|---|---|---|
| Synthetic peptides | sequences | mass | (μg) |
| Peptide 1 | KGGKGGKSERSYVPSAEQI | 1978.2 | 29.0 |
| Peptide 2 | SERSYVPSAEQI | 1365.5 | 20.0 |
| PyCS 252-260 | SYVPSAEQI | 993.1 | 14.5 |

TABLE 2
Experimental groups, immunization regimen and sampling

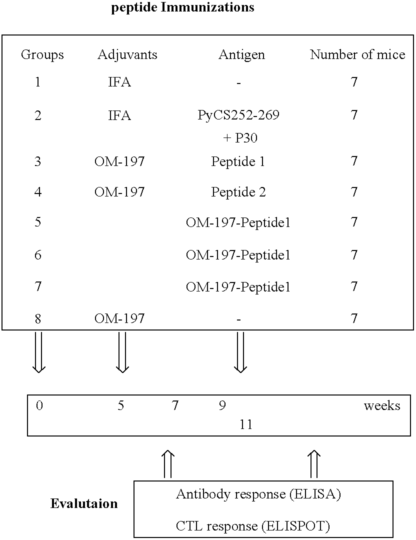

Lymphoid Organ and Blood Sampling

Serum Sampling:

Blood sampling is performed on all mice after 7 and 11 weeks. Blood is allowed to stand for 6 min. at 37° C., then is kept overnight at 4° C. Serum is subsequently frozen at −80° C. until time of antibody assay.

Recovery of Inguinal Lymph Nodes and Spleen:

Two animals belonging to each group are killed after 7 and 11 weeks, respectively. Inguinal lymph nodes and the spleen are surgically removed and the cells are cultured to assay CTL activity by ELISPOT (γ IFN response)

Determination of anti-peptide 1 Antibody Titer:

Assay of antibodies specifically raised against peptide 1 (KGGKGGKSERSYVPSAEQI) is performed by ELISA. Binding of antigen is done in 96-well microtiter plates (Maxisorp F96, Nunc, DK) by conducting an overnight incubation in a moist chamber at 4° C. with each well containing 0.05 ml of PBS (phosphate buffered saline) containing 0.001 mg/ml of peptide 1. Plates are washed with PBS-0.05% Tween 20 (Sigma, St-Louis, Mo., U.S.A.) (PBS-T) and blocking of the plate is made with 5% skimmed milk in PBS-T for 1 hour at room temperature. Individual serum samples (A, B, C, D, E, F, G) of mice taken at weeks 9 and 11 are serially diluted with dilution buffer (PBS containing 2.5% of skimmed milk powder and 0.05% of Tween 20), then transferred into the microtiter plate and allowed to stand for 1 hr. at room temperature (RT). Plates are then washed with PBS and a diluted solution containing goat polyclonal anti-mouse immunoglobulin antibody coupled to alkaline phosphatase (Sigma, St. Louis, Mo., USA) is then dispensed into those plates and incubated for 1 hr. at RT. Plates are washed with PBS and specific antibodies are revealed by a color reaction through adding the alkaline phosphatase substrate, p-nitrophenylphosphate (Sigma, St. Louis, Mo., USA). Absorbance at 405 nm is read with a microtiter plate reader (Dynatech 25000 ELISA reader, Ashford, Middlesex, UK), each serum sample is measured in duplicate. Results stand for the mean of all measurements relating to mice in each group. The antibody titer is given by the highest dilution resuling in a significantly positive response, i.e. an OD greater to background noise level±3 SD.

γ-IFN ELISPOT Assay:

Antibodies specifically directed to murine γ-interferon (O1E703B2) are bound by running an overnight incubation at 4° C. in a moist chamber, adding an antibody solution at 50 μg/ml in an ELISPOT microtiter plate with the well bottom being covered by nitrocellulose (Millipore, Molsheim, France). The blocking step is effected by adding DMEM medium (Life Technologies, Grand Island, N.Y., USA) containing 10% of foetal calf serum (FCS, Fakola, Switzerland) and letting stand for 2 hours at 37° C. Cells obtained from lymphoid organs (inguinal lymph nodes and spleen) are cultured in microtiter plates at a density of 200 000 or 400 000 cells/well, then co-cultured during 24 hours at 37° C. with 100 000 antigen presenting cells (which had been primed with PyCS 252-260 peptide at 37° C. for 1 hr. and irradiated (10 Krad) and washed 3 times) or in presence of Concanavalin A as control. After incubation, cells are removed and following the washing step, a second anti-mouse γ IFN antibody-biotin complex (ANI, 2 μg/ml in PBS with 1% BSA) is added and incubated for 2 hr. A streptavidin-alkaline phosphatase conjugate (Boehringer Mannheim, Mannheim, GFR) diluted 1000-fold is added and incubated for 1 hr at 37° C., and thereafter 3 washings are effected with PBS containing 0.05% Tween 20, followed by 3 washings with PBS. Presence of anti-γ IFN immune complexes is demonstrated by adding BCIP/NBT substrate (Sigma, St. Louis, Mo., USA). This reaction is stopped by washing with tap water. Spots which are positive for γ IFN are then counted under a Biosys GmbH model automatic reader (Karben, Germany). Specific spot count is the-difference between spots counted in presence of cells primed with the peptide and spots counted in absence of peptide. Results are given as mean measurement values recorded for mice in each group. They are expressed as the number of spots per million of cultured cells.

Results

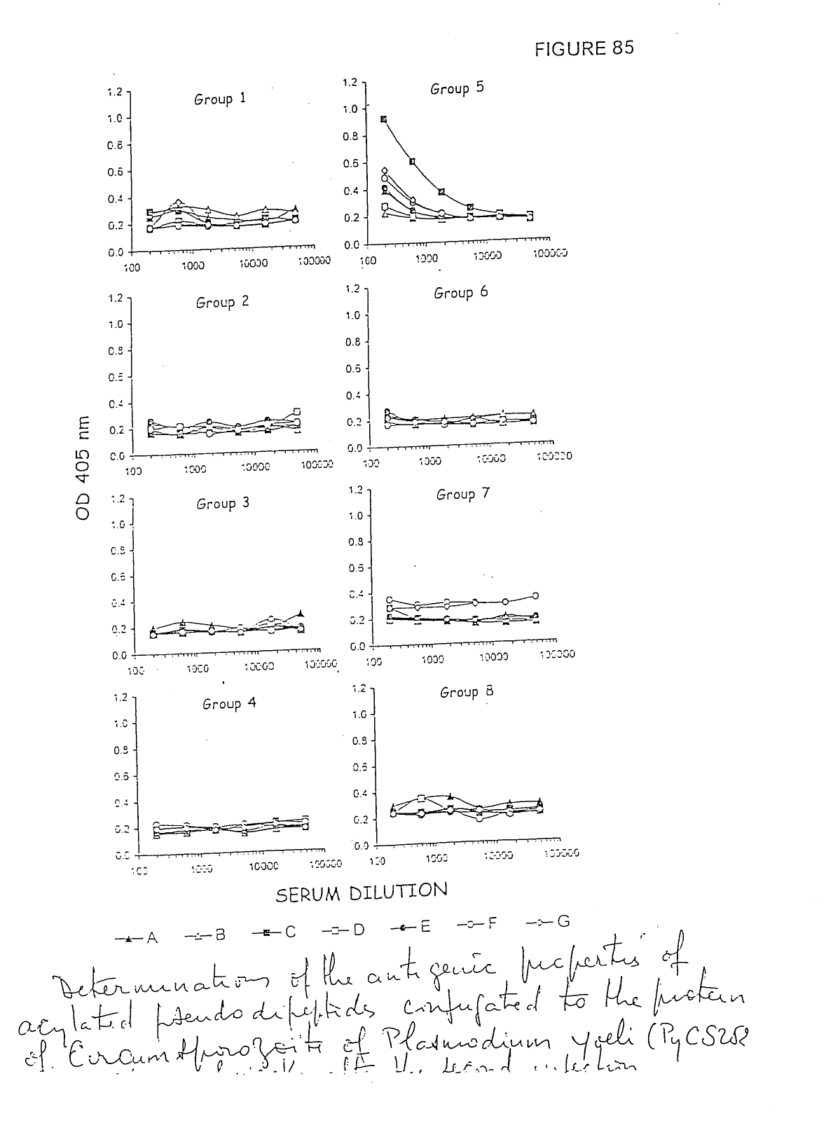

The antibody response is depicted on FIG. 85 (2 weeks after the 2^{nd} shot) and on FIG. 86 (2 weeks after the 3^{rd} shot). After 2 immunization shots, positive responses are observed in 5 out of 7 animals for group 5 (animals previously immunized with peptide 1 coupled to 4 units of acyl-dipeptide-like compound through an accessory side chain spacer). In this group, the percentage of responsive mice increases following the 3^{rd} injection since mice B and D show a moderate but significant antibody titer. Group 3 immunized with the same peptide in admixture with the same adjuvant does not show any significant antibody titer. A nascent response is observed after 3 immunizations shots in group 7 (peptide 2 monoconjugate supplemented with free OM-197 adjuvant). Other groups which were administered the short peptide or no peptide did not display any antibody response.

In conclusion, OM-197-peptide 2 tetraconjuguate is capable of inducing a low to moderate titer antibody response in all immunized mice

Cell activity assay is based on the frequency of CTL secreting γIFN, specifically directed to PyCS 252-260 epitope. Results are recorded in Table 3. The positive control is represented by group 2 and the negative control is represented by adjuvant alone (groups 1 and 8). Cell stimulation control based on Concanavalin A is positive for all mice.

Groups 5 (tetraconjugate) and 7 (monoconjugate+adjuvant) show positive CTL responses.

In conclusion, the peptide conjugated to 4 molecules of acyl dipeptide-like compound (OM-197-peptide 1) is effective whereas the monoconjugate (OM-197-peptide 2) induces no CTL response. By contrast, the monoconjugate supplemented with free OM-197 does induce a CTL response, whereas it does not elicit an antibody response, which fact is probably due to the small size of peptide 2 (12 amino acids).

TABLE 3
CTL Response to PyCS 252-260 peptide

| | | CTL number per | |
|---|---|---|---|
| | | million of splenic cells | |
| | | After 2^{nd} | After 3^{rd} |
| Group | Adjuvant . . . Antigen | injection | injection |
| 1 | IFA (Freund's incomplet adjuvant) | 0.0 ± 0.0 | 0.2 ± 0.2 |
| 2 | IFA + PyCS 252-260 + P30 | 36.9 ± 2.7 | 4.4 ± 6.2 |
| 3 | OM-197 + Peptide1 | 0.0 ± 0.0 | 0.7 ± 1.0 |
| 4 | OM-197 + Peptide2 | 0.0 ± 0.0 | 0.0 ± 0.0 |
| 5 | OM-197 − Peptide1 | 11.3 ± 12.4 | 36.9 ± 25.6 |
| 6 | OM-197 − Peptide2 | 0.0 ± 0.0 | 0.6 ± 0.9 |
| 7 | OM-197 + OM-197 − Peptide2 | 28.8 ± 7.1 | 20.0 ± 14.1 |
| 8 | OM-197 | 0.0 ± 0.0 | 0.6 ± 0.9 |

### Example 5.6

### Assessing Pyrogenicity of acyl-dipeptide-Like Compounds in Rabbit Model

This experiment is aimed at determining the highest concentration of acyl-dipeptide-like compound bearing a grafted accessory side chain spacer which does not induce fever in rabbits administered an iv. injection of the compound.

Procedure:

15 New Zealand white rabbits are divided into the following groups:

Group 1: 1.0 mg/kg

Group 2: 0.1 mg/kg

Group 3: 0.01 mg/kg

Group 4: 0.001 mg/kg

Group 5: water for injection

On the day of testing, rabbits are tightly held and weighed in boxes designed for this purpose. Temperature probes are inserted in the rectum are

1 hour after probe insertion, temperatures (preinjection) are recorded for 90 minutes and the mean value is calculated.

Each rabbit is then administered an i.v. injection of product or control (sterile water in 0.9% NaCl) via the marginal (lateral) ear vein. Temperature is measured at 30 minute intervals for 3 hours. Difference between the preinjection average temperature and maximum temperature is calculated and reported.

Taking 3 rabbits per group, test products are considered non pyrogenic if the sum of 3 responses does not exceed 1.15° C., or else pyrogenic if said sum exceeds 2.65° C. If the result obtained lies in-betweeen, the test is repeated with 3 more rabbits.

Results

The results of pyrogenicity testing conducted on OM-197-MC-MP and OM-197-MC-MP compounds are reported hereinafter:

| | response in ° C. | |
|---|---|---|
| OM-197-MC-MP dose | average (individual values) | results |
| 1 mg/Kg | 3.40 (1.50, 0.60, 1.30) | pyrogenic |
| 0.1 mg/Kg | 2.00 (1.45, 0.05, 0.50) | non pyrogenic |
| | | (1 high value |
| | | recorded) |
| 0.01 mg/Kg | 0.65 (0.00, 0.45, 0.20) | non pyrogenic |
| 0.001 mg/Kg | 0.35 (0.10, 0.05, 0.20) | non pyrogenic |
| control | 0.20 (0.20, 0.00, 0.00) | non pyrogenic |

| | response in ° C. | |
|---|---|---|
| OM-197-MC-Asp dose | average (individual values) | results |
| 1 mg/Kg | 3.25 (1.25, 1.45, 0.55) | pyrogenic |
| 0.1 mg/Kg | 2.85 (1.10, 1.20, 0.55) | pyrogenic |
| 0.01 mg/Kg | 0.35 (0.20, 0.15, 0.00) | non pyrogenic |
| 0.001 mg/Kg | 0.25 (0.00, 0.10, 0.15) | non pyrogenic |
| control | 0.40 (0.25, 0.05, 0.10) | non pyrogenic |

Compounds in accordance with the invention are non pyrogenic at doses below 0.1 mg/kg.

### Example 2.28

### (3S, 9R) 3-[(R)-3-Dodecanoyloxytetradecanoylamino]-4-oxo-5-aza-9-[(R)-3-hydroxytetradecanoylamino]-decan-1,10-diol 1-(6-aminohexanoate) 10-dihydrogen Phosphate (=OM-197-AC MP) (FIG. 1)

2.28.1. (3S, 9R) 3-[(R)-3-Dodecanoyloxytetradecanoylamino]-4-oxo-5-aza-9-[(R)-3-benzyloxytetradecanoylamino]-decan-1,10-diol 10-(2-tetrahydropyranyl) 1-(6-benzyloxycarbonylaminohexanoate)

Commercial 1-(3-dimethylaminopropyl)-3-ethylcarbodiimide hydrochloride (393 mg; 2.04 mmol; 3 eq) and 4-dimethylaminopyridine (26 mg, 0.21 mmol; 0.3 eq) are successively added to a solution of (3S, 9R) 3-[(R)-3-Dodecanoyloxytetradecanoylamino]-4-oxo-5-aza-9-[(R)-3-benzyloxytetradecanoyl amino]-10-(2-tetrahydropyranyl)oxy-decan-1-ol (Ex. 2.7) (700 mg, 0.68 mmol) and 6-(benzyloxycarbonylamino)hexanoic acid (541 mg, 2.04 mmol) in dry dichloromethane (30 ml) at 0° C. and under argon. The reaction mixture is then stirred for 30 minutes at 0° C. then overnight at ambient temperature. The reaction medium is then diluted with dichloromethane (approximately 40 ml), washed with water, then with a 1N solution of HCl, then with water until the aqueous phase reaches a neutral pH. The organic phase is separated, dried over MgSO₄, filtered and concentrated. Purification by Flash chromatography on silica gel (eluting with CH₂Cl₂/Acetone 7/1 then 5/1) allows the coupling product (711 mg; 81%) to be collected. (Rf=0.75 in CH₂Cl₂/Acetone 7/1; U.V. developer phosphomolybdic acid). ¹³C-NMR (62.89 MHz, CDCl₃), δ in ppm 173.7, 173.3, 171.1, 170.7, 169.7, 156.4, 138.2, 136.6, 127.5-128.3, 100.1, 98.7, 77.0, 71.2, 70.9, 69.8, 68.5, 66.3, 63.2, 62.3, 60.5, 50.1, 48.6, 48.3, 41.8, 41.5, 40.6, 39.2, 34.2, 33.9, 31.7, 30.5, 30.3, 29.0-29.5, 26.0, 25.1, 24.9, 24.4, 22.5, 14.0.

2.28.2. (3S, 9R) 3-[(R)-3-Dodecanoyloxytetradecanoylamino]-4-oxo-5-aza-9-[(R)-3-benzyloxytetradecanoylamino]-decan-1,10-diol 1-(6-benzyloxycarbonylaminohexanoate)

A solution of the compound prepared above (700 mg, 0.55 mmol) in CH₂Cl₂ (7 ml) is added to a 1% solution of HCl in methanol (21 ml) at 0° C. After stirring for 45 minutes at 0° C., the reaction medium is neutralized with a 20% solution of NaHCO₃, diluted with CH₂Cl₂ (50 mL) then the organic phase is separated. The resulting aqueous phase is then extracted with CH₂Cl₂ (3×) then the organic phases are collected together. The organic phase is dried over MgSO₄, filtered and evaporated in order to produce the crude alcohol (651 mg; 99%). (Rf=0.55 in CH₂Cl₂/Acetone 4/1; U.V. and phosphomolybdic developer).

2.28.3. (3S, 9R) 3-[(R)-3-Dodecanoyloxytetradecanoylamino]-4-oxo-5-aza-9-[(R)-3-benzyloxytetradecanoylamino]-decan-1,10-diol 1-(6-benzyloxycarbonylaminohexanoate) 10-(dibenzyl phosphate)

85% dibenzyl-diethyl phosphoramidite (453 μl; 1.25 mmol; 2.3 eq) is added at ambient temperature and under argon to a solution of the alcohol prepared above (650 mg; 0.55 mmol) and 1H-tetrazole (115 mg; 1.64 mmol; 3 eq) in anhydrous THF (25 ml). The formation of white crystals in the reaction medium rapidly becomes apparent. After stirring for 30 minutes, the reaction mixture is taken to −18° C. then a solution of mCPBA (57-86%; 335 mg; 2.0 mmol; 3.7 eq) in CH₂Cl₂ (20 ml) is added. The disappearance of the crystals is observed. After stirring for 45 minutes at ambient temperature, a saturated solution of Na₂S₂O₃ (20 ml) is added then the reaction mixture is stirred for 10 minutes. The solution is then diluted with ether (40 ml), then the organic phase is separated and washed with a saturated solution of Na₂S₂O₃ (5×), and with a saturated solution of NaHCO₃ (2×).

The organic phase is dried over MgSO₄, filtered and evaporated. Purification by Flash chromatography on silica gel (eluting with CH₂Cl₂/Acetone 5/1, 4/1, 2/1 then 1/1) allows dibenzylphosphate (645 mg; 82%) to be obtained in the form of an amorphous solid. (Rf=0.40 in CH₂Cl₂/Acetone 4/1; U.V. and phosphomolybdic developer). ¹³C-NMR (62.89 MHz, CDCl₃), δ in ppm 173.6, 173.4, 171.0, 170.7, 169.7, 156.4, 138.2, 136.7, 135.5, 127.6-128.6, ˜77.0, 71.1, 71.0, 69.5, 68.6, 66.4, 60.6, 50.0, 48.4, 41.6, 41.3, 40.7, 38.8, 34.3, 33.9, 31.8, 31.6, 29.0-29.5, 27.7, 26.1, 25.0, 24.4, 22.6, 14.1. C₈₄H₁₃₁N₄O₁₄P. IS/MS: m/z 1453.0 ([M+H]⁺), 1469.0 ([M+NH₄]⁺), 1475.0 ([M+Na]⁺).

2.28.4. (3S, 9R) 3-[(R)-3-Dodecanoyloxytetradecanoylamino]-4-oxo-5-aza-9-[(R)-3-hydroxytetradecanoylamino]-decan-1,10-diol 1-(6-aminohexanoate) 10-dihydrogen phosphate

A solution of the compound prepared above (540 mg, 0.37 mmol) in a CH₂Cl₂/ethanol mixture 5/2 (70 ml) containing acetic acid (10 ml) is hydrogenated in the presence of 10% Pd on carbon (270 mg) at ambient temperature and under atmospheric pressure of hydrogen for 48 hours. The catalyst is eliminated by filtration. The filtrate is evaporated to dryness, coevaporated with toluene (2×) then the residue is dried with a vane pump in order to produce 3S, 9R) 3-[(R)-3-Dodecanoyloxytetradecanoylamino]-4-oxo-5-aza-9-[(R)-3-hydroxy-tetradecanoyl amino]-decan-1,10-diol-1-(6-aminohexanoate) 10-dihydrogen phosphate (380 mg, 98%). C₅₅H₁₀₇N₄O₁₂P. ES/MS: m/z 1047.9 [M+H]⁺. (FIG. 2)

### Example 2.29

### N-[(R)-3-dodecanoyloxytetradecanoyl]-L-aspartic Aid, α-N-{(4R)-5-(6-aminohexanoyloxy)-4-[(R)-3-hydroxytetradecanoylamino]pentyl}amide β-N-(6-aminohexyl)amide (=OM-197-AH AC) (FIG. 3)

The same sequence of reactions (cf section 1.2.1; section 1.2.2; section 2.7.1 and section 2.7.2) applied to commercial H-L-Asp(OBn)-OH (Fluka, Buchs, Switzerland) leads to the triethylammonium salt of N-[(R)-3-dodecanoyloxytetradecanoyl]-L-aspartic acid, α-N-{(4R)-5-(2-tetrahydropyranyl)oxy-4-[(R)-3-benzyloxytetradecanoyl amino]pentyl}amide.

2.29.1 N-[(R)-3-dodecanoyloxytetradecanoyl]-L-aspartic acid, α-N-{(4R)-5-(2-tetrahydropyranyl)oxy-4-[(R)-3-benzyloxytetradecanoylamino]pentyl}amide β-N-(6-benzyloxycarbonylaminohexyl)amide)

IIDQ (2-isobutoxy-1-isobutoxycarbonyl-1,2-dihydroquinoline) (101 mg, 0.33 mmol) is added at 0° C. and under argon to a solution of the triethylammonium salt of N-[(R)-3-dodecanoyloxytetradecanoyl]-L-aspartic acid, α-N-{(4R)-5-(2-tetrahydropyranyl)oxy-4-[(R)-3-benzyloxytetradecanoylamino] pentyl} amide (250 mg, 0.22 mmol) and commercial 6-benzyloxycarbonylamino-hexylamine hydrochloride (70 mg, 0.24 mmol) in anhydrous CH₂Cl₂ (15 ml). The reaction mixture is stirred for 5 hours at 0° C. then the solution is evaporated to dryness. Purification by Flash chromatography on silica gel (eluting with CH₂Cl₂/Acetone 3/1) allows the coupling product (128 mg; 46% over the 2 stages) to be obtained in the form of an amorphous solid. (Rf=0.48 in CH₂Cl₂/Methanol 9/1; U.V. and phosphomolybdic developer). C₇₅H₁₂₇N₅O₁₁. IS/MS: m/z 1275.0 ([M+H]⁺), 1289.0 ([M+NH₄]⁺), 1297.0 ([M+Na]⁺).

2.29.2 N-[(R)-3-dodecanoyloxytetradecanoyl]-L-aspartic acid, α-N-{(4R)-5-hydroxy-4-[(R)-3-benzyloxytetradecanoylamino]pentyl}amide β-N-(6-benzyloxycarbonylaminohexyl)amide)

A solution of the compound prepared above (900 mg, 0.70 mmol) in CH₂Cl₂ (3 ml) is added at 0° C. to a 1% solution of HCl in methanol (30 ml). After stirring for 45 minutes at 0° C., the reaction medium is neutralized with a 10% solution of NaHCO₃, diluted with CH₂Cl₂ (100 mL) then the organic phase is separated. The resulting aqueous phase, is then extracted with CH₂Cl₂ (3×) then the organic phases are collected together. The organic phase is dried over MgSO₄, filtered and evaporated in order to produce the crude alcohol (826 mg; 98%). (Rf=0.15 in CH₂Cl₂/Acetone 3/1; U.V. and phosphomolybdic developer). C₇₀H₁₁₉N₅O₁₀. IS/MS: m/z 1191.0 ([M+H]⁺), 1213.0 ([M+Na]⁺). mp=148-149° C.

29.3 N-[(R)-3-dodecanoyloxytetradecanoyl]-L-aspartic Acid, α-N-{(4R)-5-[6-(benzyloxycarbonylamino)hexanoyloxy]-4-[(R)-3-benzyloxytetradecanoylamino]pentyl}amide β-N-(6-benzyloxycarbonylaminohexyl) amide)

Commercial 1-(3-dimethylaminopropyl)-3-ethylcarbodiimide hydrochloride (309 mg, 1.62 mmol) and 4-dimethylaminopyridine (20 mg, 0.17 mmol) are successively added at ambient temperature and under argon to a solution of N-[(R)-3-dodecanoyloxytetradecanoyl]-L-aspartic acid, α-N-{(4R)-5-hydroxy-4-[(R)-3-benzyloxytetradecanoylamino]pentyl}amide β-N-(6-benzyloxycarbonylaminohexyl) amide) (641 mg, 0.54 mmol) and 6-(benzyloxy carbonylamino)hexanoic acid (428 mg, 1.62 mmol) in dry CH₂Cl₂ (30 ml). The reaction mixture is then stirred overnight at ambient temperature. The reaction medium is then washed with water then with a 1N solution of HCl then the phases are separated. The organic phase is dried over MgSO₄, filtered and evaporated. Purification by recrystallization (Ethyl acetate) allows the coupling product to be collected (717 mg; 100%). (Rf=0.66 in CH₂Cl₂/acetone 3/1; U.V. and phosphomolybdic developer). mp=150-152° C. ¹³C-NMR (62.89 MHz, CDCl₃), δ in ppm 173.64, 173.26, 171.32, 171.11, 170.60, 170.00, 156.42, 138.20, 136.59 (d), 128.38, 128.33, 127.95, 127.61, 127.52, 71.24, 70.99, 66.42, 65.71, 49.89, 47.80, 41.97, 41.45, 40.72, 40.67, 39.23, 39.11, 37.21, 34.49, 34.37, 33.90, 31.81, 29.54, 29.43, 29.25, 29.07, 28.40, 26.11, 26.00, 25.74, 25.15, 25.05, 24.90, 24.31, 22.59, 14.02.

2.29.4 N-[(R)-3-dodecanoyloxytetradecanoyl]-L-aspartic Acid, α-N-{(4R)-5-(6-aminohexanoyloxy)-4-[(R)-3-hydroxytetradecanoylamino]pentyl}amide β-N-(6-aminohexyl)amide)

A solution of the compound prepared above (690 mg, 0.48 mmol) in a CH₂Cl₂/ethanol mixture 7/3 (100 ml) containing acetic acid (15 ml), is hydrogenated in the presence of 10% Pd on carbon at ambient temperature and under atmospheric pressure of hydrogen for 3 to 8 days. The catalyst is eliminated by filtration. The filtrate is evaporated to dryness, the residue is coevaporated with toluene then dried with a vane pump in order to produce N-[(R)-3-dodecanoyloxytetradecanoyl]-L-aspartic acid, α-N-{(4R)-5-(6-aminohexanoyloxy)-4-[(R)-3-hydroxytetradecanoyl amino]pentyl}amide β-N-(6-aminohexyl)amide) (420 mg, 75%). C₆₁H₁₁₈N₆O₉. ES/MS: m/z 1080.0 ([M+H]⁺). (FIG. 4)

### Example 2.30

### N-[(R)-3-dodecanoyloxytetradecanoyl]-L-aspartic Acid, α-N-{(4R)-5-dihydroxyphosphoryloxy-4-[(R)-3-hydroxytetradecanoylamino]pentyl}amide β-N-(6-aminohexyl)amide (=OM-197-AH MP) (FIG. 5)

2.30.1 N-[(R)-3-dodecanoyloxytetradecanoyl]-L-aspartic Acid, α-N-{(4R)-5-dibenzyloxyphosphoryloxy-4-[(R)-3-benzyloxytetradecanoylamino]pentyl}amide β-N-(6-benzyloxycarbonylaminohexyl)amide)

85% dibenzyl-diethyl phosphoramidite (120 μl; 0.34 mmol) is added at ambient temperature and under argon to a solution of N-[(R)-3-dodecanoyloxytetradecanoyl]-L-aspartic acid, α-N-{(4R)-5-hydroxy-4-[(R)-3-benzyloxytetradecanoylamino]pentyl}amide β-N-(6-benzyloxycarbonylaminohexyl) amide) (section 2.29.2) (174 mg; 0.15 mmol) and 1H-tetrazole (31 mg; 0.44 mmol) in anhydrous THF (8 ml). The formation of white crystals in the reaction medium rapidly becomes apparent. After stirring for 1 hour 15 minutes, the reaction mixture is taken to 0° C. then a solution of mCPBA (57-86%; 93 mg; 0.54 mmol) in CH₂Cl₂ (4 ml) is added. The disappearance of the crystals is observed. After stirring for 1 hour 15 minutes at ambient temperature, a saturated solution of Na₂S₂O₃ (6 ml) is added then the reaction mixture is stirred for 10 minutes. The solution is then diluted with ether, then the organic phase is separated and washed with a saturated solution of Na₂S₂O₃ (5×), a saturated solution of NaHCO₃ (2×). The organic phase is dried over MgSO₄, filtered and evaporated. Purification by Flash chromatography on silica gel (eluting with CH₂Cl₂/acetone 2.5/1 then 1/1) allows N-[(R)-3-dodecanoyloxytetradecanoyl]-L-aspartic acid, α-N-{(4R)-5-dibenzyloxyphosphoryloxy-4-[(R)-3-benzyloxytetradecanoylamino]pentyl}amide β-N-(6-benzyloxycarbonylaminohexyl)amide) (168 mg; 79%) to be obtained in the form of an amorphous solid. (Rf=0.44 in CH₂Cl₂/acetone 3/1; U.V. and phosphomolybdic developer). C₈₄H₁₃₂N₅O₁₃P. IS/MS: m/z 1451.0 ([M+H]⁺), 1474.0 ([M+Na]⁺). mp=109-112° C. ¹³C-NMR (62.89 MHz, CDCl₃), δ0 in ppm 173.67, 171.37, 171.16, 170.73, 170.04, 156.48, 138.38, 136.69, 135.69, 135.59, 128.60, 128.48, 128.36, 128.02, 127.98, 127.73, 127.61, 71.22, 71.05, 69.47, 69.12, 66.51, 53.40, 50.05, 48.66, 47.55, 48.46, 41.99, 41.44, 40.71, 39.34, 39.02, 37.52, 34.52, 34.46, 34.00, 31.90, 29.61, 29.51, 29.34, 29.14, 27.53, 26.20, 26.08, 25.72, 25.23, 24.97, 22.66, 14.10.

2.30.2 N-[(R)-3-dodecanoyloxytetradecanoyl]-L-aspartic Acid, α-N-{(4R)-5-dihydroxyphosphoryloxy-4-[(R)-3-hydroxytetradecanoylamino]pentyl}amide β-N-(6-aminohexyl)amide)

A solution of the compound prepared above (140 mg, 96 μmol) in a mixture of CH₂Cl₂ (14 ml) and ethanol (6 ml) containing acetic acid (3 ml) is hydrogenated in the presence of 10% Pd on carbon, at ambient temperature and under atmospheric pressure of hydrogen for 3 to 8 days. The catalyst is eliminated by filtration. The filtrate is evaporated to dryness, the residue is coevaporated with toluene then dried with a vane pump in order to produce N-[(R)-3-dodecanoyloxytetradecanoyl]-L-aspartic acid, α-N-{(4R)-5-dihydroxyphosphoryloxy-4-[(R)-3-hydroxytetradecanoyl amino]pentyl}amide β-N-(6-aminohexyl)amide) (98 mg, 97%). C₅₅H₁₀₈N₅O₁₁P. IS/MS: m/z 1046.7 ([M+H]⁺). (FIG. 7)

### Example 2.31

### N-[(R)-3-hydroxytetradecanoyl]-L-aspartic Acid, α-N-{(4R)-5-(6-amino hexanoyloxy)-4-[(R)-3-dodecanoyloxytetradecanoylamino]pentyl}amide (=OM-197-MC-AC inv) (FIG. 8)

2.31.1 (2R)-5-(Benzyloxycarbonylamino)-2-amino-pentan-1-ol, hydrochloride

A 1% solution of HCl (w/v) in methanol (200 ml) is added to a solution of [(2R)-5-(benzyloxycarbonylamino)-2-(terbutoxycarbonylamino)pentan-1-ol] compound (section 1.1.2.d) (2.5 g, 7.09 mmol) in HPLC quality ethyl acetate (25 ml) at 0° C. After stirring for 14 hours at ambient temperature, the solvent is evaporated in order to produce (2R)-5-(Benzyloxycarbonylamino)-2-amino-pentan-1-ol hydrochloride (1.98 g, 97%) in the form of a yellowish syrup.

2.31.2 (2R)-5-(Benzyloxycarbonylamino)-2-[(R)-3-dodecanoyloxytetradecanoyl amino]pentan-1-ol

N-methylmorpholine (0.78 ml; 7.09 mmol) and isobutyl chloroformate (0.92 ml; 7.09 mmol) are added to a solution cooled down to −15° C. of (R)-3-dodecanoyl oxytetradecanoic acid (3.03 g, 7.09 mmol) in anhydrous THF (40 ml). After stirring at −10° C. for 1 hour, a solution of (2R)-5-(Benzyloxycarbonylamino)-2-amino-pentan-1-ol hydrochloride (section 2.31.1) (1.98 g; 7.09 mmol) in anhydrous THF (1 ml) and NaHCO₃ (1.19 g, 14.2 mmol) is added. Stirring is continued at ambient temperature for 16 hours then the reaction mixture is diluted with H₂O and ether. The organic phase is separated, washed with H₂O (2×) then dried over MgSO₄ before evaporation is carried out under vacuum. (2R)-5-(Benzyloxycarbonylamino)-2-[(R)-3-dodecanoyloxytetradecanoylamino]pentan-1-ol is thus obtained (4.54 g; 97%), without other purification, in the form of an amorphous solid. (Rf=0.22 in CH₂Cl₂/Acetone 6/1; U.V. and phosphomolybdic developer). ¹H-NMR (CDCl₃, 250 MHz), δ in ppm: 7.4 (s, 5H); 6.1 (m, 1H, NH); 5.2 (m, 1H); 5.1 (s, 2H); 4.9 (m, 1H, NH); 4.0-3.8 (m, 1H); 3.8-3.5 (m, 2H); 3.2 (m, 2H); 2.5 (d, 2H); 2.3 (t, 2H); 1.7-1.5 (m, 8H); 1.4-1.2 (m, 34H); 0.9 (m, 6H). C₃₉H₆₈N₂O₆. IS/MS: m/z 661.5 ([M+H]⁺), 683.5 ([M+Na]⁺), 699.5 ([M+K]⁺).

2.31.3 (2R)-5-amino-2-[(R)-3-dodecanoyloxytetradecanoylamino]pentan-1-ol

A solution of (2R)-5-(Benzyloxycarbonylamino)-2-[(R)-3-dodecanoyloxytetradecanoylamino]pentan-1-ol (189 mg; 0.29 mmol) in ethanol (17 ml) is hydrogenated in the presence of 10% Pd on carbon at ambient temperature and under atmospheric pressure of hydrogen for 2 hours 30 minutes. The catalyst is eliminated by filtration, followed by washing with ethanol and the filtrate is evaporated to dryness then dried with a vane pump in order to produce the free amine (123 mg; 81%) in the form of a translucent amorphous solid. ¹H-NMR (CDCl₃, 250 MHz), δ in ppm: 5.2 (m, 1H); 3.9 (m, 1H); 3.6 (m, 2H); 3.1 (m, 2H); 2.5 (m, 2H); 2.3 (t, 2H); 1.9-1.5 (m, 8H); 1.3 (m, 34H); 0.9 (m, 6H).

2.31.4. N-[(R)-3-benzyloxytetradecanoylamino]-L-aspartic Acid, β-benzyl Ester

N-methylmorpholine (250 μl; 2.2 mmol; 1 eq) and isobutyl chloroformate (291 μl; 2.2 mmol; 1 eq) are added successively to a solution of (R)-3-benzyloxytetradecanoic acid (750 mg; 2.2 mmol) [Bull. Chem. Soc. Jpn, 60 (1987), 2197-2204] in anhydrous THF (7.2 ml) at −15° C. and under argon. After stirring for 45 minutes at −15° C., a solution of commercial H-L-Asp(OBn)-OH (Fluka, Buchs, Switzerland) (500 mg; 2.2 mmol; 1 eq) in a CH₃CN/H₂O mixture 3.5/1 (24 ml) containing Et₃N (1 ml) is then added. The reaction mixture is then stirred overnight at ambient temperature. The organic solvent is then evaporated, then the aqueous phase is cooled down to 0° C., acidified to pH=3 with a 10% aqueous solution of citric acid followed by extraction with AcOEt (3×). The organic phase is dried over MgSO₄, filtered and evaporated. Purification by Flash chromatography on silica gel (eluting with petroleum ether/AcOEt 2/1 containing 2% acetic acid) followed by coevaporation with toluene allows the β-benzyl ester of N-[(R)-3-benzyloxytetradecanoylamino]-L-aspartic acid (1.19 g; 99%) to be collected in the form of a white crystalline solid (Rf=0.27 in petroleum ether/EtOAc 1/1 containing 2% acetic acid; U.V. and phosphomolybdic developers). ¹H-NMR (CDCl₃, 250 MHz), δ in ppm: 7.3 (m, 10H); 5.0 (s, 2H); 4.9 (m, 1H); 4.5 (q, 2H); 3.8 (m, 1H); 3.1-2.8 (dd, 2H); 2.5 (m, 2H); 1.6 (m, 2H); 1.3 (m, 18H); 0.9 (m, 3H).

2.31.5. N-[(R)-3-benzyloxytetradecanoyl]-L-aspartic Acid, α-N-{(4R)-5-hydroxy-4-[(R)-3-dodecanoyloxytetradecanoylamino]pentyl}amide β-benzyl Ester Commercial HOOBt (3-hydroxy-1,2,3-benzotriazin-4(3H)-one) (ACROS, Basel, Switzerland) (19 mg, 114 μmol, 1 eq) then commercial N,N′-diisopropylcarbodiimide (23 μl, 114 μmol, 1 eq) are added successively to a solution of the β-benzyl ester obtained above (61 mg; 114 μmol) and (2R)-5-amino-2-[(R)-3-dodecanoyloxytetradecanoylamino]pentan-1-ol (Section 2.31.3) (60 mg; 114 μmol; 1.0 eq) in anhydrous CH₂Cl₂ (1.4 ml) at ambient temperature and. under argon. The reaction mixture is stirred overnight at ambient temperature. The reaction medium is then diluted with CH₂Cl₂, washed with water, with a 1N solution of HCl, with a saturated solution of NaHCO₃ then the phases are separated. The organic phase is dried over MgSO₄, filtered and evaporated. The residue is taken up in CH₂Cl₂ then the solution is cooled down to 0° C. for 2 hours. The diisopropylurea precipitate is then eliminated by filtration, washed with cold CH₂Cl₂ and the solvent is evaporated off. Purification by Flash chromatography on silica gel (eluting with CH₂Cl₂/acetone 3/1) allows the coupling product (58 mg; 49%) to be collected in the form of a white crystalline solid (Rf=0.50 in CH₂Cl₂/acetone 3/1; U.V. and phosphomolybdic developers). ¹³C-NMR (62.89 MHz, CDCl₃), δ in ppm 174.21; 171.91; 170.78; 170.62; 138.25; 135.67; 128.9; 128.8; 128.7; 128.5; 128.21; 76.88; 71.78; 71.66; 67.1; 64.89; 51.71; 49.79; 42.31; 41.62; 39.34; 35.78; 34.86; 34.7; 32.22; 29.95; 29.66; 29.49; 28.02; 26.24; 25.6; 25.54; 25.33; 23.75; 22.99; 14.43.

2.31.6. N-[(R)-3-benzyloxytetradecanoyl]-L-aspartic Acid, α-N-{(4R)-5-(6-(benzyloxycarbonylamino)hexanoyloxy)-4-[(R)-3-dodecanoyloxytetradecanoylamino]pentyl}amide β-benzyl Ester

Commercial 1-(3-dimethylaminopropyl)-3-ethylcarbodiimide hydrochloride (33 mg, 168 μmol) and 4-dimethylaminopyridine (3 mg, 17 μmol) are added successively to a solution of the β-benzyl ester obtained above (58 mg, 56 βmol) and 6-(benzyloxy carbonylamino)hexanoic acid (45 mg, 168 μmol) in dry CH₂Cl₂ (2.6 ml) at 0° C. and under argon. The reaction mixture is stirred for 30 minutes at 0° C. and then overnight at ambient temperature. The reaction medium is then diluted with CH₂Cl₂, washed with water with a 1N solution of HCl then the phases are separated. The organic phase is dried over MgSO₄, filtered and evaporated. Purification by Flash chromatography on silica gel (eluting with CH₂Cl₂/acetone 5/1) allows the coupling product (57 mg; 79%) to be collected in the form of a white crystalline solid (Rf=0.80 in CH₂Cl₂/acetone 3/1; U.V. and phosphomolybdic developer). ¹³C-NMR (62.89 MHz, CDCl₃), δ in ppm 173.83; 173.77; 171.9; 171.72; 170.79; 169.94; 156.79; 138.32; 136.96; 135.71; 128.89; 128.81; 128.68; 128.49; 128.37; 128.20; 76.89; 71.81; 71.56; 67.04; 66.87; 66.03; 49.62; 48.52; 42.06; 41.65; 41.1; 39.25; 35.83; 34.86; 34.53; 34.19; 34.08; 32.22; 29.95; 29.72; 29.66; 29.50; 28.43; 26.42; 26.23; 26.0; 25.58; 25.36; 22.99; 14.43.

2.31.7. N-[(R)-3-hydroxytetradecanoyl]-L-aspartic Acid, α-N-{(4R)-5-(6-aminon hexanoyloxy)-4-[(R)-3-dodecanoyloxytetradecanoylamino]pentyl}amide

A solution of the compound prepared above (47 mg, 36 μmol) in a CH₂Cl₂/methanol mixture 5/1 (3.3 ml) containing acetic acid (280 μl) is hydrogenated in the presence of 10% Pd on carbon at ambient temperature and under atmospheric pressure of hydrogen for 72 hours. The catalyst is eliminated by filtration. The filtrate is evaporated to dryness then dried with a vane pump in order to produce N-[(R)-3-hydroxytetradecanoyl]-L-aspartic acid, α-N-{(4R)-5-(6-aminohexanoyloxy)-4-[(R)-3-dodecanoyloxytetradecanoylamino]pentyl}amide (35 mg, quantitative yield). C₅₅H₁₀₄N₄O₁₀. ES/MS: m/z 981.9 ([M+H]⁺). (FIG. 9)

### Example 2.32

### N-[(R)-3-dodecanoyloxytetradecanoyl]-L-aspartic Acid, α-N-{(4R)-5-(6-amino hexanoyloxy)-4-[(R)-3-dodecanoyloxytetradecanoylamino]pentyl}amide (=OM-197-MC-AC tetracyl) (FIG. 10)

The same reaction (cf section 1.2.1) applied to commercial H-L-Asp(OBn)-OH (Fluka, Buchs, Switzerland) leads to the β-benzyl ester of N-[(R)-3-dodecanoyloxytetradecanoyl]-L-aspartic acid.

2.32.1. N-[(R)-3-dodecanoyloxytetradecanoyl]-L-aspartic Acid, α-N-{(4R)-5-hydroxy-4-[(R)-3-dodecanoyloxytetradecanoylamino]pentyl}amide β-benzyl Ester

Commercial HOOBt (3-hydroxy-1,2,3-benzotriazin-4(3H)-one) (ACROS, Basel, Switzerland) (349 mg, 207 μmol, 1 eq) then commercial N,N′-diisopropylcarbodiimide (41 μl, 207 μmol, 1 eq) are added successively to a solution of the β-benzyl ester of N-[(R)-3-dodecanoyloxytetradecanoyl]-L-aspartic acid (131 mg; 207 μmol) and (2R)-5-amino-2-[(R)-3-dodecanoyloxytetradecanoylamino]pentan-1-ol (Section 2.31.3) (109 mg; 207 μmol; 1.0 eq) in anhydrous CH₂Cl₂ (2.6 ml) at ambient temperature and under argon. The reaction mixture is stirred for 4 hours at ambient temperature. The reaction medium is then diluted with CH₂Cl₂, washed with water, with a 1 N solution of HCl, with a saturated solution of NaHCO₃ then the phases are separated. The organic phase is dried over MgSO₄, filtered and evaporated. Purification by Flash chromatography on silica gel (eluting with CH₂Cl₂/acetone 3/1 then 2/1) allows the coupling product (122 mg; 52%) to be collected in the form of a white crystalline solid (Rf=0.55 in CH₂Cl₂/acetone-3/1; U.V. and phosphomolybdic developers). ¹³C-NMR (62.89 MHz, CDCl₃), δ in ppm 174.18; 172.02; 170.72; 170.60; 157.53; 135.67; 128.87; 128.65; 128.43; 71.69; 71.36; 67.07; 64.81; 51.68; 49.63; 42.33; 42.15; 39.58; 36.08; 34.87; 34.77; 32.21; 29.94; 29.83; 29.72; 29.65; 29.49; 26.09; 25.6; 25.54; 25.34; 25.29; 22.98; 14.41.

2.32.2. N-[(R)-3-dodecanoyloxytetradecanoyl]-L-aspartic Acid, α-N-{(4R)-5-(6-(benzyloxycarbonylamino)hexanoyloxy)-4-[(R)-3-dodecanoyloxytetra decanoylamino]pentyl}amide β-benzyl Ester

Commercial 1-(3-dimethylaminopropyl)-3-ethylcarbodiimide hydrochloride (85 mg, 321 μmol) and 4-dimethylaminopyridine (4 mg, 32 μmol) are successively added to a solution of the β-benzyl ester obtained above (122 mg, 107 μmol) and 6-(benzyloxycarbonylamino)hexanoic acid (86 mg, 107 μmol) in dry CH₂Cl₂ (5 ml) at 0° C. and under argon. The reaction mixture is stirred for 30 minutes at 0° C. and then stirred overnight at ambient temperature. The reaction medium is then diluted with CH₂Cl₂, washed with water then with a 1N solution of HCl then the phases are separated. The organic phase is dried over MgSO₄, filtered and evaporated. Purification by Flash chromatography on silica gel (eluting with CH₂Cl₂/acetone 5/1) allows the coupling product (97 mg; 65%) to be collected in the form of a white crystalline solid (Rf=0.77 in CH₂Cl₂/acetone 3/1; U.V. and phosphomolybdic developer). ¹³C-NMR (62.89 MHz, CDCl₃), δ in ppm 172.13; 170.7; 170.43; 169.93; 156.76; 136.93; 135.68; 128.88; 128.78; 128.64; 128.4; 128.34; 71.60; 71.34; 67.03; 66.83; 65.97; 49.46; 48.55; 42.20; 42.10; 41.08; 39.35; 36.00; 34.86; 34.74; 34.51; 34.17; 32.20; 29.93; 29.81; 29.70; 29.63; 29.48; 29.44; 28.57; 26.39; 26.10; 25.60; 25.56; 25.35; 25.27; 24.71; 22.97; 14.40.

2.32.3. N-[(R)-3-dodecanoyloxytetradecanoyl]-L-aspartic Acid, α-N-{(4R)-5-(6-aminohexanoyloxy)-4-[(R)-3-dodecanoyloxytetradecanoylamino]pentyl}amide

A solution of the compound prepared above (90 mg, 65 μmol) in a CH₂Cl₂/methanol mixture 5/1 (6 ml) containing acetic acid (500 μl) is hydrogenated in the presence of 10% Pd on carbon at ambient temperature and under atmospheric pressure of hydrogen for 72 hours. The catalyst is eliminated by filtration. The filtrate is evaporated to dryness then dried with a vane pump in order to produce N-[(R)-3-dodecanoyloxytetradecanoyl]-L-aspartic acid, α-N-{(4R)-5-(6-aminohexanoyloxy)-4-[(R)-3-dodecanoyloxytetradecanoylamino]pentyl}amide (76 mg, quantitative yield). C₆₇H₁₂₆N₄O₁₁. ES/MS: m/z 1163.9 ([M+H]⁺). (FIG. 11)

### Example 2.33

### (3S, 9R)-3-[(R)-3-Dodecanoyloxytetradecanoylamino]-4-oxo-5-aza-9-[(R)-3-hydroxytetradecanoylamino]-decan-1,10-diol 1-dihydrogen phosphate 10-(3-aminopropanoate) (=OM-197-MP-AP) (FIG. 12)

2.33.1. (2R)-5-(Benzyloxycarbonylamino)-2-[(R)-3-benzyloxytetradecanoylamino]-1-(2-tetrahydropyranyloxy)-pentane

3,4-Dihydro-2H-pyrane (DHP) (1.4 ml, 15.38 mmol) then pyridinium p-toluenesulphonate (PPTS) (441 mg, 1.75 mmol) are added successively to a solution of (2R)-5-(Benzyloxycarbonylamino)-2-[(R)-3-benzyloxytetradecanoylamino]pentan-1-ol (section 1.1.2.f) (2.5 g; 4.39 mmol) in anhydrous CH₂Cl₂ (83 ml) at ambient temperature and under argon. The solution is then stirred for 18 hours at ambient temperature then diluted with CH₂Cl₂ (100 ml), washed with a 5% solution of NaHCO₃ then with H₂O. The organic phase is dried over MgSO₄, filtered and evaporated. Purification by Flash chromatography on silica gel (eluting with AcOEt/petroleum ether 4/1) allows (2R)-5-(Benzyloxycarbonylamino)-2-[(R)-3-benzyloxytetradecanoylamino]-1-(2-tetrahydropyranyloxy)-pentane (2.86 g; 100%) to be collected in the form of a white crystalline solid. (Rf=0.66 in AcOEt/petroleum ether 4/1; U.V. and phosphomolybdic developer). C₃₉H₆₀N₂O₆. IS/MS: m/z 653.5 ([M+H]⁺), 675.5 ([M+Na]⁺). mp=84-86° C.

2.33.2. (2R)-5-amino-2-[(R)-3-benzyloxytetradecanoylamino]-1-(2-tetrahydropyranyloxy)-pentane

A solution of (2R)-5-(Benzyloxycarbonylamino)-2-[(R)-3-benzyloxytetradecanoylamino]-1-(2-tetrahydropyranyloxy)-pentane (2.5 g; 4.4 mmol) in ethanol (150 ml) containing triethylamine (4 ml) is hydrogenated in the presence of 10% Pd on carbon at ambient temperature and under atmospheric pressure of hydrogen for 3 hours 30 minutes. The catalyst is eliminated by filtration, followed by washing with ethanol and the filtrate is evaporated to dryness then dried with a vane pump in order to produce the free amine (2.15 g; 96%) in the form of an amorphous white solid. C₃₁H₅₄N₂O₄. IS/MS: m/z 519.5 ([M+H]⁺).

2.33.3. (S)-(α)-[(R)-3-dodecanoyloxytetradecanoylamino]-γ-butyrolactone

N-methylmorpholine (0.56 ml; 5.1 mmol; 1 eq) and isobutyl chloroformate (657 μl; 5.1 mmol; 1 eq) are added successively to a solution of (R)-3-dodecanoyloxytetradecanoic acid (2.16 g; 5.1 mmol) in anhydrous THF (28 ml) at −15° C. and under argon. After stirring for 1 hour at −15° C., commercial L-homoserine lactone hydrobromide (916 mg; 5.1 mmol; 1 eq) dissolved in a 0.72 M solution of NaHCO₃ (14 ml, 2 eq) is then added. The reaction mixture is then stirred for 20 hours at ambient temperature. The reaction medium is diluted with Et₂O (130 ml), the organic phase is separated and washed with H₂O then dried over MgSO₄, filtered and concentrated under reduced pressure. Purification by crystallization (minimum amount of CH₂Cl₂ and excess pentane at 0° C.) allows the coupling product (2.17 g; 85%) to be collected in the form of a white solid. (Rf=0.41 in petroleum ether/EtOAc 1/1; phosphomolybdic developer). C₃₀H₅₅NO₅. IS/MS: m/z 510.5 ([M+H]⁺), 532.5 ([M+Na]⁺). mp=79-80° C.

2.33.4. (3S,9R)-3-[(R)-3-Dodecanoyloxytetradecanoylamino]-4-oxo-5-aza-9-[(R)-3-benzyloxytetradecanoylamino]-10-(2-tetrahydropyranyl)oxy-decan-1-ol

(S)-(α)-[(R)-3-dodecanoyloxytetradecanoylamino]-γ-butyrolactone (section 2.33.3) (483 mg, 0.95 mmol) is added at 20-21° C. to a solution of (2R)-5-amino-2-[(R)-3-benzyloxytetradecanoylamino]-1-(2-tetrahydropyranyloxy)-pentane (section 2.33.2) (638 mg, 1.23 mmol, 1.3 eq) in anhydrous CH₂Cl₂ (1.5 ml). The solution is stirred for 3 days at 20-21° C. then the solvent is evaporated off under reduced pressure. Purification by Flash chromatography on silica gel (eluting with CH₂Cl₂/acetone 5/1 to 1/1) allows the alcohol (829 mg, 85%) to be collected in the form of a white solid. (Rf=0.37 in CH₂Cl₂/Acetone 2/1; U.V. and phosphomolybdic developers). C₆₁H₁₀₉N₃O₉. IS/MS: m/z 1029.0 ([M+H]⁺), 1051.0 ([M+Na]⁺). mp=81-82° C.

2.33.5. (3S,9R)-3-[(R)-3-Dodecanoyloxytetradecanoylamino]-4-oxo-5-aza-9-[(R)-3-benzyloxytetradecanoylamino]-decan-1,10-diol 1-(dibenzyl phosphate) 10-(3-benzyloxycarbonylaminopropanoate)

The same reaction sequence (cf section 2.7.4 and section 2.7.5) applied to the product prepared above, (3S, 9R)-3-[(R)-3-Dodecanoyloxytetradecanoylamino]-4-oxo-5-aza-9-[(R)-3-benzyloxytetradecanoylamino]-10-(2-tetrahydropyranyl)oxy-decan-1-ol (section 2.33.4) leads to (3S, 9R)-3-[(R)-3-Dodecanoyloxytetradecanoylamino]-4-oxo-5-aza-9-[(R)-3-benzyloxytetradecanoylamino]-decan-1,10-diol 1-(dibenzyl phosphate).

Commercial 1-(3-dimethylaminopropyl)-3-ethylcarbodiimide hydrochloride (239 mg, 1.25 mmol) and 4-dimethylaminopyridine (15 mg, 125 μmol) are successively added to a solution of this compound (500 mg, 0.41 mmol) and commercial N-CBz-β-alanine (278 mg, 1.24 mmol) in dry CH₂Cl₂ (20 ml) at 0° C. and under argon. The reaction mixture is then stirred for 30 minutes at 0° C. then overnight at ambient temperature. The reaction medium is then washed with water then with a 1N solution of HCl then the phases are separated. The organic phase is dried over MgSO₄, filtered and evaporated. Purification by Flash chromatography on silica gel (eluting with CH₂Cl₂/acetone 5/1 then 1/2) allows the coupling product (562 mg; 96%) to be collected. (Rf=0.68 in CH₂Cl₂/Acetone 2/1; U.V. and phosphomolybdic developers). C₈₄H₁₂₅N₄O₁₄P. IS/MS: m/z 1410.0 ([M+H]⁺), 1432.0 ([M+Na]⁺).

2.33.6. (3S, 9R)-3-[(R)-3-Dodecanoyloxytetradecanoylamino]-4-oxo-5-aza-9-[(R)-3-hydroxytetradecanoylamino]-decan-1,10-diol 1-dihydrogen Phosphate 10-(3-aminopropanoate).

A solution of the compound prepared above (540 mg, 0.38 mmol) in a CH₂Cl₂ (54 ml) and ethanol (20 ml) mixture containing acetic acid (10.8 ml) is hydrogenated in the presence of 10% Pd on carbon at ambient temperature and under atmospheric pressure of hydrogen for 36 hours. The catalyst is eliminated by filtration. The filtrate is evaporated to dryness then dried with a vane pump in order to produce (3S, 9R)-3-[(R)-3-Dodecanoyloxytetradecanoylamino]-4-oxo-5-aza-9-[(R)-3-hydroxytetradecanoylamino]-decan-1,10-diol 1-dihydrogen phosphate 10-(3-aminopropanoate) (358 mg, 93%). C₅₂H₁₀₁N₄O₁₂P. ES/MS: m/z 1005.6 ([M+H]⁺). (FIG. 13)

### Example 2.34

### (3S, 9R)-3-[(R)-3-Dodecanoyloxytetradecanoylamino]-4-oxo-5-aza-9-[(R)-3-hydroxytetradecanoylamino]-decan-1,10-diol 1-dihydrogen Phosphate 10-(12-aminododecanoate) (=OM-197-MP-AD) (FIG. 14)

2.34.1. (3S,9R)-3-[(R)-3-Dodecanoyloxytetradecanoylamino]-4-oxo-5-aza-9-[(R)-3-benzyloxytetradecanoylamino]-decan-1,10-diol 1-(dibenzyl Phosphate) 10-(12-benzyloxycarbonylaminododecanoate)

Commercial 1-(3-dimethylaminopropyl)-3-ethylcarbodiimide hydrochloride (157 mg, 0.82 mmol) and 4-dimethylaminopyridine (10 mg, 82 μmol) are successively added to a solution of (3S, 9R)-3-[(R)-3-Dodecanoyloxytetradecanoylamino]-4-oxo-5-aza-9-[(R)-3-benzyloxytetradecanoylamino]-decan-1,10-diol 1-(dibenzyl phosphate) (section 2.33.5) (659 mg, 0.55 mmol) and commercial 12-(benzyloxycarbonylamino)dodecanoic acid (287 mg, 0.82 mmol) in dry CH₂Cl₂ (25 ml) at 0° C. and under argon,. The reaction mixture is then stirred for 30 minutes at 0° C. then overnight at ambient temperature. The reaction medium is then washed with water then with a 1N solution of HCl then the phases are separated. The organic phase is dried over MgSO₄, filtered and evaporated. Purification by Flash chromatography on silica gel (eluting with CH₂Cl₂/acetone 4/1 then 2/1) allows the coupling product (840 mg; 80%) to be collected. (Rf=0.70 in CH₂Cl₂/Acetone 3/1; U.V. and phosphomolybdic developers). ¹³C-NMR (62.89 MHz, CDCl₃), δ in ppm: 173.35, 171.18, 170.49, 169.40, 156.31, 138.28, 136.61, 135.53, 135.43, 135.36, 128.54, 128.41, 128.33, 127.97, 127.88, 127.57, 76.49, 71.25, 71.01, 69.74, 69.63, 69.52, 66.46, 65.64, 64.67, 64.58, 53.36, 49.85, 47.89, 41.62, 41.52, 41.03, 39.15, 34.41, 34.33, 34.00, 33.91, 33.57, 33.49, 31.82, 30.84, 29.87, 29.56, 29.42, 29.27, 29.17, 29.09, 29.04, 28.59, 26.64, 25.39, 25.15, 25.09, 24.94, 24.72, 22.61, 14.05.

2.34.2. (3S,9R)-3-[(R)-3-Dodecanoyloxytetradecanoylamino]-4-oxo-5-aza-9-[(R)-3-hydroxytetradecanoylamino]-decan-1,10-diol 1-dihydrogen Phosphate 10-(12-aminododecanoate).

A solution of the compound prepared above (607 mg, 0.40 mmol) in a CH₂Cl₂/ethanol mixture 5/2 (70 ml) containing acetic acid (12 ml) is hydrogenated in the presence of 10% Pd on carbon at ambient temperature and under atmospheric pressure of hydrogen for 12 to 24 hours. The catalyst is eliminated by filtration. The filtrate is evaporated to dryness then dried with a vane pump in order to produce the (3S, 9R)-3-[(R)-3-Dodecanoyloxytetradecanoylamino]-4-oxo-5-aza-9-[(R)-3-hydroxytetradecanoylamino]-decan-1,10-diol 1-dihydrogen phosphate 10-(12-aminododecanoate) (429 mg, 96%). C₆₁H₁₁₉N₄O₁₂P. ES/MS: m/z 1131.8 ([M+H]⁺), 1153.8 ([M+Na]⁺). (FIG. 15)

### Example 2.35

### N-[(R)-3-dodecanoyloxytetradecanoyl]-L-aspartic Acid, α-N-{(4R)-5-hydroxy-4-[(R)-3-hydroxytetradecanoylamino]pentyl}amide β-N-(6-aminohexyl)amide (=OM-197-AH OH) (FIG. 5)

2.35.1 N-[(R)-3-dodecanoyloxytetradecanoyl]-L-aspartic Acid, α-N-{(4R)-5-hydroxy-4-[(R)-3-hydroxytetradecanoylamino]pentyl}amide β-N-(6-aminohexyl)amide)

A solution of N-[(R)-3-dodecanoyloxytetradecanoyl]-L-aspartic acid, α-N-{(4R)-5-hydroxy-4-[(R)-3-benzyloxytetradecanoylamino]pentyl}amide β-N-(6-benzyloxycarbonylaminohexyl)amide) (section 2.29.2) (80 mg, 0.67 mmol) in a CH₂Cl₂/ethanol mixture 1/1 (10 ml) containing acetic acid (1 ml) is hydrogenated in the presence of 10% Pd on carbon at ambient temperature and under atmospheric pressure of hydrogen for 18 hours. The catalyst is eliminated by filtration. The filtrate is evaporated to dryness, the residue is coevaporated with toluene then dried with a vane pump in order to produce N-[(R)-3-dodecanoyloxytetradecanoyl]-L-aspartic acid, α-N-{(4R)-5-hydroxy-4-[(R)-3-hydroxytetradecanoylamino]pentyl}amide β-N-(6-aminohexyl)amide) (65 mg, 100%). C₅₅H₁₀₇N₅O₈. ES/MS: m/z 967.0 ([M+H]⁺), 989.0 ([M+Na]⁺). (FIG. 6)

### Example 2.36

### (3S, 9R)-3-[(R)-3-hydroxytetradecanoylamino]-4-oxo-5-aza-9-[(R)-3-dodecanoyloxytetradecanoylamino]-decan-1,10-diol 1-dihydrogen Phosphate 10-(6-aminohexanoate) (=OM-197-MP AC-inv) (FIG. 16)

2.36.1. (2R)-5-(Benzyloxycarbonylamino)-2-[(R)-3-dodecanoyloxytetradecanoylamino]-1-(2-tetrahydropyranyloxy)-pentane

3,4-Dihydro-2 H-pyran (DHP) (2.19 ml, 0.024 mol) then pyridinium p-toluenesulphonate (PPTS) (961 mg, 2.75 mmol) are added successively to a solution of (2R)-5-(Benzyloxycarbonylamino)-2-[(R)-3-dodecanoyloxytetradecanoylamino]pentan-1-ol (section 2.31.2) (4.54 g, 6.87 mmol) in anhydrous CH₂Cl₂ (130 ml) at ambient temperature and under argon. The solution is then stirred for 16 hours at ambient temperature then diluted with CH₂Cl₂ (100 ml), washed with a 5% solution of NaHCO₃ then with H₂O. The organic phase is dried over MgSO₄, filtered and evaporated. Purification by Flash chromatography on silica gel (eluting with CH₂Cl₂/acetone 9/1) allows (2R)-5-(Benzyloxycarbonylamino)-2-[(R)-3-dodecanoyloxytetradecanoylamino]-1-(2-tetrahydropyranyloxy)-pentane (3.88 g; 76%) to be collected in the form of an amorphous solid. (Rf=0.74 in CH₂Cl₂/acetone 5/1; U.V. and phosphomolybdic developer). C₄₄H₇₆N₂O₇. IS/MS: m/z. 745.5 ([M+H]⁺), 768.0 ([M+Na]⁺), 783.5 ([M+K])⁺

2.36.2. (2R)-5-amino-2-[(R)-3-dodecanoyloxytetradecanoylamino]-1-(2-tetrahydropyranyloxy)-pentane

A solution of (2R)-5-(Benzyloxycarbonylamino)-2-[(R)-3-dodecanoyloxytetradecanoylamino]-1-(2-tetrahydropyranyloxy)-pentane (3.79 g, 5.21 mmol) in ethanol (150 ml) containing triethylamine (4.6 ml) is hydrogenated in the presence of 10% Pd on carbon at ambient temperature and under atmospheric pressure of hydrogen for 3 hours. The catalyst is eliminated by filtration, washed with ethanol and the filtrate is evaporated to dryness then dried with a vane pump in order to produce the free amine (3.2 g, 100%) in the form of a pale yellow solid. C₃₆H₇₀N₂O₅. IS/MS: m/z. 611.5 ([M+H]⁺), 633.5 ([M+Na]⁺).

2.36.3. (S)-(α)-[(R)-3-Benzyloxytetradecanoylamino]-γ-butyrolactone

N-methylmorpholine (497 μl, 4.48 mmol; 1 eq) and isobutyl chloroformate (585 μl, 4.48 mmol; 1 eq) are added successively to a solution of (R)-3-benzyloxytetradecanoic acid (1.5 g, 4.48 mmol) [Bull. Chem. Soc. Jpn, 60 (1987), 2197-2204] in anhydrous THF (25 ml) at −15° C. and under argon. After stirring for 1 hour 15 minutes at −15° C., commercial L-homoserine lactone hydrobromide (815 mg, 4.48 mmol) then a solution of NaHCO₃ (757 mg) in H₂O (12 ml) are then added. The reaction mixture is then stirred for 16 hours at ambient temperature. The reaction medium is diluted with Et₂O (50 mL), the organic phase is separated and washed with H₂O then dried over MgSO₄, filtered and concentrated under reduced pressure. Purification by crystallization (minimum quantity of CH₂Cl₂ and excess pentane at 0° C.) allows the coupling product (1.59 g; 85%) to be collected in the form of a white solid. (Rf=0.80 in CH₂Cl₂/Acetone 2/1; phosphomolybdic developer). C₂₅H₃₉NO₄. IS/MS: m/z 418.5 ([M+H]⁺), 440.5 ([M+Na]⁺).

2.36.4. (3S, 9R)-3-[(R)-3-Benzyloxytetradecanoylamino]-4-oxo-5-aza-9-[(R)-3-dodecanoyloxytetradecanoylamino]-10-(2-tetrahydropyranyl)oxy-decan-1-ol

(S)-(α)-[(R)-3-benzyloxytetradecanoylamino]-γ-butyrolactone (section 2.36.3) (421 mg, 1 mmol) is added at 25° C. to a solution of (2R)-5-amino-2-[(R)-3-30 dodecanoyloxytetradecanoylamino]-1-(2-tetrahydropyranyloxy)-pentane (section 2.36.2) (800 mg, 1.3 mmol, 1.3 eq) in anhydrous CH₂Cl₂ (2 ml). The solution is stirred for 72 hours at 25° C. then triethylamine (140 μl, 1 mmol, 1 eq) is added. The reaction mixture is stirred at this temperature for 10 days then the solvent is evaporated off under reduced pressure. Purification by Flash chromatography on silica gel (eluting with CH₂Cl₂/acetone 4/3 then acetone) allows the alcohol (587 mg, 57%) to be collected in the form of an amorphous solid. (Rf=0.52 in CH₂Cl₂/Acetone 2/1; U.V. and phosphomolybdic developers). C₆₁H₁₀₉N₃O₉. IS/MS: m/z 1029.0 ([M+H]⁺), 1151.0 ([M+Na]⁺).

2.36.5. (3S,9R)-3-[(R)-3-Benzyloxytetradecanoylamino]-4-oxo-5-aza-9-[(R)-3-dodecanoyloxytetradecanoylamino]-10-(2-tetrahydropyranyl)oxy-decan-1-ol 1-(dibenzylphosphate)

85% dibenzyl-diethyl phosphoramidite (392 μl; 1.31 mmol; 2.3 eq) is added to a solution of the alcohol prepared above (585 mg; 0.57 mmol) and z1H-tetrazole (120 mg; 1.71 mmol; 3 eq) in anhydrous THF (23 ml) at ambient temperature and under argon. The formation of white crystals in the reaction medium rapidly becomes apparent. After stirring for 45 minutes at ambient temperature, a solution of mCPBA (57-86%; 360 mg; 2.10 mmol; 3.7 eq) in CH₂Cl₂ (15 ml) is added. The disappearance of the crystals is observed. After stirring for 45 minutes at ambient temperature, a saturated solution of Na₂S₂O₃ (20 ml) is added then the reaction mixture is stirred for 10 minutes. The solution is then diluted with ether, the organic phase is separated and washed with a saturated solution of Na₂S₂O₃ (5×), and with a saturated solution of NaHCO₃ (2×).

The organic phase is dried over MgSO₄, filtered and evaporated. Purification by Flash chromatography on silica gel (eluting with CH₂Cl₂/acetone 6/1, 5/1 then 1/1) allows the dibenzylphosphate (644 mg; 88%) to be collected in the form of an amorphous solid. (Rf=0.25 in CH₂Cl₂/Acetone 4/1; U.V. and phosphomolybdic developer). C₇₅H₁₂₂N₃O₁₂P. IS/MS: m/z 1289.0 ([M+H]⁺), 1312.0 ([M+Na]⁺).

2.36.6. (3S, 9R)-3-[(R)-3-Benzyloxytetradecanoylamino]-4-oxo-5-aza-9-[(R)-3-dodecanoyloxytetradecanoylamino]-decan-1,10-diol 1-(dibenzyl Phosphate)

A solution of the compound prepared above (634 mg, 0.49 mmol) in CH₂Cl₂ (2 ml) is added at 0° C. to a 1% solution of HCl in methanol (23 ml). After stirring for 1 hour at 0° C., the reaction medium is neutralized with a 10% solution of NaHCO₃, diluted with CH₂Cl₂ then the organic phase is separated. The resulting aqueous phase is then extracted with CH₂Cl₂ (3×) then the organic phases are collected together. The organic phase is dried over MgSO₄, filtered and evaporated in order to produce the crude alcohol (579 mg; 98%) in the form of an amorphous solid. (Rf=0.29 in CH₂Cl₂/Acetone 3/1; U.V. and phosphomolybdic developer). C₇₀H₁₁₄N₃O₁₁P. IS/MS: m/z 1204.5 ([M+H]⁺).

2.36.7. (3S,9R)-3-[(R)-3-Benzyloxytetradecanoylamino]-4-oxo-5-aza-9-[(R)-3-dodecanoyloxytetradecanoylamino]-decan-1,10-diol 1-(dibenzylphosphate) 10-(6-benzyloxycarbonylaminohexanoate)

Commercial 1-(3-dimethylaminopropyl)-3-ethylcarbodiimide hydrochloride (173 mg, 0.90 mmol) and 4-dimethylaminopyridine (11 mg, 90 μmol) are successively added to a solution of the compound prepared above (542 mg, 0.45 mmol) and commercial 6-(benzyloxycarbonylamino)hexanoic acid (238 mg, 0.90 mmol) in dry CH₂Cl₂ (15 ml) at 0° C. and under argon. The reaction mixture is then stirred for 30 minutes at 0° C. then overnight at ambient temperature. The reaction medium is then washed with water then with a 1N solution of HCl, the phases are then separated. The organic phase is dried over MgSO₄, filtered and evaporated. Purification by Flash chromatography on silica gel (eluting in petroleum ether/EtOAc 1/2) allows the coupling product (537 mg; 82%) to be collected in the form of an amorphous solid. (Rf=0.69 in CH₂Cl₂/Acetone 3/1; U.V. and phosphomolybdic developer). C₈₄H₁₃₁N₄O₁₄P. IS/MS: m/z 1451.8 ([M+H]⁺), 1475.0 ([M+Na]⁺), 1174.0 ([M-(BnO)₂OPOH])⁺.

2.36.8. (3S, 9R)-3-[(R)-3-hydroxytetradecanoylamino]-4-oxo-5-aza-9-[(R)-3-dodecanoyloxytetradecanoylamino]-decan-1,10-diol 1-dihydrogen phosphate 10-(6-aminohexanoate)

A solution of the compound prepared above (500 mg, 0.34 mmol) in a CH₂Cl₂/ethanol mixture 5/2 (70 ml) containing acetic acid (10 ml) is hydrogenated in the presence of 10% Pd on carbon at ambient temperature and under atmospheric pressure of hydrogen for 36 hours. The catalyst is eliminated by filtration. The filtrate is evaporated to dryness then dried with a vane pump in order to produce (3S, 9R)-3-[(R)-3-hydroxytetradecanoylamino]-4-oxo-5-aza-9-[(R)-3-dodecanoyloxytetradecanoylamino]-decan-1,10-diol 1-dihydrogen phosphate 10-(6-aminohexanoate) (301 mg, 84%) in the form of an amorphous solid. C₅₅H₁₀₆N₄O₁₂P. ES/MS: m/z 1047.8 ([M+H]⁺), 1069.8 ([M+Na]⁺). (FIG. 17)

### Example 2.37

### (3S, 9R)-3-[(R)-3-Dodecanoyloxytetradecanoylamino]-4-oxo-5-aza-9-[(R)-3-dodecanoyloxytetradecanoylamino]-decan-1,10-diol 1-dihydrogen Phosphate 10-(6-aminohexanoate) (=OM-197-MP AC-tetracyl) (FIG. 18)

2.37.1. (3S, 9R)-3-[(R)-3-Dodecanoyloxytetradecanoylamino]-4-oxo-5-aza-9-[(R)-3-dodecanoyloxytetradecanoylamino]-10-(2-tetrahydropyranyl)oxy-decan-1-ol

(2R)-5-amino-2-[(R)-3-dodecanoyloxytetradecanoylamino]-1-(2-tetrahydropyranyloxy)-pentane (section 2.36.2) (800 mg, 1.3 mmol, 1.3 eq) is added at 25° C. to a solution of (S)-(α)-[(R)-3-dodecanoyloxytetradecanoylamino]-γ-butyrolactone (section 2.33.3) (514 mg, 1.00 mmol) in anhydrous CH₂Cl₂ (2 mL). The solution is stirred for 6 days at 25° C. (no change in the reaction is noted) then isopropanol (2 ml) and triethylamine (140 μl, 1 mmol, 1 eq ) are added. The solution is stirred for another 10 days at 25° C. then the solvent is evaporated off under reduced pressure. Purification by Flash chromatography on silica gel (eluting with CH₂Cl₂/acetone 3.5/1 to 1/1) allows the alcohol (394 mg, 35%) to be collected in the form of an amorphous solid. (Rf=0.34 in CH₂Cl₂/Acetone 3/1; U.V. and phosphomolybdic developers). C₆₆H₁₂₅N₃O₁₀. IS/MS: m/z. 1121.0 ([M+H]⁺).

2.37.2. (3S, 9R)-3-[(R)-3-Dodecanoyloxytetradecanoylamino]-4-oxo-5-aza-9-[(R)-3-dodecanoyloxytetradecanoylamino]-10-(2-tetrahydropyranyl)oxy-decan-1-ol 1-(dibenzylphosphate)

85% dibenzyl-diethyl phosphoramidite (242 μl, 0.807 mmol, 2.3 equiv) is added to a solution of the alcohol prepared above (393 mg, 0.35 mmol) and 1H-tetrazole (74 mg, 1.05 mmol, 3 eq) in anhydrous THF (15 ml) at ambient temperature and under argon. The formation of white crystals in the reaction medium rapidly becomes apparent. After stirring for 1 hour 45 minutes at ambient temperature, a solution of mCPBA (57-86%; 221 mg, 1.3 mmol, 3.7 eq) in CH₂Cl₂ (10 ml) is added. The disappearance of the crystals is observed. After stirring for 1 hour at ambient temperature, a saturated solution of Na₂S₂O₃ (20 ml) is added then the reaction mixture is stirred for 10 minutes. The solution is then diluted with ether, then the organic phase is separated and washed with a saturated solution of Na₂S₂O₃ (5×), and with a saturated solution of NaHCO₃ (2×). The organic phase is dried over MgSO₄, filtered and evaporated. Purification by Flash chromatography on silica gel (eluting with CH₂Cl₂/acetone 6/1, 5/1 then 1/1) allows the dibenzylphosphate (450 mg; 92%) to be collected in the form of an amorphous solid. (Rf=0.29 in CH₂Cl₂/Acetone 4/1; U.V. and phosphomolybdic-developer). C₈₀H₁₃₈N₃O₁₃P. IS/MS: m/z 1381.0 ([M+H]⁺), 1403.0 ([M+Na]⁺).

2.37.3. (3S, 9R)-3-[(R)-3-Dodecanoyloxytetradecanoylamino]-4-oxo-5-aza-9-[(R)-3-dodecanoyloxytetradecanoylamino]-decan-1,10-diol 1-(dibenzyl Phosphate)

A solution of the compound prepared above (429 mg, 0.31 mmol) in CH₂Cl₂ (15 ml) is added at ambient temperature to a 1% solution of HCl in methanol (14 ml). After stirring for 2 hours at ambient temperature, the reaction medium is neutralized with a 10% solution of NaHCO₃, diluted with CH₂Cl₂ then the organic phase is separated. The resulting aqueous phase is then extracted with CH₂Cl₂ (3×) then the organic phases are combined. The organic phase is dried over MgSO₄, filtered and evaporated. Purification by Flash chromatography on silica gel (eluting with CH₂Cl₂/acetone 4/1, 2/1 then 1/1) allows the dibenzylphosphate (186 mg; 46%) to be collected in the form of an amorphous solid. (Rf=0.39 in CH₂Cl₂/Acetone 3/1; U.V. and phosphomolybdic developer). C₇₅H₁₃₀N₃O₁₂P. IS/MS: m/z 1297.0 ([M+H]⁺), 1319.0 ([M+Na]⁺), 1019.0 ([M-(BnO)₂OPOH]⁺)

2.37.4. (3S,9R)-3-[(R)-3-dodecanoyloxytetradecanoylamino]-4-oxo-5-aza-9-[(R)-3-dodecanoyloxytetradecanoylamino]-decan-1,10-diol 1-(dibenzylphosphate) 10-(6-benzyloxycarbonylaminohexanoate)

Commercial 1-(3-dimethylaminopropyl)-3-ethylcarbodiimide hydrochloride (46 mg; 0.24 mmol, 2 eq) and 4-dimethylaminopyridine (3 mg, 0.02 mmol, 0.2 eq) are successively added to a solution of the compound prepared above (154 mg, 0.12 mmol) and commercial 6-(benzyloxycarbonylamino)hexanoic acid (63 mg, 0.24 mmol, 2 eq) in dry CH₂Cl₂ (5 ml) at 0° C. and under argon. The reaction mixture is then stirred for 30 minutes at 0° C. then overnight at ambient temperature. The reaction medium is then diluted with CH₂Cl₂, washed with water then with a 1N solution of HCl then the phases are separated. The organic phase is dried over MgSO₄, filtered and evaporated. Purification by Flash chromatography on silica gel (eluting in CH₂Cl₂/acetone 3/1) allows the coupling product (149 mg; 81%) to be collected in the form of an amorphous solid. (Rf=0.75 in CH₂Cl₂/acetone 3/1; U.V. and phosphomolybdic developer). C₈₉H₁₄₇N₄O₁₅P. IS/MS: m/z 1545.5 ([M+H]⁺).

2.37.5. (3S, 9R)-3-[(R)-3-dodecanoyloxytetradecanoylamino]-4-oxo-5-aza-9-[(R)-3-dodecanoyloxytetradecanoylamino]-decan-1,10-diol 1-dihydrogen Phosphate 10-(6-aminohexanoate)

A solution of the compound prepared above (140 mg; 0.091 mmol) in a CH₂Cl₂/ethanol mixture 3/1 (20 ml) is hydrogenated in the presence of 10% Pd on carbon at ambient temperature and under atmospheric pressure of hydrogen for 24 hours. The catalyst is eliminated by filtration. The filtrate is evaporated to dryness then dried with a vane pump in order to produce (3S, 9R)-3-[(R)-3-Dodecanoyloxytetradecanoylamino]-4-oxo-5-aza-9-[(R)-3-dodecanoyloxytetradecanoylamino]-decan-1,10-diol 1-dihydrogen phosphate 10-(6-aminohexanoate) (92 mg, 83%). C₆₇H₁₂₉N₄O₁₃P. ES/MS: m/z 1229.9 ([M+H]⁺). (FIG. 19)

### Example 2.38

### (3S, 9R)-3-Octadecanoylamino-4-oxo-5-aza-9-[(R)-3-dodecanoyloxytetradecanoylamino]-decan-1,10-diol 1-dihydrogen Phosphate 10-(6-amino Hexanoate) (=OM-197-MP AC-inv-C₁₈) (FIG. 20)

2.38.1. (S)-(α)-Octadecanoylamino-γ-butyrolactone

N-methylmorpholine (604 μl, 5.49 mmol; 1 eq) and isobutyl chloroformate (713 μl, 5.49 mmol; 1 eq) are added successively to a solution of commercial octadecanoic acid (1.56 g, 5.49 mmol) in anhydrous THF (30 ml) at −15° C. and under argon. After stirring for 1 hour 15 minutes at −10° C., commercial L-homoserine lactone hydrobromide (1 g, 5.49 mmol) then a solution of NaHCO₃ (923 mg, 11 mmol, 2 eq) in H₂O (14 ml) are then added. The reaction mixture is then stirred for 16 hours at ambient temperature. The reaction medium is diluted with Et₂O (50 ml), the organic phase is separated and washed with H₂O then dried over MgSO₄, filtered and concentrated under reduced pressure. Purification by crystallization (minimum quantity of CH₂Cl₂ and excess pentane at 0° C.) allows the coupling product (1.38 g, 68%) to be collected in the form of a white solid. (Rf=0.31 in CH₂Cl₂/Acetone 2/1 phosphomolybdic developer). C₂₂H₄₁NO₃. IS/MS 368.5 ((M+H]⁺), 390.5 ([M+Na]⁺).

2.38.2. (3S,9R)-3-Octadecanoylamino-4-oxo-5-aza-9-[(R)-3-dodecanoyloxytetradecanoylamino]-10-(2-tetrahydropyranyl)oxy-decan-1-ol

(2R)-5-amino-2-[(R)-3-dodecanoyloxytetradecanoylamino]-1-(2-tetrahydropyranyloxy)-pentane (section 2.36.2) (800 mg, 1.3 mmol, 1.3 eq) is added at 25° C. to a solution of the (S)-(α)-Octadecanoylamino-γ-butyrolactone prepared above (370 mg, 1 mmol) in anhydrous CH₂Cl₂ (2 ml). The solution is stirred for 72 hours at 25° C. (no change in the reaction is noted) then triethylamine (140 μl, 1 mmol, 1 eq ) is added. The solution is stirred for 40 more days at 25° C. then the solvent is evaporated off under reduced pressure. Purification by Flash chromatography on silica gel (eluting with CH₂Cl₂/acetone 4/3 to 1/1) allows the alcohol (306 mg, 31%) to be collected in the form of a white solid. (Rf=0.17 in CH₂Cl₂/Acetone 3/1; U.V. and phosphomolybdic developers). C₅₈H₁₁₁N₃O₈. IS/MS: m/z. 979.0 ([M+H]⁺), 1001.0 ([M+Na]⁺).

2.38.3. (3S,9R)-3-Octadecanoylamino-4-oxo-5-aza-9-[(R)-3-dodecanoyloxytetradecanoylamino]-10-(2-tetrahydropyranyl)oxy-decan-1-ol 1-(dibenzyl phosphate)

85% dibenzyl-diethyl phosphoramidite (209 μl, 0.7 mmol, 2.3 equiv) is added at ambient temperature and under argon to a solution of the alcohol prepared above (296 mg, 0.303 mmol) and 1H-tetrazole (296 mg, 0.303 mmol, 3 eq) in anhydrous THF (12 ml). The formation of white crystals in the reaction medium rapidly becomes apparent. After stirring for 45 minutes at ambient temperature, a solution of mCPBA (57-86%; 191 mg, 1.12 mmol, 3.7 eq) in CH₂Cl₂ (17 ml) is added. The disappearance of the crystals is observed. After stirring for 45 minutes at ambient temperature, a saturated solution of Na₂S₂O₃ (20 ml) is added then the reaction mixture is stirred for 10 minutes. The solution is then diluted with ether, the organic phase is separated and washed with a saturated solution of Na₂S₂O₃ (5×), and with a saturated solution of NaHCO₃ (2×). The organic phase is dried over MgSO₄, filtered and evaporated. Purification by Flash chromatography on silica gel (eluting with CH₂Cl₂/acetone 3/1) allows dibenzylphosphate (330 mg; 89%) to be collected in the form of an amorphous solid. (Rf=0.40 in CH₂Cl₂/Acetone 2/1; U.V. and phosphomolybdic developer). C₇₂H₁₂₄N₃O₁₁P. IS/MS: m/z 1239.0 ([M+H]⁺), 1261.0 ([M+Na]⁺).

2.38.4. (3S, 9R)-3-Octadecanoylamino-4-oxo-5-aza-9-[(R)-3-dodecanoyloxytetradecanoylamino]-decan-1,10-diol 1-(dibenzylphosphate)

A solution of the compound prepared above (320 mg; 0.258 mmol) in CH₂Cl₂ (2 ml) is added at 0° C. to a 1% solution of HCl in methanol (11 ml). After stirring for 1 hour at 0° C., the reaction medium is neutralized with a 10% solution of NaHCO₃, diluted with CH₂Cl₂ then the organic phase is separated. The resulting aqueous phase is then extracted with CH₂Cl₂ (3×) then the organic phases are collected together. The organic phase is dried over MgSO₄, filtered and evaporated in order to produce the alcohol (254 mg; 85%) in the form of an amorphous solid. (Rf=0.20 in CH₂Cl₂/Acetone 3/1; U.V. and phosphomolybdic developer).

2.38.5. (3S, 9R)-3-Octadecanoylamino-4-oxo-5-aza-9-[(R)-3-dodecanoyloxytetradecanoylamino]-decan-1,10-diol 1-(dibenzylphosphate) 10-(6-benzyloxy Carbonylaminohexanoate)

Commercial 1-(3-dimethylaminopropyl)-3-ethylcarbodiimide hydrochloride (73 mg; 0.379 mmol; 1.8 eq) and 4-dimethylaminopyridine (1 mg, 0.038 mmol; 0.18 eq) are successively added to a solution of the compound prepared above (243 mg, 0.211 mmol) and commercial 6-(benzyloxycarbonylamino)hexanoic acid (101 mg, 0.379 mmol; 1.8 eq) in dry CH₂Cl₂ (9.5 ml) at 0° C. and under argon. The reaction mixture is then stirred for 30 minutes at 0° C. then overnight at ambient temperature. The reaction medium is then diluted with CH₂Cl₂, washed with water then with a 1N solution of HCl then the phases are separated. The organic phase is dried over MgSO₄, filtered and evaporated. Purification by Flash chromatography on silica gel (eluting with AcOEt/petroleum ether 2/1) allows the coupling product (226 mg; 77%) to be collected in the form of an amorphous solid. (Rf=0.53 in CH₂Cl₂/Acetone 3/1; U.V. and phosphomolybdic developer). C₈₁H₁₃₃N₄O₁₃P. IS/MS: m/z 1402.0 ([M+H]⁺), 1425.0 ([M+Na]⁺).

2.38.6. (3S, 9R)-3-Octadecanoylamino-4-oxo-5-aza-9-[(R)-3-dodecanoyloxytetradecanoylamino]-decan-1,10-diol 1-dihydrogen phosphate 10-(6-amino Hexanoate)

A solution of the compound prepared above (220 mg; 0.157 mmol) in a CH₂Cl₂ (20 ml) /ethanol (9 ml) mixture containing acetic acid (10 ml) is hydrogenated in the presence of 10% Pd on carbon at ambient temperature and under atmospheric pressure of hydrogen for 24 hours. The catalyst is eliminated by filtration. The filtrate is evaporated to dryness then dried with a vane pump in order to produce (3S, 9R)-3-Octadecanoylamino]-4-oxo-5-aza-9-[(R)-3-dodecanoyloxytetradecanoyl amino]-decan-1,10-diol 1-dihydrogen phosphate 10-(6-aminohexanoate) (134 mg; 81%). C₅₉H₁₁₅N₄O₁₁P. ES/MS: m/z 1087.7 ([M+H]⁺). (FIG. 21)

### Example 2.39

### (3S, 9R)-3-[(R)-3-Dodecanoyloxytetradecanoylamino]-4-oxo-5-aza-9-[(R)-3-hydroxytetradecanoylamino]-decan-1,10-diol 1-dihydrogen Phosphate 10-succinate (=OM-197-MP-Succ) (FIG. 22)

2.39.1. (3S,9R)-3-[(R)-3-dodecanoyloxytetradecanoylamino]-4-oxo-5-aza-9-[(R)-3-benzyloxytetradecanoylamino]-decan-1,10-diol 1-(dibenzyl Phosphate) 10-succinate

The same reaction sequence (cf section 2.7.4 and section 2.7.5) applied to (3S,9R)-3-[(R)-3-Dodecanoyloxytetradecanoylamino]-4-oxo-5-aza-9-[(R)-3-benzyloxytetradecanoylamino]-10-(2-tetrahydropyranyl)oxy-decan-1-ol (section 2.33.4) leads to (3S,9R)-3-[(R)-3-Dodecanoyloxytetradecanoylamino]-4-oxo-5-aza-9-[(R)-3-benzyloxytetradecanoylamino]-decan-1,10-diol 1-(dibenzyl phosphate).

Succinic anhydride (22 mg, 0.22 mmol) is added at ambient temperature to a solution of (3S, 9R)-3-[(R)-3-Dodecanoyloxytetradecanoylamino]-4-oxo-5-aza-9-[(R)-3-benzyloxytetradecanoyl amino]-decan-1,10-diol 1-(dibenzyl phosphate) (176 mg, 0.15 mmol) in dry CH₂Cl₂ (5 ml) in the presence of Et₃N (41 μl, 0.29 mmol). The reaction medium is stirred overnight at ambient temperature, diluted with CH₂Cl₂, washed with a 1N solution of HCl then the phases are separated. The organic phase is dried over MgSO₄, filtered and evaporated. Purification by Flash chromatography on silica gel (eluting with CH₂Cl₂/Acetone=4/1 containing 1% of acetic acid then CH₂Cl₂/Acetone=2/1 containing 1% of acetic acid) allows the acid (186 mg, 97%) to be collected in the form of a white solid. (Rf=0.65 in CH₂Cl₂/acetone 2/1 containing 1% acetic acid; U.V. and phosphomolybdic developer). C₇₄H₁₁₈N₃O₁₄P. ES/MS: m/z 1327.0 [M+Na]⁺, 1027 [M-(BnO)₂P(O)OH+H]⁺

2.39.2. (3S,9R)-3-[(R)-3-dodecanoyloxytetradecanoylamino]-4-oxo-5-aza-9-[(R)-3-hydroxytetradecanoylamino]-decan-1,10-diol 1-dihydrogen Phosphate 10-succinate

A solution of the compound prepared above (54 mg; 0.041 mmol) in ethanol (6 ml) is hydrogenated in the presence of 10% Pd on carbon at ambient temperature and under atmospheric pressure of hydrogen for 4 hours 30 minutes. The catalyst is eliminated by filtration. The filtrate is evaporated to dryness then dried with a vane pump in order to produce (3S, 9R)-3-[(R)-3-Dodecanoyloxytetradecanoylamino]-4-oxo-5-aza-9-[(R)-3-hydroxytetradecanoylamino]-decan-1,10-diol 1-dihydrogen phosphate 10-succinate (33 mg, 77%) in the form of a white solid. C₅₃H₁₀₀N₃O₁₄P. ES/MS: m/z 1034.5 [M+H]⁺. (FIG. 23)

### Example 2.40

### N-[(R)-3-Dodecanoyloxytetradecanoyl]-O-(phosphono)-DL-homoserine, O-{(2R)-2-[(R)-3-hydroxytetradecanoylamino]-5-(6-aminohexanoylamino)-pentyl} Ester (=OM-197-MP-AC-ester) (FIG. 24)

2.40.1. (2R)-5-[6-(Benzyloxycarbonylamino)hexanoylamino]-2-[(R)-3-benzyloxytetradecanoylamino]-pentan-1-ol

A solution of IIDQ (181 mg, 0.598 mmol, 1.3 eq) in anhydrous CH₂Cl₂ (5 ml) is added to a solution of (2R)-5-amino-2-[(R)-3-benzyloxytetradecanoylamino]pentan-1-ol (section 1.1.2.g) (200 mg, 0.46 mmol) in anhydrous CH₂Cl₂ (15 ml) and under argon. The reaction mixture is stirred for 15 minutes at ambient temperature then a solution of 6-(benzyloxycarbonylamino)hexanoic acid (122 mg, 0.46 mmol, 1 eq) in anhydrous CH₂Cl₂ (5 ml) is added under argon. The reaction medium is then stirred for 48 hours at ambient temperature, then the solvent is evaporated off. Purification of the residue by Flash chromatography on silica gel (eluting with CH₂Cl₂/acetone 3/2) allows the coupling product (216 mg; 69%) to be collected.

2.40.2. N-[(R)-3-Dodecanoyloxytetradecanoyl]-O-(diphenyloxyphosphoryl)-DL-homoserine, O-{(2R)-2-[(R)-3-benzyloxytetradecanoylamino]-5-[6-(benzyloxycarbonylamino)hexanoylamino]-pentyl}ester

Dicyclohexylcarbodiimide (34 mg, 0.164 mmol, 1.02 eq) and 4-dimethylaminopyridine (3 mg, 0.024 mmol, 0.15 eq) are successively added to a solution of the compound prepared above (110 mg, 0.161 mmol) and 4-(diphenyloxyphosphoryloxy)-2-[(R)-3-dodecanoyloxytetradecanoyl amino] butanoic acid (section 1.1.1.f) (122 mg, 0.161 mmol) in anhydrous CH₂Cl₂ (5 ml). The reaction mixture is stirred for 24 hours at ambient temperature, diluted with ether, cooled down to 0° C., then filtered on millipore in order to eliminate the dicyclohexylurea precipitate. The solvent is then evaporated off. Purification by Flash chromatography on silica gel (eluting with CH₂Cl₂/acetone 3/1) allows the expected coupling product (70 mg; 30%) to be collected.

2.40.3. N-[(R)-3-Dodecanoyloxytetradecanoyl]-O-(diphenyloxyphosphoryl)-DL-homoserine, O-{(2R)-2-[(R)-3-hydroxytetradecanoylamino]-5-(6-aminohexanoylamino)-pentyl}ester

A solution of the compound prepared above (100 mg, 0.070 mmol) in HPLC ethanol (10 ml) containing acetic acid (8 μl, 2 eq) is hydrogenated in the presence of an excess of palladiated carbon (10%) at ambient temperature and under atmospheric pressure of hydrogen for 18 hours. The catalyst is then eliminated by filtration. The filtrate is evaporated to dryness then the residue is dried with a pump in order to produce N-[(R)-3-Dodecanoyloxytetradecanoyl]-O-(diphenyloxyphosphoryl)-DL-homoserine, O-{(2R)-2-[(R)-3-hydroxytetradecanoylamino]-5-(6-aminohexanoyl amino)-pentyl}ester (65 mg, 77%). C₆₇H₁₁₅N₄O₁₂P. IS/MS: m/z 1222.5 [M+Na]⁺, 1200.0 [M+H]⁺

2.40.4. N-[(R)-3-Dodecanoyloxytetradecanoyl]-O-(phosphono)-DL-homoserine, O-{(2R)-2-[(R)-3-hydroxytetradecanoylamino]-5-(6-aminohexanoylamino)-pentyl}ester

The PtO₂ is first activated in platinum black, in suspension in HPLC ethanol (1 ml) by treatment with hydrogen at ambient temperature and under atmospheric pressure for 15 minutes. This activated platinum suspension is then transferred to a solution of the compound obtained above (60 mg) in HPLC MeOH (10 ml) containing acetic acid (10 ml). The reaction mixture is degassed under vacuum then placed under atmospheric pressure of hydrogen at ambient temperature for 48 hours. The hydrogen of the reaction flask is then replaced with an inert gas such as argon in order to avoid any risk of the platinum igniting, then the catalyst is filtered on millipore and the solvent is evaporated in order to produce N-[(R)-3-Dodecanoyloxytetradecanoyl]-O-(phosphono)-DL-homoserine, O-{(2R)-2-[(R)-3-hydroxytetradecanoylamino]-5-(6-aminohexanoylamino)-pentyl}ester (40 mg, 76%).

### 4th Series of Additional Examples

### Pharmacological Study of the Compounds According to the Invention (In Vitro)

### Additional Example 4.2

### Determination of the Proliferation of Stem Cells in Mouse Bone Marrow Stimulated by the Products of the Invention

4.2.1 . Proliferation Experiment

6-week-old male C57/BL6 mice are sacrificed by inhalation of CO₂. The bones of the rear limbs, the hip, the tibia and the femur are removed. The marrow is extracted from the osseous lumen by injection of Dulbecco's modified Eagle's medium (DH medium) through the previously sectioned extremities. The stem cells are washed and resuspended in a DH medium completed with 20% fetal calf serum (FCS). The cell concentration-is adjusted to 500,000 cells/ml.

The products in solution in the DH medium with added 20% FCS, amino acids and antibiotics, are diluted in succession directly in the microplate. The products are tested in triplicate and each microplate comprises a negative control comprising the medium alone. The final volume in each well is 100 μl.

100 μl of cell suspension is added to the product dilutions and the cells are incubated for 8 days in an oven at 37° C., under 8% CO₂, saturated in humidity. The proliferation is determined by measurement of the oxidation of an XTT chromogenic substrate (2,3-bis[2-methoxy-4-nitro-5-sulphophenyl]-2H-tetrazolium-5-caboxanilide) in the mitochondria of living cells.

At the end of the incubation, 50 μl of the XTT substrate solution (1 mg/ml of XTT+0.008 mg/ml phenazine methosulphate) is added to each well. Incubation is continued for a further 8 hours and the microplates are read with a spectrophotometer at 490 nm against a reference of 690 nm.

The results are expressed as a mean±standard deviation in the form of a dose-response curve. The values of the negative control comprising the DH medium (mean±standard deviation) are also indicated in the graphic form. From the dose-response curve, 3 particular points of the curve are calculated:

| | |
|---|---|
| Max: | maximum amplitude of the curve and its corresponding |
| | concentration |
| EC50: | concentration corresponding to 50% of the maximum |
| | amplitude |
| Min: | lowest concentration inducing a significant proliferation |
| | corresponding to blank + 3 × standard deviation |

The concentrations corresponding to EC50 and min. are determined from the straight line segments connecting the different points of the curve. If the curve does not reach a maximum but continues rising for the highest concentration tested, a “>” sign is placed before the “Max” and “EC50” values.

4.2.2. Presentation of the Results

Proliferation of Stem Cells of the Bone Marrow Induced by Acylated Pseudodipeptides Carrying Functionalized Auxiliary Arms

FIG. 25 shows the activity of different acylated pseudodipeptides and demonstrates the influence of the functionalized auxiliary arms and the distribution of the acyl chains on the ability to induce the proliferation of mouse marrow stem cells. Modifications at the level of the acyl chains significantly affect the ability of the pseudodipeptides to induce proliferation of stem cells. The tetracylated compounds as well as the inverse compounds have a marginal activity in this model. The type of bond of the group functionalized on the pseudodipeptide also modifies the dose-response curve for the products.

The minimum concentration of product capable of inducing a significant proliferation of stem cells is greater than that of the positive control, however certain pseudodipeptides are capable of inducing a response as great as that of E. coli LPS. Table showing induction of the proliferation of marrow stem cells by the different acylated pseudodipeptides carrying functionalized auxiliary arms corresponding to general formula I:

| | Max | EC50 | Min |
|---|---|---|---|
| | Amplitude/ | Amplitude/ | Amplitude/ |
| | O.D. conc./ | O.D. conc./ | O.D. conc./ |
| Products | μg/ml | μg/ml | μg/ml |
| DH medium | 0.47/0 | — | — |
| (negative control) | | | |
| E. coli LPS | 1.46/15.82 | 0.96/2.53 | 0.57/0.21 |
| (positive control) | | | |
| OM-197-AC-MP | 1.25/25 | 0.86/3.98 | 0.57/1.93 |
| OM-197-AH-AC | 0.64/6.25 | n.d. | 0.57/4.45 |
| OM-197-AH-MP | >1.52/>100 | >0.99/>34.74 | 0.57/6.27 |
| OM-197-MC-AC-inv | >1.07/>25 | >0.77/>16.78 | 0.57/6.04 |
| OM-197-MC-AC-tetracyl | >0.60/>25 | n.d. | 0.57/22.45 |
| OM-197-MP-AP | >1.03/>50 | >0.75/>7.54 | 0.57/1.96 |
| OM-197-MP-AD | >0.70/>25 | >0.58/>14.49 | 0.57/13.65 |
| OM-197-AH-OH | >0.74/>100 | >0.60/>41.07 | 0.57/29.16 |
| OM-197-MP-AC-inv | 0.68/50 | n.d. | 0.57/15.35 |
| OM-197-MP-AC-tetracyl | 0.66/6.25 | n.d. | n.d. |

nd = not determinable

When the induced activity is low it is not possible to determine the EC50 activity as it is lower than the minimum activity.

The acylated pseudodipeptides of the invention show large differences in their ability to induce the proliferation of marrow stem cells. Certain compounds even have marginal activities. Unlike products derived from lipid A, the increase in the number of acyl chains is not correlated to the activity of the products.

### Additional Example 4.3

### Determination of the Production of Nitric Oxide by Murine Macrophages Stimulated by the Products of the Invention

4.3.1. Experiment in Producing Nitric Oxide:

6-week-old male C57/BL6 mice are sacrificed by inhalation of CO₂. The bones of the rear limbs, the hip, the tibia and the femur are removed. The marrow is extracted from the osseous lumen by injection of Dulbecco's modified Eagle's medium (DH medium) through the extremities which have previously been sectioned. The stem cells are washed and resuspended (cell concentration approximately 40,000 cells/ml) in a DH medium completed with 20% horse serum (HS) and 30% of L929 supernatant. L929 is a line of murine fibroblasts the supernatant of which is rich in macrophage growth factor (M-CSF). The cell suspension is distributed in Petri dishes which are incubated for 8 days at 37° C. under 8% of CO₂ in a humidity-saturated oven.

After 8 days the stem cells are differentiated to mature macrophages. The macrophages are detached by exposure to cold, washed and resuspended in a DH medium completed with 5% fetal calf serum (FCS), aminoacids and antibiotics (DHE medium). The cell concentration is adjusted to 700,000 cells per ml.

The products are diluted in succession in the DHE medium directly in the microplate. The products are tested in triplicate and each microplate includes a negative control comprising the medium alone. The final volume in each well is 100 μl.

100 μl of cell suspension is added to the product dilutions and the cells are incubated for 22 hours in an oven at 37° C., under 8% of CO₂, saturated in humidity. At the end of the incubation with the products, 100 μl of supernatant is removed and the nitrite concentration is determined by Griess reaction.

100 μl of Griess reagent (5 mg/ml of sulphanilamide+0.5 mg/ml of N-(1-naphthylethylene diamine) hydrochloride in 2.5% aqueous phosphoric acid), are added to each well. The microplates are read with a spectrophotometer at 562 nm against a reference of 690 nm. The nitrite concentration is proportional to that of the nitric oxide produced. The nitrite concentration is determined with respect to a standard curve.

The results are expressed as a mean±standard deviation in the form of a dose/response curve.

From the dose-response curve, 3 particular points of the curve are calculated:

| | |
|---|---|
| Max: | maximum amplitude of the curve and its corresponding |
| | concentration |
| EC50: | concentration corresponding to 50% of the maximum |
| | amplitude |
| Min: | lowest concentration inducing a significant proliferation |
| | corresponding to blank + 3 × standard deviation |

The concentrations corresponding to EC50 and min. are determined from the straight line segments connecting the different points of the curve.

If the curve does not reach a maximum but continues rising for the highest concentration tested, a “>” sign is placed before the “Max” and “EC50” values.

If the curve does not descend below the limit of the blank+3 standard deviation, the minimum concentration is given as “<” the lowest concentration tested.

4.3.2. Presentation of the Results

Production of Nitric Oxide by Murine Macrophages Induced by Acylated Pseudodipeptides Carrying Functionalized Auxiliary Arms

FIG. 26 shows the activity of different acylated pseudodipeptides and demonstrates the influence of the functionalized auxiliary arm and of the modulation of the acyl chains on the ability to induce the production of NO by murine macrophages.

Modifications at the level of the acyl chains significantly affect the ability of the pseudodipeptides to induce the production of NO by murine macrophages both at the level of the amplitude and the minimum concentration capable of inducing the response. The type of bond of the functionalized group to the pseudodipeptides also modifies the dose-response curve for the products.

The minimum concentration of product capable of inducing a significant production of NO by the murine macrophages is much greater than that of the positive control. This concentration threshold can vary significantly among the compounds of the invention. The pseudodipeptides induce a response lower in amplitude with respect to the E. coli LPS.

Result table showing the induction of the production of nitric oxide by murine macrophages by the different acylated pseudodipeptides carrying functionalized auxiliary arms corresponding to general formula I:

| | Max | EC50 | Min |
|---|---|---|---|
| | Amplitude/ | Amplitude/ | Amplitude/ |
| | conc. | conc. | conc. |
| | μM nitrite/ | μM nitrite/ | μM nitrite/ |
| Products | μg/ml | μg/ml | μg/ml |
| DH medium | 0.05/0 | — | — |
| (negative control) | | | |
| E. coli LPS | 17.89/12.5 | 8.97/0.04 | <0.98/<0.001 |
| (positive control) | | | |
| OM-197-AC-MP | 12.69/12.5 | 6.37/1.91 | 0.38/0.54 |
| OM-197-AH-AC | 5.94/25 | 2.99/9.61 | 0.38/6.38 |
| OM-197-AH-MP | >8.68/>100 | >4.37/>20.9 | 0.38/5.18 |
| OM-197-MC-AC-inv | 8.64/50 | 4.34/14.17 | 0.38/1.72 |
| OM-197-MC-AC-tetracyl | >3.13/>50 | >1.59/>18.53 | 0.38/6.84 |
| OM-197-MP-AP | 12.50/25 | 6.27/4.43 | <0.61/<0.39 |
| OM-197-MP-AD | >7.98/>50 | >4.02/>25.76 | 0.38/9.64 |
| OM-197-AH-OH | >5.02/>50 | >2.53/>13.95 | 0.38/5.01 |
| OM-197-MP-AC-inv | >8.05/>100 | >4.05/>22.25 | 0.38/4.31 |
| OM-197-MP-AC-tetracyl | 8.94/12.5 | 4.49/3.19 | 0.38/0.44 |
| OM-197-MP-AC-inv- | 7.34/50 | 3.70/20.91 | 0.38/8.13 |
| C18 | | | |
| OM-197-MP-Succ | 10.90/16 | 5.47/0.89 | 0.38/0.19 |

The production of NO by the murine macrophages is strongly influenced by the functionalized auxiliary arms as well as by the modulation of the acyl chains carried by the pseudodipeptides. The diversity of the functions carried by the functionalized arms allows the activity of the compounds to be modulated even if it is difficult to draw precise conclusions concerning the structure-activity relationships. Owing to their low activity, certain compounds could act as an antagonist, whereas those which are more active would be powerful agonists.

### Additional Example 4.4

### Differentiation Test on Dendritic Cells

The ability of the products of the invention to induce the maturation of pre-dendritic cells to dendritic cells was evaluated. The following parameters were measured by flow cytometry: loss of ability to phagocytize the Dextran marked with fluorescein, and expression of the CD86 surface marker.

4.4.1. Experimental Protocol

The mononucleic cells of peripheral blood are isolated from “buffy coats” of healthy donors. The monocytes purified by adherence are resuspended in an RPMI-1640 medium containing 10% fetal calf serum (FCS), GM-CSF and IL-4 to 10 ng/ml at a rate of 1×10⁶ cells/ml. The cells are distributed in Petri dishes at a rate of 10×10⁶ cells per dish and cultivated for 6 days with a change of medium after 3 days. The cells thus obtained are called predendritic cells (DC-6). The maturation of predendritic cells to mature dendritic cells is achieved by incubation with the products at 10 μg/ml or LPS (positive control at 1 or 0.1 μg/ml) for 3 more days. On day 9 (DC-9), the cells are harvested, and the parameters indicating the maturation of the dendritic cells (characterized by their large size and by a complex internal architecture −>diffraction) are measured by flow cytometry (FACSCalibur BD Biosciences). The percentage of cells expressing the surface marker CD86 or cells not incorporating Dextran-FITC is evaluated.

4.4.2. Evaluation of the Results

The immature dendritic cells (DC-6) originating from the differentiation of monocytes, by the conjugated effect of GM-CSF and IL-4, are capable of incorporating Dextran-FITC. During the maturation process, the cells lose this ability to incorporate Dextran-FITC. Analyses are carried out during the differentiation stage DC-9.

Phagocytosis of the dextran marked by the fluorescein and expression of the CD86 after stimulation of the predendritic cells (DC6) by the acylated pseudodipeptides carrying a functionalized auxiliary arm corresponding to general formula I Differentiation of the predendritic cells to mature dendritic cells.

| | Dextran | CD86 |
|---|---|---|
| Product | % DC/Fluorescence | % DC/Fluorescence |
| 37° C. (negative control) | 20.2/32.4 | 17.4/131.8 |
| E. coli LPS 1 μg/ml | 98.1/17.4 | 54.3/132.1 |
| E. coli LPS 0.1 μg/ml | 98.2/17.6 | 81.0/144.4 |
| OM-197-AC-MP | 45.3/44.9 | 13.0/46.9 |
| OM-197-AH-AC | 52.1/21.8 | 17.6/81.6 |
| OM-197-AH-MP | 17.0/18.4 | 52.4/71.1 |
| OM-197-MC-AC-inv | 90.8/24.1 | 53.5/21.1 |
| OM-197-MC-AC-tetracyl | 11.7/18.6 | 18.0/57.4 |
| OM-197-MP-AP | 93.4/24.5 | 60.4/48.1 |
| OM-197-MP-AD | 8.9/20.0 | 27.8/61.4 |
| OM-197-AH-OH | 25.2/15.0 | 37.1/74.2 |
| OM-197-MP-AC-inv | 85.8/29.5 | 45.7/92.0 |
| OM-197-MP-AC-tetracyl | 23.3/38.6 | 14.5/84.5 |

The differences in average fluorescence measured in particular for the marking with CD86 are due to the quality of the cells (of donors) and the results presented originate from several experiments.

At a concentration of 10 μg/ml certain acylated pseudodipeptides are capable of inducing the differentiation of predendritic cells to dendritic cells. The functionality provided by the auxiliary arm modulates this ability to activate predendritic cells. The inversion between X and Y of the same functionality can also modulate the ability to induce the maturation of dendritic cells. Compound OM-197-AC-MP is practically inactive in this model, while compound OM-197-MP-AC (old example) was very active.

Modification of the number of acyl chains is yet more significant, the tetracylateds having a marginal activity in this model.

The effect of the formulation of the acylated pseudodipeptides is particularly significant on the ability to induce the differentiation of predendritic cells to dendritic cells, and certain tetracylated compounds or with very long functionalized arms such as OM-197-MP-AD are very slightly soluble, and they are very slightly active.

### 5th Series of Examples

### Additional Pharmacological Study of the Compounds According to the Invention (In Vivo)

### Additional Example 5.6

### Evaluation of the Pyrogenicity of Acylated Pseudodipeptides in the Rabbit Model.

The purpose of this experiment is to determine the highest concentration of acylated pseudodipeptide carrying a grafted auxiliary arm which does not induce fever in a rabbit injected intravenously.

Protocol:

15 rabbits (New Zealand White Rabbits) were divided into the following groups:
Group 1: 0.9 mg/kg
Group 2: 0.09 mg/kg
Group 3: 0.009 mg/kg
Group 4: 0.0009 mg/kg
Group 5: water for injection

On the day of the test the rabbits are weighed and immobilized in boxes provided for this purpose. Temperature probes are introduced into their rectums. 1 hour after introduction of the probes the (pre-injection) temperatures are recorded for 90 minutes and the average is determined.

Each rabbit then receives an intravenous injection of product or of the control (sterile water with 0.9% NaCl) via the marginal (lateral) ear vein. The temperature is measured every 30 minutes for 3 hours. The difference between the pre-injection average and the maximum temperature is calculated and noted.

For 3 rabbits per group the test is considered as non pyrogenic if the sum of the 3 responses does not exceed 1.15° C., and pyrogenic if the sum exceeds 2.65° C. If the value obtained is between the two, the test is repeated with 3 more rabbits.

Result:

Pyrogenicity test of compound OM-197-MP-AC:

| OM-197-MP-AC | response in ° C. | |
|---|---|---|
| dose | sum/(individual value) | results |
| 0.9 mg/kg | 0.95/(0.75; 0.20; 0.00) | non-pyrogenic |
| 0.09 mg/kg | 0.70/(0.40; 0.00; 0.30) | non-pyrogenic |
| 0.009 mg/kg | 1.85/(1.00; 0.65; 0.20) | non-pyrogenic (inconclusive) |
| 0.0009 mg/kg | 0.80/(0.45; 0.15; 0.20) | non-pyrogenic |
| control | 0.55/(0.35; 0.10; 0.10) | non-pyrogenic |

The compound according to the invention is not pyrogenic at the highest tested dose 5 of 0.9 mg/kg.

The intermediate dose of 0.009 mg/kg was not repeated since the 2 higher doses were not pyrogenic.

The low pyrogenicity of the compounds of the invention confirms the in vitro endotoxicity data determined by LAL.

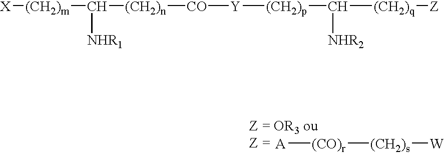

| Ex. # | X | *, ** | Y | m, n, p, q | R₁ | R₂ | R₃ | A | r | s | W |
|---|---|---|---|---|---|---|---|---|---|---|---|
| 2.28 | H₂NC₅OCO | S, R | NH | 2, 0, 3, 1 | 3(C₁₂O)C₁₄ | 3(HO)C₁₄ | P(O)(OH)₂ | — | — | — | — |
| 2.29 | H₂NC₆NHCO | S, R | NH | 1, 0, 3, 1 | 3(C₁₂O)C₁₄ | 3(HO)C₁₄ | — | O | 1 | 5 | NH₂ |
| 2.30 | H₂NC₆NHCO | S, R | NH | 1, O, 3, 1 | 3(C₁₂O)C₁₄ | 3(HO)C₁₄ | P(O)(OH)₂ | — | | | |
| 2.31 | HOOC | S, R | NH | 1, 0, 3, 1 | 3(HO)C₁₄ | 3(C₁₂O)C₁₄ | — | O | 1 | 5 | NH₂ |
| 2.32 | HOOC | S, R | NH | 1, 0, 3, 1 | 3(C₁₂O)C₁₄ | 3(C₁₂O)C₁₄ | — | O | 1 | 5 | NH₂ |
| 2.33 | (HO)₂P(O)—O | S, R | NH | 2, 0, 3, 1 | 3(C₁₂O)C₁₄ | 3(HO)C₁₄ | — | O | 1 | 2 | NH₂ |
| 2.34 | (HO)₂P(O)—O | S, R | NH | 2, 0, 3, 1 | 3(C₁₂O)C₁₄ | 3(HO)C₁₄ | — | O | 1 | 11 | NH₂ |
| 2.35 | H₂NC₆NHCO | S,R | NH | 1, 0, 3, 1 | 3(C₁₂O)C₁₄ | 3(HO)C₁₄ | H | — | — | — | — |
| 2.36 | (HO)₂P(O)—O | S, R | NH | 2, 0, 3, 1 | 3(HO)C₁₄ | 3(C₁₂O)C₁₄ | — | O | 1 | 5 | NH₂ |
| 2.37 | (HO)₂P(O)—O | S, R | NH | 2, 0, 3, 1 | 3(C₁₂O)C₁₄ | 3(C₁₂O)C₁₄ | — | O | 1 | 5 | NH₂ |
| 2.38 | (HO)₂P(O)—O | S, R | NH | 2, 0, 3, 1 | C₁₈ | 3(C₁₂O)C₁₄ | — | O | 1 | 5 | NH₂ |
| 2.39 | (HO)₂P(O)—O | S, R | NH | 2, 0, 3, 1 | 3(C₁₂O)C₁₄ | 3(HO)C₁₄ | — | O | 1 | 2 | COOH |
| 2.40 | (HO)₂P(O)—O | R/S, R | O | 2, 0, 1, 3 | 3(C₁₂O)C₁₄ | 3(HO)C₁₄ | — | NH | 1 | 5 | NH₂ |

1. A N-acyl-dipeptide-like compound bearing an acid group in neutral or charges state, at one end portion of aid dipeptide-like compounds and bearing an accessory functional side chain spacer at the other end portion thereof, having the formula
X—(CH₂)ₘ—CH—(CH₂)ₙ—CO—Y—(CH₂)ₚ—CH—(CH₂)_{q}-∠ (I)
wherein R₁ and R₂ each designate an acyl group derived from a saturated or unsaturated carboxylic acid having 2 to 24 carbon atoms, which is unsubstituted or bears at least one substituent selected from the group consisting of hydroxyl, alkyl, alkoxy, acyloxy, amino, acylamino, acylthio and alkylthio of up to 24 carbon atoms,
m, n are integers from 0 to 10,
p, q are integers from 1 to 10,
Y is O or NH,
X and Z each designate an accessory functional side chain spacer or an acid group either in neutral or charged state selected from the group consisting of
-carboxyl,
-carboxy [(C₁₋₅) alkoxy],
-carboxy [(C-15)alkylthio],
-phosphono [(C₁₋₅) alkoxy],
-phosphono [(C₁₋₅)alkylthio],
-dihydroxyphosphoryloxy[(C₁₋₅) alkoxy],
-dihydroxyphosphoryloxy[C₁₋₅],
-dihydroxyphosphoryloxy,
-hydroxysulfonyloxy,
-hydroxysulfonyl[C₁₋₅)alkoxy],
-hydroxysulfonyl[(C₁₋₅) alkylthio],
-hydroxysulfonyloxy [(C-1₁₋₅)alkoxy],
-[carboxy(C₁₋₅) ]aminocarbonyl,
-[dicaroby(C₁₋₅)alkyl]aminocarbonyl and
-{carboxy[amino(C₁₋₅)alkyl}aminocarbonyl,
provided that at least one of X or Z designates an accessory functional side chain spacer.

2. A compound of claim 1, wherein the accessory group denoted by X or Z, has the formula
A-(CO)ᵣ—(CH₂)ₛ—W (II)
where A is O or S or NH
r is 0 or 1
s is an integer from 1 to 10
W is selected from the group consisting of
-formyl,
-acetyl,
-cyano,
-halo,
-amino,
-bromo-, or iodo-acetamido,
-acylamido,
-diacylimido,
-sulfhydril,
-alkylthio,
-hydroxyl,
-1,2-dihydroxyethyl,
-alkoxy,
-acyloxy,
-vinyl,
-ethynyl,
-free carboxyl,
-esterified carboxyl or in the form of a mixed anhydride, amide or hydrazide,
-azido and
-thiocyano.

3. A N-acyl-dipeptide like compound bearing an acid group in neutral or charged state, at one end portion of said dipeptide-like compounds and bearing an accessory functional side chain spacer at the other end portion thereof, having the formula: 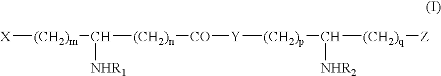
Wherein R₁ and R₂ each designate an acyl group derived from a saturated or unsaturated, carboxylic acid having from 2 to 24 carbon atoms, which is unsubstituted or substituted with at least one substituent selected from the group consisting of hydroxyl, alkyl, alkoxy, acyloxy, amino, acylamino, acylthio and alkylthio of up to 24 carbon atoms,
m, n are integers from 0 to 10,
p, q are integers from 1 to 10,
Y is O or NH,
X and Z each designate an acid group either in neutral or charged state or an accessory functional side chain spacer,
provided that at least one of X or Z is an accessory functional side chain spacer selected from the group consisting of
-carboxyl,
-carboxyl[(C₁₋₅) alkoxy],
-carboxy[(C₁₋₅)alkylthio],
-phosphono[(C₁₋₅) alkoxy],
-phosphono[(C₁₋₅) alkylthio],
-dihydroxyphosphoryloxy[C₁₋₅) alkoxy],
-hydroxysulfonyloxy,
-hydroxysulfonyl[(C₁₋₅) alkoxy],
-hydroxysulfonyl[(C₁₋₅) alkylthio],
-hydroxysulfonyloxy [(C₁₋₅)alkoxy] and
-hydroxysulfonyloxy [(C₁₋₅))alkylthio]
and the accessory functional side chain spacer of X or Z is as specified above, excluding 1,2-dihydroxyethyl.

4. A compound of claim 1 wherein X or Z designate an acid group in neutral state, said compound is meant to be the free carboxylic, sulfonic phospohonic or phosphoric compound of said acid.

5. A compound of claim 1 wherein X or Z designate an acid group in charged state, said compound is meant to be the carboxylic, sulfonic, phosphonic or phosphoric salt form, by addition of a pharmaceutical base.

6. A compound of claim 1 wherein X or Z designate an accessory group of the formula:
O—CO—(CH₂)ₛ-W (III)
s is an integer from 1 to 10,
and W is selected from the group consisting of
-formyl,
-amino,
-hydroxyl,
-1,2-dihydroxyethyl
-and carboxyl.

7. A compound of claim 1 having the formula: 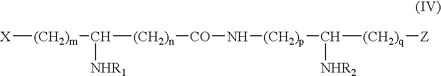
wherein R₁ and R₂ each are an acyl group derived from a saturated or unsaturated chain-carboxylic acid of 2 to 24 carbon atoms, unsubstituted or substituted with at least one member selected from the group consisting of hydroxyl, alkyl, alkoxy, acyloxy, amino, acylamino, acylthio and alkylthio of u to 24 carbon atoms,
m and n are integers from 0 to 10
p and q are integers from 1 to 10
and wherein X or Z is an acid functional group selected
from the group consisting of carboxyl, dihydroxyphosphoryloxy, carboxy[(C₁₋₅)alkoxy], carboxyl[(C₁₋₅)alkylthio], carboxy[(C₁₋₅) alkyl]aminocarbonyl, [dicarboxy(C₁₋₅)alkyl]aminocarbonyl and {carboxy[amino (C₁₋₅) alkyl}-aminocarbonyl and wherein the other is acyloxy selected from the group consisting of 6-aminohexanoylocy, 6-oxohexanoyloxy, 6-hydroxyhexanoyloxy, 6,7-dihydroxylheptanoyloxy and 3-carboxypropanoyloxy.

8. A compound of claim 1 selected from the group consisting of
-N[(R)-3-dodecanoyloxytetradecanoylamino]-C-aspartic acid, _{α}-N-{(4R)-5-hydroxy-4-[(R)-3-hydroxytetradecanoyl-amino]pentyl}amide and addition salts with a base thereof
-3-[(R)-3-dodecanoyloxytetradecanoylamino]-4-oxo-5-aza-9-[(R)-3-dodecanoyloxytetradecanoylamino]-4-oxo-5-aza-9-[(R)-3-hydroxytetradecanoylamino-decan-1,10-diol 1-dihydrogenphosphate 10-(6,7-dihydroxyheptanoate) and addition salts with a base thereof
-3[R (R) -3-dodecanoyloxytetradecanoylamino]-4-oxo-5-aza-9-[(R)-3-hydroxytetradecanoylamino]-decan-1,10-diol 1-dihydrogen-phosphate 10-(6-oxohexanoate) and addition salts with a base thereof
-N-[(R)-3-dodecanoyloxytetradecanoylamino]-D-aspartic acid, _{α}-N-{(4R-5-hydroxy-4-[(R)-3-hydroxytetradecanoylamino]pentyl}amide 5-0-(6,7dihydroxyheptanoate) and addition salts with a base thereof
-N-[(R)-3-dodecanoyloxyletradecanoylamino]-D-aspartic acid,_{α}-N-{(R)-5-hydroxy-4-[(R)-3-hydroxytetradecanoylamino]pentyl}amide 5-0-(6-oxohexanoate) and addition salts with a base thereof
-N-[(R)-3-dodecanoyloxytetradecanoylamino]-D-aspartic acid, _{α}-N-{(4-R)-5-hydroxy-4-[(R)-3-hydroxytetradecanoyl-amino]pentyl}amide 5-0-(6-hydroxyhesanoate) and addition salts with a base thereof
-(3RS,9R)-3-[(R)-3-dodecanoyloxytetradecanoylamino]-4-oxo-5-aza-9-[R(R)-3-hydroxy-tetra-decanoylamino]-decan-1,10-diol 1-dihydrogenphosphate 10-(6-aminohexanoate) and addition salts with a base thereof
-(3R,9R)-3-[(R)-3-dodecanoyloxytetradecanoylamino]-4-oxo-5-aza-9-[(R)-3-hydroxy-tetradecanoylamino]-decan-1,10-diol 1-dihydrogenphosphate 10(6-aminohexanoate) and addition salts with a base thereof
-(3S,9R)-3-[(R)-3-dodecanoyloxytetradecanoylamino]-4-oxo-5-aza-9-[(R)-3-hydroxy-tetra-decanoylamino]decan-1,10-diol 1-dihydro-tetra-decanoylamino]decan-1,10-diol 1-dihyrogenphosphate 10-(6-aminohexanoate) and addition salts with a base thereof -3-[(R)-3-dodecanoyloxytetradecanolylamino]-4-oxo-5-aza-9-[(R) -3-hydroxytetradecanoylamino]-decan-1,10-diol 1-dihydrogenphosphate 10-(6-hydroxyhexanoate) and addition salts with a base thereof -2-[(R)-3-hydroxytetradecanolamino]-5-(6-xohexyl)amino}pentyl 2-[R(R)-3-dodecanoyloxytetradecanoylamino]-r-dihydroxyphosphoryloxy)-butanoate and addition salts with a base thereof
-(2R,8R)-2-[(R)-3-dodecanoyloxytetradecanoylamino]-3-oxo-4-aza-8-[(R)-3-hydroxytetradecanoylamino]-nonane-1,9-diol 1-0-carboxymethyl ether 9-0-)6-oxohexanoate) and addition salts with a base thereof
-(2S,8R)-1-(carboxymethyl)-thio-2-[(R)-3-tetra-decanoyloxy-tetradecanoylamino]-3-oxo-4-aza-8-[(R)-3-hydroxytetradecanoylamino}-nonan-9-ol 9-0-(7-aminoheptanoate) and addition salts with a base thereof
-(2R, 8R)-2-[(R)-3-dodecanoyloxytetradecanoylamino]-3-oxo-4-aza-8-[(R)-3-hydroxytetradecanoylamino}-nonan-1,9-ol 1-0-(2,2-dicarboxyethyl)ether 9-0-(6-exanoate) and addition salts with a base thereof
-3-[(R)-3-dodecanoyloxytetradecanoylamino]-4-oxo-5-aza-9-[((R)-3-hydroxytetradecanoylamino]-1,10-diol 1-dihydrogenphosphate 10-(6-bromoacetamidohexanoate) and addition salts with a base thereof
-(3RS, 9R)-3-[(R)-3-hydroxytetradecanoylamino]-4-oxo-5-aza-9-[(R)-3-dodecanoyloxytetradecanoylamino]-decan1,10-diol 1-dihydrogenphosphate 10-0-(6-oxohexanoage) and addition salts with a base thereof
-N-[(R)-3-dodecanoyloxytetradecanoyl-D-aspartic acid, _{α}-N-{(4R) 5-(6-aminohexanoyloxy)-4-[(R) -3-hydroxytetradecanoyl-amino]pentyl}amide and addition salts with a base thereof
-N-[(R)-3-dodecanoyloxytetradecanoyl]-D-aspartic acid, _{α}-N-{(4R)-5-(6-succinylamidohexanoyloxy)-4-[(R)-3-hydroxytetradecanoyl-amino]pentyl}amide and addition salts with a base thereof
-N-[(R)-3-dodecanoyloxytetradecanoyl]-D-aspartic acid, _{α}-N-{(4R)-5-glycinyloxy-4-[(R)-3-hydroxytetradecanoyl-amino]pentyl}amide and addition salts with a base thereof
-N-[(R)-3-dodecanoyloxytetradecanoyl]-D-aspartic acid, _{α}-N-{(4R)-5-succinyloxy-4-[(R)-3-hydroxytetradecanoylamino]-pentyl}amide and addition salts with a base thereof
-N-[(R)-3-dodecanoyloxytetradecanoyl]-D-aspartic acid, _{α}-N-{(4r-5-hydroxy-4-[(R)-3-hydroxytetradecanoylamino]penthyl}amide _{β}-N-(3-aminopropyl)amide and addition salts with a base thereof
-N-[(R)-3-dodecanoyloxytetradecanoyl]-D-aspartic acid, _{α}-N-{(4R)-5-hydroxy-4-[(R(+)-3-hydroxytetradecanoylamino]pentyl}amide _{β}-N-[(1S)-1-carboxy-5-aminopentyl]amide and addition salts with a base thereof
-N-[(R)-3-dodecanoyloxytetradecanoyl]-D-aspartic acid, _{α}-N-{(4R)-5-(6-aminohexanoyloxy)-4-[(R)-3-hydroxytetradecanoylamino]-pentyl}amide _{β}-N-[(1S)-1-carboxy-5-aminopentyl]amide and addition salts with a base thereof
-N-[(R)-3-dodecanoyloxytetradecanoyl]-D-aspartic acid, _{α}-N-{(4R)-5-hydroxy-4-[(R)-3-hydroxytetradecanoylamino]pentyl}amide _{β}-N-[(1S0-1,2-dicarboxyethyl]amide and addition salts with a base thereof
-N-[(R)-3-dodecanoyloxytetradecanoyl]-D-aspartic acid, _{α}-N-{(4R)-5-(6-aminohexanoyloxy)-4-[(R)-3-hydroxytetra-decanoylamino]pentyl}amide-_{β}-N-[(1S)-1,2-dicarboxyethyl]amide and addition salts with a base thereof
-N-acetyl-D-aspartic acid, _{α}-N-{(4R)-5-(6-aminohexanoyloxy)-4-[(R)-3-hydroxytetradecanoylamino]pentyl}amide and addition salts with a base thereof
-N-(2-decanoyloxyoctanoyl)-D-aspartic acid, _{α}-N-{(4R)-5-(6-aminoexanoyloxy)-4-[(R)-3-hydroxytetradecanoylamino]pentyl}amide and addition salts with a base thereof.

9. A compound of claim 1 selected from the group consisting of:
-N-[(R)-3-dodecanoyloxytetradecanoylamino]-D-aspartic acid, _{α}-N-{(4R)-5-hydroxy-4-[(R)-3-hydroxytetradecanoylamino]pentyl}amide and addition salts with a base thereof
-3-[(R)-3-dodecanoyloxytetradecanoylamino]-4-oxo-5-aza-9-[(R)-3-hydroxytetradecanoylamino]-decan-1,10-diol 1-dihydrogenphosphate 10-(6, 7-dihydroxyhexanoate) and addition salts with a base thereof
-3-[(R)-3-dodecanoyloxytetradecanoylamino]-4-oxo-5-aza-9-[(R)-3-hydroxytetradecanoylamino]-decan-1,10-diol 1-dihydro-genphosphate 10-diol 1-dihydrogenphosphate 10-(6-oxohexanoate) and addition salts with a base thereof
-N-[(R) -3-dodecanoyloxytetradecanoylamino]-D-aspartic acid, _{α}-N-{(4R)-5-hydroxy-4-[(R) -3-hydroxytetra-decanoylamino]pentyl}amide 5,-0-(6,7-dihyroxyheptanoate) and addition salts with a base thereof
-N-[(R)-3-dodecanoyloxytetradecanoylamino]-D-aspartic acid, _{α}-N-{(4R)-5-hydroxy-4-[(R)-3-hydroxytetradecanoylamino]-pentyl}amide 5-0-(6-oxohexanoate) and addition salts with a base thereof
-(3RS, 9R), (3R, 9R) and (3S, 9R)-3-[(R)-3-dodecanoyloxytetradecanoylamino]-4-oxo-5-aza-9-[(R)-3-hydroxytetradecanoylamino]decan-1,10-diol 1-dihydrogenphosphoate 10-(6-aminohexanoate) compounds and addition salts with a base thereof
-3-[(R)-3-dodecanoyloxytetradecanoylamino]-4-oxo-5-aza-9-[(R)-3-hydroxytetradecanoylamino]-decan-1,10-diol 1-dihydrogenphosphate 10-(6-hydroxyhexanoate) compounds and addition salts with a base thereof
-{2-[(R) -3-hydroxytetradecanoylamino]-5-(6-oxohexyl) amino}pentyl-2[(R)-3-dodecanoyloxytetradecanoylamino]-4-(dihycroxyphosphoryloxy)-butanoate and addition salts with a base thereof
-N-[(R)-3-dodecanoyloxytetradecanoyl]-D-aspartic acid, _{α}-N-{(4-R)-5-(6-aminohexanoyloxy)-4-[(R)-3-hydroxytetradecanoyl-amino]pentyl}amide and addition salts with a base thereof
-N-[(R)-3-dodecanoyloxytetradecanoyl]-D-aspartic acid, _{α}-N-{(4R)-5-succinyloxy-4-[(R)-3-hydroxytetradecanoylamino]pentyl}amide and addition salts with a base thereof
-N-[(R)-3-dodecanoyloxytetradecanoyl]-D-aspartic acid, _{α}-N-{(4R)-5-(6-aminohexanoyloxy)-4-{(R)-3-hydroxytetradecanoylamino]-pentyl}amide _{β}-N-(1S)-1-carboxy-5-aminopentyl]amide and addition salts with a base thereof
-N-[(R)-3-dodecanoyloxytetradecanoyl]-D-aspartic acid, _{α}-N-{(4R)-5-(6-aminohexanoyloxy)-4-[(R) -3-hydroxytetradecanoylamino]-pentyl}aide β-N-[(1S)-1, dicarboyethyl]amide and addition salts with a base thereof.

10. A process for the preparation of a N-acyl-peptide-like compound of claim 1, bearing an acid group either in neutral or charged state at one end portion thereof and bearing an accessory functional side chain spacer at the other, having the general formula I, comprising blocking amine functional groups in position (q+1) and YH in position; of an ;-functionally substituted amino acid with transverse blocking reagents, reacting the. still free carboxylic functional group with a reducing agent to obtain the corresponding alcohol, freeing the amine functional group in position (q+1) and acylating the same with a carboxylic acid functional derivative of formula R₂OH wherein R₂ is defined in claim 1 freeing thereafter the terminal alcohol or amino functional group to provide the functionally derivatized amino alcohol of the formula: 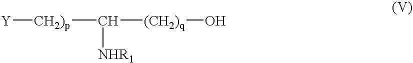
wherein Y is HO or NH₂,
R₂ is an acyl of a saturated or unsaturated carboxylic acid of 2 to 24 carbon atoms which is unsubstituted or bears at least one substituent as defined in claim 1
p and q are integers from 1 to 10,
condensing the amino alcohol in the presence of a peptide condensing agent in an inert solvent, together with a ;-functionally derivatized amino acid compound of the formula: 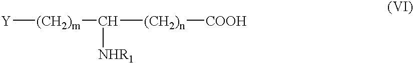
wherein R₁ is an acyl of a saturated or unsaturated carboxylic acid of 2 to 24 carbon atoms, which is unsubstituted or bears at least one substituent as defined in claim 1,
m and n are integers from 0 to 10,
and X is an acid group as defined above in free or esterified form,
to obtain a dipeptide-like compound of the formula: 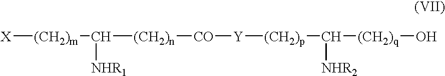
wherein R₁ and R₂ and m, n, p and q are as defined above, the free terminal alcohol functional group of which can otpionally be alkyl, acyl or otherwise substituted by an alkyl-,
acyl- or a substitution reagent of the formula:
A-(CO)₁—(CH₂)ₛ-W (VIII)
(VIII)
wherein A is selected from the. group consisting of a leaving group, OH, SH or NH₂,
r is equal to 1 or 0,
s is from 1 to 10,
W is selected from the group consisting of -formyl, -cyano, -halo, -amino, -bromo- or iodoacetamido, -acylamido, -diacylimido, -sulfhydril, -alkylthio, -hydroxyl, -1,2-dihydroxyethyl, -acyloxy, -vinyl, ethynyl, free or esterified carboxyl or in the form a mixed anhydride, amide or hydrazide, azido and -thiocyano optionally in the presence of a coupling agent, and subjecting the product to a catalytic hydrogenation to obtain the compound of the formula: 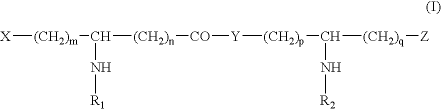
wherein X. U. Z. R₁, R₂, n, m p and q have the same meanings as those given above.

11. A process for the preparation of an acyl-dipeptide-like compound of the formula: 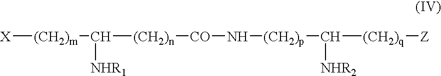
wherein R₁ and R₂ each designate an acyl of a saturated or unsaturated, carboxylic acid of 2 to 24 carbon atms, which is unsubstituted or substituted by at least one member of the group consisting of hydroxyl, alkyl, alkoxy, acyloxy, amino, acylamino, acylthio and alkylthio of up to 24 carbon atoms,
n is an integer from 0 to 10,
wherein X and Z each are an acid group either in neutral or charged state or an accessory functional side chain spacer comprising
blocking amine functional groups in positions (q+1) and of a diamino acid of the formula H₂N(CH₂)ₚCHNH₂(CH₂)_{q-1}COOH with a blocking reagent which readily undergoes acidolysis and hydrogenolysis, respectively, reacting the still free carboxylic functional group with a reducing agent to obtain the corresponding alcohol, freeing the amine functional group in position (q+1), acylating the same with a carboxylic acid functional derivative of the formula R₂OH wherein R₂ is as defined in claim 1, freeing the terminal amine functional group by hydrogenolysis to obtain an amino alcohol of the formula: 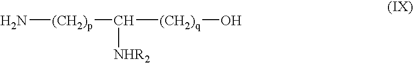
wherein R₂ is an acyl group derived from a saturated or unsaturated carboxylic acid of 2 to 24 carbon atoms, unsubstituted or substituted with at least one substitutent specified above,
p and q are an integer from 1 to 10 condensing amino alcohol in the presence of a peptide condensing agent in an inert solvent with an ;-hydroxyl amino acid of the formula: 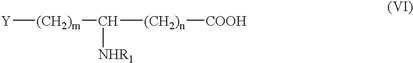
wherein R₁ is an acyl of a saturated or unsaturated carboxylic acid of 2 to 24 carbon atoms, unsubstituted or substituted with at least one substituent as defined above
m is an integer from 1 to 10,
n is an integer from 0 to 10,
and X is a dialkyloxy- or diaryloxy-phosphoryloxy of the formula: 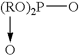
to yield the phosphoryl-dipeptide-like compound of the formula: 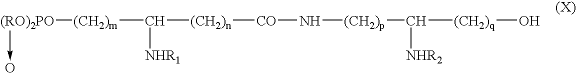
wherein R₁, R₂, m, n, p and q are as defined above, and R is a group which readily undergoes hydrogenolysis, the free terminal alcohol functional group of which can be—if desired—alkyl or acyl- substituted by an alkyl- or acyl- or a substitution reagent of the formula:
A-(CO)ᵣ—(CH₂)ₛ-W (VIII)
wherein A is selected from the group consisting of a leaving group, OH, SH or NH₂
r is 0 or 1,
s is an integer of 1 to 10,
W is selected from the group consisting of
-formyl, -acetyl, -cyano, -halo, -amino, -bromo- or iodo-acetamido, -acylamido, diacylimido, -sulfhydril, -alkylthio, -hydroxyl, 1,2-dihydroxyethyl, -acyloxy, -vinyl, -ethynyl, free or esterified carboxyl or in the form of a mixed anhydride, amide or hydrazide, -azido and thiocyano,
optionally in the presence of a coupling agent, and subjecting the product to a catalytic hydrogenation to obtain a compound of the formula: 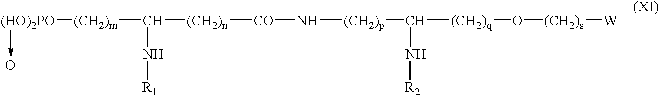
wherein W, R₁, R₂, m, n, p, q, r, s have the meanings above.

12. The process of claim 10 for the preparation of a compound of the formula: 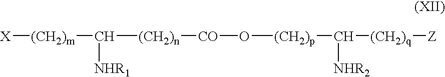
wherein R₁ and R₂ each are an acyl group of a saturated or unsaturated, carboxylic acid of 2 to 24 carbon atoms, unsubstituted or substituted with at Least one member selected from the group consisting of hydroxyl, alkyl, alkoxy, acyloxy, amino, acylamino, acylthio and alkylthio of up to 24 carbon atoms,
m, p and q are integers from 1 to 10,
n is an integer from 0 to 10,
X and Z each designate an acid group or an accessory functional side chain spacer comprising
blocking amine functional groups in positions (q+1) and of a diamino acid of formula H₂N(CH₂)ₚCHNH₂(CH₂)_{q-1}COOH with blocking reagents which readily undeergo acidolysis and hydrogenolysis, respectively, reacting the still free carboxylic group with a reducing agent to obtain the corresponding alcohol, freeing the amine group in position (q+1), acylating the same with a carboxylic acid functional derivative of the formula R₂OH wherein R₂ is as defined in claim 10, freeing the terminal amine group by hydrogenolysis and alkyl substituting the amine-group with an alkyl substitution agent to obtain an amino alcohol of the formula: 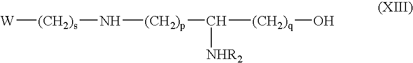
wherein R₂ is acyl of a saturated or unsaturated carboxylic acid of 2 to 24 carbon atoms, which is unsubstituted or substituted with at least one substituent as specified above,
p and q are an integer from 1 to 10,
s is an integer from 1 to 10, condensing the amino alcohol in the presence of a condensing agent in an inert solvent, with an ;-hydroxy amino acid of the formula: 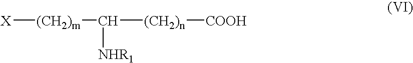
wherein R₁ is an acyl of a saturated or unsaturated carboxylic acid of 2 to 24 carbon atoms, unsubstituted or substituted with at least one substituent,
m is an integer from 1 to 10,
n is an integer from 0 to 10,
and X is a dialkyloxy- or diaryloxy- phosphoryloxy of the formula: 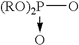
to obtain a dipeptide-like compound of the formula: 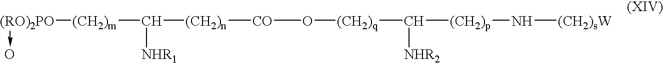
wherein R₁, R₂, m n, p, q and s are as defined above, and R is a group which readily undergoes hydrogenolysis,
subjecting the product to a catalytic hydrogenation to obtain a compound of the formula: 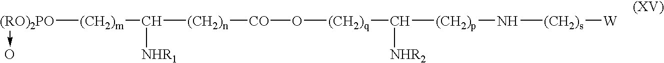
wherein W, R₁, R₂, m, n, p, q, r and s are as specified above and w is selected from the group consisting of -formyl, -acetyl, -cyano, -halo, -bromo- or iodoacetamido, -acylamido, -diacylimido, -acyloxy, -vinyl, ethynyl, free or esterified carboxyl or a mixed anhydride, amide, hydrazide, -azido and -thiocyano.

13. The process of claim 10 to obtain a carboxydipeptide-like compound of the formula: 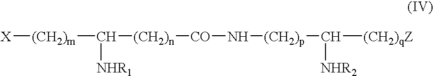
wherein R₁ and R₂ each are acylof a saturated or unsaturated, carboxylic acid of 2 to 24 carbon atoms, unsubstituted or substituted with at least one member selected from the group consisting of hydroxyl, alkyl, alkoxy, acyloxy, amino, acylamino, acylthio and alkylthio of up to 24 carbon atoms,
m, p and q are integers from 1 to 10,
n is an integer from 0 to 10,
X and Z each are an acid group or an accessory functional side chain spacer comprising
blocking amine functional groups in positions (q+1) and ;of a diamino acid of the formula H₂N(CH₂)ₚCHNH₂(CH₂)_{q-1}COOH with the blocking reagents which readily undergo acidolysis and hydrogenolysis, respectively, reacting the still free carboxylic functional group with a reducing agent to obtain the corresponding alcohol, freeing the amine functional group in position (q+1), acylating the same with a carboxylic acid functional derivative of formula R₂OH wherein R₂ is as defined above, freeing the terminal amine functional group by hydrogenolysis to obtain an amino alcohol of the formula: 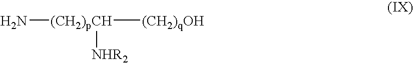
wherein R₂ is acyl of a saturated or unsaturated, carboxylic acid of 2 to 24 carbon atoms, unsubstituted or substituted with at least one substituent as specified above,
p and q are an integer from 1 to 10,
condensing the amino alcohol in the presence of a peptide condensing agent in an inert solvent, with an ;-carboxy amino acid of the formula: 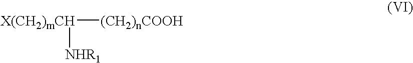
wherein R₁ is acyl of a saturated or unsaturated,. carboxylic acid of 2 to 24 carbon atoms, unsubstituted or substituted with at least one substitutent,
m is an integer from 1 to 10,
n is an integer from 0 to 10,
X is RO—CO—, wherein R is alkyl to yield dipeptide-like compounds of the formula: 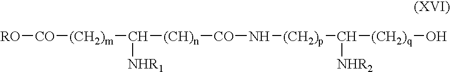
wherein R₁, R₂, m, n, p and q have the above meanings and R is a group which readily undergoes hydrogenolysis,
the free terminal alcohol functional group of which may be, if needed, alkyl- or acyl- or otherwise substituted by an alkyl- or acyl- or otherwise substitution reagent of the formula,
A-(CO)ᵣ—(CH₂)ₛ-W (VIII)
wherein A is selected from the group consisting of a leaving group, OH, SH and NH₂,
r is 1 or 0,
s is an integer from 1 to 10, preferably from 2 to 6,
W is selected from the group consisting of formyl, -acetyl, -cyano, -halo, -amino, -bromo- or iodo-acetamido, -acylamido, -diacylimido, -sulfhydril, -alkylthio, -hydroyl, 1,2-dihydroxyethyl, -acyloxy, -vinyl, -ethynyl, -free or esterified or carboxyl or in the form of a mixed anhydride, amide or hydrazide, azido and -thiocyano optionally in the presence of a coupling agent, and subjecting the same to a deprotection process, to obtain a compound of the formula 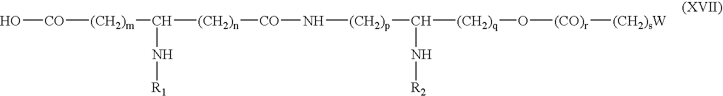
wherein substituents W, R₁, R₂, m, n, p, 2, r, and s are as defined above.

14. The process of claim 10, to obtain a compound of the formula: 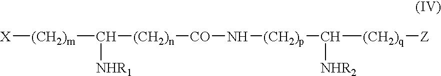
wherein R₁ and R₂ each are acyl of a saturated or unsaturated, carboxylic acid of 2 to 24 carbon atoms, unsubstituted or substituted with at least one member selected from the group consisting of hydroxyl, alkyl, alkoxy, acyloxy, amino, acylamino, acylthio and alkylthio of up to 24 carbon atoms,
m, p and q are integers from 1 to 10,
n is an integer from 0 to 10,
X and Z each are an acid group or an accessory functional side chain spacer comprising blocking the free hydroxyl functional group of a dipeptide-like compound of the formula: 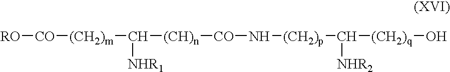
wherein R₁, R₂ m,n,p and q have the above meanings and R is a group which readily undergoes hydrogenolysis,
with a protective group which readily undergoes acidolysis, freeing the esterified carboxyl functional group, reacting the optionally activated carboxyl functional group with a reducing agent to obtain the corresponding primary alcohol, converting said alcohol functional group to a phosphoric ester by reaction with a phosphorylating agent; freeing the protected hydroxyl group by acid treatment subjecting the same to an acyl, alkyl or otherwise substitution reaction with a reagent of the. formula:
A-(CO)ᵣ—(CH₂)ₛ-W (VIII)
wherein A is- selected from the group consisting a leaving group, OH, SH and NH₂,
r is 1 or 0.
s is an integer from 1 to 10.
W is selected from the group consisting of formyl, -acetyl, -cyano,-halo, -amino, -bromo- or iodo-acetamido, -acylamido, -diacylmido, -sulfhydril, -alkylthio, -hydroxyl, 1,2-dihydroxyethyl-, -acyloxy, -vinyl, - ethynyl, and free or esterified carboxyl.
optionally, in the presence of an activating agent, and deprotecting the product by hydrogenolysis to obtain a compound of the formula: 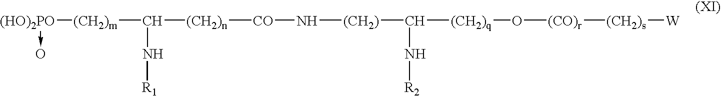
wherein substituents W, R₁, R₂, m,n,p,q,r,s have the above meanings.

15. The process of claim 10, to obtain a compound of the Formula: 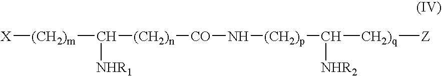
wherein R1 and R2 each are acyl of a saturated or unsaturated, carboxylic acid of 2 to 24 carbon atoms, unsubstituted or substituted with at least one member selected from the group consisting of hydroxyl, alkyl, alkoxy, acyloxy, amino, acylamino, acylthio and alkylthio 4 up to 24 carbon atoms.
m, p and q are integers from 1 to 10.
n is an integer from 0 to 10.
X and Z each are an acid group or an accessory functional side chain spacer, comprising in deprotecting the esterified carboxyl functional group of a dipeptide-like compound of the formula 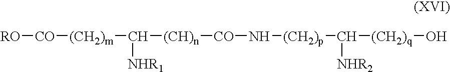
wherein R₁, R₂, m, n, p and q have the above meanings, and R is a group which readily undergoes hydrogenolysis, subjecting the resulting acid to a peptide coupling reaction with a partially protected amino acid or diaminoalkane in the presence of a peptide coupling agent, subjecting, if needed, the coupling reaction product to an acyl-, alkyl- or otherwise substitution reaction with a reagent of the formula:
A-(CO)ᵣ—(CH₂)ₛ-W (VIII)
where A is selected from the group consisting of a leaving group, OH, SH and NH₂,
r is 1 to 10,
s is an integer from 1 to 10,
W is selected from the group consisting of formyl, acetyl, -cyano, -halo, -amino, -bromo- or ido-acetamido, diacylimido, -sulfhydril, -alkylthio, -hydroxyl, 1,2-dihydroxyethyl-, -acyloxy, vinyl, -ethynyl and -free or esterified carboxyl.
Optionally, in the presence of an activating agent and deprotecting the resulting product, by hydrogenolysis, to obtain a compound of the formula: 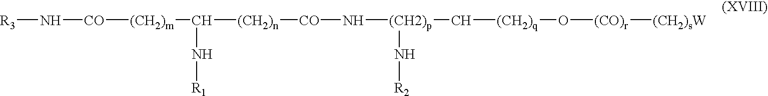
wherein W, R₁, R₂, m,n,p,q,r and s are specified above and R3 is selected from the group consisting of aminoalkyl, carboxyalkyl, discarboxylalkyl and aminocarboxyalkyl.

16. An enantiomer and diastereoisomer of a compound of claim 1, of general formula IV in accordance with claim 7, of general formula XI in accordance with claim 11, of general formula XII in accordance with claim 12, of general formula XVI in accordance with claim 13 or of general formula (XVIII) in accordance with claim 15.

17. The process of claim 10, wherein blocking of the amine Functional group in so positions of an ornithine or lysine derivative chain is effected with N-benzyloxycarbonylation, after initially reacting the acid functional group with a copper salt in an alkaline medium, reacting this copper carboylate with benzyl chloroformate and freeing the carboxylic functional group by chelating copper in an acidic medium by a-N-benzyloxycarbonyl compound.

18. The process of claim 10, wherein reduction of the free carboxylic group is effected with a borane-dimethylsulfur complex or by reacting the carboxylic with an alkyl chloroformate to form a mixed anhydride reducing the product with an alkali or alkaline earth metal botohydride to obtain the corresponding hydroxyl having a primary alcohol.

19. A process of claim 10 wherein removal of the benzyloxycarbonyl group is effected by hydrogenolysis in an alcohol solvent containing triethylamine.

20. The process of claim 10 wherein the freed amine is coupled with an α-phosphoryl carboxylic acid in the presence of a peptide coupling reagent to obtain a protected phosphoryl-dipeptide-like compound.

21. The process of claim 10 wherein the dipeptide-like compound is acyl substituted at the alcohol group with an ω-functionally substituted acid in the presence of an esterifying agent to yield an alcenyl ester.

22. The process of claim 21 wherien the alcenyl functional group of the alcenyl ester undergoes a dihydroxylation reaction at a vicinal diol group which is then subjected, after deprotetion of functional groups of the dipeptide-like compound, to a periodic oxidation reaction to obtain a compound having an aldehyde functional group.

23. The process of claim 10 wherein the dipeptide-like compound is acyl substituted at the alcohol group with an ω-aminoalkanoic acid.

24. The process of claim 10 wherein a full deprotection reaction through hydrogenolysis in the presence of a catalyst is conducted to obtain a dipeptide-like compound bearing an accessory aminoalkyl side chain spacer.

25. The process of claim 10 wherein the acid partner used in the coupling reaction is aspartic acid or glutamic acid obtained by acylating the amine group acid β-benzyl ester.

26. The process of claim 10, wherein the N-acyl aspartic or glutamic acid derivative is coupled to an α-N-acyl amino alcohol derived from ornithine or lysine followed by subjecting the resulting dipeptide-like compound to a hydrogenolysis reaction in the presence of a catalyst to provide the dipeptide-like bearing a carboxylic functional group.

27. The process of claim 10, wherein the hydroxyl group of the dipeptide-like compound is acylated with ω-alcenyl acid and the alcenyl group undergoes a dihydroxylation reaction followed by a deprotection and a perioidic oxidation reaction to provide a compound of formula XVII.

28. The process of claim 10, wherein the hydroxyl group of the dipeptide-like compound is acylated with an \acute{ω}-aminoalkanoic acid and the product is subjected to deprotection reactions, to provide a compound of formula XVII.

29. The process of claim 10, wherein the amino alcohol obtained by reduction of ornithine or lysine is alkyl substituted by an \acute{ω}-alcenyl triflate coupled with an α-acylamino \acute{ω}-phosphoryl carboxylic acid in the presence of an acylating agent followed by a dihydroxylation reaction of the alcenyl group in the presence of osmium tetraoxide, removal of the protective groups, and oxidation of the vicinal diol group by sodium periodate to obtain a compound having an aldehyde group.

30. The process of claim 10, wherein the N protected serine derivative is subjected to an O-alkyl substitution reaction, the protective group of the amine group is removed by acidolysis, the amine group is acylated with a 3-hydroxy fatty acid compound in the presence of an acylating agent to obtain an N-acyl O-alkyl serine which is coupled with an amino alcohol in the presence of a peptide coupling agent followed by acylation of the free OH group with an \acute{ω}-functionally alkanoic acid, to provide a compound of formula I wherein X is carboxyalkoxy.

31. The process of claim 10, wherein the starting amino acid is cystein or homocystein alkyl substituted by p-methoxybenzyl bromoacetate and then acylated through bonding to a nitrogen atom with a 3-hydroxy fatty acid, the S-alkyl N-acyl substituted derivative thus obtained is coupled with 5-amino-2-[3-hydroxytetradecanoylamino]pentan-1-ol in the presence of a peptide coupling agent to obtain a condensation product, the free primary alcohol group of which is acylated by an \acute{ω}-functionally alkanoic acid, and the resulting product is optionally subjected to a deprotection reaction to provide a compound of formula I wherein X is carboxyalkylthio.

32. The process of claim 10, wherein the serine p-methoxybenzyloxycarbonyl compound is subjected to an O-alkyl-substitution with dibenzyl methylene malonate in an alkaline medium, the amine group is freed by acid treatment and the freed amine group is acylated with a 3-hydroxy fatty acid in the presence of an acylating agent, followed by coupling the N-acyl-O-alkyl-compound with an amino alcohol in the presence of a peptide coupling agent to form a dipeptide-like compound which is acylated at the free OH group with an \acute{ω}-alcenyl- or amino-alkanoic acid, the alcenyl is subjected to a dihydroxylation, then a deprotection reaction through hydrogenolysis followed by a periodic oxidation reaction to obtain a compound of formula I wherein X is dicarboxyalkyloxy.

33. The process of claim 10, wherein O-phosphoryl homoserine benzyl ester is used as a starting compound, carrying out an N-acylation reaction with 3 benzyloxytetradecanoic acid, subjecting the resulting benzyl ester to a selective hydrogenolysis, coupling said acid to an α-N-dodecanoyloxy-tetradecanoic ornithine in the presence of a peptide coupling agent to obtain a dipeptide-like compound which is acylated at the still free hydroxyl group with an \acute{ω}-alcenyl or amino substituted alkanoic acid, in the presence of a carbodiimide and subjecting the reaction product, in case of an alcenyl derivative, to a dihydroxylation reaction and a deprotection reaction through hydrogenolysis in the presence of a catalyst followed by periodic oxidation, to obtain a compound of formula I wherein R1 is hydroxyalkanoyl and R2 is acyloxyalkanoyl.

34. The process of claim 10, wherein the intermediate dipeptide-like compound obtained from aspartic or glutamic acid is subjected to a blocking reaction at the free hydroxyl group with a group which readily undergoes acidolysis, the esterified carboxyl functional group is deprotected by another deprotection method, which group is then reduced, after activation into a mixed anhydride, with a reducing agent, the resulting hydroxyl group is subjected to a phosphorylation reaction and the hydroxyl protective group is then hydrolyzed in an acidic medium and the hydroxyl group thus regenerated is subjected to an acylation reaction with an \acute{ω}-functionally substituted carboxylic acid, followed by subjecting the same to a dihydroxylation reaction and a deprotection reaction through hydrogenolysis in the presence of a catalyst and a periodic oxidation reaction to. provide a compound of formula XI bearing an aldehyde group.

35. The process of claim 10, wherein the intermediate dipeptide-like compound from aspartic or glutamic acid is subjected to a deprotection reaction at the esterified carboxyl group, said carboxyl group is coupled to a partially protected aminoalkane or amino acid, in the presence of a coupling agent, the coupling reaction product is then subjected either to a deprotection reaction or an acylation reaction with an \acute{ω}-functionally substituted alkanoic acid and the product is subjected in succession, if an alcenyl derivative is used, to a dihydroxylation reaction and finally to a periodic oxidation reaction to obtain a compound of formula I or formula XVII.

36. A compound having a formula selected from the group consisting of 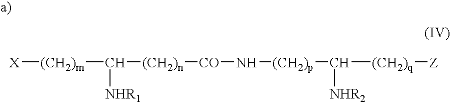
wherein R₁ and R₂ each are acyl of saturated or unsaturated carboxylic acid of 2 to 24 carbon atoms, unsubstituted or substituted with at. least one member selected from the group consisting of hydroxyl, alkyl, alkoxy, acyloxy, amino, acylamino, acylthio and alkylthio of up to 24 carbon atoms,
m, n and p are integers from 1 to 10,
n is an integer from 0 to 10,
X and Z each are an acid group or an accessory functional side chain spacer; 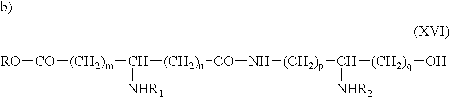
wherein substituents R₁, R₂, m, n, p and q have the above meanings and R is a group which readily undergoes hydrogenolysis and 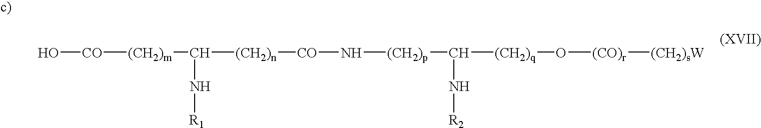
wherein A is oxygen, W, R₁, R₂, m, n, p, q, r and s are as specified above,
the said compounds being in the form of pure enantiomers or in the form of a mixture of stereoisomers.

37. A pharmaceutical composition as the active ingredient a compound of formula I of claim 1, either as a racemic mixture or an optically active form, in the form of pure diastereoisomers of a mixture thereof, in neutral or charged state, and an inert, non-toxic pharmaceutically acceptable carrier.

38. A compound of claim 1, grafted on an antigen to modulate the immune response.

39. A method of modulating an immune-response in warm-blooded animals comprising administering to warm-blooded animals in need thereof an amount of a conjugate of claim 4 sufficient to modulate an immune response.
